SEQUENCE LISTING (29 sequences)

SEQ ID NO: 1 - DNA, 7 bp, Homo sapiens
gagacag

SEQ ID NO: 2 - DNA, 10 bp, Homo sapiens
taatcccagc

SEQ ID NO: 3 - DNA, 9 bp, Homo sapiens
gagaaagag

SEQ ID NO: 4 - DNA, 23 bp, Artificial Sequence
  EV1 isoform forward primer
ggttccttgcagcatgcaagacc

SEQ ID NO: 5 - DNA, 21 bp, Artificial Sequence
  EV1 isoform reverse primer
gttctctgatcaggcagttgg

SEQ ID NO: 6 - DNA, 20 bp, Artificial Sequence
  EV1 isoform probe
tacttgaggccttctccagg

SEQ ID NO: 7 - DNA, 24 bp, Artificial Sequence
  beta actin forward primer
cagccatgtacgttgctatccagg

SEQ ID NO: 8 - DNA, 23 bp, Artificial Sequence
  beta actin reverse primer
aggtccagacgcaggatggcatg

SEQ ID NO: 9 - DNA, 20 bp, Artificial Sequence
  beta actin probe
actggcatcgtgatggactc

SEQ ID NO: 10 - DNA, 48 bp, Artificial Sequence
  probeGAGACAGF
caaaatctctgtttcagtttgagacagagtttccctcttttcgtcagg

SEQ ID NO: 11 - DNA, 48 bp, Artificial Sequence
  probeGAGACAGR
cctgacgaaaagagggaaactctgtctcaaactgaaacagagattttg

SEQ ID NO: 12 - DNA, 48 bp, Artificial Sequence
  probeGAGACAGmtF
caaaatctctgtttcagtttgaaacagagtttccctcttttcgtcagg

SEQ ID NO: 13 - DNA, 48 bp, Artificial Sequence
  probeGAGACAGmtR
cctgacgaaaagagggaaactctgtttcaaactgaaacagagattttg

SEQ ID NO: 14 - DNA, 48 bp, Artificial Sequence
  probeGAGACAGFmt1
caaaatctctgtttcagtttcccttttagtttccctcttttcgtcagg

SEQ ID NO: 15 - DNA, 48 bp, Artificial Sequence
  probeGAGACAGRmt1
cctgacgaaaagagggaaactaaaagggaaactgaaacagagattttg

SEQ ID NO: 16 - DNA, 48 bp, Artificial Sequence
  probeGAGACAGFmt2
caaaatctctgtttcagtttcccccagagtttccctcttttcgtcagg

SEQ ID NO: 17 - DNA, 48 bp, Artificial Sequence
  probeGAGACAGRmt2
cctgacgaaaagagggaaactctgggggaaactgaaacagagattttg

SEQ ID NO: 18 - DNA, 48 bp, Artificial Sequence
  probeGAGACAGFmt3
caaaatctctgtttcagtttgaaacgacccttccctcttttcgtcagg

SEQ ID NO: 19 - DNA, 48 bp, Artificial Sequence
  probeGAGACAGRmt3
cctgacgaaaagagggaagggtcgtttcaaactgaaacagagattttg

SEQ ID NO: 20 - DNA, 48 bp, Artificial Sequence
  probeGAGACAGFmt4
caaaatcgtccatacagtttcccttttagtttccctcttttcgtcagg

SEQ ID NO: 21 - DNA, 48 bp, Artificial Sequence
  probeGAGACAGRmt4
cctgacgaaaagagggaaactaaaagggaaactgtatggacgattttg

SEQ ID NO: 22 - DNA, 49 bp, Artificial Sequence
  TAATCCCAGCF
caaaagtggctcacgcctgtaatcccagcactttgggaggctgaggcag

SEQ ID NO: 23 - DNA, 49 bp, Artificial Sequence
  TAATCCCAGCR
ctgcctcagcctcccaaagtgctgggattacaggcgtgagccacttttg

SEQ ID NO: 24 - DNA, 42 bp, Artificial Sequence
  pureTAATsurrF
caaagtggctcacgcctgtaatcccagcactttgggaggtag

SEQ ID NO: 25 - DNA, 42 bp, Artificial Sequence
  pureTAATsurrR
ctacctcccaaagtgctgggattacaggcgtgagccactttg

SEQ ID NO: 26 - DNA, 49 bp, Artificial Sequence
  TAATCCCAGCmtF
caaaagtggctcacgcctgtaagtacagcactttgggaggctgaggcag

SEQ ID NO: 27 - DNA, 49 bp, Artificial Sequence
  TAATCCCAGCmtR
ctgcctcagcctcccaaagtgctgtacttacaggcgtgagccacttttg

SEQ ID NO: 28 - DNA, 49 bp, Artificial Sequence
  TAATCCCAGCmt2F
caaaagtggctcacgcctggctgtacctgactttgggaggctgaggcag

SEQ ID NO: 29 - DNA, 49 bp, Artificial Sequence
  TAATCCCAGCmt2R
ctgcctcagcctcccaaagtcaggtacagccaggcgtgagccacttttg

(19) United States
(12) Patent Application Publication
(10) Pub. No.: US 2017/0322217 A1
(43) Pub. Date: Nov. 9, 2017

(54) A METHOD FOR PROGNOSIS OF OVARIAN CANCER, PATIENT'S STRATIFICATION

(71) Applicant: AGENCY FOR SCIENCE, TECHNOLOGY AND RESEARCH, Singapore (SG)

(72) Inventors: Arsen Olegovich BATAGOV, Singapore (SG); Anna Vladimirovna IVSHINA, Singapore (SG); Surya Pavan YENAMANDRA, Singapore (SG); Vladimir Andreevich KUZNETSOV, Singapore (SG)

(21) Appl. No.: 15/503,386

(22) PCT Filed: Aug. 11, 2015
(86) PCT No.: PCT/SG2015/050253

§ 371 Date: Feb. 10, 2017

(30) Foreign Application Priority Data
Aug. 11, 2014 (SG) 10201404800X

(51) Int. Cl. G01N 33/574; G06F 19/24; C12N 15/115; C12Q 1/68

(52) U.S. Cl. CPC G01N 33/57449; C12Q 1/6886; G01N 33/5748; C12N 15/115; G06F 19/24; C12Q 2600/112; C12Q 2600/158

(57) ABSTRACT

There are no reliable clinical bio-markers of survival prognosis, patient's risk stratification and treatment prediction for epithelial ovarian cancers(EOC). The most common type of the human EOC is a high grade serous EOC. This cancer is characterized with one of the lowest survival rates compared to other cancers. The present invention relates to an method for a prognosis of survival of a subject diagnosed with EOC, the method comprising determining in a sample of the subject gene expression level of at least one gene in the list of Evi1 pathway genes; and/or copy number of at least one gene in the MECOM locus; wherein the level against at least one expression threshold value will define the risk group of the subject and/or a risk of the disease progression after surgery treatment, and/or an effectiveness of post-surgery chemotherapy. The quantification method of Evi1/MECOM locus regulatory pathway provides a set of multigene prognostic signatures representing EVI1 pathway modules, which collectively provided a framwork of high-confidence, sensitive and specific prognosis assay(s) of EOC and stratification method for the EOC patient stratification according to disease relapse.

### TECHNICAL FIELD

The present invention relates to method(s) for prognosis of cancer in a subject, stratification of the patients according to the disease prognosis in particular but not exclusively by analyzing Evi1/MECOM pathway genes.

### BACKGROUND

Ovarian cancer (OC) is a very common cancer that affects women worldwide. Out of all the cancers that affect women, epithelial ovarian cancer (EOC) ranks fifth when considering woman cancer mortality in the world and fourth for the age group 40 to 59 years old, with overall a 5-year survival rate of women inflicted by EOC of only 46%. This is despite improvements in surgical techniques and the advent of more targeted therapeutics such as bevacizimab. The low survival rate is explained by the lack of diagnosis of EOC at an early stage, acquired resistance to chemotherapy in a significant number of patients over the years and the lack of effective therapies for advanced refractory disease. The 5-year survival rate is up to 95% if EOC diagnosis is made in the early stage of the disease.

EOC comprises three major histological subtypes; serous, mucinous and endometrioid. Serous EOC includes serous cystomas, serous benign cystadenomas, serous cystadenomas with proliferating activity of the epithelial cells and nuclear abnormalities but with no infiltrative destructive growth (low potential or borderline malignancy), and serous cystadenocarcinomas. Mucinous EOC includes mucinous cystomas, mucinous benign cystadenomas, mucinous cystadenomas with proliferating activity of the epithelial cells and nuclear abnormalities but with no infiltrative destructive growth (low potential or borderline malignancy), and mucinous cystadenocarcinomas. Endometrioid EOC includes endometrioid tumors (similar to adenocarcinomas in the endometrium), endometrioid benign cysts, endometrioid tumors with proliferating activity of the epithelial cells and nuclear abnormalities but with no infiltrative destructive growth (low malignant potential or borderline malignancy), and endometrioid adenocarcinomas. Two further, less-prevalent histological subtypes also exist, clear cell and undifferentiated.

EOC may also be categorized by “stages”, depending upon how far they have spread beyond the ovary. Thus, Stage I is defined as EOC that is confined to one or both ovaries. Stage II is defined as EOC that has spread to pelvic organs (e.g., uterus, fallopian tubes), but has not spread to abdominal organs. Stage III is defined as EOC that has spread to abdominal organs or the lymphatic system (e.g., pelvic or abdominal lymph nodes, on the liver, on the bowel). Finally, Stage IV is defined as EOC that has spread to distant sites (e.g., lung, inside the liver, brain, lymph nodes in the neck).

EOCs may be graded according to the appearance of the cancer cells. Low-grade (or Grade 1) means that the cancer cells look very like the normal cells of the ovary; they usually grow slowly and are less likely to spread. Moderate-grade (or Grade 2) means that the cells look more abnormal than low-grade cells. High-grade (or Grade 3) means that the cells look very abnormal. They are likely to grow more quickly and are more likely to spread. A high grade serous OC (EOC) is most common OC disease.

EOC, like most other cancers, is thus a complex heterogeneous disease, influenced and controlled by multiple genetic and epigenetic alterations leading to an increasingly aggressive phenotype. It is now well recognised that the characteristics of an individual tumor and its life course results from multiple somatic mutations acquired over time (e.g. TP53, PTEN, RAS) and continual evolution of the host responses to environmental factors. From a therapeutic standpoint EOC is best considered a collection of complex inter-related diseases represented by an immense natural heterogeneity in tumor phenotypes, disease outcomes, and response to treatment.

A major challenge is thus to identify and thoroughly validate diagnostic and prognostic biomarkers that can accurately describe the heterogeneity ascribed to EOC. In addition, accurate predictive biomarkers are required to guide current treatment protocols, as well as to guide the development and application of new targeted therapies. CA-125 (MUC16, Cancer antigen 125) protein is currently considered the best diagnostic marker of EOC. However, the true positive rate of MUC16 test is only about 50% of stage I EOC patients, while it returns more than 80% of true positives for patients at stages I-IV. About 25% of EOCs especially at the early stages, do not produce reliably-detectable CA-125 and therefore its application in clinical settings is limited. It has been reported that CA-125 in combination with human epididymis protein 4 (HE4) is elevated in greater than 90% of EOC. Such poor prognostic statistics indicate that there is an urgent need to improve understanding of the molecular mechanisms underlying EOC, so as to develop better prognostic and predictive assays and identify new therapeutic targets.

Currently there are about 15 oncogenes considered particularly important for EOCs and 11 of them, including EVI1 have been shown to be amplified in the genome of cancer cells. Proto-oncogene EVI1 (ectopic viral integration site 1), encoded by the MECOM locus (Entrez GeneID: 2122) located at the 3q26 chromosome region, is amplified in many cancers. EVI1 protein was identified as an evolutionary conserved transcription factor sharing 94% amino acid sequence homology between human and mice. In the adult human tissues it is highly expressed in kidney, lung, pancreas, brain and ovaries. In mouse embryos it is highly expressed in the urinary system, lungs and heart and its activity is vital for the embryonic development. The majority of research of EVI1 describes its significance in pathology. If over-expressed in blood cells, EVI1 has been shown to produce a number of alternatively spliced transcripts and causes various hematopoietic disorders, including myeloid leukemias. EVI1 was found to be overexpressed in the blood of up to 21% patients with acute myeloid leukemia (AML). In 4% of AML cases chromosome region 3q is aberrated. High expression of EVI1, regardless the amplification of MECOM locus alone was recently found to be a significant survival factor for EOC patients. Chromosome region 3q25-27 is amplified in cancers in a various organs: ovary, cervix, lung, oesophagus, colon, head and neck and prostate. Amplification of MECOM is also associated with resistance to chemotherapy in EOC.

### SUMMARY

Evi1 protein encoded in MECOM locus controls expression of a specific regulatory pathway active in EOC tumors. The genes of the pathway are effectors of Evi1 transcriptional regulation and mediate of Evi1 effects in OC tumors. The pathway comprises all six hallmarks of cancer [1] and thus promotes OC progression. Evi1 activity drives OC progressesion via enhanced variations in the genotypes of the tumor cells, which are shedded and disseminated in the peritoneal cavity of the patient. Thus, the disseminated tumor clones carrying different genotypes slowly grow in the peritoneal cavity of the patient in parallel [2]. The clones containing amplifications of MECOM locus, as well as the genes of Evi1 pathway are likely to disseminate more efficiently and to survive the debulking procedure at the time of surgery, as well as post-operational chemotherapy. The inherent variability of their genotypes makes the tumor and the patient classification difficult. At present, it leads to patient stratification into either a) large subgroups with poor diagnostic and prognostic value; or b) small subgroups (less than 10% of the whole patient cohort) with poor reproducibility across patient cohorts. Since MECOM is amplified (mean copy number >2.5) in the majority of the patients, the copy number of this locus is a source of a feasible stratification of the patients, where the size of the resulting subgroups is large. Since MECOM amplification reflects the activation of Evi1 pathway in a given tumor, it increases the prognostic significance of Evi1 pathway signatures, while being applicable to a large fraction of the patient cohort. On the other hand, if total size of the patient cohort is relatively small (less than 100 patients), it might be feasible to maximize the number of the patients involved in the clinical program and therefore MECOM copy number might be ignored. Additionally, the technical measures to quantify gene copy number may be unavailable in some clinics.

The present invention is based on studies in relation to the Evi1 associated genes/proteins, hereinafter termed Evi1 pathway signature gene(s) or protein(s) and how this may be used in terms of, for example, providing a prognosis to a pateint, or facilitating in the determination of a suitable treatment for a patient.

In a first aspect there is provided a method for obtaining information in relation to a medical condition of a subject, the method comprising:

determining in a sample of the subject an expression level of at least one Evi1 pathway signature gene or protein;

wherein the expression level of the, or each, gene/protein, when compared to a respective expression threshold or reference value, is indicative of said information, and wherein the information is selected from the group consisting of:

whether the subject has EOC and/or a predisposition to EOC; survival prognosis of the subject when the subject has EOC; and

effectiveness of treatment of EOC in the subject.

Typically the obtained information may be used by a clinician so a to facilitate pateint management. For example, the information may be used to direct a treatment, such as whether or not surgery should be conducted, or the type of drug which should be administered. Aggressive treatments may be warranted where the prognosis is poor, but the clinician may only consider such aggressive treatment when he/she is aware of the poor prognosis. Thus, it is important to know when the prognosis is poor.

The present inventors have observed the expression of certain Evi1 signature pathway gene(s)/protein(s) can be associated with the effectiveness of particular EOC treatments.

Thus, in a further aspect there is provided a method of determining an effectiveness of treatment of EOC in a subject, the method comprising:

determining in a sample of the subject an expression level of at least one Evi1 pathway signature gene/protein;

wherein the expression level of the, or each, gene/protein, when compared to a respective expression threshold value, is indicative of the effectiveness of the treatment.

By determining the expression level of the at least one Evi1 pathway signature gene/protein, the clinician may be provided with information which will facilitate in his/her determination of the most suitable treatment for the particular patient.

In a further aspect there is provided a method for determining survival prognosis of a subject with EOC, the method comprising:

determining in a sample of the subject a copy number (typically DNA copy number) of at least one Evi1 pathway signature gene locus;

wherein the (DNA) copy number of the, or each, locus, when compared to a respective copy number threshold value, is indicative of survival prognosis of the subject with EOC.

In a further aspect there is provided a method of determining effectiveness of treatment of EOC in a subject, the method comprising:

determining in a sample of the subject the copy number (typically DNA copy number) of at least one Evi1 pathway signature gene locus;

wherein the (DNA) copy number of the, or each, locus, when compared to a respective copy number threshold value, is indicative of the effectiveness of the treatment.

In accordance with any one of the preceding statements, the method may further comprise determining the copy number of at least one MECOM locus gene in the subject, and wherein the copy number, when compared to a respective MECOM locus copy number threshold/reference value, is further indicative of survival prognosis or effectiveness of treatment in the subject. For example, wherein determining survival prognosis and/or determining effectiveness of treatment further the method may comprise the following steps for each said Evi1 pathway signature gene: comparing the copy number of the at least one MECOM locus gene in the subject against the MECOM locus copy number threshold value;

if the copy number of the at least one MECOM locus gene in the subject exceeds the MECOM locus copy number threshold, further comparing the gene/protein expression level of the Evi1 pathway signature gene in the subject against a gene/protein expression threshold value determined for a first cohort of reference subjects, the first cohort of reference subjects having copy numbers of the at least one MECOM locus gene which are above the MECOM locus copy number threshold value;

otherwise, comparing the gene/protein expression level of the Evi1 pathway signature gene in the subject against a gene/protein expression threshold value determined for a second cohort of reference subjects, the second cohort of reference subjects having copy numbers of the at least one MECOM locus gene which are equal to or below the copy number MECOM locus copy number threshold value; and

determining the survival prognosis and/or determining effectiveness of treatment based on the comparison between the respective expression levels of the Evi1 pathway signature gene(s)/protein(s) in the subject and the respective gene expression threshold values.

In accordance with the invention, the Evi1 pathway signature gene(s)/protein(s) may be selected from genes/proteins having at least one Evi1 binding site.

Alternatively, the Evi1 pathway signature genes/proteins may be selected from the genes/proteins listed in any of Tables 1-11, singly or in any combination. Typically the expression level of 2 or more, such as 3, 4, 5, 6, 7, 8 or more genes/proteins may be determined in order to provide a signature.

The inventors analysed a number of markers for their ability to stratify patients by prognosis. Table 1 identifies these markers. It is to be understood that the individual markers identified in Table 1 are not ranked in order for their ability to stratify patients by prognosis independent of other variables. Rather, the skilled person will understand that the various tables simply provide a list of markers, any of which may be suitable for stratifying patients. The skilled person may, therefore, select any marker or markers from said Table for the purposes of stratification. Further, the skilled person may select at least one marker from the Table together with a marker not provided in the Table. For example, the skilled reader may select one or more marker from the tables together with determining Evi1. The inventors propose that their use of a marker or markers identified in the Tables provides increased accuracy of prognosis.

It will be appreciated that “prognosis” is distinct from “diagnosis”. “Prognosis” refers to a prediction about how a disease will develop, for example, the lifespan of the subject. In contrast, “diagnosis” refers to the identification of a disease. The prognosis of subjects may be categorised into low, medium or high risk, which equates to a good, medium or poor prognosis. Consequently, “prognosis” may be understood to mean a predicted survival time.

Survival prognosis and/or effectiveness of treatment is determined by using a voting rule, a consensus rule, or a majority rule.

The EOC may be a high grade adenocarcinoma in the ovary, in ascites or in metastases, or a primary EOC or serous tubal intraepithelial carcinoma.

In accordance with the invention the gene expression level I may be determined by measuring, for example, the mRNA or protein expression of the or each Evi1 pathway signature gene in the sample.

In accordance with the invention the method may further comprise determining the gene expression level of at least one MECOM locus gene in the sample.

The methods described herein may further comprise a training stage prior to determining survival prognosis and/or determining effectiveness of treatment, the training stage comprising the following steps for each gene of the one or more Evi1 pathway signature genes:

for each of a plurality of training subjects with known outcome relating to EOC, determining a gene/protein expression level of the gene/protein in the training subject; and

determining an expression threshold value which divides the training subjects into two or more patient groups according to whether the gene/protein expression level of the gene/protein in each training subject exceeds the expression threshold value, and/or a copy number threshold value which divides the training subjects into two or more patient groups according to whether the copy number of the gene/protein in each training subject exceeds the copy number threshold value;

wherein the determined expression threshold value or copy number threshold value maximizes a measure of difference between the training subjects into the said two groups.

The methods as described herein may further comprise a training stage prior to determining survival prognosis and/or determining effectiveness of treatment, the training stage comprising the following steps for one or more Evi1 pathway signature genes/proteins:
(i) for each of a plurality of training subjects with known outcome relating to EOC, determining copy number of at least one MECOM locus gene in the training subject;
(ii) estimating a sample copy number of at least one MECOM locus gene and dividing the training subjects into two cohorts according to whether the copy number of a MECOM locus gene in each training subject is above or below the sample copy number;
(iii) for each said cohort, determining a sample expression value which divides the training subjects in the cohort into two or more groups according to whether the gene/protein expression level of the Evi1 pathway signature in each training subject exceeds the sample expression value, wherein the sample expression value achieves a maximum measure of difference between the two or more groups;
(iv) repeating steps (ii)-(iii) by varying the sample copy number in a range of copy numbers identified in the training subjects and obtaining a copy number distribution curve for the genes; and
(v) selecting a copy number threshold value as the copy number associated with the largest maximum measure of difference between the two or more groups of a cohort and selecting expression threshold values as the expression values determined for the groups obtained with the copy number threshold value.

The methods described may include a measure of difference between the two or more groups comprising a measure of difference between survival curves of the two or more groups.

The methods for determining survival prognosis of the subject, may further comprise the steps of:
(i) parametrization of a dependence between a patient cohort fraction and the copy number of EVI1 in the set of training subjects;
(ii) parametrization of a dependence between the patient cohort fraction and the survival time in the set of training subjects; and
(iii) using the copy number of the subject to determine the patient cohort fraction from the dependence of (i) and using the patient cohort fraction of the subject to determine an estimated survival time of the subject from dependence (ii).

The survival time of the training subject may be based on the last follow-up time for the training subject.

The methods described herein may comprise:
(i) for each of a plurality of Evi1 pathway signature genes, obtaining an indication score of survival prognosis or treatment effectiveness; and
(ii) determining a consensus score from the indication scores of step (i), using an independent voting method.

In accordance with the invention the M expression signature threshold values and/or M copy number signature threshold values, may be consensus threshold values derived from each of N training groups of EOC patient tumor samples; the M consensus threshold values classifying the samples of each training group into two or more survival risk sub-groups according to the methods for determining survival prognosis mentioned or treatment effectiveness mentioned; the consensus threshold values may be generated by:
i) generating, for each of the N training groups, a set of M threshold values for a set of M Evi1 pathway signature genes, the M threshold values dividing the samples of each training group into two or more survival risk sub-groups; the N*M evaluated threshold values representing M consensus thresholds defined in an N-dimensional space by the following approximation procedure;
ii) generating a best-fit approximating function of the M threshold values in the N-dimentional space;
iii) generating M evaluated threshold values derived by orthogonal projection of the M threshold values in the N-dimentional space onto the best-fit approximating function; the yielded M points on the approximating function represent the consensus threshold value.

The method may further comprise the steps of:
(i) measuring the M expression signature values or M copy number signature values in the subject sample;
(ii) determining the coefficient (one for all the measured signature values) that scales the measurement in the subject sample with the measurements of the same M signatures in each of the N training groups, yielding N scaling coefficients and N*M scaled signature values;
(iii) for each of the M scaled signature values, each represented as a point in the N-dimentional space of signature measurements, determining the orthogonal projection of the signature value onto the best-fit approximating function, yielding M subject points on the best-fit approximating function;
(iv) for each of the M subject points on the best-fit approximating function, determining the difference along the approximating function from the given point to the consensus threshold value of the same signature, yeielding the M-dimensional prognostic score of the subject;
(v) based on a given voting rule, determine the classification of the subject into of the given survival prognosis or treatment effectiveness groups;

The best-fit approximating function may be a linear function.

Each consensus threshold may be obtained as an arithmetic or geometric mean of the threshold values obtained for each of said groups.

Patients may be stratified into classes by their diagnostic features and/or stratified into classes by their treatment outcomes.

The threshold values may be copy number threshold values or consensus threshold values in Table 12 and/or Table 13.

The level of marker expression may be normalised. In some embodiments, marker expression may be normalised against the expression of another endogenous, regulated reference marker obtained from the sample. In an alternative embodiment, marker expression may be normalised against total cellular DNA from the sample. In some embodiments, marker expression may be normalised against total cellular RNA from the sample. In some embodiments, marker expression may be normalised against the length of the marker nucleotide transcript. For the purposes of this invention the term “transcript” relates to RNA, in particular mRNA, and DNA, in particular cDNA. The skilled addressee will be aware that the total number of reads for a given transcript is proportional to the expression level of the transcript multiplied by the length of the transcript. For example, a long transcript will have more reads mapping to it compared to a short gene of similar expression. Various normalisation methods are known in the art, and it is to be appreciated that the above normalisation methods are in no way limiting to the skilled reader. Thus, alternative normalisation techniques not described within this invention may also be used.

It will be appreciated that said markers may be provided for use in a kit, or be a feature of a kit. For example, said markers may be provided bound to a substrate in a kit. The substrate may comprise probes capable of specifically binding said markers. Alternatively, the substrate may comprise primers capable of specifically binding said markers, or antibodies capable of specifically binding said markers. The substrate may comprise any combination of probes, antibodies and/or primers.

Conveniently, the markers may be any marker identified in the tables. It is to be understood that the markers detected in the present invention relate to genes. Consequently, the markers may comprise DNA, RNA or the protein/polypeptide product of the gene. Variants of the gene will also be known in the art, and will also be included in the term marker. The term thus includes mutant nucleotide DNA, RNA or polypeptide sequences, allelic, splice and post translationally modified forms which are known in the art, or may be discovered in the future. In particular, the term marker includes mRNA and cDNA. Optionally, the markers to be assayed may comprise protein/polypeptide. Preferably, the markers may comprise RNA. Alternatively, the markers may comprise DNA. In one embodiment the markers may comprise cDNA. The cDNA may be synthesized from mRNA. In one embodiment the markers may comprise DNA and RNA. Although the remainder of this disclosure will be directed to RNA markers and resulting cDNA, this should not be construed as limiting in any way, as other expression products, as listed above, may alternatively be detected.

In accordance with the present invention, the sample is any appropriate tissue sample obtained from the subject. In one embodiment the sample is tissue such as ovarian tissue sample obtained from the subject. In another aspect the sample is any appropriate fluid sample obtained from the subject. Tissue samples may be obtained by biopsy during surgery. By “biopsy” we include excisional and incisional biopsies. The term “biopsy” further includes partial or gross resection. Samples may alternatively be obtained by other methods known in the art.

In one embodiment of the invention is provided a method of facilitating treatment for a subject, said method comprising detecting a level of expression of at least one marker identified in the Tables identified herein in a sample from the subject and providing a prognosis based upon the expression level of said marker or markers and selecting and/or administering a treatment based on the prognosis. The expression level values obtained may be used by the clinician in assessing any of the following (a) probable or likely suitability of a subject to initially receive treatment; (b) probable or likely unsuitability of an individual to initially receive treatment; (c) dosage of treatment; (d) start date to begin treatment; (e) duration of treatment course; (f) type of treatment to be administered. Example treatments may include, but are not limited to radiotherapy, chemotherapy, anti-angiogenic compounds and/or surgery.

In accordance with the present invention, assay systems are provided. The assay systems may comprise a measurement device that measures marker expression levels. The system may further comprise a data transformation device that acquires marker expression level data and performs data transformation to calculate whether or not the level determined is increased, decreased or equal to a threshold or reference value for the marker in question from the sample.

In some embodiments, the assay system may also comprise an output interface device such as a user interface output device to output data to a user. Preferably, the assay system also includes a database of threshold or reference values, wherein the device identifies a good, medium or poor prognosis upon analysis of the collective expression of the markers. In one embodiment the device provides treatment information in the database for the good, medium or poor prognosis and outputs the treatment information to the user interface output device. In one embodiment the user interface output device may provide an output to the user, comprising notification such that the subject's gene expression is increased or decreased to the threshold/reference value, that this relates to a good, medium or poor prognosis and if they should administer a suitable therapy, such as radiotherapy, chemotherapy, anti-angiogenic compounds or surgery. In an alternative embodiment, the user interface output device may provide an output to the user, providing information on a good, medium or a poor prognosis and, if treatment is suitable, a time deadline by which treatment should begin.

In one embodiment, the output interface device is remote from the user of the input device. For example, a subject's sample directed by a clinician/health worker may be analysed in a local clinic or laboratory, but the results are transmitted remotely to the clinician or health care worker remote from the interface output device. Thus, results can immediately be transmitted, ensuring the timely release of information to ensure the relevant treatment is started as soon as possible, particularly when information is provided about a poor prognosis. By ensuring the most suitable treatment starts at the most relevant time, such an assay may provide subjects given a poor prognosis with better treatment options and in doing so a potentially longer life span and/or quality of life.

In a further aspect there is provided a kit for use in the present methods. The kit can comprise at least one antibody, probe and/or primer which is capable of specifically binding at least one of the markers identified in the Tables. Probes may be detectably labelled for example with a fluorescent or luminescent label. The kit may further comprise instructions for use, such as with an assay system. Kits for use in the detection of RNA or DNA markers may comprise at least two probes or primers per marker to be detected. Kits for use in the present methods may comprise reagents for the synthesis of cDNA.

Assessment of DNA, RNA or polypeptide/protein expression levels is routine in the art. One example of a method of measuring protein levels, is Western blotting or immunohistochemistry using antibodies to particular markers. Other protein assays may include radioimmunoassay (RIA), enzyme-linked immunosorbent assay (ELISA) or flow cytometry. Suitable RNA detection methods may include nucleic acid hybridisation (Northern blotting) or nucleic acid amplification. In some embodiments, the nucleic acid hybridization is performed using a solid-phase nucleic acid molecule array. In other preferred embodiments, the nucleic acid amplification method is reverse transcriptase PCR (RT-PCR). Two common methods for the detection of products in RT-PCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence to quantify messenger RNA (mRNA). Further details with regards to RT-PCR will be known to those skilled in the art and can be found in common laboratory manuals (e.g. Sambrook and Russell, Molecular Cloning: A laboratory Manual, CSHL Press, 2001). In some embodiments, DNA detection methods may include nucleic acid hybridization (Southern blotting) or nucleic acid amplification. Preferably, the nucleic acid amplification method is PCR. In some embodiments the nucleic acid detection method comprises a DNA microarray. In one embodiment the nucleic acid detection method is next-generation sequencing (NGS). It will be appreciated that nucleic acid transcripts detected by next-generation sequencing may be normalized by length of transcript. Further details with regards to DNA detection techniques will be known to skilled addressees and can be found in common laboratory manuals, for example Sambrook and Russell, Molecular Cloning: A laboratory Manual, CSHL Press, 2001. Various methods of next-generation sequencing are known to the skilled addressee, who will look to NGS system providers' websites for reference (including, but not limited to: 5http://res.illumina.com/documents/products/illumina sequencing introduction.pdf; https://www.qiagen.com/gb/products/next-gen-sequencing).

In one embodiment there is provided a diagnostic chip for use in the present methods. The chip may comprise at least one probe and/or primer which is capable of specifically binding at least one of the markers identified in the Tables. In some embodiments there is provided a diagnostic chip for use in the present methods, wherein said chip comprises a plurality of probes and/or primers which are collectively capable of specifically binding all of the markers identified in each, a selection or all of the tables. Thus, there is provided a diagnostic chip for use in the present methods, said method comprising detecting a level of expression of all markers identified in each, a selection or all tables identified therein in a sample from the subject and providing a prognosis based upon the expression level of said markers. Preferably, there is provided a diagnostic chip for use in the present methods, wherein said chip comprises a plurality of probes and/or primers which are collectively capable of specifically binding all of the following markers identified in Tables 1 -12. In some embodiments, the diagnostic chip comprises a plurality of probes and/or primers which are collectively capable of specifically binding at least two markers identified in Tables 1 - 12. The diagnostic chip may comprise a traditional, solid phase array. Alternatively, the diagnostic chip may comprise an alternative bead array. Diagnostic chips may also be referred to as DNA microarrays. Various diagnostic chips, are known to those skilled in the art, and may include, but not be limited to Affymetrix chips, Agilent products, and/or Illumine products. In accordance with the invention, the probes and/or primers for the diagnostic chip may be bound to a surface. In one embodiment, the preferred surface is silica or glass. In one embodiment, the preferred surface is plastic. In some embodiments, the probes and/or primers for the diagnostic chip may be bound to polystyrene beads.

Preferably, oligonucleotides may be used as probes or primers. Oligonucleotides for use within a kit may be labelled in order to be detected. Fluorescent labels may be used to enable direct detection. Alternatively, labels may be detected indirectly. Indirect detection methods are known in the art and may comprise, but not be limited to, biotin-avidin interactions and antibody binding. Fluorescently labelled oligonucleotides may also contain a quenching molecule.

There is also provided a kit for determining survival prognosis of a subject with EOC, the kit comprising at least one probe capable of specifically hybridising with an Evi1 pathway signature gene and/or a MECOM locus expression product in a sample of the subject, and instructions for carrying out a method according described herein.

The kit may further comprise at least one probe that can identify copy number of at least one MECOM locus gene in the sample. The copy number may be determined using at least one method selected from the group consisting of: quantitative PCR assay, in situ hybridization, Southern blotting, multiplex ligation-dependent probe amplification (MLPA) and Quantitative Multiplex PCR of Short Fluorescent Fragments (QMPSF).

The at least one probe may comprise at least one aptamer that binds to at least one MECOM locus-encoded protein and/or one or more protein corresponding to the Evi1 pathway signature genes identified herein.

Alternatively the at least one probe comprises at least one antibody or protein that binds to at least one MECOM locus-encoded protein and/or one or more proteins corresponding to Evi1 pathway signature genes.

The kits may include instructions for using the consensus threshold values as the threshold values for determining survival prognosis.

The markers currently being used for detection of EOC lack adequate sensitivity and specificity to be applicable in large populations particularly during the early stages of EOC. The present invention attempts to fill this gap in the clinical biomarker field by the method detectingthe modules of the Evi1 pathway as a source of more sensitive and specific prognostic markers than widely accepted biomarkers of HD-EOC patient' biomarkers markers. In particular, the present invention relates to identification of clinically distinct sub-groups of EOC patients differentially characterized in one of the following aspects:
stratification of patient's according survival prognosis, and
prediction of personalized teraputic targets
improvement of effectiveness of treatment.

We claim that an identification of relatively homogeneous patient groups (patient's startification), a personalization of theraputic targets in context of patient survival and treatment could be achieved in the integrative analysis of DNA copy number variations and/or gene expression level of at least one gene from the MDS1 and/or EVI1 complex locus (also known as MECOM locus) in the tumor samples and expression level of at least one gene belonging to EVI1 patwhay (Evi1 pathway gene, abbreviated here as EPG; more than one EPG are abbreviated as EPGs). Threshold values for DNA copy number values of MECOM locus and expression levels of EVI1 and MDS1 genes belonging to this locus and EPGs maximally separating the patient groups by the chosen criteria are obtained separately for MECOM locus genes (EVI1 and MDS1) and EPGs

The identification of patient groups is achieved in the integrative analysis of DNA copy number variations and/or gene expression level of at least one gene from the MDS1 and/or EVI1 complex locus (also known as MECOM locus) in the tumor samples. Threshold values for DNA copy number values of MECOM locus and expression levels of EVI1 and MDS1 genes belonging to this locus maximally separating the patient groups by the chosen criteria are obtained separately for MECOM locus genes: EVI1 and MDS1.

In particular, the diagnostic procedure for individual patients may comprise the steps of:

1. obtaining and QC control tumor sample material and completeness of medecal record, epidemiological, clinical data and treatment information;

2. measurement of DNA copy number value of at least one MECOM locus gene;

3. measurement of gene expression level (using a non-limiting example of mRNA) of an EPG or EPGs;

4. comparison of the values obtained in the measurements of the above steps against the DNA copy number and/or expression threshold values respectively.

5. interative clinical bioinformatics and statistical prediction of the diseases, stages etc.

According to one aspect, the present invention relates to an in vitro method for determining survival prognosis of a subject with EOC, determining appropriate (critical pathways), and prediction of effectiveness of teraputic intervention against EOC, the method comprising determining in a sample of the subject gene expression level (using a non-limiting example of mRNA) of at least one EPG ; and/or

1. DNA copy number value of at least one gene in MECOM locus

2. gene expression level of, at least one of EPGs

wherein the level against at least one expression threshold value and/or DNA copy number against at least one DNA copy number threshold value are indicative of the subject having EOC, or predisposition to EOC, or the survival prognosis of the subject or the effectiveness of the treatment on the subject.

According to other aspects, the present invention provides kits, computer programs, and computer systems using the method according to any aspect of the present invention.

It will be appreciated by persons skilled in the art that the binding moieties of the invention may be used for the diagnosis or prognosis of EOC of any histological subtype (for example, serous, mucinous, endometrioid, clear cell, undifferentiated or unclassifiable).

The term “sample” is herein defined to include but is not limited to be blood, sputum, saliva, mucosal scraping, tissue biopsy and the like. The sample may be an isolated cell sample which may refer to a single cell, multiple cells, more than one type of cell, cells from tissues, cells from organs and/or cells from tumors.

A person skilled in the art will appreciate that the present invention may be practiced without undue experimentation according to the method given herein. The methods, techniques and chemicals are as described in the references given or from protocols in standard biotechnology and molecular biology text books.

According to one aspect, there is provided at least one method for prognosing survival of a subject diagnosed with or predisposed to EOC, and/or determining the effectiveness of treatment of EOC, and/or determining if an EOC in a subject is of primary origin or secondary origin, the method comprising determining in a sample of the subject: (i) gene expression level of at least one gene in the MECOM locus; and/or (ii) copy number of at least one gene in the MECOM locus; and/or (iii) gene expression level of at least one EPG. wherein the gene expression level and/or copy number against at least one expression and copy number threshold value respectively is indicative of the subject having EOC or predisposition to EOC and/or survival prognosis of a subject with EOC, and/or determining the effectiveness of treatment of EOC.

The method according to any aspect of the present invention may be in vitro, or in vivo. In particular, the method may be in vitro, where the steps are carried out on a sample isolated from the subject. The sample may be taken from a subject by any method known in the art. For example but not limiting, ovarian tumor material may be extracted from ovaries, fallopian tubes, uterus, vagina and the like. Metastatic tumor may be extracted from peritoneal cavity, other body organs, tissues and the like. Cancer cells may be extracted from non limiting examples such as biological fluids, which include but are not limited to peritoneal liquid, blood, lymph, urine, products of body secretion and the like.

Quantifying of expression ecotropic virus integration site 1 protein homolog (EVI1), Myelodysplasia syndrome-I (MDSI), EPGs and other gene transcripts used according to any aspect of the present invention may be done using any technique of gene expression quantification. Such techniques include, but are not limited to quantitative PCR, semi-quantitative PCR, gene expression microarray, next generation RNA sequencing and the like.

The copy number of MECOM, EVI1, MDSI, EPGs and/or other genes used according to any aspect of the present invention may be determined using any technique of gene copy number quantification. Such techniques include, but are not limited to quantitative PCR, semi-quantitative PCR, SNP microarrays, next-generation sequencing, cytogenetic techniques (such as in-situ hybridization, comparative genomic hybridization, comparative genomic hybridization), Southern blotting, multiplex ligation-dependent probe amplification (MLPA) and Quantitative Multiplex PCR of Short Fluorescent Fragments (QMPSF) and the like.

For convenience, certain terms employed in the specification, examples and appended claims are collected here.

The term “aptamer” is herein defined to be oligonucleic acid or peptide molecule that binds to a specific target molecule. In particular, an aptamer used in the present invention may be generated using different technologies known in the art which include but is not limited to systematic evolution of ligands by exponential enrichment (SELEX) and the like. The term “comprising” is herein defined to be that where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the method according to any aspect of the present invention “substantially” comprises the indicated step as “essential” element. Additional steps may be included.

The term “difference” between two groups of patients is herein defined to be the statistical significance (p-value) of a partitioning of the patients within the two groups. Thus, achieving a “maximum difference” means finding a partition of maximal statistical significance (i.e. minimal p-value).

The term “label” or “label containing moiety” refers in a moiety capable of detection, such as a radioactive isotope or group containing same and nonisotopic labels, such as enzymes, biotin, avidin, streptavidin, digoxygenin, luminescent agents, dyes, haptens, and the like. Luminescent agents, depending upon the source of exciting energy, can be classified as radio luminescent, chemiluminescent, bio luminescent, and photo luminescent (including fluorescent and phosphorescent). A probe described herein can be bound, for example, chemically bound to label-containing moieties or can be suitable to be so bound. The probe can be directly or indirectly labelled. The term “locus” is herein defined to be a specific location of a gene or DNA sequence on a chromosome. A variant of the DNA sequence at a given locus is called an allele. The ordered list of loci known for a particular genome is called a genetic map. Gene mapping is the process of determining the locus for a particular biological trait. For example, the MECOM locus comprises at least two genes MDS1 and EVI1, which expression results in transcription of, at least two corresponding transcripts, i.e. mRNA variants. The two transcripts may be the longer transcript of MDS1 and the shorter transcript of EVI1.

The term “MECOM locus” is herein defined according to the definition provided in the RefSeq

NCBI database as “MDS1 and EVI1 complex locus (MECOM)” or “MDS1 and EVI1 complex locus” (Unigene Hs.659873) and may be essentially characterized by its genomic coordinates hg18.chr3:170,283,981-170,864,257 and by its non-limiting longest transcripts NM_004991.3 and NM_001205194.1.

The term “MECOM locus gene” defines a gene which genomic coordinates overlap with the genomic coordinates of MECOM locus by at least one nucleotide.

The term “copy number (CN) value ” or “DNA copy number value” is herein defined to refer to the number of copies of at least one DNA segment (locus) in the genome. The genome comprises DNA segments that may range from a small segment, the size of a single base pair to a large chromosome segment covering more than one gene. This number may be used to measure DNA structural variations, such as insertions, deletions and inversions occurring in a given genomic segment in a cell or a group of cells. In particular, the CN value may be determined in a cell or a group of cells by several methods known in the art including but not limited to comparative genomic hybridisation (CGH) microarray, qPCR, electrophoretic separation and the like. CN value may be used as a measure of the copy number of a given DNA segment in a genome. In a single cell, the CN value may be defined by discrete values (0, 1, 2, 3 etc.). In a group of cells it may be a continuous variable, for example, a measure of DNA fragment CN ranging around 2 plus/minus increment d (theoretically or empirically defined variations). This number may be larger than 2+d or smaller than 2-d in the cells with a gain or loss of the nucleotides in a given locus, respectively.

With respect to associations between disease and CN value, a level of variation (deviation) in a DNA segment CN might be important. A level of positive or negative increment of the CN from normal dynamical range in a DNA sample of a given cell group or a single cell may be called CN variation.

The term “diagnosing” and “diagnosis” is herein defined to include the act or process of identifying the existence and/or type of cancer from which an individual may be suffering. Thus, in one embodiment, diagnosis includes the differentiation of a particular cancer type, namely EOC, from one or more other cancers. In an alternative embodiment, binding moieties of the invention are for use in classifying EOC patients into clinically relevant groups based on overall survival and/or cancer-specific survival.

The term “prognosis” is herein defined to include the act or process of predicting the probable course and outcome of a cancer, e.g. determining survival probability or overall survival (OC) probability.

The term “binding moiety” is herein defined to be a molecule or entity that is capable of binding to target genomic DNA, a target protein or mRNA encoding the same. For example, a binding moiety can be a probe such as a single stranded oligonucleotide at the time of hybridization to a target protein. Probes include but are not limited to primers, i.e., oligonucleotides that can be used to prime a reaction, for example at least in a PCR reaction. In particular, the probe may be capable of binding to ENV1 or MDS1 protein or mRNA encoding the same and be used to quantify the gene expression level of ENV1 or MDS1. In another embodiment, the probe may capable of binding to the genomic DNA of MECOM locus (i.e. ENV1 or MDS1 genomic DNA) and may be capable of determining the copy number of ENV1 or MDS1.

Evi1 is a transcription factor is any protein isoforms encoded in MECOM locus.

The term ‘Evi1 functions’ is defines any biological and/or pathological function that are affected by alterations (including, but not limited to expression, copy number chemical modification, concentration) of any gene in the MECOM locus and/or any product of such gene.

The term “signature” defines a set of the molecular features, whose value could be directly or indirectly detected and used for the method of detection of molecular characterization of certain normal and/or pathological cells, tissues, organisms functions, processes and biomedical conditions.

The term “gene expression signature”(GES) is defined as a set of gene products (RNAs, proteins), whose count or/and activity value are used as a signature. For a medical condition, the gene expression signature can be defined as a single gene product (s) or a combination of distinct gene products, which after detection, processing data and its interpretation could be used in further analysis in the terms of biomarker discovery and method development for diagnosis, prognosis of disease and prediction, monitoring of therapeutic response.

The term “copy number signature” defines a set of loci that may or may not encode one or more genes, whose DNA copy counts are used as a components of the signature.

The term “signature gene” defines a gene, which feature (s) or product(s) belong to a given signature.

The term “signature gene expression” or “signature expression” defines a list of expression values of all genes belonging the given gene expression signature and the rules (e.g. specify thresholds) and charateristics differentiating medical condition(s)

The term “signature copy number” defines the copy number values of a of a subset of loci belonging to a copy number signature.

The term “signature thresholds” defined a list of the values, each of which is a threshold (or cut-off) value of one and only one feature of a given gene in the signature. These features can be gene expression or copy number.

The term ‘patient survival’, or ‘survival’ is defined as overall patient survival.

The term ‘nucleic acid sequence motif (motif) is a probabilistic model of occurrence of A,G,C and T nucleic acid in a specific sequence representing DNA sequence which could be specifically bound transcription factor/protein. In the text format, the motif could be represented by the most frequent nucleotides in a given DNA sequence.

The term ‘nucleic acid sequence motif’ or “protein-binding nucleic acid sequence motif” is defined as the motif of the nucleotides representing a specific family of nucleic acid sequences, each of which could be bound by a given protein.

The term ‘Evi1 motif’ or ‘DNA motif of Evi1’ is defined as one of nucleic acid sequence motifs representing by DNA sequences which could specifically bound by Evi1 protein. They are 2 major Evi1 motifs (which we identified for the first time and used in this work). In particular the motifs are: M1(GAGACAG) and M2(TAATCCCAGC) . In these sequences only the most probable nucleotides are presented.

The term “co-localization of a protein binding site with a motif at a specific distance” or “motif co-localization” is defined as an event there can be found such minimal genomic span than includes more than 50% of both the protein binding sites and the motif locations.

The term “co-localization of a given motif with a given gene at a specific distance” is defined as an event, when the motif represents a sequence within the gene boundaries extended from both ends by the given distance. As an example, the distance was chosen to equal 20 kb.

The term “co-localization of a given protein binding site with a given gene at a specific distance” is defined as an event, when the protein binding site is localized within the gene boundaries extended from both ends by the given distance. As an example, the distance was chosen to equal 20 kb.

The term “co-localization of a given protein binding site with a given gene” is defined as an event, when the protein binding site is localized withing the gene boundaries extended by 20 kb from both ends.

The term ‘Evi1 pathway genes’ defines the human genes simultaneously satisfying the following criteria: 1) co-localized with at least one Evi1-binding DNA motifs; 2) co-localized with experimentally identified Evi1 binding sites; 3) responding to variation of EVill gene expression with variation of their products (the products including but not limited to RNA or protein levels). Examples of Evi1 pathway genes are given in Table 1 and the MECOM locus genes, as well as the other tables identified herein. For the avoidance of doubt, the term Evi1 pathway gene does not include the Evi1 gene itself .

“Evi1 pathway signature” is a signature defined on the list of Evi1 pathway genes (those listed in Table 1, MDS1 and EVI1 genes) and their loci (including MECOM locus), whose features are detected as the features of the corresponding gene expression and/or copy number signatures. Evi1 pathway signature is defined on single genes or on gene combinations for which specific rules of medical condition(s) are identified/specified and estimated parametrically.

The term ‘Evi1 pathway module’ defines a subset of Evi1 pathway genes and their products (e.g. RNAs and proteins), whose activity or molecular features could be involed in a known biological or/and pathological pathway or process. The Evi1 pathway module represents a functional-related gene group of the EVI1 pathway. The term “Evi1 pathway module signature” is a signature of a given Evi1 pathway module with a defined biological and/or pathological function and/or molecular process. Examples of Evi1 pathway module signatures are given in Tables 2-11.

The term “training set” or “training group” or “training group of samples” defines a set of samples used for detection of any measurable features, which are subsequently used to computationally derive generalized features, that can be shared with other samples and sample sets. The samples can be represented by, but are not limited to, tumors, tumor pieces, or patients. The measurable features include, but are not limited to clinical characteristics, gene expression or gene copy number values. The generalized features include, but are not limited to expression threshold values, signatures, functions, or their parameters. The clinical characterististics can be represented by, but are not limited to patient age, survival status, tumor type, disease aggressiveness, succeptibilty to certain chemotherapy.

The term “testing set” or “testing group” or “testing group of samples” defines a set of samples for which clinical predictions are made based on observations of their measurable features, combined with their comparison with generalized features obtained from a training set. The samples can be represented by, but are not limited to, tumors, tumor pieces, or patients. The clinical predictions can be represented by, but are not limited to predictions of survival status, tumor type, disease aggressiveness, or succeptibilty to certain chemotherapy. The measurable features include, but are not limited to clinical characteristics, gene expression or gene copy number values. The generalized features include, but are not limited to expression threshold values, signatures, functions, or their parameters.

The term ‘survival time’ is defined here as the duration of patient overall survival in a given patient group since the moment of diagnosis.

Comparisons of expression values distributions across the groups defined by the clinical qualitative variables (such as tumor type—‘primary’ or ‘metastasis’) were performed with Kruskal-Wallis test. Correlations between quantitative variables (such as expression values) were assessed with Kendall correlation coeffictient. To assess the differences between the survival functions of the patient groups the Wald metric of the Cox-proportional hazard model was used in the implementation of the standard library survival of R statistical environment.

The term ‘survival per cent’ defines here overall survival fraction, estimated with Kaplan-Meyer model, by the given survival time in a given group of patients.

Comparisons of expression values distributions across the groups defined by the clinical qualitative variables (such as tumor type - ‘primary’ or ‘metastasis’) were performed with Kruskal-Wallis test [12]. Correlations between quantitative variables (such as expression values) were assessed with Kendall correlation coefficient [13]. To assess the differences between the survival functions of the patient groups the Wald metric of the Cox-proportional hazard model was [14]. Kaplan-Meier model [15] was used to assess the survival curves and calculate the survival predictions. In the cases where expression value was tested for survival significance, the patients were separated by the best threshold of the expression value found as a result of a P-value minimization with an exhaustive search for the best patient stratification, as described in [6].

For all the Evi1 pathway signatures the patients were stratified into three risk groups (low-medium- and high-risk). Each of the three risk groups could not be reduced to two, since the each possible pair of the groups demonstrates a significant difference. The null-hypothesis that any two of the three proposed strata belong to the same surivival group was rejected at significance level α=0.05 for every possible combination of the paired group for each signature.

The term “binary classification score” defines a binary vector of dimention M, each component of which contains a value assigning a subject into one of two classification groups. In the case of two risk groups, values 1 and 2 can assign the subject to the low and high risk groups, respectively.

The term “voting” defines a sample classification procedure with input and output. For input it accepts i) a set of readings of parameters (e.g. gene expression values) of a given sample, ii) a sample classification system (e.g. disease relapse, treatment outcome, or favorable/unfavorable survival prognosis), and iii) a voting rule. As an output, the procedure returns a qualitative value classifying the sample. The procedure transforms the parameter reading to qualitative variables, according to the given sample classification system and applies the voting rule to them.

The term “voting rule” defines a procedure with input and output. As an input, the procedure accepts i) a sample classification system , ii) a set of sample classifications according to this system. As an output, the procedure integrates the set of sample classifications and returns a single classification of the sample in the given classification system.

Examples of voting rules include, but are not limited to consensus rule and majority rule.

The term “consensus rule” defines such voting rule that returns the such sample classification that matches each member of the input sample classification set.

The term “majority rule” defines such voting rule that returns the such sample classification that matches, at least, half of the members of the input sample classification set.

Examples of voting procedures inlude, but are not limited to gene voting and gene expression signature voting.

The term “prognostic score” defines the M-dimensional vector of qualitative values, returned by a function with K+1 arguments, including K binary vectors of dimention M, and one voting rule. The function applies the voting rule to each of the M components of the K binary vectors and returns the results of their voting with the given voting rule. For example, in the case of the prognostic score function arguments represented by two (K=2) M-dimentional binary classifications C¹ and C² of a subject into one of two prognostic risk groups (low-risk and high-risk) and a consensus voting rule (classifying the subject as low-risk' for all i, where C¹ᵢ=C²ᵢ=‘low-risk’; ‘high-risk’ for all i, where C¹ᵢ=C²ᵢ=‘high-risk’; or medium-risk , where C¹ᵢ≠C²ᵢ), the prognostic score function returns the M-dimesional prognostic score of the subject classified into one of the three groups (‘low-risk’, ‘high-risk’ and ‘medium-risk’), according to each of its M components.

The term “gene expression voting” defines such voting, where the gene expression values obtained from the sample for each member of a gene set are treated as the input parameter readings.

The term “signature voting” defines such voting, where for a given sample a set of prognostic scores, each defined by an individual signature, are treated as the input set of parameter readings.

The term “gene expression signature voting” defines such voting, where for a given sample a set of prognostic scores, each defined by an individual gene expression signature, are treated as the input set of parameter readings.The term “Evi1 binding region” or “Evi1 transcription factor binding region” defines a region on the DNA enclosing a given genomic locus with surrounding regions 20 thousand base pairs long.

The term “distance” defines a measure of simularity of the vectors in N-dimensional vector space, and can be euclidean distance, Manhattan distance or any other distance measure based a structure and matematical nature of the cornering vectors. Here, we used this term to estimate a measure of a similarity between the N-dimentisional vectors of the threshold values of expressed genes found in two sets of expression micro array datasets, representative by distinct clinical groups.

The term “approximating function” defines a function f(x,p) with N-dimentional argument x with such parameters p that minimize the summarized distance between a selected set of points X to f(X,p), where the sumarization can be sum, product, or other corresponding operator. The argument x can be a measurement of a biological feature, such as gene expression or copy number values. Then N can be the number of sample groups, where the given feature is detected. The N sample groups can be N distinct training groups of patient samples.

The term “least square approximation of points X” or “finding the best-fit approximating function” defines finding such approximating function f(x,p) with parameters p of particular value that minimize the sum of squared difference between each point X and its correspoding value f(X,p).

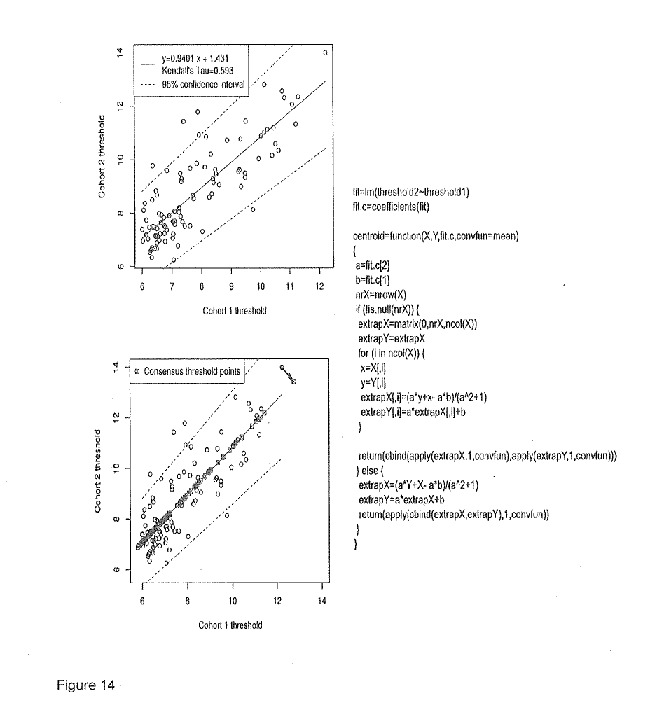

The term “scaling coefficient between approximating function f(x,p) and axis A” on FIG. 14 defines such coefficient K, for which K*f(x,p)=A. This coefficient can be estimated from a small set of points Y on axis A, where the values of f(Y,p) are defined. In particular cases K can be taken as an arithmetic mean value of Y/f(Y,p). In more general cases K can be a function defined on Y.

The present inventors have identified new motifs to which the Evi1 protein is expected to bind and which are identified as being close to the nucleic acid sequences of the Evi1 pathway genes identified herein. It is expected that disrupting the binding of Evi1 to these sequences may find therapeutic application in treating EOC.

Thus, in a further aspect there is provided a method of treating EOC, the method comprising administering one or more binding agents which are capable of specifically binding to a nucleic acid sequence comprising the sequence GAGACAG or TAATCCAGC and disrupting the binding of Evi1 to said sequence(s).

There is also provided one or more binding agents which are capable of specifically binding to a nucleic acid sequence comprising the sequence GAGACAG or TAATCCAGC and disrupting the binding of Evi1 to said sequence(s) for use in a method of treating EOC, or in the manufacture of a medicament for treating EOC.

Typically the binding agent is an aptamer or an antibody or antibody fragment. The skilled addressee is well versed in being able to develop such aptamers or antibody/antibody fragments.

The terms “antibody” and “antibody fragments” as used herein refer to monoclonal antibodies, bispecific antibodies, multispecific antibodies, human antibodies, humanized antibodies, chimeric antibodies, camelised antibodies, single domain antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. Particular antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgGI, IgG2, IgG3, IgG4, IgAI and IgA2) or subclass.

There is also provided a pharmaceutical formulation comprising an aptamer, antibody or antibody fragment together with a pharmaceutically acceptable excipient, wherein the aptamer, antibody or antibody fragment is capable of specifically binding to a nucleic acid sequence comprising the sequence GAGACAG or TAATCCAGC and disrupting the binding of Evi1 to said sequence(s)

The term “excipient” as used herein refers to inert substances which are commonly used as a diluent, vehicle, preservatives, binders, or stabilizing agent for drags and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, PA), which is hereby incorporated in its entirety.

### Signature Tables

Each entry (row) in Tables 1-11 includes the Affymetrix Human Gene 1.0 ST microarray probeset index, the official gene name and the symbol, the most closely matching Refseq ID, and the most closely matching Affymetrix U133-Plus-2.0 probeset index (where not available, NA symbol is placed).

TABLE 1
The genes, transcripts and respective probesets used to define a signature for
the Evi1 pathway

38093644-Homo sapiens myelin protein zero-like 3 (MPZL3): NM_198275, NA
355594752-Homo sapiens clusterin (CLU): NM_001831, NA
22907059-Homo sapiens solute carrier family 47: NM_018242, 219525_at
206725431-Homo sapiens zinc finger and SCAN domain containing 31 (ZSCAN31):
NM_030899, NA
157419139-Homo sapiens laminin: NM_018891, NA
15619002-Homo sapiens chorionic gonadotropin: NM_033378, NA
356874799-Homo sapiens chorionic gonadotropin: NM_000737, NA
15619000-Homo sapiens chorionic gonadotropin: NM_033377, NA
146229337-Homo sapiens chorionic gonadotropin: NM_033183, NA
312922375-Homo sapiens serum/glucocorticoid regulated kinase 2 (SGK2):
NM_170693, 220357_s_at
184172391-Homo sapiens complement factor H (CFH): NM_001014975, NA
91199549-Homo sapiens CD68 molecule (CD68): NM_001040059, NA
342349317-Homo sapiens interleukin 18 (interferon-gamma-inducing factor) (IL18):
NM_001562, 206295_at
12738833-Homo sapiens FRAME family member 2 (PRAMEF2): NM_023014, NA
157738636-Homo sapiens small nucleolar RNA: NR_003943, NA
74315928-Homo sapiens small nucleolar RNA: NR_002562, NA
74315931-Homo sapiens small nucleolar RNA: NR_002564, NA
212720629-Homo sapiens transmembrane protein 120B (TMEM120B): NM_001080825,
NA
164663799-Homo sapiens antagonist of mitotic exit network 1 homolog (S. cerevisiae)
(AMN1): NR_004854, NA
77735358-Homo sapiens small nucleolar RNA: NR_002574, NA
83816916-Homo sapiens deleted in lymphocytic leukemia 2 (non-protein coding)
(DLEU2): NR_002612, NA
121582464-Homo sapiens metallothionein 1M (MT1M): NM_176870, 217546_at
239835752-Homo sapiens metallothionein 1D: NR_003658, NA
142388655-Homo sapiens metallothionein 1B (MT1B): NM_005947, NA
363000014-Homo sapiens brain expressed: NM_001136106, NA
164663779-Homo sapiens zinc finger protein 385B (ZNF385B): NM_001113398, NA
364023807-Homo sapiens prostate transmembrane protein: NM_020182, NA
325974473-Homo sapiens transmembrane 4 L six family member 19 (TM4SF19):
NM_138461, NA
393290851-Homo sapiens actin: NM_001017992, NA
162287218-Homo sapiens prune homolog 2 (Drosophila) (PRUNE2): NM_015225,
212805_at
313851094-Homo sapiens claudin 12 (CLDN12): NM_012129, NA
255683381-Homo sapiens MDS1 and EVI1 complex locus (MECOM): NM_005241, NA
226423948-Homo sapiens neutral cholesterol ester hydrolase 1 (NCEH1): NM_020792,
NA
194018465-Homo sapiens ets variant 5 (ETV5): NM_004454, NA
62241034-Homo sapiens lipase: NM_139248, NA
217035114-Homo sapiens ADP-ribosylation factor-like 14 (ARL14): NM_025047, NA
296785063-Homo sapiens claudin 1 (CLDN1): NM_021101, 218182_s_at
197333816-Homo sapiens Mab-21 domain containing 2 (MB21D2): NM_178496, NA
325652058-Homo sapiens hairy and enhancer of split 1: NM_005524, 203393_at
296040512-Homo sapiens transmembrane 4 L six family member 18 (TM4SF18):
NM_138786, NA
46411167-Homo sapiens muscleblind-like splicing regulator 1 (MBNL1): NM_207294,
NA
62422580-Homo sapiens transmembrane 4 L six family member 1 (TM4SF1):
NM_014220, 209386_at
325974483-Homo sapiens transmembrane 4 L six family member 4 (TM4SF4):
NM_004617, NA
344925865-Homo sapiens carbohydrate (N-acetylglucosamine-6-O) sulfotransferase 2
(CHST2): NM_004267, NA
153792109-Homo sapiens HEG homolog 1 (zebrafish) (HEG1): NM_020733, NA
145580598-Homo sapiens solute carrier family 12 (potassium/chloride transporters):
NM_024628, NA
197313729-Homo sapiens pleckstrin homology-like domain: NM_001134438, NA
344313164-Homo sapiens zinc finger: NM_024508, NA
187761321-Homo sapiens 5-hydroxytryptamine (serotonin) receptor 1D: NM_000864,
207368_at
115529441-Homo sapiens RAB: NM_173825, NA
153792304-Homo sapiens leucine rich repeat containing 58 (LRRC58): NM_001099678,
NA
186928843-Homo sapiens Rho GTPase activating protein 31 (ARHGAP31):
NM_020754, NA
222080101-Homo sapiens PERP: NM_022121, NA
40068517-Homo sapiens phosphogluconate dehydrogenase (PGD): NM_002631, NA
116014343-Homo sapiens absent in melanoma 1 (AIM1): NM_001624, 212543_at
189095282-Homo sapiens G protein-coupled receptor 126 (GPR126): NM_198569, NA
224809581-Homo sapiens RAP1 GTPase activating protein (RAP1GAP): NM_002885,
NA
57242789-Homo sapiens phosphodiesterase 7B (PDE7B): NM_018945, NA
44890066-Homo sapiens jun proto-oncogene (JUN): NM_002228, 201464_x_at
56790938-Homo sapiens regulator of G-protein signaling 17 (RGS17): NM_012419, NA
257195179-Homo sapiens FXYD domain containing ion transport regulator 5 (FXYD5):
NM_144779, NA
314122155-Homo sapiens nuclear receptor coactivator 7 (NCOA7): NM_181782, NA
163644253-Homo sapiens serum/glucocorticoid regulated kinase 1 (SGK1):
NM_005627, NA
340545554-Homo sapiens FYN binding protein (FYB): NM_199335, NA
299890852-Homo sapiens nuclear factor of kappa light polypeptide gene enhancer in
B-cells inhibitor: NM_001005474, NA
47519615-Homo sapiens tropomyosin 2 (beta) (TPM2): NM_213674, 204083_s_at
208431814-Homo sapiens protein tyrosine phosphatase: NM_001135648, 203038_at
56699489-Homo sapiens osteopetrosis associated transmembrane protein 1 (OSTM1):
NM_014028, NA
197927427-Homo sapiens A kinase (PRKA) anchor protein 12 (AKAP12): NM_005100,
NA
333440431-Homo sapiens cadherin 6: NM_004932, NA
166706910-Homo sapiens interferon-induced protein 44 (IFI44): NM_006417, NA
166706908-Homo sapiens interferon-induced protein 44-like (IFI44L): NM_006820, NA
380503850-Homo sapiens laminin: NM_001105209, 202202_s_at
330864766-Homo sapiens DnaJ (Hsp40) homolog: NM_001135004, NA
296278255-Homo sapiens RAB3B: NM_002867, NA
196162714-Homo sapiens zinc finger: NM_024786, NA
61743937-Homo sapiens G protein-coupled receptor 110 (GPR110): NM_025048, NA
51873051-Homo sapiens importin 5 (IPO5): NM_002271, 211952_at
15834622-Homo sapiens secretory leukocyte peptidase inhibitor (SLPI): NM_003064,
203021_at
61676177-Homo sapiens prostaglandin I2 (prostacyclin) synthase (PTGIS): NM_000961,
208131_s_at
25777676-Homo sapiens Ras association (RaIGDS/AF-6) domain family member 2
(RASSF2): NM_170774, NA
296010910-Homo sapiens coagulation factor III (thromboplastin: NM_001993, NA
170014742-Homo sapiens protease: NM_002771, NA
110349741-Homo sapiens chromosome 2 open reading frame 88 (C2orf88):
NM_032321, NA
122939163-Homo sapiens gap junction protein: NM_000165, NA
274317624-Homo sapiens tetraspanin 1 (TSPAN1): NM_005727, 209114_at
41327719-Homo sapiens CAP: NM_006366, 212551_at
55774979-Homo sapiens myeloid/lymphoid or mixed-lineage leukemia (trithorax
homolog: NM_006818, NA
222418609-Homo sapiens lysosomal protein transmembrane 5 (LAPTM5): NM_006762,
NA
157502202-Homo sapiens ATP-binding cassette: NM_001105515, NA
231568449-Homo sapiens claudin 10 (CLDN10): NM_182848, 205328_at
221316604-Homo sapiens BH3-like motif containing: NM_001001786, NA
41152089-Homo sapiens PDZK1 interacting protein 1 (PDZK1IP1): NM_005764, NA
62912480-Homo sapiens high mobility group AT-hook 2 (HMGA2): NM_003483,
208025_s_at
208973218-Homo sapiens endothelin 2 (EDN2): NM_001956, NA
221139796-Homo sapiens RNA binding motif protein 24 (RBM24): NM_153020, NA
115529447-Homo sapiens solute carrier family 8 (sodium/calcium exchanger):
NM_021097, NA
359806986-Homo sapiens aldo-keto reductase family 1: NM_003739, 209160_at
39777596-Homo sapiens transglutaminase 2 (C polypeptide: NM_004613, 201042_at
324120948-Homo sapiens mucin 1: NM_002456, NA
164414436-Homo sapiens netrin G1 (NTNG1): NM_001113228, 206713_at
300796806-Homo sapiens phosphodiesterase 1C: NM_005020, NA
215272329-Homo sapiens carboxypeptidase M (CPM): NM_001005502, NA
116812578-Homo sapiens transmembrane BAX inhibitor motif containing 4 (TMBIM4):
NM_016056, NA
385719211-Homo sapiens solute carrier family 4: NM_001039960, NA
63054843-Homo sapiens vitamin D (1: NM_000376, NA
168480078-Homo sapiens annexin A4 (ANXA4): NM_001153, NA
285395212-Homo sapiens tubulin: NM_024803, NA
358030328-Homo sapiens GULP: NM_016315, NA
83281448-Homo sapiens phytanoyl-CoA 2-hydroxylase (PHYH): NM_006214, NA
213972645-Homo sapiens cellular repressor of E1A-stimulated genes 2 (CREG2):
NM_153836, NA
148536860-Homo sapiens solute carrier family 5 (sodium/myo-inositol cotransporter):
NM_006933, NA
209862795-Homo sapiens solute carrier family 48 (heme transporter): NM_017842,
218416_s_at
55770901-Homo sapiens opioid growth factor receptor-like 1 (OGFRL1): NM_024576,
NA
301336132-Homo sapiens snail homolog 1 (Drosophila) (SNAI1): NM_005985,
219480_at
281182827-Homo sapiens intraflagellar transport 46 homolog (Chlamydomonas) (IFT46):
NM_020153, NA
301129274-Homo sapiens major histocompatibility complex: NM_002118, NA
109255244-Homo sapiens serine/threonine kinase 17a (STK17A): NM_004760, NA
288557346-Homo sapiens family with sequence similarity 49: NM_030797, NA
194473743-Homo sapiens four and a half LIM domains 2 (FHL2): NM_001039492, NA
191251776-Homo sapiens breast carcinoma amplified sequence 1 (BCAS1):
NM_003657, NA
19743807-Homo sapiens hydroxysteroid (17-beta) dehydrogenase 6 homolog (mouse)
(HSD17B6): NM_003725, NA
156616283-Homo sapiens pregnancy specific beta-1-glycoprotein 9 (PSG9):
NM_002784, NA
209862910-Homo sapiens periostin: NM_001135934, NA
181336848-Homo sapiens G0/G1switch 2 (G0S2): NM_015714, NA
41327740-Homo sapiens ethylmalonic encephalopathy 1 (ETHE1): NM_014297, NA
62243067-Homo sapiens insulin-like growth factor binding protein 3 (IGFBP3):
NM_000598, 210095_s_at
145275188-Homo sapiens chromosome 1 open reading frame 116 (C1orf116):
NM_023938, NA
253735771-Homo sapiens Rho/Rac guanine nucleotide exchange factor (GEF) 2
(ARHGEF2): NM_004723, 207629_s_at
198386337-Homo sapiens lysophosphatidylglycerol acyltransferase 1 (LPGAT1):
NM_014873, NA
54792080-Homo sapiens phosphoglucomutase 2-like 1 (PGM2L1): NM_173582, NA
48375182-Homo sapiens chloride intracellular channel 1 (CLIC1): NM_001288,
208659_at
6552331-Homo sapiens flotillin 1 (FLOT1): NM_005803, 208748_s_at
157909851-Homo sapiens TAF3 RNA polymerase II: NM_031923, NA
55925657-Homo sapiens syndecan 1 (SDC1): NM_002997, NA
196259787-Homo sapiens Rap guanine nucleotide exchange factor (GEF) 3
(RAPGEF3): NM_001098531, 210051_at
281427130-Homo sapiens toll-like receptor 5 (TLR5): NM_003268, NA
153792494-Homo sapiens growth differentiation factor 15 (GDF15): NM_004864, NA
167860125-Homo sapiens serpin peptidase inhibitor: NM_002639, NA
25777651-Homo sapiens potassium intermediate/small conductance calcium-activated
channel: NM_002250, 204401_at
21071023-Homo sapiens histone cluster 1: NM_003539, NA
161377466-Homo sapiens cyclin A1 (CCNA1): NM_003914, NA
21359934-Homo sapiens Down syndrome cell adhesion molecule like 1 (DSCAML1):
NM_020693, NA
301336135-Homo sapiens transmembrane and tetratricopeptide repeat containing 1
(TMTC1): NM_175861, NA
109826574-Homo sapiens tripartite motif containing 29 (TRIM29): NM_012101, NA
353523858-Homo sapiens kynurenine 3-monooxygenase (kynurenine 3-hydroxylase)
(KMO): NM_003679, NA
47078291-Homo sapiens integrin: NM_000212, NA
223941942-Homo sapiens carboxypeptidase A4 (CPA4): NM_016352, NA
331028575-Homo sapiens chimerin 1 (CHN1): NM_001025201, NA
8923525-Homo sapiens HRAS-like suppressor 2 (HRASLS2): NM_017878, 216759_at
149588790-Homo sapiens retinoic acid receptor responder (tazarotene induced) 3
(RARRES3): NM_004585, 204070_at
148596991-Homo sapiens alpha-kinase 2 (ALPK2): NM_052947, NA
148664185-Homo sapiens plakophilin 2 (PKP2): NM_001005242, 207717_s_at
112421012-Homo sapiens signal-induced proliferation-associated 1 like 2 (SIPA1L2):
NM_020808, NA
223468626-Homo sapiens component of oligomeric golgi complex 6 (COG6):
NM_020751, NA
221218982-Homo sapiens integrator complex subunit 2 (INTS2): NM_020748, NA
222352156-Homo sapiens succinate dehydrogenase complex: NM_003002, NA
41393588-Homo sapiens CXADR-like membrane protein (CLMP): NM_024769, NA
4503056-Homo sapiens crystallin: NM_001885, NA
116256359-Homo sapiens histone cluster 1: NM_005325, NA
148792969-Homo sapiens TAF4b RNA polymerase II: NM_005640, NA
211904132-Homo sapiens DAZ associated protein 2 (DAZAP2): NM_014764, NA
342307084-Homo sapiens baculoviral IAP repeat containing 3 (BIRC3): NM_001165,
210538_s_at
29826337-Homo sapiens interferon: NM_006332, NA
71143103-Homo sapiens potassium channel tetramerisation domain containing 21
(KCTD21): NM_001029859, NA
371502123-Homo sapiens capping protein (actin filament): NM_001747, NA
214832379-Homo sapiens aspartate beta-hydroxylase (ASPH): NM_004318,
205808_at
194097372-Homo sapiens calcyphosine (CAPS): NM_004058, NA
73808272-Homo sapiens matrix metallopeptidase 3 (stromelysin 1: NM_002422, NA
62868229-Homo sapiens mindbomb E3 ubiquitin protein ligase 1 (MIB1): NM_020774,
NA
257470982-Homo sapiens homeodomain interacting protein kinase 2 (HIPK2):
NM_022740, NA
150417972-Homo sapiens supervillin (SVIL): NM_021738, NA
255653000-Homo sapiens phosphorylase: NM_002863, NA
189027089-Homo sapiens cytoplasmic polyadenylation element binding protein 4
(CPEB4): NM_030627, NA
93204870-Homo sapiens netrin 4 (NTN4): NM_021229, NA
4506762-Homo sapiens S100 calcium binding protein A3 (S100A3): NM_002960,
206027_at
385298699-Homo sapiens hippocalcin-like 1 (HPCAL1): NM_134421, NA
195546923-Homo sapiens lymphocyte cytosolic protein 1 (L-plastin) (LCP1):
NM_002298, NA
312176372-Homo sapiens solute carrier organic anion transporter family: NM_007256,
NA
327365348-Homo sapiens solute carrier family 20 (phosphate transporter): NM_005415,
201920_at
41056258-Homo sapiens solute carrier family 43: NM_199329, NA
313760623-Homo sapiens platelet/endothelial cell adhesion molecule 1 (PECAM1):
NM_000442, NA
359339003-Homo sapiens cAMP responsive element binding protein 3-like 2
(CREB3L2): NM_194071, NA
208973264-Homo sapiens lactamase: NM_016027, NA
114155157-Homo sapiens Ras association (RaIGDS/AF-6) domain family (N-terminal)
member 9 (RASSF9): NM_005447, NA
154689645-Homo sapiens spindle and kinetochore associated complex subunit 2
(SKA2): NM_182620, NA
197927092-Homo sapiens thymine-DNA glycosylase (TDG): NM_003211, NA
323639470-Homo sapiens SEC14-like 2 (S. cerevisiae) (SEC14L2): NM_033382, NA
71773479-Homo sapiens aldehyde oxidase 1 (AOX1): NM_001159, NA
13259540-Homo sapiens uncoupling protein 2 (mitochondrial: NM_003355, NA
47132584-Homo sapiens protein kinase: NM_002736, 203680_at
71051597-Homo sapiens vacuolar protein sorting 36 homolog (S. cerevisiae) (VPS36):
NM_016075, NA
253683425-Homo sapiens ets variant 1 (ETV1): NM_004956, NA
62244043-Homo sapiens OCIA domain containing 2 (OCIAD2): NM_001014446, NA
118918396-Homo sapiens fibronectin type III domain containing 3A (FNDC3A):
NM_014923, 202304_at
313851098-Homo sapiens frizzled family receptor 2 (FZD2): NM_001466, NA
144953894-Homo sapiens nidogen 2 (osteonidogen) (NID2): NM_007361, NA
239582716-Homo sapiens leucine rich repeat containing 17 (LRRC17): NM_001031692,
NA
359385701-Homo sapiens S100 calcium binding protein A14 (S100A14): NM_020672,
218677_at
15431286-Homo sapiens luteinizing hormone beta polypeptide (LHB): NM_000894, NA
332000002-Homo sapiens mitochondrial ribosomal protein L42 (MRPL42): NM_014050,
NA
65786660-Homo sapiens BTB (POZ) domain containing 11 (BTBD11): NM_001018072,
NA
55953134-Homo sapiens immunoglobulin superfamily: NM_001007237, NA
93004077-Homo sapiens 2-hydroxyacyl-CoA lyase 1 (HACL1): NM_012260, NA
315139002-Homo sapiens UDP-N-acetyl-alpha-D-galactosamine: polypeptide
N-acetylgalactosaminyltransferase 4 (GalNAc-T4) (GALNT4): NM_003774, NA
260436906-Homo sapiens catalase (CAT): NM_001752, NA
399498567-Homo sapiens Rho GTPase activating protein 1 (ARHGAP1): NM_004308,
202117_at
116284367-Homo sapiens peroxisome proliferator-activated receptor gamma (PPARG):
NM_005037, NA
7262372-Homo sapiens bone marrow stromal cell antigen 2 (BST2): NM_004335, NA
209862741-Homo sapiens tetratricopeptide repeat domain 39C (TTC39C): NM_153211,
NA
169234615-Homo sapiens nephroblastoma overexpressed (NOV): NM_002514, NA
153082695-Homo sapiens intercellular adhesion molecule 2 (ICAM2): NM_000873, NA
40217830-Homo sapiens G protein-coupled receptor: NM_022036, NA
131412244-Homo sapiens keratin 19 (KRT19): NM_002276, NA
209969817-Homo sapiens heat shock 27 kDa protein 1 (HSPB1): NM_001540, NA
221316554-Homo sapiens ATP-binding cassette: NM_003786, NA
41872561-Homo sapiens neuropilin 2 (NRP2): NM_201266, NA
148612845-Homo sapiens metastasis suppressor 1 (MTSS1): NM_014751, NA
373432598-Homo sapiens chemokine (C-X-C motif) ligand 1 (melanoma growth
stimulating activity: NM_001511, 204470_at
105990525-Homo sapiens hairy/enhancer-of-split related with YRPW motif 1 (HEY1):
NM_001040708, 218839_at
14195611-Homo sapiens protocadherin beta 5 (PCDHB5): NM_015669, NA
333440479-Homo sapiens protocadherin beta 8 (PCDHB8): NM_019120, 221319_at
293336490-Homo sapiens UDP-N-acetyl-alpha-D-galactosamine: polypeptide
N-acetylgalactosaminyltransferase 10 (GalNAc-T10) (GALNT10): NM_198321,
207357_s_at
7657068-Homo sapiens ERO1-like (S. cerevisiae) (ERO1L): NM_014584, NA
148298657-Homo sapiens chemokine (C-X-C motif) ligand 2 (CXCL2): NM_002089,
209774_x_at
205277317-Homo sapiens dedicator of cytokinesis 2 (DOCK2): NM_004946, NA
170671735-Homo sapiens solute carrier family 19 (thiamine transporter): NM_006996,
209681_at
166362726-Homo sapiens drebrin 1 (DBN1): NM_080881, NA
56682960-Homo sapiens ferritin: NM_000146, 212788_x_at
208431794-Homo sapiens dynactin 4 (p62) (DCTN4): NM_001135644, NA
148227043-Homo sapiens ecotropic viral integration site 2B (EVI2B): NM_006495, NA
261878540-Homo sapiens ecotropic viral integration site 2A (EVI2A): NM_014210, NA
19923980-Homo sapiens IQ motif containing D (IQCD): NM_138451, NA
142360336-Homo sapiens glutamine-fructose-6-phosphate transaminase 2 (GFPT2):
NM_005110, NA
192807326-Homo sapiens Ras-related associated with diabetes (RRAD):
NM_001128850, 204802_at
5031560-Homo sapiens glycoprotein A33 (transmembrane) (GPA33): NM_005814, NA
325910871-Homo sapiens microseminoprotein: NM_138634, NA
219689111-Homo sapiens two pore segment channel 1 (TPCN1): NM_017901,
217914_at
149274647-Homo sapiens DEAD (Asp-Glu-Ala-Asp) box polypeptide 60-like (DDX60L):
NM_001012967, NA
163644281-Homo sapiens serine/threonine kinase 32A (STK32A): NM_145001, NA
197313688-Homo sapiens microtubule associated tumor suppressor 1 (MTUS1):
NM_001001925, NA
87578393-Homo sapiens microtubule-associated protein 2 (MAP2): NM_001039538,
NA
194248076-Homo sapiens Kruppel-like factor 4 (gut) (KLF4): NM_004235,
220266_s_at
349732177-Homo sapiens polypyrimidine tract binding protein 3 (PTBP3): NM_005156,
NA
114431235-Homo sapiens zinc finger protein 462 (ZNF462): NM_021224, NA
55769586-Homo sapiens NOP14 nucleolar protein (NOP14): NM_003703, NA
238550097-Homo sapiens transmembrane channel-like 7 (TMC7): NM_024847, NA
56699460-Homo sapiens paired related homeobox 1 (PRRX1): NM_022716, NA
403310636-Homo sapiens prostaglandin-endoperoxide synthase 1 (prostaglandin G/H
synthase and cyclooxygenase) (PTGS1): NM_000962, 205127_at
153945727-Homo sapiens microtubule-associated protein 1B (MAP1B): NM_005909,
212233_at
10835229-Homo sapiens metallothionein 1G (MT1G): NM_005950, 204745_x_at
194239656-Homo sapiens metallothionein 1X (MT1X): NM_005952, 204326_x_at
57863296-Homo sapiens pleckstrin homology-like domain: NM_003311, NA
108773786-Homo sapiens retinoblastoma 1 (RBI): NM_000321, 203132_at
318037612-Homo sapiens N-acetylneuraminate pyruvate lyase (dihydrodipicolinate
synthase) (NPL): NM_030769, NA
256222399-Homo sapiens filamin B: NM_001457, NA
396578174-Homo sapiens stomatin (STOM): NM_004099, NA
62420878-Homo sapiens electron-transfer-flavoprotein: NM_001985, NA
229577237-Homo sapiens tetratricopeptide repeat domain 18 (TTC18): NM_145170,
NA
223671914-Homo sapiens solute carrier family 35: NM_018389, 218485_s_at
260593648-Homo sapiens endothelin receptor type A (EDNRA): NM_001957,
204463_s_at
80861465-Homo sapiens solute carrier family 7 (anionic amino acid transporter light
chain: NM_014331, 207528_s_at
154813194-Homo sapiens DENN/MADD domain containing 2A (DENND2A):
NM_015689, NA
166851826-Homo sapiens dehydrogenase/reductase (SDR family) member 2 (DHRS2):
NM_005794, NA
34328912-Homo sapiens wingless-type MMTV integration site family: NM_004625,
210248_at
153792563-Homo sapiens solute carrier family 47: NM_001099646, NA
110349771-Homo sapiens collagen: NM_000088, NA
109389361-Homo sapiens arylsulfatase family: NM_024590, NA
325651887-Homo sapiens dual specificity phosphatase 4 (DUSP4): NM_001394, NA
205277384-Homo sapiens metallothionein 2A (MT2A): NM_005953, 212185_x_at
130509310-Homo sapiens egl nine homolog 3 (C. elegans) (EGLN3): NM_022073, NA
356582333-Homo sapiens polo-like kinase 2 (PLK2): NM_006622, NA
100913029-Homo sapiens solute carrier family 26 (sulfate transporter): NM_000112,
205097_at
186972141-Homo sapiens cold inducible RNA binding protein (CIRBP): NR_023313,
200810_s_at
5579451-Homo sapiens calbindin 1: NM_004929, NA
194097399-Homo sapiens metallothionein 1F (MT1F): NM_005949, 213629_x_at
145580599-Homo sapiens schlafen family member 5 (SLFN5): NM_144975, NA
223029497-Homo sapiens EF-hand calcium binding domain 5 (EFCAB5): NM_198529,
NA
222418795-Homo sapiens guanine nucleotide binding protein (G protein): NM_004297,
NA
260763920-Homo sapiens tropomodulin 1 (TMOD1): NM_003275, NA
206597438-Homo sapiens aldehyde dehydrogenase 3 family: NM_001135167,
205623_at
297206809-Homo sapiens claudin 7 (CLDN7): NM_001307, 202790_at
209413739-Homo sapiens prostaglandin E synthase (PTGES): NM_004878,
207388_s_at
73622122-Homo sapiens caspase 3: NM_032991, 202763_at
166362739-Homo sapiens coagulation factor II (thrombin) receptor (F2R): NM_001992,
NA
332078496-Homo sapiens fucosyltransferase 8 (alpha (1: NM_004480, 203988_s_at
68508964-Homo sapiens carboxylesterase 1 (CES1): NM_001025194, NA
124487398-Homo sapiens melanoregulin (MREG): NM_018000, NA
351722337-Homo sapiens nudix (nucleoside diphosphate linked moiety X)-type motif 9
(NUDT9): NM_198038, NA
239735514-Homo sapiens storkhead box 1 (STOX1): NM_152709, NA
301336149-Homo sapiens tumor necrosis factor receptor superfamily: NM_001065,
207643_s_at
67782348-Homo sapiens lysyl oxidase-like 4 (LOXL4): NM_032211, NA
224809464-Homo sapiens anthrax toxin receptor 2 (ANTXR2): NM_058172, NA
151301062-Homo sapiens actin binding LIM protein family: NM_014945, NA
224967098-Homo sapiens complement component 1: NM_201442, 208747_s_at
223278367-Homo sapiens neuralized homolog (Drosophila) (NEURL): NM_004210,
204888_s_at
34147536-Homo sapiens chromosome 12 open reading frame 57 (C12orf57):
NM_138425, NA
372220086-Homo sapiens myopalladin (MYPN): NM_032578, NA
113204624-Homo sapiens argininosuccinate synthase 1 (ASS1): NM_054012, NA
387763630-Homo sapiens general transcription factor IIA: NM_015859, NA
355330265-Homo sapiens solute carrier family 9: NM_001130012, NA
223972680-Homo sapiens metallophosphoesterase domain containing 2 (MPPED2):
NM_001584, NA
111154091-Homo sapiens Rho GTPase activating protein 24 (ARHGAP24):
NM_001025616, 221030_s_at
356461041-Homo sapiens enhancer of mRNA decapping 4 (EDC4): NM_014329, NA
211904150-Homo sapiens serpin peptidase inhibitor: NM_006216, NA
148277030-Homo sapiens glucosaminyl (N-acetyl) transferase 1: NM_001097634,
205505_at
87080808-Homo sapiens UBX domain protein 8 (UBXN8): NM_005671, NA
225690599-Homo sapiens synuclein: NM_000345, 204466_s_at
62865652-Homo sapiens regulator of G-protein signaling 3 (RGS3): NM_021106,
203823_at
201860266-Homo sapiens matrix-remodelling associated 5 (MXRA5): NM_015419,
209596_at
71774196-Homo sapiens ubiquitin specific peptidase 47 (USP47): NM_017944, NA
61676924-Homo sapiens dickkopf 1 homolog (Xenopus laevis) (DKK1): NM_012242,
204602_at
318067953-Homo sapiens toll-like receptor 6 (TLR6): NM_006068, NA
150010588-Homo sapiens interferon induced transmembrane protein 1 (IFITM1):
NM_003641, NA
270483739-Homo sapiens MAP7 domain containing 2 (MAP7D2): NM_152780, NA
153285460-Homo sapiens fibroblast growth factor 2 (basic) (FGF2): NM_002006,
204421_s_at
156523267-Homo sapiens transcriptional adaptor 2B (TADA2B): NM_152293, NA
111494226-Homo sapiens eukaryotic translation initiation factor 4 gamma:
NM_001042559, 200004_at
54792144-Homo sapiens tetraspanin 15 (TSPAN15): NM_012339, NA
291575129-Homo sapiens solute carrier family 29 (nucleoside transporters):
NM_018344, 219344_at
45935370-Homo sapiens serglycin (SRGN): NM_002727, NA
257196278-Homo sapiens secreted frizzled-related protein 1 (SFRP1): NM_003012,
202035_s_at
208879409-Homo sapiens eukaryotic translation initiation factor 4E binding protein 2
(EIF4EBP2): NM_004096, 208769_at
153252272-Homo sapiens steroid sulfatase (microsomal): NM_000351, NA
168693431-Homo sapiens dicer 1: NM_030621, 206061_s_at
221136818-Homo sapiens lin-7 homolog C (C. elegans) (LIN7C): NM_018362,
219399_at
190343007-Homo sapiens SP110 nuclear body protein (SP110): NM_004510,
208012_x_at
190886441-Homo sapiens arrestin domain containing 4 (ARRDC4): NM_183376, NA
109702905-Homo sapiens inositol polyphosphate-5-phosphatase: NM_005539, NA
163644312-Homo sapiens calcium and integrin binding 1 (calmyrin) (CIB1): NM_006384,
201953_at
116235484-Homo sapiens delta/notch-like EGF repeat containing (DNER): NM_139072,
NA
219842238-Homo sapiens semaphorin 7A: NM_003612, NA
47419915-Homo sapiens tryptophanyl-tRNA synthetase (WARS): NM_173701, NA
356883060-Homo sapiens creatine kinase: NM_001823, NA
134244286-Homo sapiens chromogranin A (parathyroid secretory protein 1) (CHGA):
NM_001275, NA
209413718-Homo sapiens dynein: NM_001376, NA
190684643-Homo sapiens RAS guanyl releasing protein 1 (calcium and DAG-regulated)
(RASGRP1): NM_005739, 205590_at
73747882-Homo sapiens ADAM metallopeptidase domain 10 (ADAM10): NM_001110,
202604_x_at
269315832-Homo sapiens zinc finger: NM_152694, NA
167830476-Homo sapiens milk fat globule-EGF factor 8 protein (MFGE8):
NM_001114614, NA
40317625-Homo sapiens thrombospondin 1 (THBS1): NM_003246, NA
356874771-Homo sapiens cingulin-like 1 (CGNL1): NM_032866, NA
51558692-Homo sapiens Bcl2 modifying factor (BMF): NM_033503, NA
193083132-Homo sapiens cellular retinoic acid binding protein 1 (CRABP1):
NM_004378, NA
94721307-Homo sapiens FERM domain containing 5 (FRMD5): NM_032892, NA
221316758-Homo sapiens L1 cell adhesion molecule (L1CAM): NM_024003, NA
399498488-Homo sapiens cysteine-rich protein 2 (CRIP2): NM_001312, NA
156938342-Homo sapiens talin 2 (TLN2): NM_015059, NA
(NB: not all genes are represented by the Affymetrix probe sets)

TABLE 2
The genes, transcripts and respective probesets used to define a
signature for the EMT module of Evi1 pathway.

HES1-Homo sapiens hairy and enhancer of split 1: NM_005524,
203393_at
SNAI1-Homo sapiens snail homolog 1 (Drosophila) (SNAI1):
NM_005985, 219480_at
ASPH-Homo sapiens aspartate beta-hydroxylase (ASPH): NM_004318,
205808_at
HEY1-Homo sapiens hairy/enhancer-of-split related with YRPW motif 1
(HEY1): NM_001040708, 218839_at
MAP2-Homo sapiens microtubule-associated protein 2 (MAP2):
NM_001039538, NA
KLF4-Homo sapiens Kruppel-like factor 4 (gut) (KLF4): NM_004235,
220266_s_at
MAP1B-Homo sapiens microtubule-associated protein 1B (MAP1B):
NM_005909, 212233_at
WNT7A-Homo sapiens wingless-type MMTV integration site family:
NM_004625, 210248_at
NEURL-Homo sapiens neuralized homolog (Drosophila) (NEURL):
NM_004210, 204888_s_at
DKK1-Homo sapiens dickkopf 1 homolog (Xenopus laevis) (DKK1):
NM_012242, 204602_at
FGF2-Homo sapiens fibroblast growth factor 2 (basic) (FGF2):
NM_002006, 204421_s_at
SFRP1-Homo sapiens secreted frizzled-related protein 1 (SFRP1):
NM_003012, 202035_s_at
ADAM10-Homo sapiens ADAM metallopeptidase domain 10
(ADAM10): NM_001110, 202604_x_at

TABLE 3
The genes, transcripts and respective probesets used to define a
signature for the apoptosis module of Evi1 pathway.

SGK2-Homo sapiens serum/glucocorticoid regulated kinase 2 (SGK2):
NM_170693, 220357_s_at
JUN-Homo sapiens jun proto-oncogene (JUN): NM_002228, 201464_x_at
CAP2-Homo sapiens CAP: NM_006366, 212551_at
HMGA2-Homo sapiens high mobility group AT-hook 2 (HMGA2):
NM_003483, 208025_s_at
IGFBP3-Homo sapiens insulin-like growth factor binding protein 3
(IGFBP3): NM_000598, 210095_s_at
RAPGEF3-Homo sapiens Rap guanine nucleotide exchange factor (GEF)
3 (RAPGEF3): NM_001098531, 210051_at
BIRC3-Homo sapiens baculoviral IAP repeat containing 3 (BIRC3):
NM_001165, 210538_s_at
ARHGAP1-Homo sapiens Rho GTPase activating protein 1 (ARHGAP1):
NM_004308, 202117_at
MAP2-Homo sapiens microtubule-associated protein 2 (MAP2):
NM_001039538, 210015_s_at
MAP1B-Homo sapiens microtubule-associated protein 1B (MAP1B):
NM_005909, 212233_at
RB1-Homo sapiens retinoblastoma 1 (RB1): NM_000321, 203132_at
CASP3-Homo sapiens caspase 3: NM_032991, 202763_at
TNFRSF1A-Homo sapiens tumor necrosis factor receptor superfamily:
NM_001065, 207643_s_at
ARHGAP24-Homo sapiens Rho GTPase activating protein 24
(ARHGAP24): NM_001025616, 221030_s_at
RASGRP1-Homo sapiens RAS guanyl releasing protein 1 (calcium and
DAG-regulated) (RASGRP1): NM_005739, 205590_at

TABLE 4
The genes, transcripts and respective probesets used to define a
signature for the immune response module of Evi1 pathway.

IL18-Homo sapiens interleukin 18 (interferon-gamma-inducing factor)
(IL18): NM_001562, 206295_at
SLPI-Homo sapiens secretory leukocyte peptidase inhibitor (SLPI):
NM_003064, 203021_at
SLC20A1-Homo sapiens solute carrier family 20 (phosphate transporter):
NM_005415, 201920_at
CXCL1-Homo sapiens chemokine (C-X-C motif) ligand 1 (melanoma
growth stimulating activity: NM_001511, 204470_at
GALNT10-Homo sapiens UDP-N-acetyl-alpha-D-galactosamine:
polypeptide N-acetylgalactosaminyltransferase 10 (GalNAc-T10)
(GALNT10): NM_198321, 207357_s_at
CXCL2-Homo sapiens chemokine (C-X-C motif) ligand 2 (CXCL2):
NM_002089, 209774_x_at
SLC35C1-Homo sapiens solute carrier family 35: NM_018389,
218485_s_at
SLC26A2-Homo sapiens solute carrier family 26 (sulfate transporter):
NM_000112, 205097_at
FUT8-Homo sapiens fucosyltransferase 8 (alpha (1: NM_004480,
203988_s_at
C1S-Homo sapiens complement component 1: NM_201442,
208747_s_at
GCNT1-Homo sapiens glucosaminyl (N-acetyl) transferase 1:
NM_001097634, 205505_at
MXRA5-Homo sapiens matrix-remodelling associated 5 (MXRA5):
NM_015419, 209596_at

TABLE 5
The genes, transcripts and respective probesets used to define a
signature for the cell survival module of Evi1 pathway.

SLC47A1-Homo sapiens solute carrier family 47: NM_018242, 219525_at
MT1M-Homo sapiens metallothionein 1M (MT1M): NM_176870,
217546_at
SLC48A1-Homo sapiens solute carrier family 48 (heme transporter):
NM_017842, 218416_s_at
FTL-Homo sapiens ferritin: NM_000146, 212788_x_at
RRAD-Homo sapiens Ras-related associated with diabetes (RRAD):
NM_001128850, 204802_at
MT1G-Homo sapiens metallothionein 1G (MT1G): NM_005950,
204745_x_at
MT1X-Homo sapiens metallothionein 1X (MT1X): NM_005952,
204326_x_at
SLC7A11-Homo sapiens solute carrier family 7 (anionic amino acid
transporter light chain: NM_014331, 207528_s_at
MT2A-Homo sapiens metallothionein 2A (MT2A): NM_005953,
212185_x_at
MT1F-Homo sapiens metallothionein 1F (MT1F): NM_005949,
213629_x_at
SLC29A3-Homo sapiens solute carrier family 29 (nucleoside transporters):
NM_018344, 219344_at

TABLE 6
The genes, transcripts and respective probesets used to define a
signature for the retinoic acid module of Evi1 pathway.

TGM2-Homo sapiens transglutaminase 2 (C polypeptide: NM_004613,
201042_at
HRASLS2-Homo sapiens HRAS-like suppressor 2 (HRASLS2):
NM_017878, 216759_at
RARRES3-Homo sapiens retinoic acid receptor responder (tazarotene
induced) 3 (RARRES3): NM_004585, 204070_at
ALDH3A1-Homo sapiens aldehyde dehydrogenase 3 family:
NM_001135167, 205623_at
FGF2-Homo sapiens fibroblast growth factor 2 (basic) (FGF2):
NM_002006, 204421_s_at
SP110-Homo sapiens SP110 nuclear body protein (SP110): NM_004510,
208012_x_at

TABLE 7
The genes, transcripts and respective probesets used to define a
signature for the signalling module module of Evi1 pathway

IL18-Homo sapiens interleukin 18 (interferon-gamma-inducing factor)
(IL18): NM_001562, 206295_at
HTR1D-Homo sapiens 5-hydroxytryptamine (serotonin) receptor 1D:
NM_000864, 207368_at
PTPRK-Homo sapiens protein tyrosine phosphatase: NM_001135648,
203038_at
PTGIS-Homo sapiens prostaglandin I2 (prostacyclin) synthase (PTGIS):
NM_000961, 208131_s_at
KCNN4-Homo sapiens potassium intermediate/small conductance
calcium-activated channel: NM_002250, 204401_at
S100A3-Homo sapiens S100 calcium binding protein A3 (S100A3):
NM_002960, 206027_at
PRKAR2B-Homo sapiens protein kinase: NM_002736, 203680_at
S100A14-Homo sapiens S100 calcium binding protein A14 (S100A14):
NM_020672, 218677_at
CXCL1-Homo sapiens chemokine (C-X-C motif) ligand 1 (melanoma
growth stimulating activity: NM_001511, 204470_at
PCDHB8-Homo sapiens protocadherin beta 8 (PCDHB8): NM_019120,
221319_at
CXCL2-Homo sapiens chemokine (C-X-C motif) ligand 2 (CXCL2):
NM_002089, 209774_x_at
TPCN1-Homo sapiens two pore segment channel 1 (TPCN1):
NM_017901, 217914_at
PTGS1-Homo sapiens prostaglandin-endoperoxide synthase 1
(prostaglandin G/H synthase and cyclooxygenase) (PTGS1):
NM_000962, 205127_at
EDNRA-Homo sapiens endothelin receptor type A (EDNRA):
NM_001957, 204463_s_at
PTGES-Homo sapiens prostaglandin E synthase (PTGES): NM_004878,
207388_s_at
FUT8-Homo sapiens fucosyltransferase 8 (alpha (1: NM_004480,
203988_s_at
SNCA-Homo sapiens synuclein: NM_000345, 204466_s_at
RGS3-Homo sapiens regulator of G-protein signaling 3 (RGS3):
NM_021106, 203823_at
FGF2-Homo sapiens fibroblast growth factor 2 (basic) (FGF2):
NM_002006, 204421_s_at

TABLE 8
The genes, transcripts and respective probesets used to define a
signature for the RNA metabolism module of Evi1 pathway.

IPO5-Homo sapiens importin 5 (IPO5): NM_002271, 211952_at
CIRBP-Homo sapiens cold inducible RNA binding protein (CIRBP):
NR_023313, 200810_s_at
CASP3-Homo sapiens caspase 3: NM_032991, 202763_at
FGF2-Homo sapiens fibroblast growth factor 2 (basic) (FGF2):
NM_002006, 204421_s_at
EIF4G2-Homo sapiens eukaryotic translation initiation factor 4 gamma:
NM_001042559, 200004_at
EIF4EBP2-Homo sapiens eukaryotic translation initiation factor 4E
binding protein 2 (EIF4EBP2): NM_004096, 208769_at
DICER1-Homo sapiens dicer 1: NM_030621, 206061_s_at

TABLE 9
The genes, transcripts and respective probesets used to define a signature
for the module of Evi1 pathway siginificant for suboptimal debulking.

TM4SF1-Homo sapiens transmembrane 4 L six family member 1
(TM4SF1): NM_014220, 209386_at
CLDN10-Homo sapiens claudin 10 (CLDN10): NM_182848, 205328_at
NTNG1-Homo sapiens netrin G1 (NTNG1): NM_001113228, 206713_at
CLIC1-Homo sapiens chloride intracellular channel 1 (CLIC1):
NM_001288, 208659_at
FLOT1-Homo sapiens flotillin 1 (FLOT1): NM_005803, 208748_s_at
RARRES3-Homo sapiens retinoic acid receptor responder (tazarotene
induced) 3 (RARRES3): NM_004585, 204070_at
SLC19A2-Homo sapiens solute carrier family 19 (thiamine transporter):
NM_006996, 209681_at
ARHGAP24-Homo sapiens Rho GTPase activating protein 24
(ARHGAP24): NM_001025616, 221030_s_at
RGS3-Homo sapiens regulator of G-protein signaling 3 (RGS3):
NM_021106, 203823_at
SLC29A3-Homo sapiens solute carrier family 29 (nucleoside transporters):
NM_018344 219344_at
CIB1-Homo sapiens calcium and integrin binding 1 (calmyrin) (CIB1):
NM_006384, 201953_at

TABLE 10
The genes, transcripts and respective probesets used to define a signature for
the interferon response module of Evi1 pathway:

IFI44L-Homo sapiens interferon-induced protein 44-like (IFI44L): NM_006820,
204439_at
IFI30-Homo sapiens interferon: NM_006332, 201422_at
ISG15-Homo sapiens ISG15 ubiquitin-like modifier (ISG15): NM_005101, 205483_s_at
IFI44L-Homo sapiens interferon-induced protein 44-like (IFI44L): NM_006820, 204439_at
IFI44-Homo sapiens interferon-induced protein 44 (IFI44): NM_006417, 214059_at
IFI6-Homo sapiens interferon: NM_022872, 204415_at
IFIT1-Homo sapiens interferon-induced protein with tetratricopeptide repeats 1 (IFIT1):
NM_001548, 203153_at
IFITM1-Homo sapiens interferon induced transmembrane protein 1 (IFITM1):
NM_003641, 201601_x_at
OAS1-Homo sapiens 2′-5′-oligoadenylate synthetase 1: NM_001032409, 202869_at
OAS3-Homo sapiens 2′-5′-oligoadenylate synthetase 3: NM_006187, 218400_at
OAS2-Homo sapiens 2′-5′-oligoadenylate synthetase 2: NM_002535, 204972_at
IFI35-Homo sapiens interferon-induced protein 35 (IFI35): NM_005533, 209417_s_at
IFI30-Homo sapiens interferon: NM_006332, 201422_at
IFIH1-Homo sapiens interferon induced with helicase C domain 1 (IFIH1): NM_022168,
216020_at
MX2-Homo sapiens myxovirus (influenza virus) resistance 2 (mouse) (MX2):
NM_002463, 204994_at
MX1-Homo sapiens myxovirus (influenza virus) resistance 1: NM_002462, 202086_at

TABLE 11
The genes, transcripts and respective probesets used to define a
signature for the module of Evi1 pathway discriminating between
fallopian tube tisssues and EOC.

LAMA4-Homo sapiens laminin: NM_001105209, 202202_s_at
SFRP1-Homo sapiens secreted frizzled-related protein 1 (SFRP1):
NM_003012, 202035_s_at
ENPP2-Homo sapiens ectonucleotide pyrophosphatase/phosphodiesterase
2 (ENPP2): NM_001040092, 209392_at
TSPAN1-Homo sapiens tetraspanin 1 (TSPAN1): NM_005727, 209114_at
TGM2-Homo sapiens transglutaminase 2 (C polypeptide: NM_004613,
201042_at
PTGIS-Homo sapiens prostaglandin I2 (prostacyclin) synthase (PTGIS):
NM_000961, 208131_s_at
FNDC3A-Homo sapiens fibronectin type III domain containing 3A
(FNDC3A): NM_014923, 202304_at
GNG11-Homo sapiens guanine nucleotide binding protein (G protein):
NM_004126, 204115_at
PRUNE2-Homo sapiens prune homolog 2 (Drosophila) (PRUNE2):
NM_015225, 212805_at
CLDN10-Homo sapiens claudin 10 (CLDN10): NM_182848, 205328_at
TPM2-Homo sapiens tropomyosin 2 (beta) (TPM2): NM_213674,
204083_s_at
TSPAN15-Homo sapiens tetraspanin 15 (TSPAN15): NM_012339,
218693_at
AKR1C3-Homo sapiens aldo-keto reductase family 1: NM_003739,
209160_at
GULP1-Homo sapiens GULP: NM_016315, NA
AIM1-Homo sapiens absent in melanoma 1 (AIM1): NM_001624,
212543_at
TSPAN12-Homo sapiens tetraspanin 12 (TSPAN12): NM_012338,
219274_at

TABLE 12
Survival significance analysis P-values and expression thresholds for the Evi1
pathway genes included in the Evi1 pathway signature. The P-value was calculated using
logrank test. The consenus threshold was calculated from extraplation of the best-fit
approximation function defined on FIG. 14. The last column indicates by symbol 1 the
genes of 39-gene expression prognostic signature, whose expression was survival
significant (p < 0.05) in the both studied cohorts. The 39-gene signatures is consided as the
reprodusible EVI1 pathway gene expression prognostic signature.

| | | | | | | | | | 39 prognostic |
|---|---|---|---|---|---|---|---|---|---|
| | | | | | | | | | genes, which |
| | | | | | | | | | expression |
| | | | | | | | | | level threshold |
| | | | | | | | | | survival |
| | | | | | | | | | significant |
| | | Cohort 1 (373 patients) | | | Cohort 2 (230 patients) | | | Consensus | in the |
| Symbol | Probe set | Threshold | P-value | Wald stat | Threshold | P-value | Wald stat | threshold | both cohorts |
| CXCL2 | 209774_x_at | 6.001 | 1.41E−01 | 2.16 | 7.39 | 3.71E−02 | 4.35 | 6.69 | |
| SFRP1 | 202035_s_at | 6.023 | 1.82E−02 | 5.58 | 6.948 | 8.21E−05 | 15.51 | 6.49 | 1 |
| IL18 | 206295_at | 6.051 | 2.89E−02 | 4.78 | 8.105 | 7.09E−02 | 3.26 | 7.06 | |
| BIRC3 | 210538_s_at | 6.098 | 6.00E−02 | 3.54 | 8.366 | 1.96E−02 | 5.45 | 7.21 | |
| RASGRP1 | 205590_at | 6.14 | 2.02E−02 | 5.39 | 7.18 | 3.86E−02 | 4.28 | 6.66 | 1 |
| PTGES | 207388_s_at | 6.145 | 4.24E−02 | 4.12 | 7.738 | 1.59E−01 | 1.98 | 6.93 | |
| EIF4EBP2 | 208769_at | 6.2 | 3.25E−02 | 4.57 | 7.037 | 2.70E−02 | 4.89 | 6.62 | 1 |
| LHB | 214471_x_at | 6.239 | 3.64E−04 | 12.71 | 7.47 | 6.06E−02 | 3.52 | 6.85 | |
| MAP2 | 210015_s_at | 6.256 | 2.61E−01 | 1.26 | 7.469 | 1.04E−02 | 6.57 | 6.86 | |
| NTNG1 | 206713_at | 6.26 | 1.20E−01 | 2.42 | 6.532 | 1.58E−01 | 2 | 6.41 | |
| ARHGAP24 | 221030_s_at | 6.292 | 5.98E−02 | 3.54 | 6.602 | 7.59E−02 | 3.15 | 6.46 | |
| TAS2R9 | 221461_at | 6.318 | 5.63E−05 | 16.22 | 6.632 | 1.71E−02 | 5.68 | 6.49 | 1 |
| HTR1D | 207368_at | 6.325 | 8.86E−02 | 2.9 | 6.338 | 1.52E−01 | 2.05 | 6.35 | |
| DKK1 | 204602_at | 6.341 | 1.33E−01 | 2.26 | 6.702 | 1.56E−01 | 2.02 | 6.53 | |
| KLF4 | 220266_s_at | 6.343 | 2.62E−03 | 9.06 | 6.691 | 1.16E−01 | 2.46 | 6.53 | |
| CLDN10 | 205328_at | 6.345 | 5.94E−03 | 7.57 | 8.494 | 1.35E−02 | 6.1 | 7.4 | 1 |
| RARRES3 | 204070_at | 6.367 | 8.26E−02 | 3.01 | 9.771 | 1.06E−02 | 6.53 | 8.03 | |
| ASPH | 205808_at | 6.371 | 5.74E−02 | 3.61 | 7.498 | 6.14E−02 | 3.5 | 6.93 | |
| HRASLS2 | 216759_at | 6.411 | 1.41E−03 | 10.19 | 7.465 | 4.02E−02 | 4.21 | 6.94 | 1 |
| SLC7A11 | 207528_s_at | 6.427 | 2.39E−03 | 9.23 | 6.677 | 1.79E−03 | 9.76 | 6.56 | 1 |
| S100A3 | 206027_at | 6.456 | 3.97E−02 | 4.23 | 7.134 | 2.73E−01 | 1.2 | 6.8 | |
| HES1 | 203393_at | 6.456 | 1.06E−01 | 2.62 | 7.157 | 8.76E−02 | 2.92 | 6.81 | |
| KCNN4 | 204401_at | 6.47 | 1.24E−01 | 2.37 | 8.828 | 4.30E−02 | 4.1 | 7.63 | |
| DNAJB5 | 207453_s_at | 6.484 | 1.63E−03 | 9.92 | 6.662 | 6.15E−03 | 7.51 | 6.59 | 1 |
| PCDHB8 | 221319_at | 6.497 | 2.67E−02 | 4.91 | 6.883 | 9.35E−02 | 2.81 | 6.7 | |
| CXCL1 | 204470_at | 6.5 | 7.69E−03 | 7.1 | 8.673 | 1.51E−01 | 2.06 | 7.57 | |
| SNAI1 | 219480_at | 6.502 | 5.20E−03 | 7.81 | 7.418 | 1.59E−03 | 9.97 | 6.96 | 1 |
| NEURL | 204888_s_at | 6.523 | 2.07E−02 | 5.35 | 7.141 | 1.53E−02 | 5.89 | 6.84 | 1 |
| SGK2 | 220357_s_at | 6.571 | 1.20E−01 | 2.42 | 7.976 | 1.76E−01 | 1.83 | 7.27 | |
| SLC47A1 | 219525_at | 6.574 | 6.91E−03 | 7.3 | 6.978 | 3.25E−02 | 4.57 | 6.79 | 1 |
| PAK3 | 214607_at | 6.6 | 1.60E−02 | 5.81 | 7.222 | 1.41E−03 | 10.19 | 6.92 | 1 |
| SLC48A1 | 218416_s_at | 6.625 | 4.88E−02 | 3.88 | 7.647 | 1.75E−02 | 5.64 | 7.14 | 1 |
| HEY1 | 218839_at | 6.652 | 1.05E−02 | 6.54 | 7.931 | 7.23E−02 | 3.23 | 7.29 | |
| SLC35C1 | 218485_s_at | 6.732 | 1.21E−01 | 2.41 | 7.827 | 5.44E−02 | 3.7 | 7.28 | |
| GFPT2 | 205100_at | 6.748 | 6.37E−04 | 11.66 | 7.517 | 1.80E−03 | 9.75 | 7.14 | 1 |
| RRAD | 204802_at | 6.748 | 1.67E−02 | 5.73 | 7.244 | 3.42E−02 | 4.49 | 7 | 1 |
| FGF2 | 204421_s_at | 6.768 | 2.50E−01 | 1.32 | 6.937 | 1.03E−01 | 2.66 | 6.87 | |
| GALNT10 | 207357_s_at | 6.785 | 1.89E−04 | 13.94 | 7.487 | 8.17E−04 | 11.2 | 7.14 | 1 |
| ROD1 | 207223_s_at | 6.826 | 4.64E−05 | 16.59 | 7.362 | 2.18E−03 | 9.39 | 7.1 | 1 |
| SLC26A2 | 205097_at | 6.837 | 8.17E−02 | 3.03 | 9.59 | 2.89E−03 | 8.88 | 8.19 | |
| PTGS1 | 205127_at | 6.912 | 6.71E−04 | 11.57 | 7.901 | 1.48E−02 | 5.94 | 7.41 | 1 |
| ALDH3A1 | 205623_at | 7.026 | 1.01E−02 | 6.62 | 7.058 | 1.91E−02 | 5.49 | 7.06 | 1 |
| FLOT1 | 208748_s_at | 7.037 | 1.68E−01 | 1.9 | 7.682 | 1.06E−01 | 2.61 | 7.37 | |
| SNCA | 204466_s_at | 7.043 | 8.44E−04 | 11.14 | 7.21 | 9.03E−04 | 11.02 | 7.14 | 1 |
| PDZK1 | 205380_at | 7.054 | 4.16E−02 | 4.15 | 6.247 | 3.82E−03 | 8.37 | 6.68 | 1 |
| ADAM10 | 202604_x_at | 7.083 | 1.16E−01 | 2.46 | 7.697 | 7.95E−03 | 7.05 | 7.4 | |
| OSTM1 | 218196_at | 7.092 | 9.23E−05 | 15.29 | 7.575 | 8.70E−03 | 6.88 | 7.34 | 1 |
| WNT7A | 210248_at | 7.092 | 3.69E−02 | 4.35 | 8.068 | 1.57E−01 | 2.01 | 7.58 | |
| MT1M | 217546_at | 7.197 | 7.52E−02 | 3.16 | 6.774 | 3.30E−02 | 4.54 | 7.01 | |
| EDNRA | 204463_s_at | 7.232 | 1.40E−02 | 6.04 | 8.038 | 3.33E−04 | 12.88 | 7.64 | 1 |
| SLC29A3 | 219344_at | 7.244 | 8.06E−02 | 3.05 | 8.197 | 1.48E−01 | 2.09 | 7.72 | |
| RGS3 | 203823_at | 7.281 | 4.25E−02 | 4.11 | 9.486 | 8.49E−02 | 2.97 | 8.36 | |
| IPO5 | 211952_at | 7.289 | 4.86E−02 | 3.89 | 7.872 | 1.58E−02 | 5.82 | 7.59 | 1 |
| IL7R | 205798_at | 7.315 | 1.63E−02 | 5.77 | 9.177 | 1.09E−01 | 2.56 | 8.23 | |
| FUT8 | 203988_s_at | 7.329 | 5.16E−07 | 25.2 | 9.277 | 4.27E−02 | 4.11 | 8.29 | 1 |
| SLC19A2 | 209681_at | 7.351 | 3.77E−02 | 4.32 | 7.679 | 1.07E−02 | 6.52 | 7.53 | 1 |
| MT1G | 204745_x_at | 7.399 | 4.88E−02 | 3.88 | 11.428 | 2.95E−01 | 1.1 | 9.37 | |
| RAPGEF3 | 210051_at | 7.43 | 4.52E−03 | 8.06 | 7.516 | 2.45E−01 | 1.35 | 7.49 | |
| GCNT1 | 205505_at | 7.611 | 4.44E−02 | 4.04 | 7.52 | 5.77E−02 | 3.6 | 7.58 | |
| MAGED2 | 208682_s_at | 7.624 | 1.37E−02 | 6.08 | 9.679 | 5.85E−02 | 3.58 | 8.63 | |
| DICER1 | 206061_s_at | 7.708 | 2.98E−03 | 8.82 | 8.656 | 1.76E−02 | 5.63 | 8.18 | 1 |
| CASP3 | 202763_at | 7.734 | 8.27E−03 | 6.97 | 8.541 | 1.60E−01 | 1.98 | 8.14 | |
| HMGA2 | 208025_s_at | 7.845 | 1.01E−01 | 2.7 | 9.86 | 2.53E−04 | 13.39 | 8.84 | |
| MT1X | 204326_x_at | 7.889 | 2.65E−02 | 4.93 | 11.786 | 2.32E−01 | 1.43 | 9.79 | |
| MT1F | 213629_x_at | 7.921 | 7.81E−02 | 3.1 | 10.923 | 2.32E−01 | 1.43 | 9.39 | |
| PRKAR2B | 203680_at | 8.028 | 1.01E−01 | 2.69 | 7.308 | 4.56E−02 | 3.99 | 7.69 | |
| AKAP12 | 210517_s_ [symbol] | 8.12 | 4.06E−04 | 12.51 | 9.718 | 3.43E−03 | 8.56 | 8.91 | 1 |
| MXRA5 | 209596_at | 8.164 | 1.24E−03 | 10.43 | 10.854 | 1.43E−02 | 6 | 9.48 | 1 |
| PIGK | 209707_at | 8.264 | 4.67E−05 | 16.58 | 8.592 | 1.02E−01 | 2.67 | 8.44 | |
| CIB1 | 201953_at | 8.323 | 1.16E−03 | 10.55 | 10.204 | 9.36E−03 | 6.75 | 9.25 | 1 |
| CAP2 | 212551_at | 8.351 | 7.96E−02 | 3.07 | 8.721 | 4.38E−03 | 8.12 | 8.54 | |
| SP110 | 208012_x_ [symbol] | 8.389 | 1.08E−01 | 2.58 | 9.285 | 1.21E−01 | 2.4 | 8.84 | |
| TNFRSF1A | 207643_s_ [symbol] | 8.406 | 1.25E−02 | 6.24 | 9.149 | 3.66E−02 | 4.37 | 8.78 | 1 |
| RB1 | 203132_at | 8.468 | 8.23E−06 | 19.88 | 9.629 | 2.52E−02 | 5.01 | 9.04 | 1 |
| SLC20A1 | 201920_at | 8.497 | 2.39E−01 | 1.38 | 9.462 | 2.97E−02 | 4.73 | 8.98 | |
| GULP1 | 204235_s_ [symbol] | 8.597 | 7.22E−03 | 7.22 | 9.059 | 5.22E−02 | 3.77 | 8.84 | |
| FAM3C | 201889_at | 8.871 | 1.19E−04 | 14.81 | 10.727 | 8.92E−04 | 11.04 | 9.78 | 1 |
| TPCN1 | 217914_at | 9.251 | 5.09E−02 | 3.81 | 9.549 | 1.85E−03 | 9.7 | 9.41 | |
| MAP1B | 212233_at | 9.294 | 9.55E−02 | 2.78 | 9.617 | 2.56E−02 | 4.98 | 9.46 | |
| ANXA4 | 201301_s_ [symbol] | 9.331 | 1.01E−06 | 23.91 | 10.772 | 5.20E−02 | 3.78 | 10.04 | |
| PTGIS | 208131_s_ [symbol] | 9.346 | 2.19E−02 | 5.25 | 8.987 | 7.80E−04 | 11.29 | 9.19 | 1 |
| CRYAB | 209283_at | 9.483 | 3.35E−05 | 17.21 | 9.492 | 1.92E−03 | 9.62 | 9.5 | 1 |
| PTPRK | 203038_at | 9.485 | 1.77E−02 | 5.62 | 9.333 | 5.17E−02 | 3.79 | 9.43 | |
| JUN | 201464_x_ [symbol] | 9.513 | 2.44E−02 | 5.07 | 11.44 | 6.21E−02 | 3.48 | 10.46 | |
| TGM2 | 201042_at | 9.749 | 6.47E−03 | 7.41 | 8.125 | 6.18E−02 | 3.49 | 8.98 | |
| S100A14 | 218677_at | 9.947 | 8.58E−02 | 2.95 | 10.036 | 1.39E−02 | 6.05 | 10 | |
| CIRBP | 200810_s_ [symbol] | 10.028 | 2.14E−02 | 5.3 | 10.889 | 1.85E−01 | 1.76 | 10.46 | |
| IGFBP3 | 210095_s_ [symbol] | 10.143 | 1.87E−01 | 1.74 | 11.028 | 1.27E−01 | 2.33 | 10.58 | |
| MT2A | 212185_x_ [symbol] | 10.148 | 3.12E−02 | 4.64 | 12.815 | 3.68E−02 | 4.36 | 11.45 | 1 |
| HSPB1 | 201841_s_ [symbol] | 10.246 | 1.91E−02 | 5.5 | 11.128 | 9.11E−02 | 2.85 | 10.69 | |
| RBP1 | 203423_at | 10.402 | 1.30E−02 | 6.17 | 10.161 | 1.63E−01 | 1.95 | 10.3 | |
| C1S | 208747_s_ [symbol] | 10.455 | 3.90E−02 | 4.26 | 11.192 | 3.33E−02 | 4.53 | 10.82 | 1 |
| ARHGAP1 | 202117_at | 10.515 | 9.00E−02 | 2.87 | 10.591 | 2.00E−01 | 1.64 | 10.56 | |
| TM4SF1 | 209386_at | 10.621 | 5.21E−02 | 3.77 | 10.339 | 3.51E−02 | 4.44 | 10.5 | |
| EIF4G2 | 200004_at | 10.747 | 3.21E−03 | 8.68 | 12.569 | 6.42E−04 | 11.65 | 11.64 | 1 |
| CLIC1 | 208659_at | 10.826 | 1.08E−01 | 2.59 | 12.316 | 1.63E−01 | 1.94 | 11.56 | |
| DAZAP2 | 200794_x_ [symbol] | 11.097 | 2.23E−02 | 5.22 | 12.074 | 3.21E−02 | 4.6 | 11.58 | 1 |
| CLU | 208791_at | 11.214 | 6.08E−02 | 3.51 | 11.328 | 1.17E−01 | 2.45 | 11.28 | |
| SLPI | 203021_at | 11.294 | 3.74E−03 | 8.41 | 12.351 | 6.87E−02 | 3.31 | 11.82 | |
| FTL | 212788_x_ [symbol] | 12.231 | 2.44E−01 | 1.36 | 13.998 | 1.58E−01 | 1.99 | 13.1 | |

[symbol]

indicates data missing or illegible when filed

TABLE 13
Survival significance analysis P-values and copy number thresholds
for the Evi1 pathway genes included in the Evi1 pathway
signature. The P-value was calculated using logrank test. The
results were obtained using 353 patients of the TCGA cohort

| | | | | survival |
|---|---|---|---|---|
| | | | | significant |
| | | | Wald | subset(1; |
| Symbol | Threshold | P-value | score | p < 0.05) |
| IL7R | 2.21365 | 1.49E−06 | 23.17 | 1 |
| MXRA5 | 1.16641 | 1.21E−05 | 19.15 | 1 |
| PRKAR2B | 2.67141 | 2.41E−05 | 17.83 | 1 |
| PAK3 | 1.32 | 3.49E−05 | 17.13 | 1 |
| RB1 | 1.980445 | 9.52E−05 | 15.23 | 1 |
| TGM2 | 2.67902 | 0.000103 | 15.08 | 1 |
| SLC7A11 | 2.15615 | 0.000282 | 13.19 | 1 |
| SFRP1 | 3.13094 | 0.000352 | 12.77 | 1 |
| MAGED2 | 1.28143 | 0.000556 | 11.92 | 1 |
| S100A3 | 2.19223 | 0.00162 | 9.94 | 1 |
| FLOT1 | 2.40195 | 0.002175 | 9.4 | 1 |
| AKAP12 | 1.40143 | 0.00283 | 8.91 | 1 |
| CASP3 | 2.21411 | 0.003182 | 8.7 | 1 |
| SGK2 | 2.800065 | 0.003816 | 8.37 | 1 |
| SLC48A1 | 2.82838 | 0.003858 | 8.35 | 1 |
| SNAI1 | 2.63933 | 0.004027 | 8.27 | 1 |
| PTGIS | 3.73315 | 0.004524 | 8.06 | 1 |
| HES1 | 3.35096 | 0.005058 | 7.86 | 1 |
| HSPB1 | 2.55025 | 0.005319 | 7.77 | 1 |
| SLPI | 2.51372 | 0.005787 | 7.62 | 1 |
| SLC19A2 | 2.82194 | 0.005897 | 7.58 | 1 |
| DICER1 | 3.00039 | 0.005944 | 7.57 | 1 |
| SNCA | 1.59198 | 0.00593 | 7.57 | 1 |
| RASGRP1 | 2.24194 | 0.006164 | 7.5 | 1 |
| CAP2 | 2.34046 | 0.006683 | 7.36 | 1 |
| EDNRA | 2.11867 | 0.006876 | 7.31 | 1 |
| ARHGAP24 | 1.509175 | 0.008503 | 6.92 | 1 |
| LHB | 1.42829 | 0.008794 | 6.86 | 1 |
| DNAJB5 | 2.15822 | 0.00922 | 6.78 | 1 |
| GFPT2 | 2.50129 | 0.009352 | 6.75 | 1 |
| TAS2R9 | 4.06135 | 0.010044 | 6.63 | 1 |
| FGF2 | 2.3539 | 0.010673 | 6.52 | 1 |
| RAPGEF3 | 2.607415 | 0.011782 | 6.34 | 1 |
| | | | | survival |
| | | | | significant |
| | | | | subset(1; |
| | | | Wald | p < 0.05; |
| Symbol | Threshold | P-value | score | 0; p > 0.05) |
| KCNN4 | 1.81039 | 0.012163 | 6.29 | 1 |
| HMGA2 | 2.72426 | 0.012307 | 6.27 | 1 |
| CLDN10 | 1.701065 | 0.01248 | 6.24 | 1 |
| HRASLS2 | 2.29436 | 0.013869 | 6.05 | 1 |
| RARRES3 | 2.29436 | 0.013869 | 6.05 | 1 |
| CLIC1 | 2.39915 | 0.014043 | 6.03 | 1 |
| PDZK1 | 2.14357 | 0.015828 | 5.82 | 1 |
| PTGS1 | 2.51412 | 0.017748 | 5.62 | 1 |
| SLC35C1 | 2.41222 | 0.017747 | 5.62 | 1 |
| CLU | 2.35829 | 0.018843 | 5.52 | 1 |
| C1S | 3.78613 | 0.020183 | 5.4 | 1 |
| CXCL1 | 2.17999 | 0.020516 | 5.37 | 1 |
| PCDHB8 | 1.47138 | 0.020776 | 5.35 | 1 |
| EIF4EBP2 | 1.55597 | 0.021505 | 5.29 | 1 |
| FTL | 1.40119 | 0.022072 | 5.24 | 1 |
| HTR1D | 1.95826 | 0.023371 | 5.14 | 1 |
| IPO5 | 1.74839 | 0.023807 | 5.11 | 1 |
| S100A14 | 2.37362 | 0.026968 | 4.89 | 1 |
| SLC29A3 | 1.46664 | 0.027039 | 4.89 | 1 |
| WNT7A | 1.68881 | 0.031664 | 4.62 | 1 |
| PTPRK | 1.53123 | 0.033155 | 4.54 | 1 |
| GULP1 | 1.88477 | 0.03331 | 4.53 | 1 |
| ANXA4 | 1.85648 | 0.037283 | 4.34 | 1 |
| DKK1 | 2.12651 | 0.037488 | 4.33 | 1 |
| KLF4 | 1.85891 | 0.045907 | 3.99 | 1 |
| DAZAP2 | 2.12005 | 0.051794 | 3.78 | 0 |
| HEY1 | 3.8496 | 0.057742 | 3.6 | 0 |
| CXCL2 | 1.43368 | 0.063371 | 3.45 | 0 |
| SLC47A1 | 1.941535 | 0.065293 | 3.4 | 0 |
| SLC20A1 | 1.80747 | 0.067219 | 3.35 | 0 |
| ALDH3A1 | 1.36175 | 0.072711 | 3.22 | 0 |
| CIB1 | 2.16959 | 0.074594 | 3.18 | 0 |
| | | | | survival |
| | | | | significant |
| | | | Wald | subset (0; |
| Symbol | Threshold | P-value | score | p > 0.05) |
| TNFRSF1A | 2.30438 | 0.076445 | 3.14 | 0 |
| SLC26A2 | 1.4659 | 0.076998 | 3.13 | 0 |
| TM4SF1 | 2.48232 | 0.081968 | 3.03 | 0 |
| IGFBP3 | 2.49365 | 0.087558 | 2.92 | 0 |
| ADAM10 | 2.47723 | 0.093196 | 2.82 | 0 |
| OSTM1 | 2.57821 | 0.093353 | 2.82 | 0 |
| NEURL | 2.06792 | 0.097443 | 2.75 | 0 |
| MAP2 | 1.84945 | 0.099613 | 2.71 | 0 |
| CRYAB | 1.53849 | 0.102423 | 2.67 | 0 |
| GALNT10 | 1.84501 | 0.102121 | 2.67 | 0 |
| RRAD | 1.53605 | 0.104733 | 2.63 | 0 |
| ROD1 | 2.30217 | 0.123416 | 2.37 | 0 |
| FAM3C | 2.810425 | 0.136044 | 2.22 | 0 |
| ASPH | 3.253015 | 0.143813 | 2.14 | 0 |
| JUN | 2.90782 | 0.143085 | 2.14 | 0 |
| TPCN1 | 2.75188 | 0.14428 | 2.13 | 0 |
| IL18 | 1.52147 | 0.146163 | 2.11 | 0 |
| MAP1B | 2.0355 | 0.156326 | 2.01 | 0 |
| MECOM | 4.06126 | 0.157911 | 1.99 | 0 |
| EIF4G2 | 2.17773 | 0.16362 | 1.94 | 0 |
| PTGES | 1.91098 | 0.175401 | 1.84 | 0 |
| ARHGAP1 | 2.23072 | 0.175882 | 1.83 | 0 |
| RGS3 | 2.01651 | 0.177717 | 1.82 | 0 |
| FUT8 | 1.563095 | 0.185593 | 1.75 | 0 |
| MT1G | 1.62815 | 0.190474 | 1.71 | 0 |
| MT1X | 1.62815 | 0.190474 | 1.71 | 0 |
| GCNT1 | 2.24942 | 0.197882 | 1.66 | 0 |
| RBP1 | 2.074335 | 0.197669 | 1.66 | 0 |
| MT2A | 1.62802 | 0.22276 | 1.49 | 0 |
| NTNG1 | 2.24203 | 0.2231 | 1.48 | 0 |
| PIGK | 2.45447 | 0.258911 | 1.27 | 0 |
| CIRBP | 1.72506 | 0.261432 | 1.26 | 0 |
| SP110 | 2.65026 | 0.266495 | 1.23 | 0 |
| BIRC3 | 3.24182 | 0.315555 | 1.01 | 0 |
| MT1F | 1.59889 | 0.36314 | 0.83 | 0 |
| MT1M | 1.59889 | 0.36314 | 0.83 | 0 |

TABLE 14
Univariate Cox proportional hazard models of the patient's 5-year overall survival, based on the
criteria available at the operation time (TCGA cohort, 356 patients). The models are characterized
with the following parameters (columns): beta—exponential coefficient of the Cox-proportional
hazard model; HR—hazard ratio; CI_L and CI_U—the lower and upper bounds of the 95% HR
confidence interval; z—Wald statistic; Pr—the survival significance calculated from the Wald statistic.

| Factor | Group | beta | HR | CI _L | Cl _U | z | Pr(>\|z\|) |
|---|---|---|---|---|---|---|---|
| immune | high risk | 2.50 | 12.14 | 7.47 | 19.73 | 10.07 | <0.00E−15 |
| immune | medium risk | 1.31 | 3.71 | 2.63 | 5.23 | 7.46 | 8.57E−14 |
| signalling | high risk | 1.91 | 6.75 | 4.04 | 11.28 | 7.29 | 3.07E−13 |
| apoptosis | high risk | 1.85 | 6.35 | 3.83 | 10.52 | 7.18 | 7.00E−13 |
| debulking | high risk | 1.63 | 5.11 | 3.20 | 8.17 | 6.81 | 9.84E−12 |
| RiskGroup | low risk | −1.37 | 0.25 | 0.17 | 0.39 | −6.47 | 1.00E−10 |
| rnametabolism | high risk | 1.49 | 4.44 | 2.74 | 7.18 | 6.07 | 1.31E−09 |
| Primary chemotherapy | PARTIAL RESPONSE | 1.21 | 3.34 | 2.26 | 4.94 | 6.05 | 1.44E−09 |
| signalling | medium risk | 0.95 | 2.60 | 1.89 | 3.57 | 5.86 | 4.68E−09 |
| emt | high risk | 1.42 | 4.15 | 2.56 | 6.72 | 5.78 | 7.62E−09 |
| Neoplasm status | TUMOR FREE | −3.02 | 0.05 | 0.02 | 0.14 | −5.60 | 2.12E−08 |
| rnametabolism | medium risk | 0.95 | 2.58 | 1.84 | 3.61 | 5.49 | 3.92E−08 |
| emt | medium risk | 0.87 | 2.39 | 1.75 | 3.26 | 5.49 | 4.10E−08 |
| debulking | medium risk | 0.85 | 2.35 | 1.72 | 3.21 | 5.34 | 9.49E−08 |
| surv | high risk | 1.65 | 5.20 | 2.83 | 9.55 | 5.31 | 1.08E−07 |
| Primary chemotherapy | PROGRESSIVE DISEASE | 1.27 | 3.56 | 2.21 | 5.74 | 5.23 | 1.72E−07 |
| RiskGroup | high risk | 1.41 | 4.08 | 2.30 | 7.22 | 4.83 | 1.39E−06 |
| apoptosis | medium risk | 0.87 | 2.40 | 1.64 | 3.51 | 4.50 | 6.89E−06 |
| Residual tumor | null | −0.89 | 0.41 | 0.26 | 0.66 | −3.72 | 1.98E−04 |
| Age | >=67 | 0.72 | 2.05 | 1.40 | 3.00 | 3.69 | 2.24E−04 |
| retinoic | medium risk | 0.66 | 1.94 | 1.35 | 2.77 | 3.62 | 2.92E−04 |
| surv | medium risk | 0.92 | 2.51 | 1.50 | 4.22 | 3.49 | 4.84E−04 |
| retinoic | high risk | 1.02 | 2.78 | 1.49 | 5.18 | 3.21 | 1.31E−03 |
| Residual tumor | No Macroscopic disease | −0.67 | 0.51 | 0.32 | 0.82 | −2.76 | 5.83E−03 |
| Tumor stage | Stages I and II | −1.16 | 0.31 | 0.13 | 0.76 | −2.56 | 1.04E−02 |
| Tumor grade | Grades 3 and 4 | 0.45 | 1.57 | 1.06 | 2.34 | 2.25 | 2.45E−02 |
| Age | 53-66 | 0.40 | 1.49 | 1.04 | 2.13 | 2.16 | 3.08E−02 |
| Primary chemotherapy | STABLE DISEASE | 0.72 | 2.05 | 1.06 | 3.97 | 2.14 | 3.27E−02 |
| Neoplasm status | WITH TUMOR | −0.40 | 0.67 | 0.44 | 1.02 | −1.87 | 6.09E−02 |
| Residual tumor | 11-20 mm | 0.10 | 1.11 | 0.66 | 1.85 | 0.39 | 6.97E−01 |
| Race | Other | 0.10 | 1.10 | 0.65 | 1.87 | 0.36 | 7.19E−01 |
| Residual tumor | >20 mm | 0.04 | 1.05 | 0.73 | 1.50 | 0.24 | 8.11E−01 |

TABLE 15
Univariate Cox proportional hazard models of the patient's 5-year overall survival, based on the
criteria available at the operation time (TCGA cohort, 356 patients). The models are characterized
with the following parameters (columns): beta—exponential coefficient of the Cox-proportional
hazard model; HR—hazard ratio; CI_L and CI_U—the lower and upper bounds of the 95% HR
confidence interval; z—Wald statistic; Pr—the survival significance calculated from the Wald statistic.

| Factor | Group | beta | HR | CI _L | Cl _U | z | Pr(>\|z\|) |
|---|---|---|---|---|---|---|---|
| immune | high risk | 2.50 | 12.14 | 7.47 | 19.73 | 10.07 | <0.00E150 |
| immune | medium risk | 1.31 | 3.71 | 2.63 | 5.23 | 7.46 | 8.57E−14 |
| signalling | high risk | 1.91 | 6.75 | 4.04 | 11.28 | 7.29 | 3.07E−13 |
| apoptosis | high risk | 1.85 | 6.35 | 3.83 | 10.52 | 7.18 | 7.00E−13 |
| debulking | high risk | 1.63 | 5.11 | 3.20 | 8.17 | 6.81 | 9.84E−12 |
| RiskGroup | low risk | −1.37 | 0.25 | 0.17 | 0.39 | −6.47 | 1.00E−10 |
| rnametabolism | high risk | 1.49 | 4.44 | 2.74 | 7.18 | 6.07 | 1.31E−09 |
| Primary chemotherapy | PARTIAL RESPONSE | 1.21 | 3.34 | 2.26 | 4.94 | 6.05 | 1.44E−09 |
| signalling | medium risk | 0.95 | 2.60 | 1.89 | 3.57 | 5.86 | 4.68E−09 |
| emt | high risk | 1.42 | 4.15 | 2.56 | 6.72 | 5.78 | 7.62E−09 |
| Neoplasm status | TUMOR FREE | −3.02 | 0.05 | 0.02 | 0.14 | −5.60 | 2.12E−08 |
| rnametabolism | medium risk | 0.95 | 2.58 | 1.84 | 3.61 | 5.49 | 3.92E−08 |
| emt | medium risk | 0.87 | 2.39 | 1.75 | 3.26 | 5.49 | 4.10E−08 |
| debulking | medium risk | 0.85 | 2.35 | 1.72 | 3.21 | 5.34 | 9.49E−08 |
| surv | high risk | 1.65 | 5.20 | 2.83 | 9.55 | 5.31 | 1.08E−07 |
| Primary chemotherapy | PROGRESSIVE DISEASE | 1.27 | 3.56 | 2.21 | 5.74 | 5.23 | 1.72E−07 |
| RiskGroup | high risk | 1.41 | 4.08 | 2.30 | 7.22 | 4.83 | 1.39E−06 |
| apoptosis | medium risk | 0.87 | 2.40 | 1.64 | 3.51 | 4.50 | 6.89E−06 |
| Residual tumor | null | −0.89 | 0.41 | 0.26 | 0.66 | −3.72 | 1.98E−04 |
| Age | >=67 | 0.72 | 2.05 | 1.40 | 3.00 | 3.69 | 2.24E−04 |
| retinoic | medium risk | 0.66 | 1.94 | 1.35 | 2.77 | 3.62 | 2.92E−04 |
| surv | medium risk | 0.92 | 2.51 | 1.50 | 4.22 | 3.49 | 4.84E−04 |
| retinoic | high risk | 1.02 | 2.78 | 1.49 | 5.18 | 3.21 | 1.31E−03 |
| Residual tumor | No Macroscopic disease | −0.67 | 0.51 | 0.32 | 0.82 | −2.76 | 5.83E−03 |
| Tumor stage | Stages I and II | −1.16 | 0.31 | 0.13 | 0.76 | −2.56 | 1.04E−02 |
| Tumor grade | Grades 3 and 4 | 0.45 | 1.57 | 1.06 | 2.34 | 2.25 | 2.45E−02 |
| Age | 53-66 | 0.40 | 1.49 | 1.04 | 2.13 | 2.16 | 3.08E−02 |
| Primary chemotherapy | STABLE DISEASE | 0.72 | 2.05 | 1.06 | 3.97 | 2.14 | 3.27E−02 |
| Neoplasm status | WITH TUMOR | −0.40 | 0.67 | 0.44 | 1.02 | −1.87 | 6.09E−02 |
| Residual tumor | 11-20 mm | 0.10 | 1.11 | 0.66 | 1.85 | 0.39 | 6.97E−01 |
| Race | Other | 0.10 | 1.10 | 0.65 | 1.87 | 0.36 | 7.19E−01 |
| Residual tumor | >20 mm | 0.04 | 1.05 | 0.73 | 1.50 | 0.24 | 8.11E−01 |

The present invention will now be further described with reference to the following figures which show

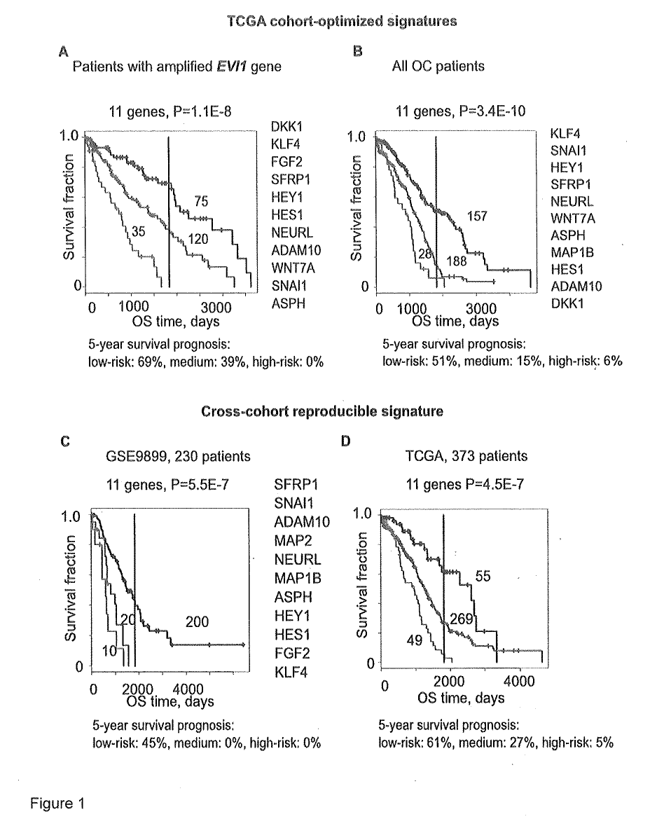

FIG. 1: EMT module of Evi1 pathway. The signature is significant for survival prognosis of the patients with OC tumors. The data represents: A) the TCGA cohort patients with EVI1 amplified in the tumor samples; B) and C) all the TCGA cohort patients; D) the GSE9899 cohort patients. The signatures were optimized for: A) and B) individual cohorts; C) and D) reproducibility across the TCGA and GSE9899 cohorts.

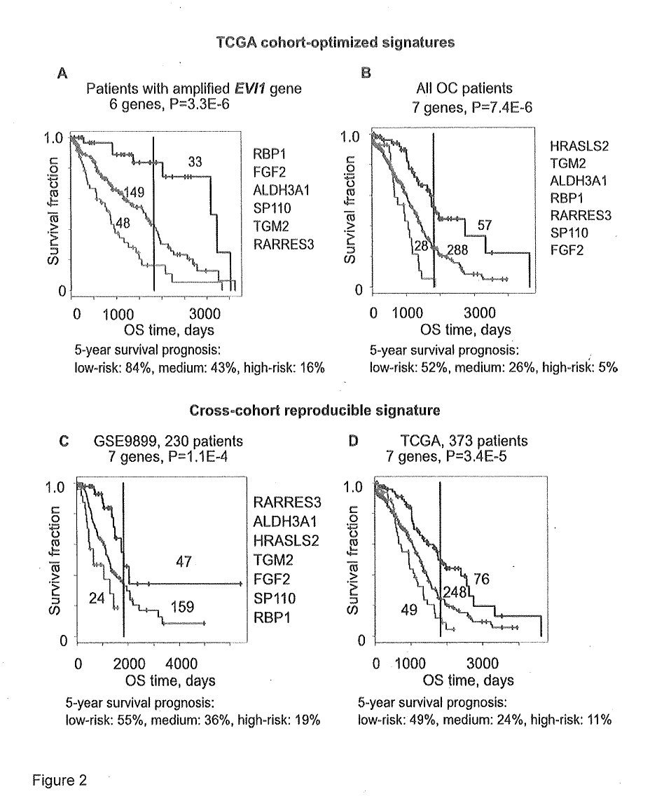

FIG. 2: Retinoic module of Evi1 pathway. The signature is significant for survival prognosis of the patients with OC tumors. The data represents: A) the TCGA cohort patients with EVI1 amplified in the tumor samples; B) and C) the GSE9899cohort patients; D) the TCGA cohort patients. The signatures were optimized for: A) and B) for individual cohorts; C) and D) the GSE9899 cohort.

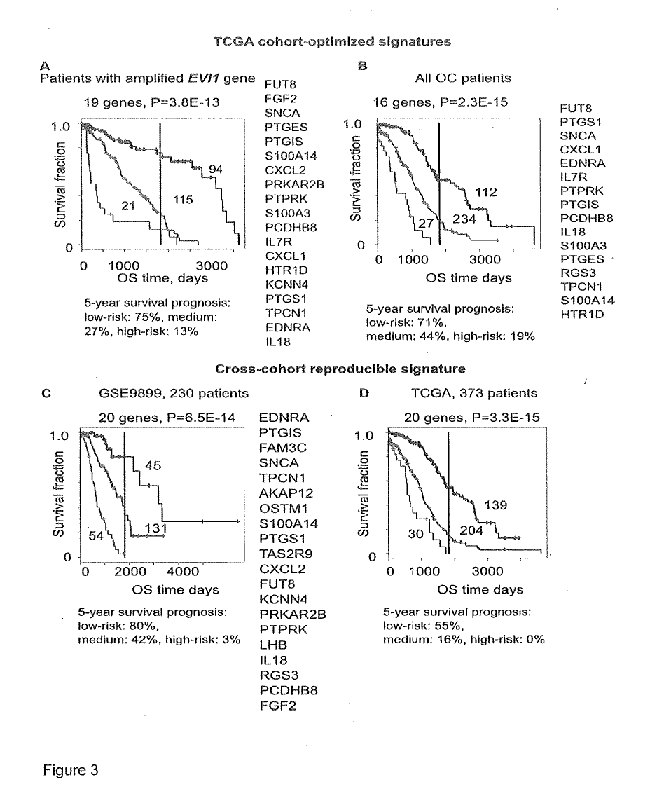

FIG. 3: Signaling module of Evi1 pathway. The signature is significant for survival prognosis of the patients with OC tumors. The data represents: A) the TCGA cohort patients with EVI1 amplified in the tumor samples; B) and C) the GSE9899cohort patients; D) the TCGA cohort patients. The signatures were optimized for: A) and B) for individual cohorts; C) and D) the GSE9899 cohort.

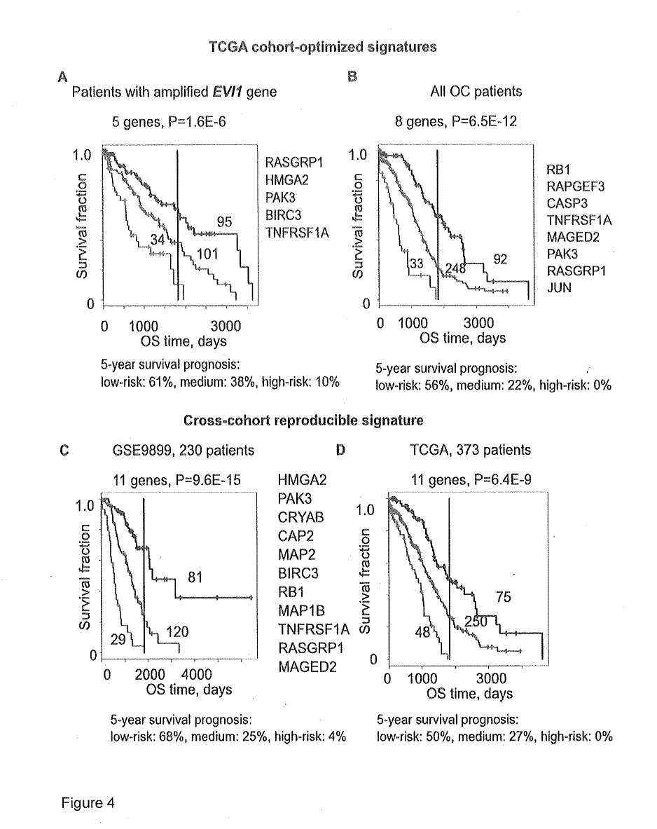

FIG. 4: Apoptosis module of Evi1 pathway. The signature is significant for survival prognosis of the patients with OC tumors. The data represents: A) the TCGA cohort patients with EVI1 amplified in the tumor samples; B) and C) the GSE9899cohort patients; D) the TCGA cohort patients. The signatures were optimized for: A) and B) for individual cohorts; C) and D) the GSE9899 cohort.

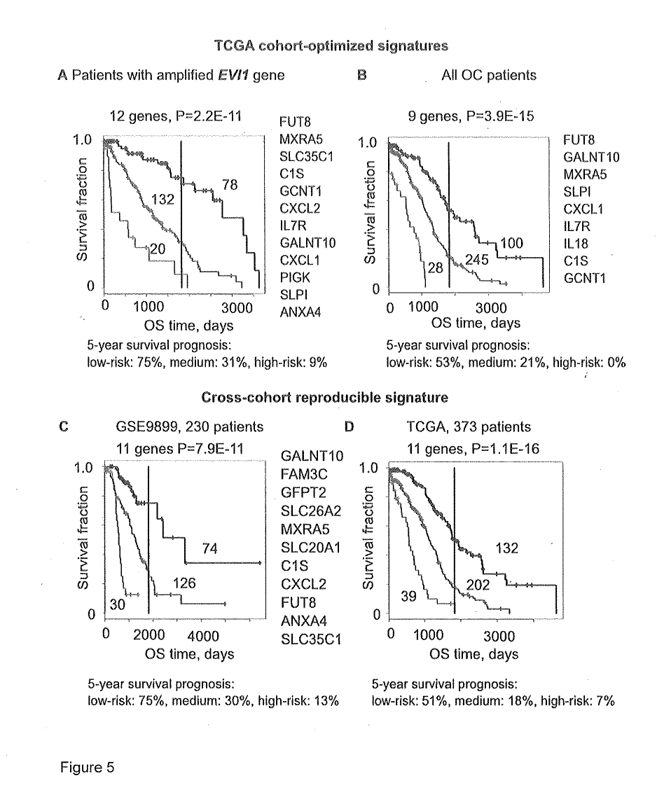

FIG. 5: Immune module of Evi1 pathway. The signature is significant for survival prognosis of the patients with OC tumors. The data represents: A) the TCGA cohort patients with EVI1 amplified in the tumor samples; B) and C) the GSE9899 cohort patients; D) the TCGA cohort patients. The signatures were optimized for: A) and B) for individual cohorts; C) and D) the GSE9899 cohort.

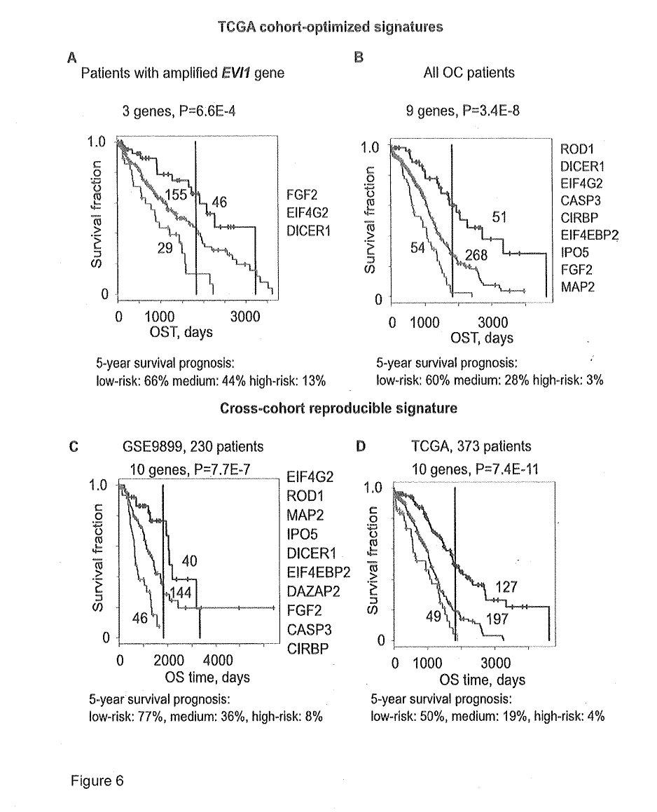

FIG. 6: RNA metabolism module of Evi1 pathway. The signature is significant for survival prognosis of the patients with OC tumors. The data represents: A) the TCGA cohort patients with EVI1 amplified in the tumor samples; B) and C) the GSE9899 cohort patients; D) the TCGA cohort patients. The signatures were optimized for: A) and B) for individual cohorts; C) and D) the GSE9899 cohort.

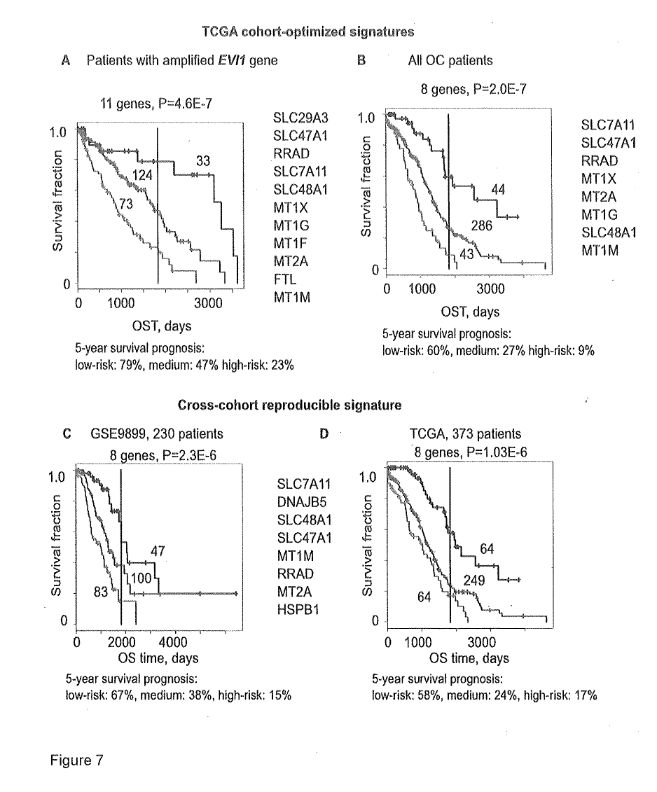

FIG. 7: Survival module of Evi1 pathway. The signature is significant for survival prognosis of the patients with OC tumors. The data represents: A) the TCGA cohort patients with EVI1 amplified in the tumor samples; B) and C) the GSE9899 cohort patients; D) the TCGA cohort patients. The signatures were optimized for: A) and B) for individual cohorts; C) and D) the GSE9899 cohort.

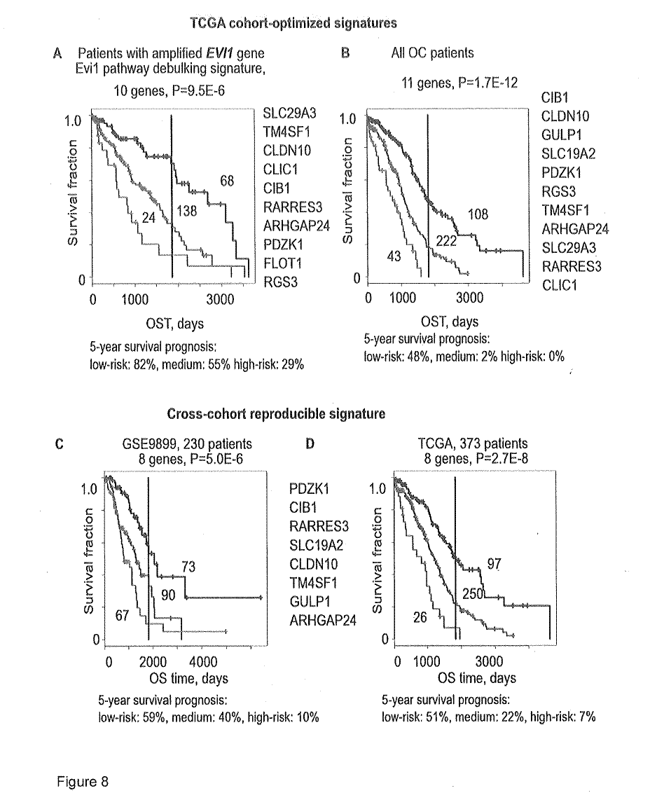

FIG. 8: Debulking-related survival significant module of Evi1 pathway. The signature is significant for survival prognosis of the patients with OC tumors having EVI1 gene amplified (left panel), as well as of all the patients in the TCGA cohort.

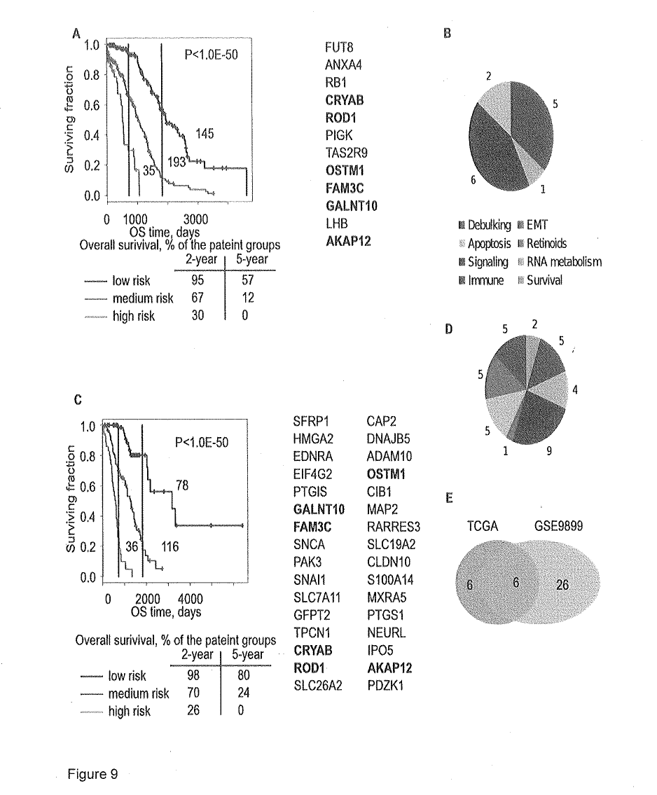

FIG. 9: Signatures composed of the most prognostically significant genes of Evi1 pathway. The signatures were indeplendently optimized for the TCGA (A,B) and the GSE9899 patient cohorts (C and D). The Venn diagram of the genes overlapping between the two signatures is given on panel E.

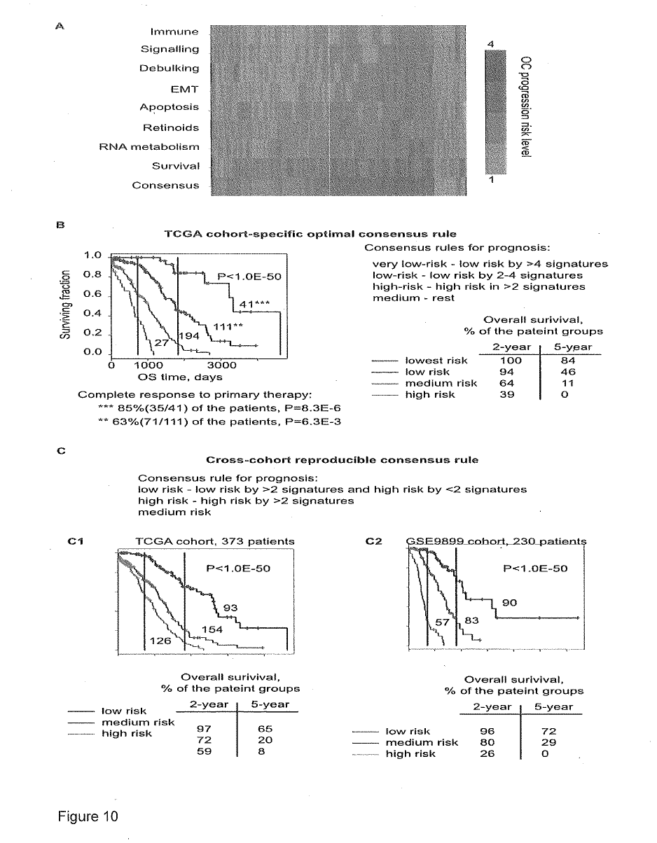

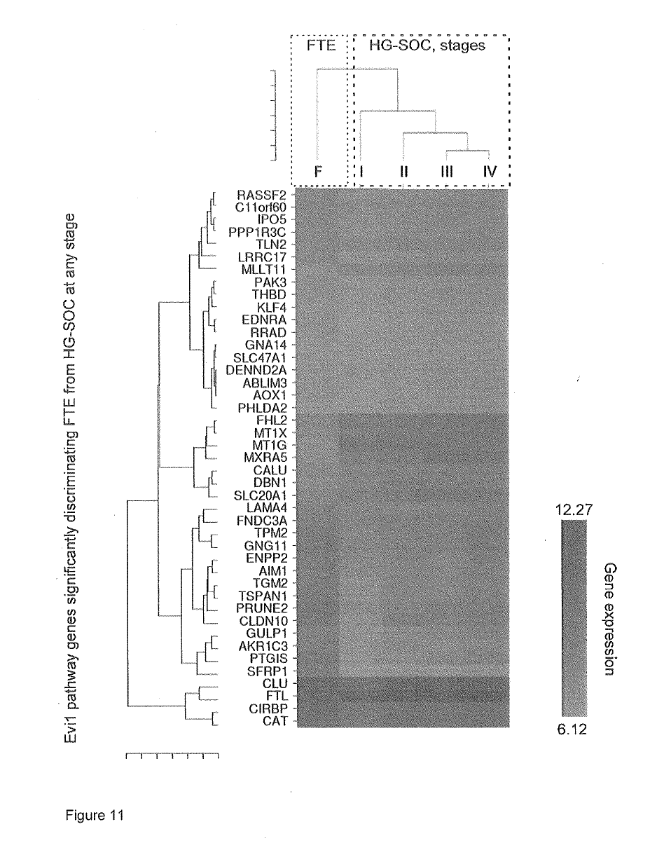

FIG. 10: Separate Evi1 pathway modules can be used as independent prognostic indicators to predict patient survival

FIG. 11: Genes of Evi1 pathway signifcantly discriminating FTE (fallopian tube epithelium) from EOC tumors in the TCGA patient cohort.

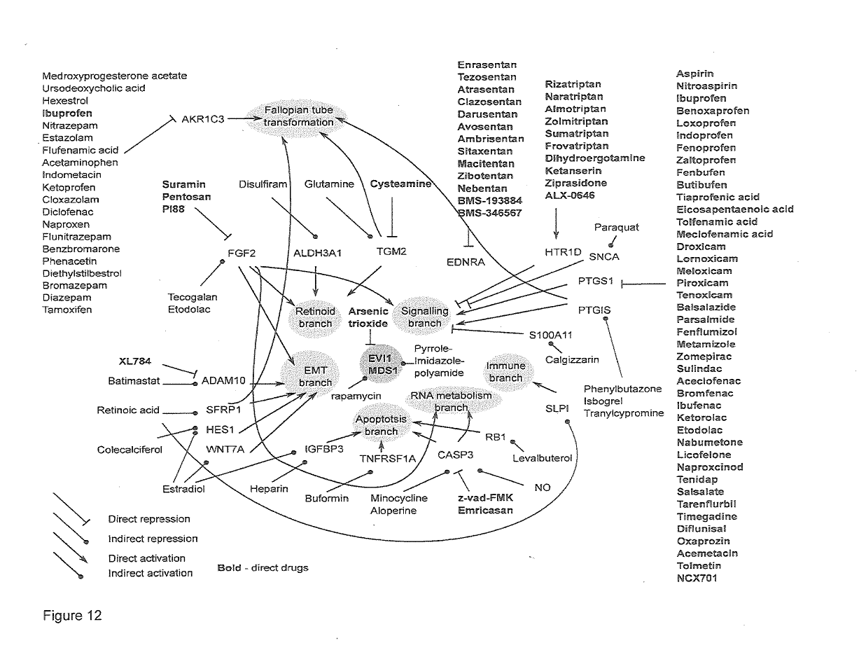

FIG. 12: The DNA motifs specifically bound by Evi1 are the mediators of Evi1 physiological action

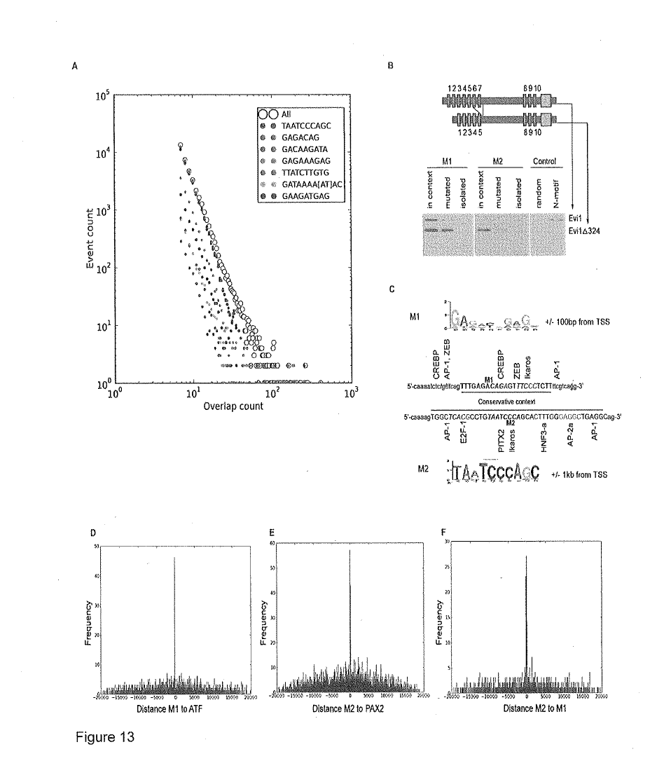

FIG. 13: A) Empirical bi-variate frequency distribution of the threshold level of a gene, which expession level was defined in two (training and testing) datasets, demostrating a consensus level between the paired threshold levels of a given gene sets when all commonly expressed genes of the two dataset are observed. The threshold levels of Evi1 direct target genes are significantly correlated (P=2.2.10-16) in training (the x-axis/cohort 1 threshold) and testing (the y-axis/cohort 2 threshold) cohorts. The two sets of threshold levels are statistically linked via the linear regression function paramerized by slope parameter 0.9401 (C.I. 95%: 0.8153, 1.065) and intercept parameter 1.431 (Cl. 95%: 0.4424, 2.419). The regression model is consided here as the best-fit approximating function of threshold level of a given gene getting consensus between any two given datasets. If both data sets are training, the consenus threshold can be estimated using the parameters of best-fit approximation line to extrapolate the threshold values. B) The algorithm implementating threshold extrapolation onto the best-fit approximation line yielding the consensus threshold points. C) The consensus threshold points (represinting paired threshold levels of a given gene) are displayed on the best-fit approximating line as crossed squares. The process of obtaining the consensus thresholds is the orthogonal projection of the individual threshold points onto the best-fit approximation line. An example of such projection for one given threshold point is shown by a black arrow.

FIG. 14. The method to stratify patients based on Evi1 pathway signature measurements in the samples.

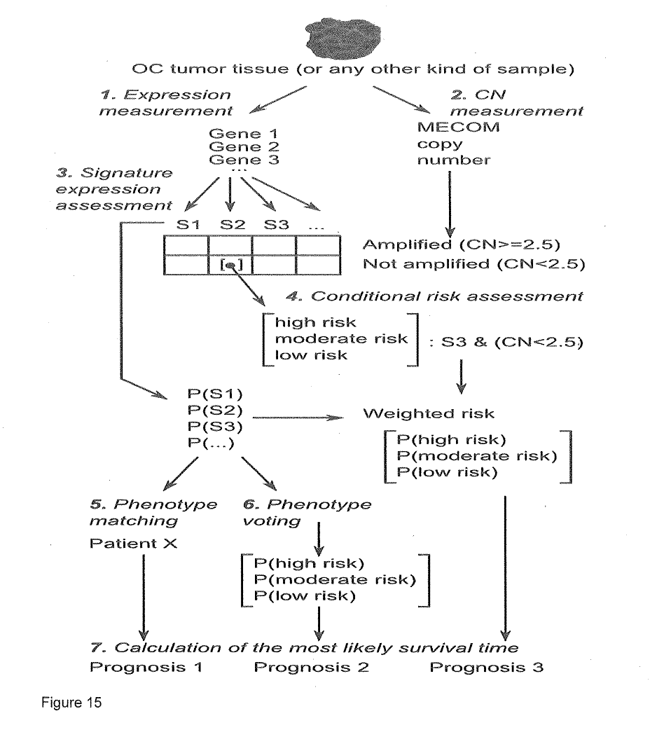

FIG. 15. Illustrates the possibility to provide a combined survival prognosis, based on both expression and copy number measurements of a given gene or a set of genes.

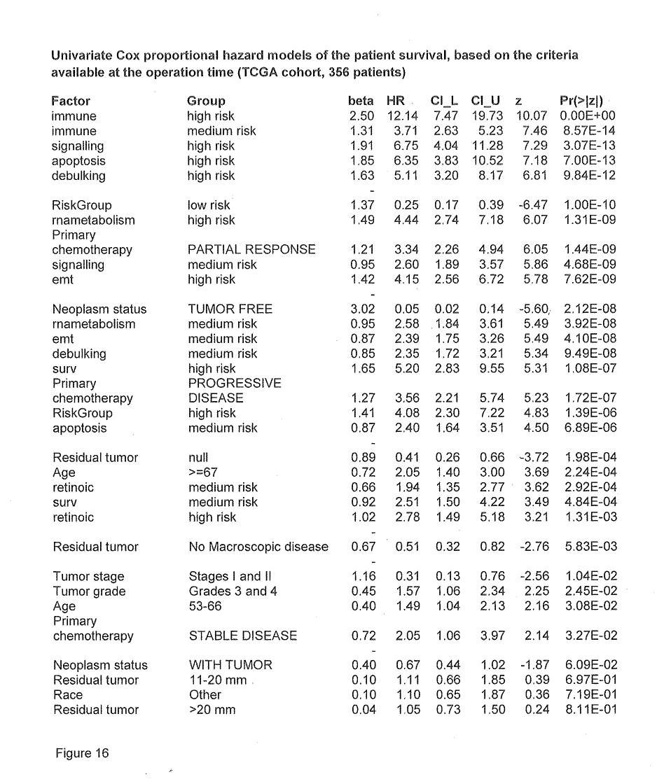

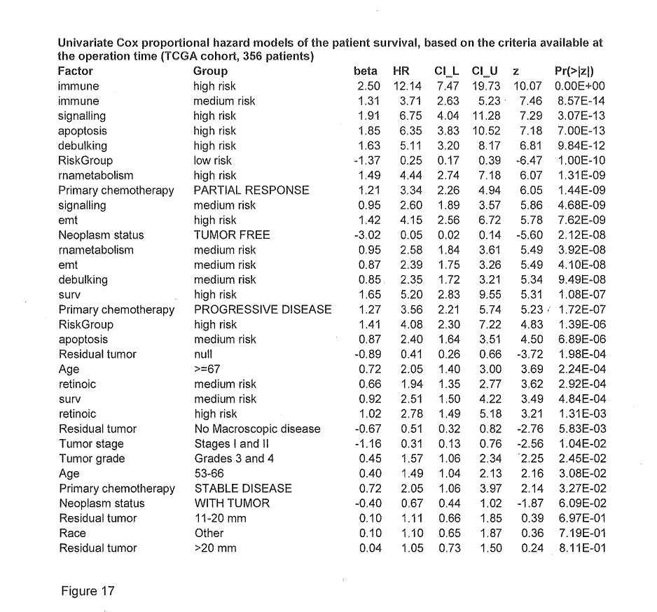

FIGS. 16 and 17. Tables to illustrate univariate Cox proportional hazard models of the patient survival, based on the criteria available at the operation time.

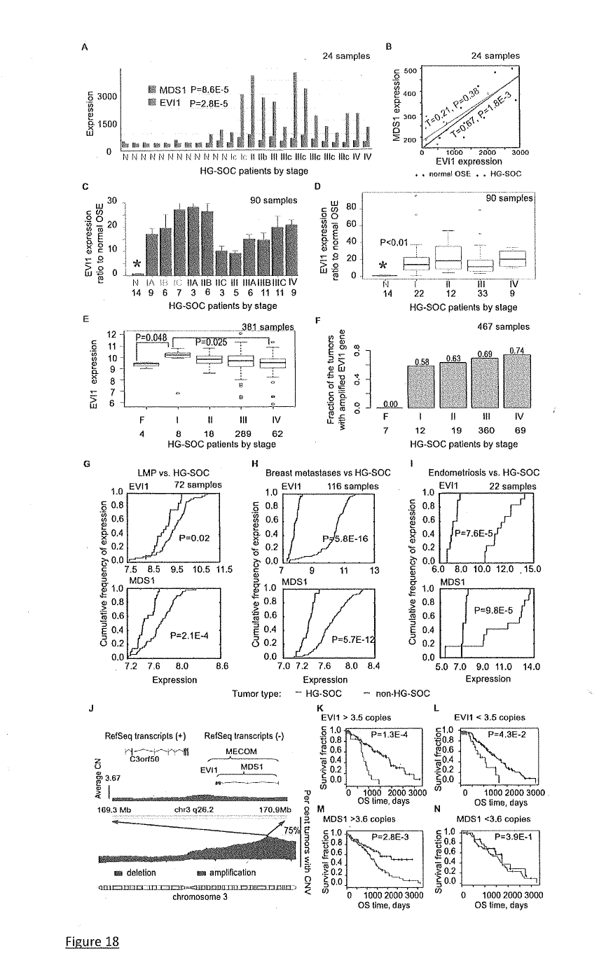

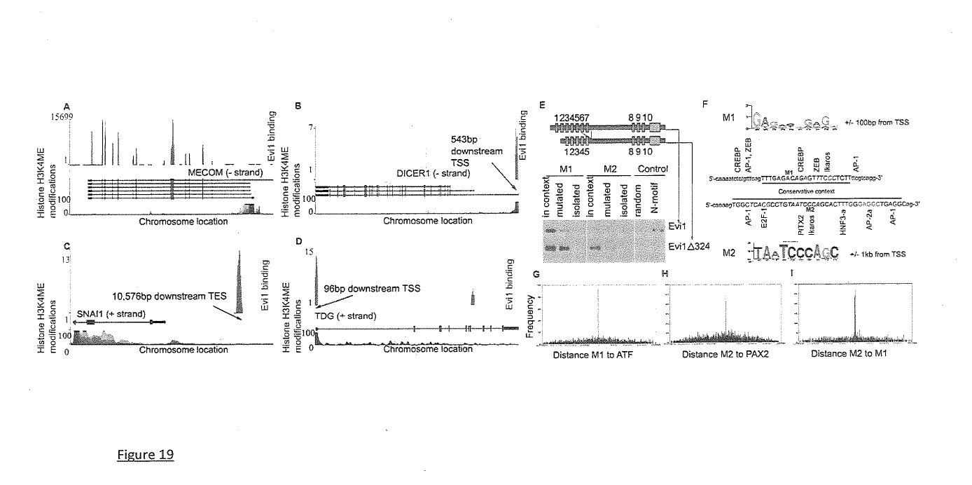

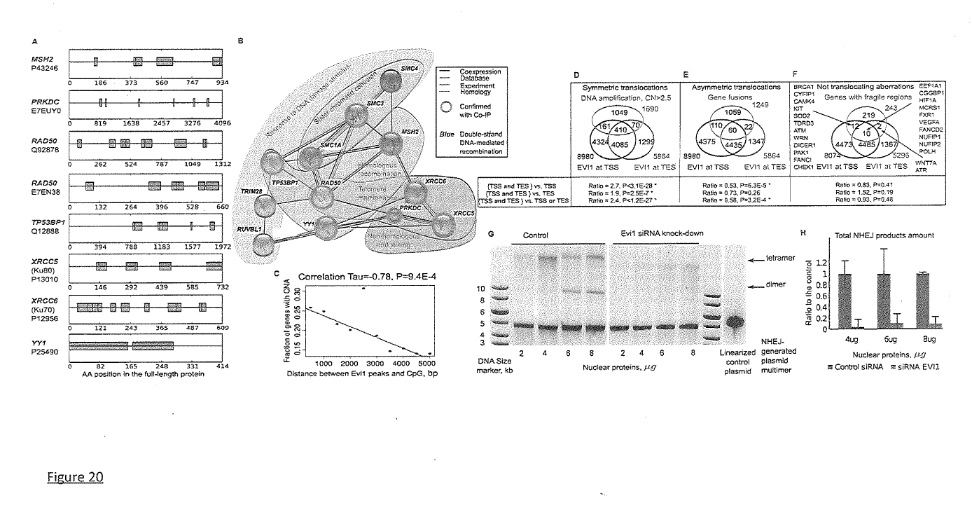

FIG. 18. Diagnostic properties of EVI1 and MDS1 transcripts of the MECOM locus:

A) EVI1 (red bars) and MDS1 (blue bars) expression measured in ovarian surface epithelium (N) and ovarian cancer tumors (stages Ito IV). Dataset GSE14407.

B) EVI1 expression correlates with MDS1 expression, and their combination provides a robust discrimination between ovarian surface epithelium (green dots) and ovarian cancer tumors (red dots). Dataset GSE14407.

C, D) EVI1 expression is significantly different between normal ovarian surface epithelium (N) and any OC tumor of any stage (I-IV) or sub-stage (IA-IIIC).

E) EVI1 expression is significantly different between normal fallopian tube epithelium (F) and stage I OC tumors (I) and between the OC tumors of the first (I) and fourth (IV) stages. Dataset TCGA.

F) The fraction of OC tumors with an amplified MECOM locus (copy number 2.5 and above) increases with OC progression.

G) The expression of EVI1 (above) and MDS1 (below) transcripts is significantly different in ovarian cancer (red line) and low malignancy potential ovarian tumors (blue line). Dataset GSE20565.

H) The expression of EVI1 (above) and MDS1 (below) transcripts is significantly different in ovarian cancer (red line) and breast cancer metastases in ovaries (blue line). Dataset GSE12172.

I) The expression of EVI1 (above) and MDS1 (below) transcripts is significantly different in ovarian cancer (red line) and endometriosis ovarian tumors (blue line). Dataset GSE14407.

J) EVI1 is the most amplified region on chromosome 3. Dataset TCGA.

K-M) The expression of EVI1 (above) and MDS1 (below) transcripts is significantly different in ovarian tumors of patients with less (red line) and more (black line) favorable survival prognoses. The more and less favorable survival prognosis patient groups are defined by the low and high expressions of EVI1 and MDS1, respectively. Dataset TCGA. The significance of the difference between the expression of EVI1 (above) and MDS1 (below) transcripts in ovarian tumors of patients in the two patient groups is higher among patients with high EVI1 and MDS1 copy numbers (more than 3.5 and 3.6 copies of EVI1 and MDS1 genes per cell, respectively). The patient prognosis groups are defined by the low and high expression of EVI1 and MDS1, respectively. The difference between the groups was insignificant if they were defined by MDS1 expression in ovarian cancer tumors with a low MDS1 copy number (less than 3.6 copies per cell).

FIG. 19. Diversity of Evi1 binding sites on DNA and the associated DNA sequence motifs:

A-D) Typical cases of Evi1 binding sites localization in the vicinity of its direct target: exons (A, B); regions downstream the transcription end sites (C); and promoter region at the transcription start sites (D).

E) Both variants of EVI1 transcripts (Evi1 and Evi1Δ324) expressed in SKOV3 cell line specifically bind to both novel motifs of Evi1 (M1 - GAGACAG and M2 - TAATCCCAGC) in their genomic context. No affinity of Evi1 protein to motifs M1 and M2 was observed if they were isolated from the genomic context.

F) The genomic contexts of motifs M1 (above) and M2 (below) used in the present work contain multiple motifs of other transcription factors (shown below and above the sequence).

G- I) - the distribution of pairwise distances between the motifs of Evi1 (M1 and M2) and the motifs of other transcription factors within 20 kb of the Refseq gene promoter regions. The distances are as follows: G) M1 and ATF; H) M3 and PAX2; I) M2 and M1. The distributions demonstrate a peak at the distance between the motifs found in the genome with the highest frequency, suggesting a non-random co-localization.

FIG. 20. Evi1 is involved in induction of copy number alterations in HG-SOC cells:

A) Proteins involved in dsDNA-mediated recombination and repair were identified in Evi1-containing protein complexes using mass-spectrometry (SILAC). Yellow boxes represent the spans of the experimentally obtained peptide sequences that uniquely matched the protein sequences. The matching amino acids (AA) positions in the protein sequences are given in the corresponding abscissa coordinate axes.

B) Evi1-containing protein complexes, which are involved in DNA recombination and repair, were reconstructed from the mass-spectrometry data using STRING database (Jensen et al., 2009). The proteins, whose interactions with Evi1 have been independently confirmed in co-immunoprecipitation (Co-IP) experiments (Bard-Chapeau et al., 2012, 2013), are marked with green circles.

C) In the SKOV3 cells genome, the distance between the Evi1 BSs and CpG islands reveal a strong negative correlation (Tau).

D-F) Symmetric binding of Evi1 to transcription start and end sites significantly co-localizes with symmetric chromosome translocations (D). The asymmetric binding of Evi1 significantly co-localizes with asymmetric chromosome translocations (E). Hot spots for chemical mutagenesis (independent on homologous recombination) have no association with either the symmetric or asymmetric binding of Evi1 (F).

G) Non-homologous end-joining (NHEJ) in vitro assay demonstrates a substantially lower NHEJ activity of the nuclear protein extracts from ovarian cancer cells (SKOV3) with EVI1 transcript knocked-down (red line), in comparison with control cells, where EVI1 expression is high (blue line). The presence of the multimers (here represented by dimers and tetramers) formed from the probe plasmid result from the NHEJ activity in each given nuclear protein extract sample. Nuclear protein extracts of the cells, where EVI1 is knocked down demonstrate very low amount of plasmid multimers, in comparison with the control, thus indicating a substantially lower NHEJ activity in these samples.

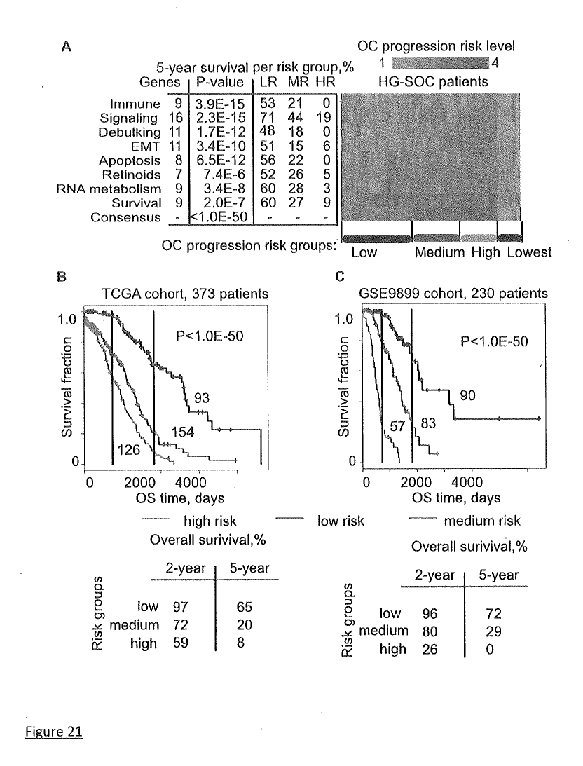

H) The quantification of the NHEJ assay using qPCR with primers specific to the plasmid multimers resulting from NHEJ activity of the nuclear protein extracts. The amount of the PCR product is directly proportional to the amount of NHEJ product. FIG. 21. The Evi1 pathway is a strong prognostic biomarker of HG-SOC:

A) Eight individual gene signatures are constructed from the genes of each of the 8 Evi1 pathway branches (immune, signaling, debulking, EMT, apoptosis, retinoic acid metabolism, RNA metabolism and tumor survival). Each signature classifies the patients by the OC progression risk into 3 groups (low, medium, and high risk, with risk level scores of 1, 2 and 3, respectively).

B-C) The consensus signature is derived as the result of the independent voting of the 8 signatures with the risk level scores to classify the patients into four groups according to the OC progression risk level using the following rules: level 1, low-risk by more than 4 signatures; level 2, low-risk by 2-4 signatures; level 4, high-risk by more than 2 signatures; level 3, the remaining patients. The consensus signature optimal for the TCGA dataset (B) reveals a strong significance for patient prognosis prediction in the GSE9899dataset (C).

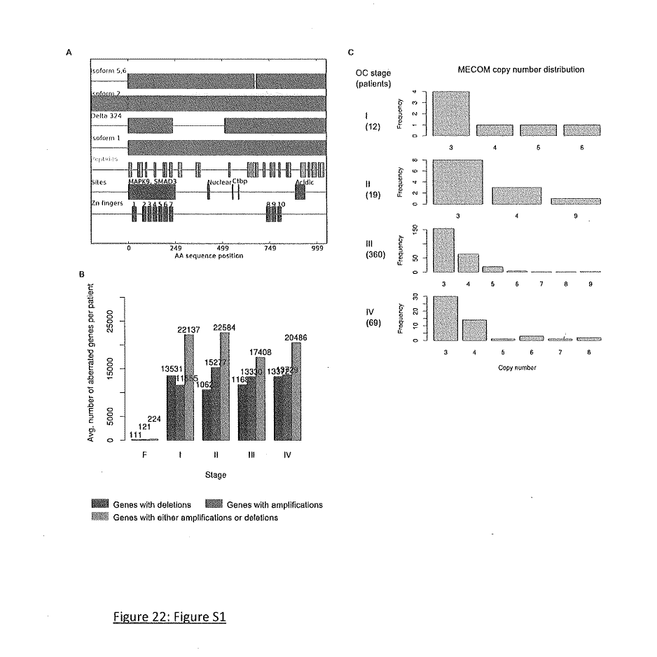

FIG. 22: Figure S1:

A) Structural and functional features of Evi1 protein mapped onto its sequence reveal the isoforms of Evi1 protein expressed in SKOV3 cells Protein sequences of four major isoforms of Evi1 are presented in red color, top to bottom: Δ324, Isoforms 5, 6 and 2 (MDS1/Evi1 protein). Evi1 protein sequence features are presented in blue color, top to bottom: Sites of protein-protein interactions and Zn-finger domains. Peptides identified by sequencing of SKOV3 proteome with tandem mass-spectrometry are presented in green color.

B) Number of genes (RefSeq, relase 58) with deletions and amplifications in OC tumors (stages I-IV), compared to fallopian tube epithelium (F).

C) Distribution of tumors by copy number of MECOM per cell for OC stages (I-IV).

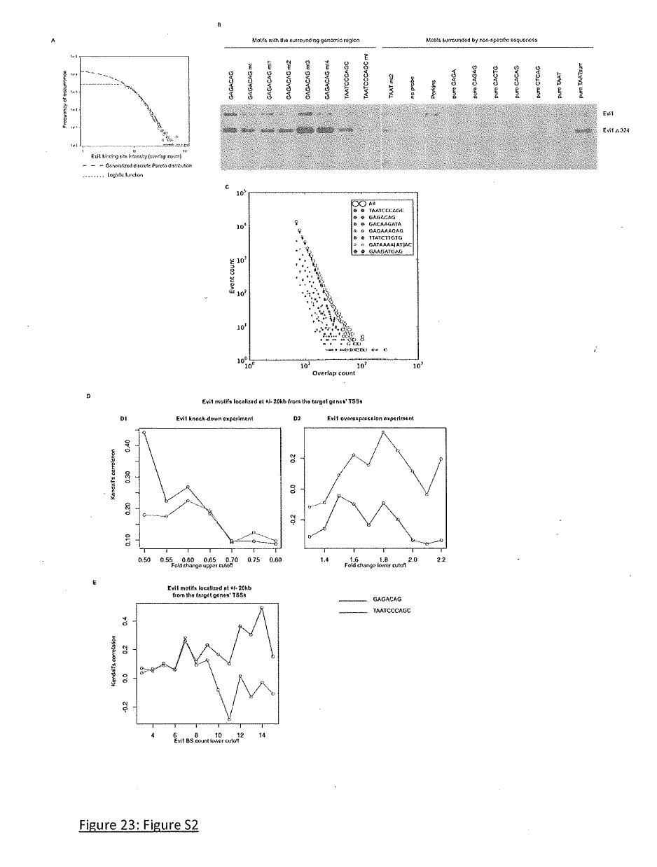

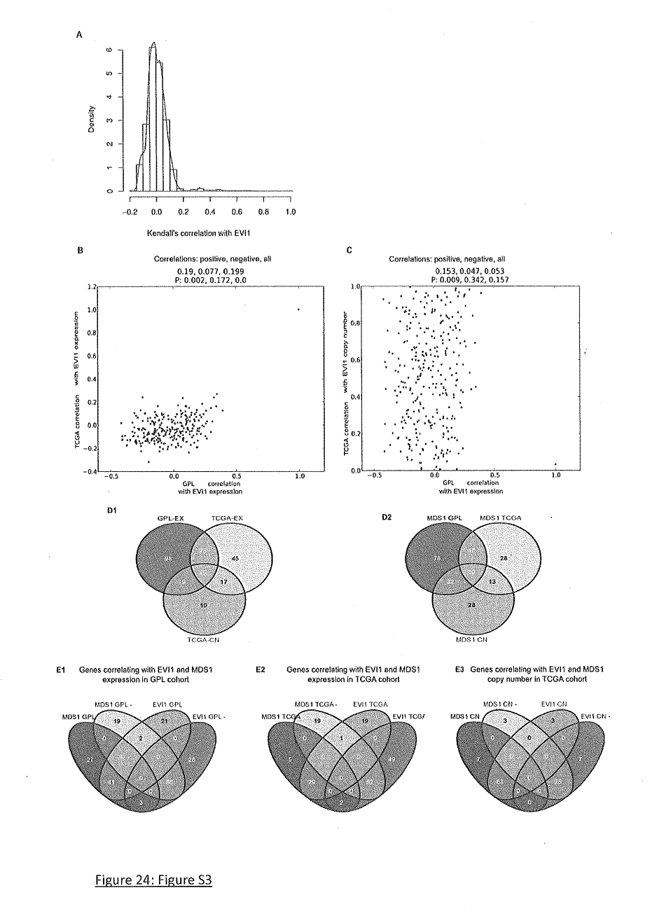

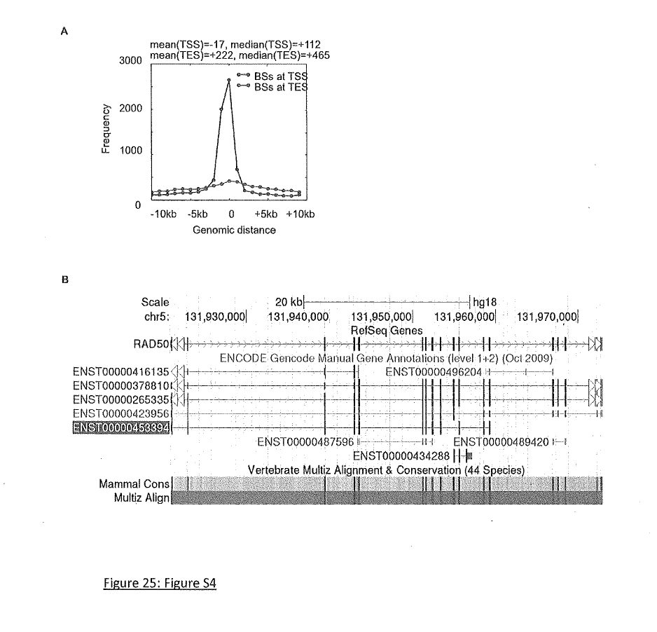

FIG. 23: Figure S2:

(The values of gene copy number and expression values in OC cells correlate with those of HG-SOC tumors):

A1 ,A2) Mean copy numbers of the genes most amplified in HG-SOC tumors (ordinate) is plotted versus their mean copy number values in SKOV3 cells (abscissa). Kendall's Tau correlation and their P-values are presented above the graphs. A1 and A2 represent the data for the top 10 and the top 100 genes respectively.

A1-B4) Mean expression value of the genes most highly expressed in HG-SOC tumors (ordinate) is plotted versus their mean copy number values in SKOV3 cells (abscissa). Kendall's Tau correlation and their P-values are presented above the graphs. B1, B2, B3 and B4 represent the data for the top 100, 300, 1000 and 2000 genes respectively.

FIG. 24: Figure S3: (Novel DNA motifs specifically bound by Evi1 in human genome):

A) Avidity distribution of all Evi1 BSs (according to T2G algorithm) in log-log scale. Number of ChIP-seq sequences overlapping in each cluster (abscissa) versus frequency (ordinate).

B) Validation of Evi1 binding specificity of DNA motifs GAGACAG (M1) and TAATCCCAGC (M2) by an electrophoretic mobility assay with Evi1 pulled-down with anti-Evi1 antibody pull-down. Left panel - the motifs (with and without mutations) cloned with the genomic region surrounding them; right panel - the motifs (with and without mutations) inserted into a DNA fragment with a simple repetitive sequence (, where M stands for a specific motif sequence).

C) Distribution of the number of Evi1 BSs (by ChIP-seq overlap count) in the genomic regions containing motifs of the given types within +/−20kb neighborhood from the ChIP-seq overlap centers. Abscissa axis - the number of overlaps in the BSs, ordinate axis - the number of the BSs neighboring the motifs (displayed in the legend box of the graph). “All” marks the distribution of all the Evi1 BSs regardless of the presence of the motifs in the neighborhood.

D1, D2) Correlation between the frequency of Evi1-specific DNA motifs (red line—M1, GAGACAG; blue line—M2, TAATCCCAGC) in the +/−20 kb vicinity from Evi1 target genes TSSs and their expression. The Evi1 target genes were identified in the EVI1 knock-down (D1) and overexpression (D2) experiments. The genes were filtered by the fold change of their differential expression in the respective EVI1 perturbation experiment. The fold change was calculated as the ratio between the median gene expression in the samples with the perturbation to the one in the control samples. The genes with the fold change lower (D1) or higher (D2) of a certain cutoff value (the abscissa) were selected, and the correlation coefficient (the ordinate) was calculated for the given set of genes.

E) Correlation between the frequency of Evi1-specific DNA motifs (red line - M1, GAGACAG; blue line—M2, TAATCCCAGC) in the +/−20 kb vicinity from Evi1 target genes TSSs and the frequency (count) of the Evi1 BSs in this region. The analysis was similar to the one displayed on D1 and D2, where the target genes were selected by the lower cutoff of the BSs count (the abscissa).

FIG. 25: Figure S4: (Evi1 (probeset 221884_at) and MDS1 (probeset 208434_at) genes expression and copy number in clinical data):

A) Distribution of Kendall's correlation coefficients of EVI1 with all other probesets in the tumors of TCGA patient cohort. The distribution is normal with mean correlation 0 and a right tail τ>0.2 of positively correlating probesets.

B) A correspondence (Kendall's correlation) between the correlation coefficients of EVI1 expression with the expression of other probesets in GSE20565 patient cohort (the abscissa axis) and the ones in the TCGA patient cohort (the ordinate axis). The relationship is significant for the all probesets (τ=0.20,P<0.001) and the probesets positively correlating with EVI1 (τ=0.19,P=0.002).

C) A correspondence (Kendall's correlation) between the correlation coefficients of EVI1 expression with the expression of other probesets in GSE20565 patient cohort (the abscissa axis) and the correlation between the EVI1 copy number with the expression of all other probesets in TCGA patient cohort (the ordinate axis). The relationship is significant for the probesets positively correlating with EVI1 copy number (τ=0.15,P=0.009).

D1, D2) Venn diagrams of the probesets, which expression correlates with the expression of EVI1 (D1) or MDS1 (D2) in the tumors of GSE20565 (blue oval) and the TCGA (yellow oval) patient cohorts, or with EVI1 or MDS1 copy number in the TCGA cohort (green oval).

E1) Venn diagrams of the probesets, which expression correlates with EVI1 or MDS1 expression in the tumors of GSE20565 patient cohort. Blue oval—positive correlation with MDS1; yellow oval—negative correlation with MDS1; green oval—positive correlation with EVI1; red oval—negative correlation with EVI1;

E2) Venn diagrams of the probesets, which expression correlates with EVI1 or MDS1 expression in the tumors of the TCGA patient cohort. Blue oval—positive correlation with MDS1; yellow oval—negative correlation with MDS1; green oval—positive correlation with EVI1; red oval—negative correlation with EVI1;

E3) Venn diagrams of the probesets, which expression correlates with EVI1 or MDS1 copy number in the tumors of the TCGA patient cohort. Blue oval—positive correlation with

MDS1; yellow oval—negative correlation with MDS1; green oval—positive correlation with EVI1; red oval—negative correlation with EVI1;

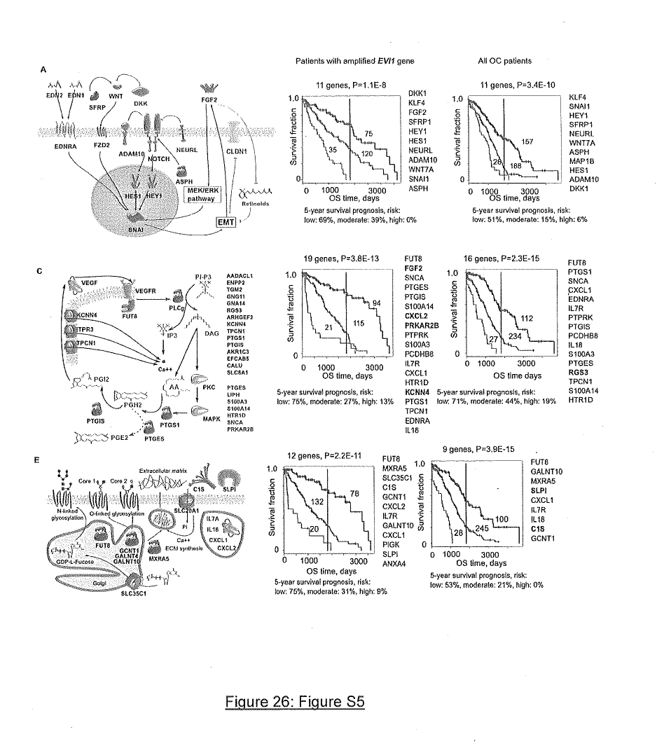

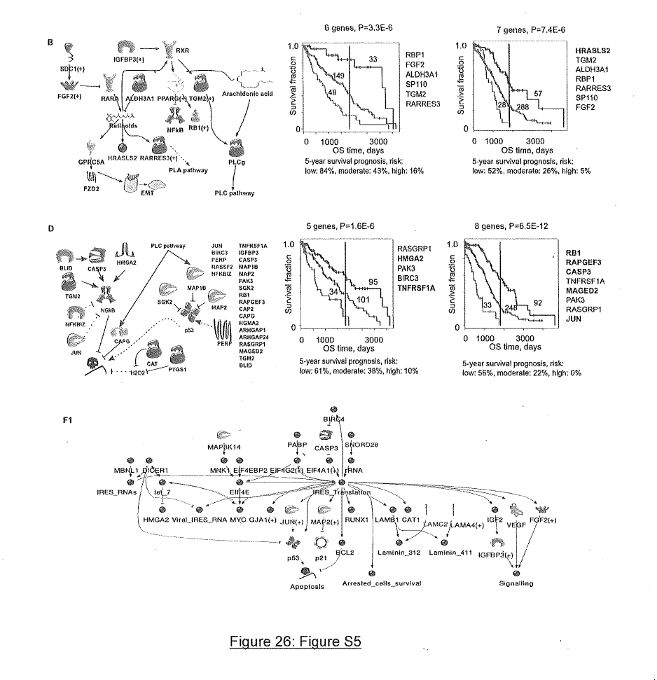

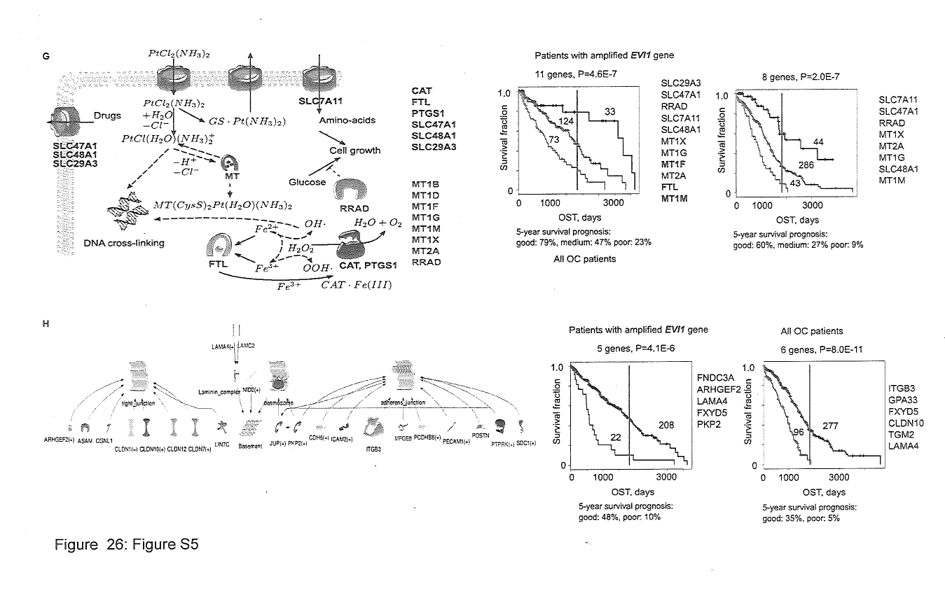

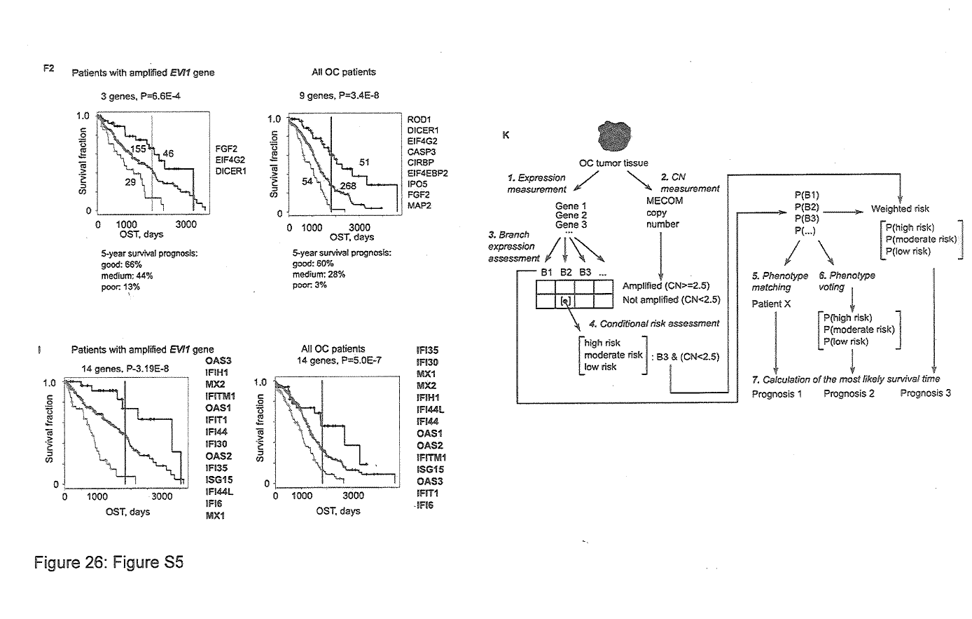

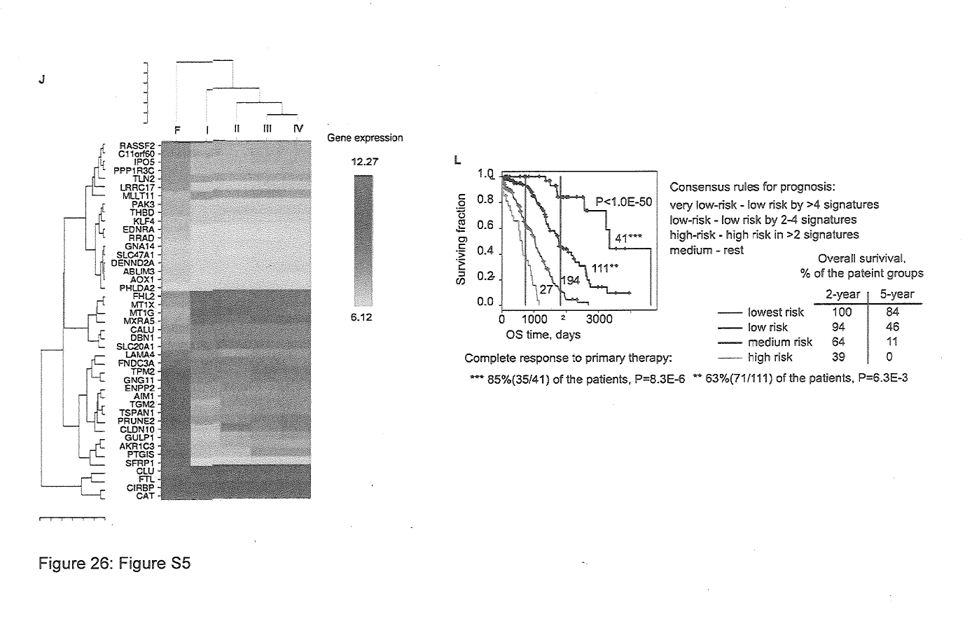

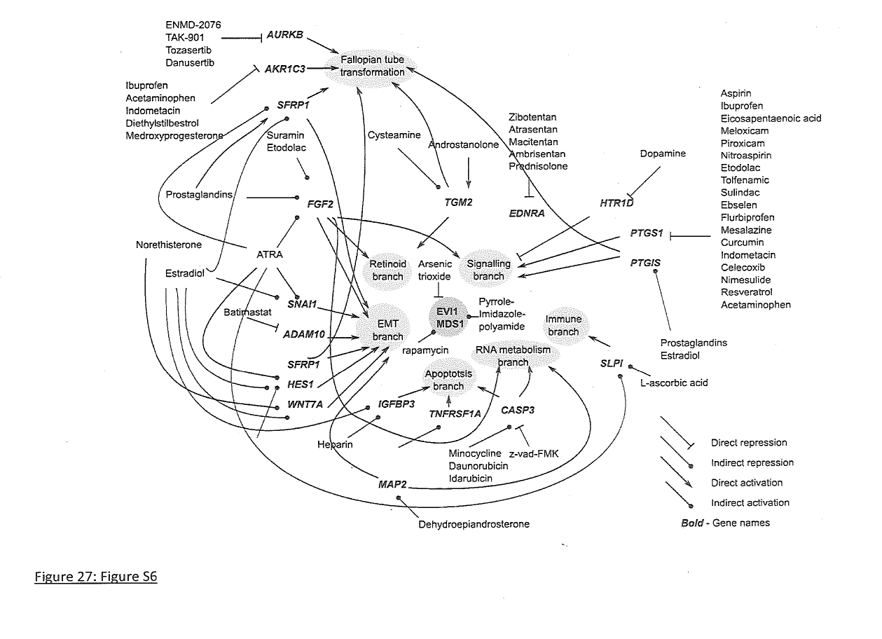

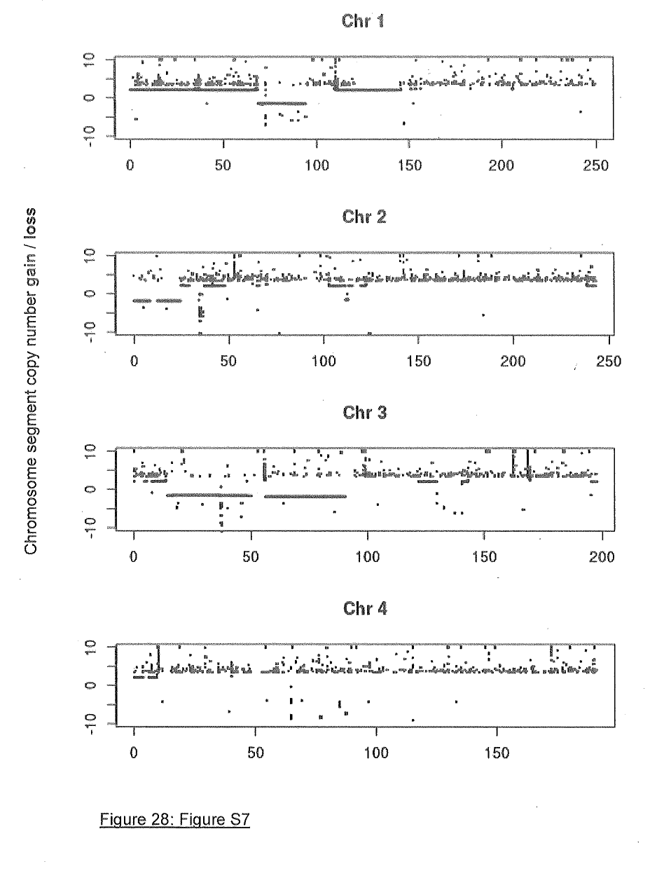

FIG. 26: Figure S5: A) Distribution of Evi1 BSs around TSS

FIG. 27: Figure S6: Additional branches of Evi1-dependent regulatory pathway driving OC progression

A-E)—the most important branches of the pathway. Each branch is illustrated with a simplified model (based on protein-protein interactions reported in the literature) , where genes activated and repressed by Evi1 are highlighted with blue and red color respectively. Activating and repressing actions in the regulatory pathways are shown with blue arrows and red T-arrows respectively. The clinical significance of each branch is demonstrated on a pair of survival graphs for the TCGA cohort patients: left —of the patients with tumors with not less than 2.5 copies of EVI1 per cell; right —all the patients in the TCGA cohort. The genes, belonging to each given branch, stratifying the patients for each patient group are given at the right of the survival graph. Genes unique for one of the two patient groups are highlighted with bold font.

F1, F2) A schema (A1) and the results of the survival analysis of the gene signatures (A2) of the RNA metabolism branch of Evi1 pathway.

G1, G2) A schema (B1) and the results of the survival analysis of the gene signatures (B2) of the cell survival branch of Evi1 pathway.

H1, H2) A schema (C1) and the results of the survival analysis of the gene signatures (C2) of the cell contact branch of Evi1 pathway.

I) The results of the survival analysis of the gene signatures of the anti-viral response branch of Evi1 pathway.

J) The 43 genes, whose expression is significantly (P<0.05) different in fallopian tubes (F), in comparison with OC tumors at each of the four stages (I-IV).

K) Pathway-centric survival analysis workflow.

L) B) The consensus signature stratifies the patients into four groups by survival prognosis according to the rules: 1) a lowest-risk group if the patient was classified as low-risk by more than four branch-specific signatures; 2) a low-risk group if the patient was classified as low-risk by two to four signatures; 3) a high-risk group if the patient was classified as high-risk by more than two branch-specific signatures; 3) otherwise, the patient was classified as medium risk. For the groups with good and very good prognosis, the signature can predict more than 60% of the complete response occurrence.

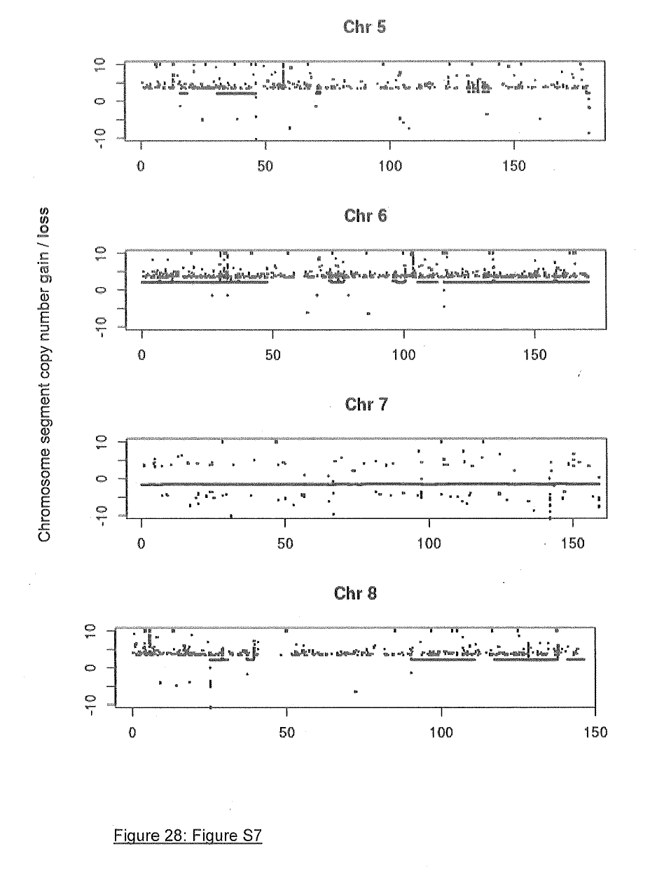

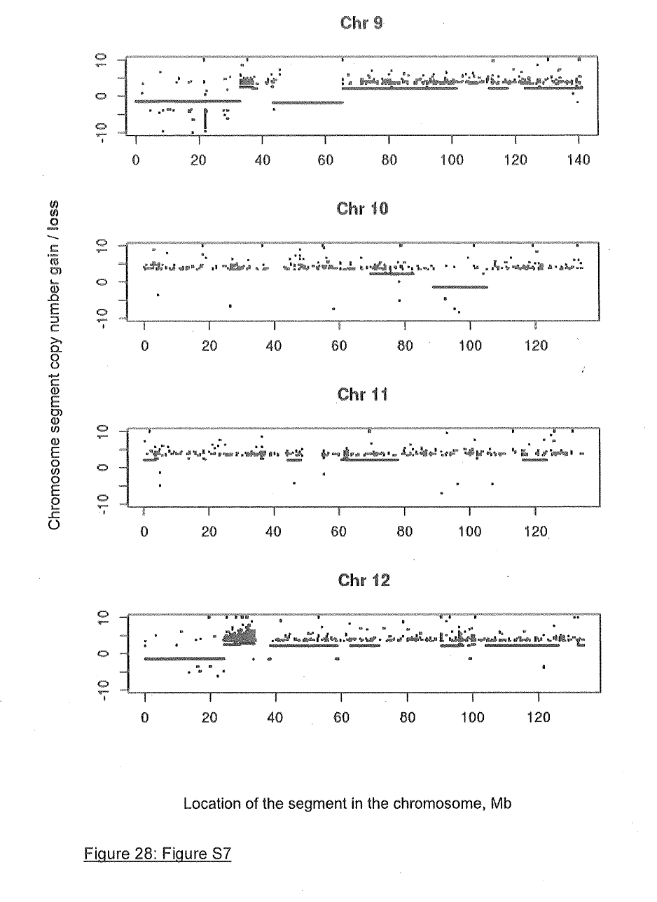

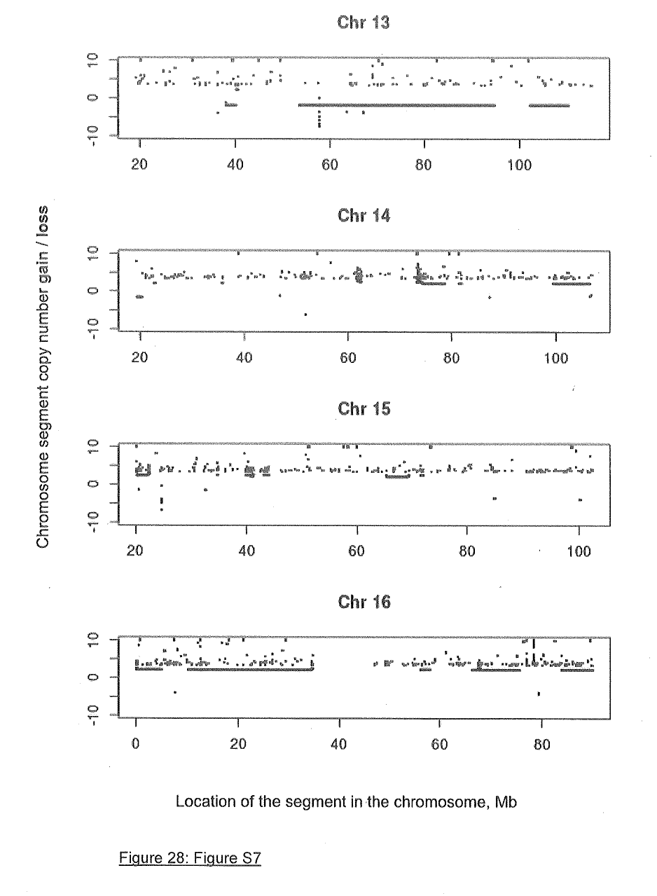

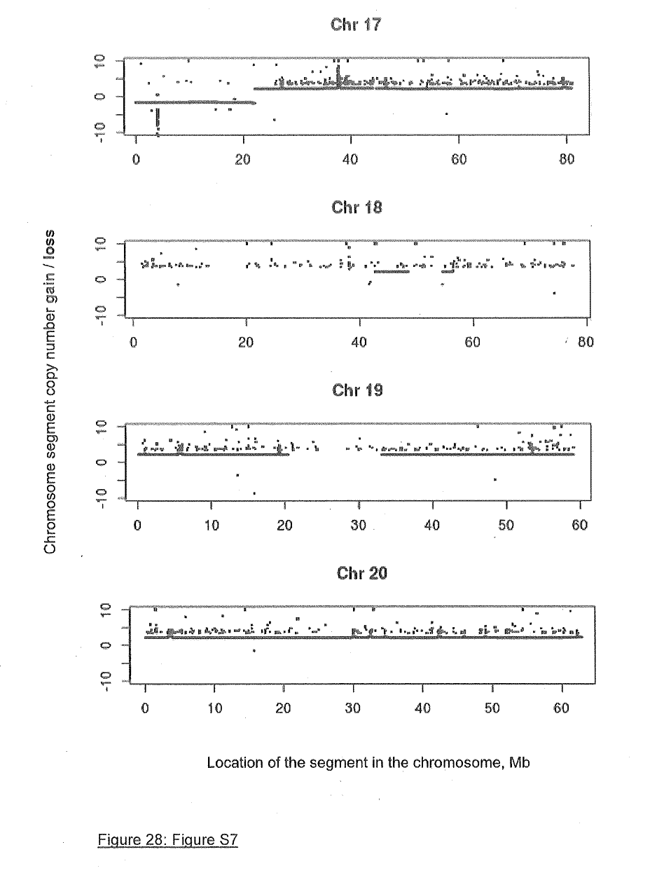

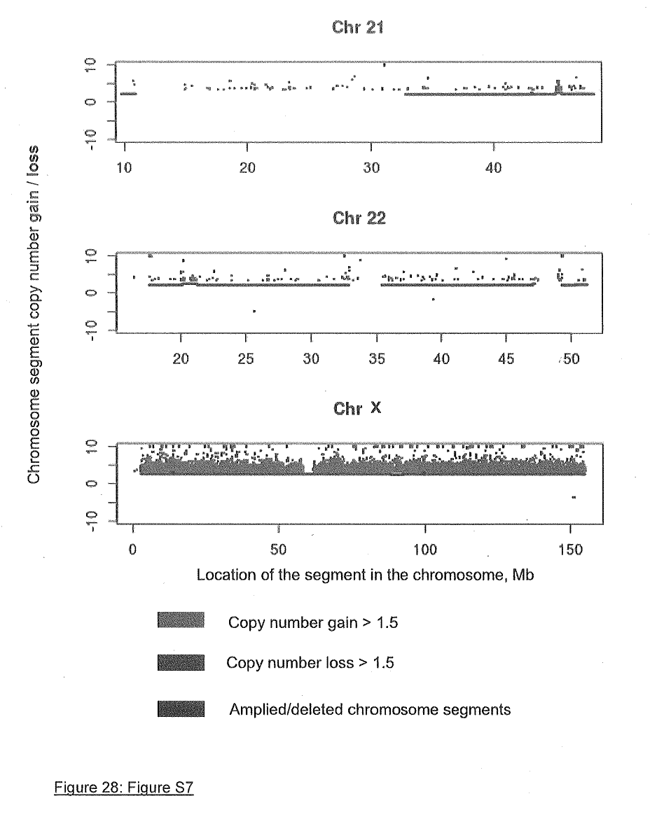

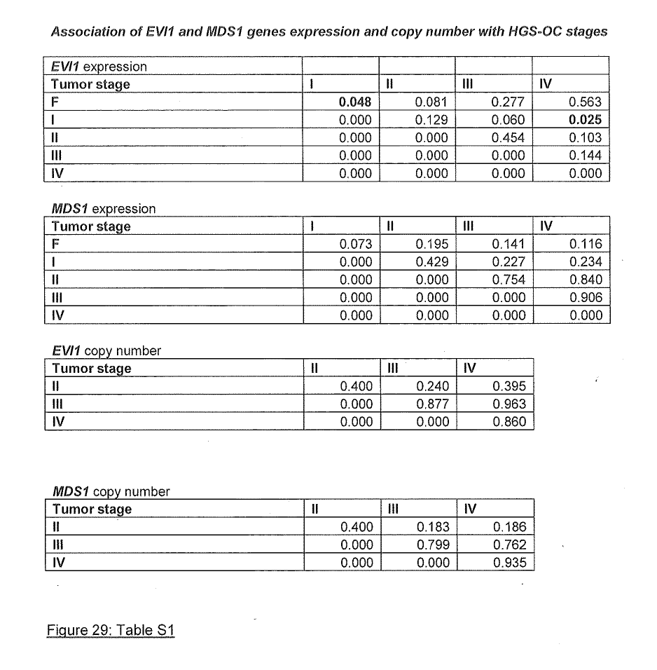

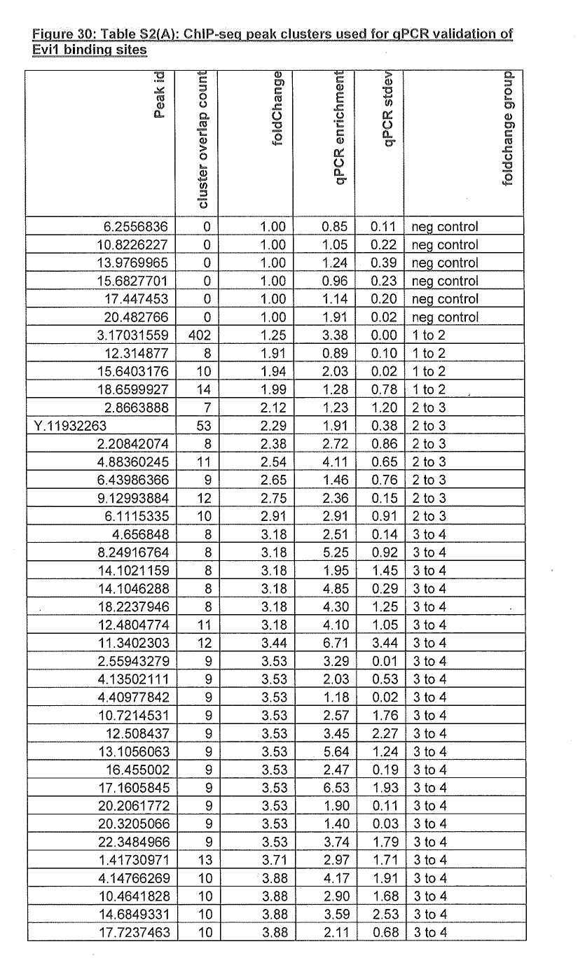

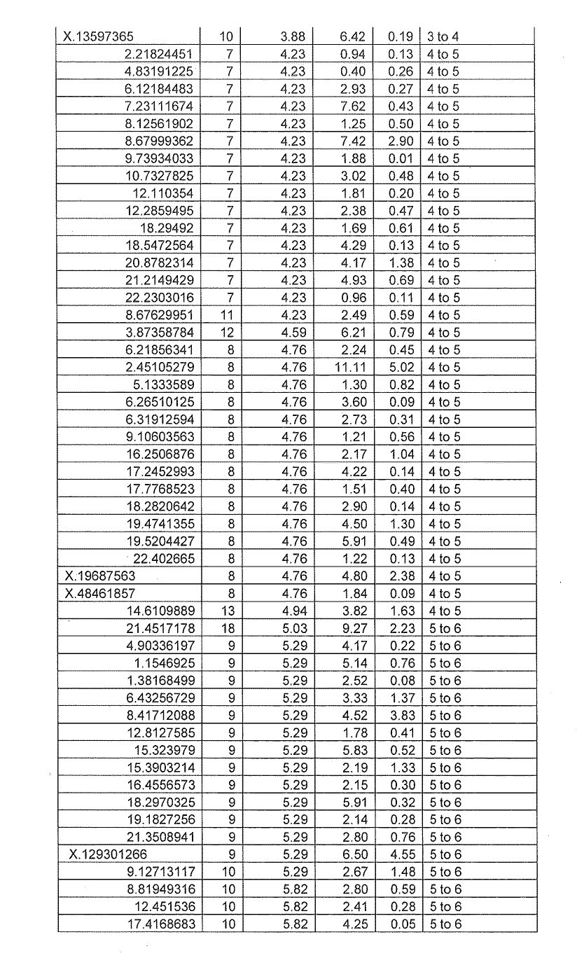

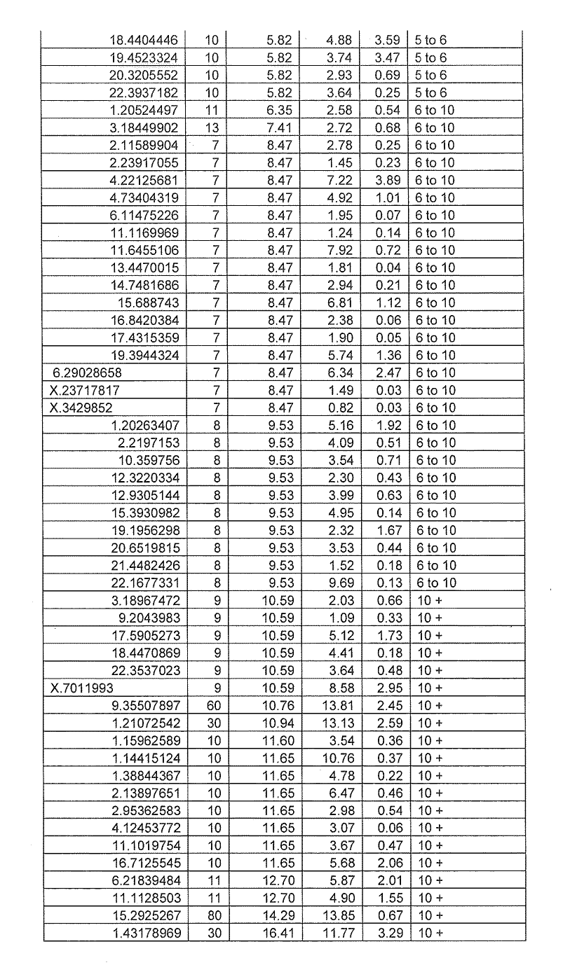

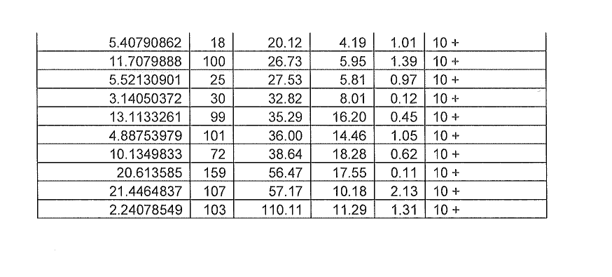

FIG. 28: Figure S7: Therapeutic agents targeting Evi1, Mds1 and the members of Evi1 pathway

FIG. 29: Table S1: Association of EVI1 and MDS1 genes expression and copy number with

HGS-OC stages

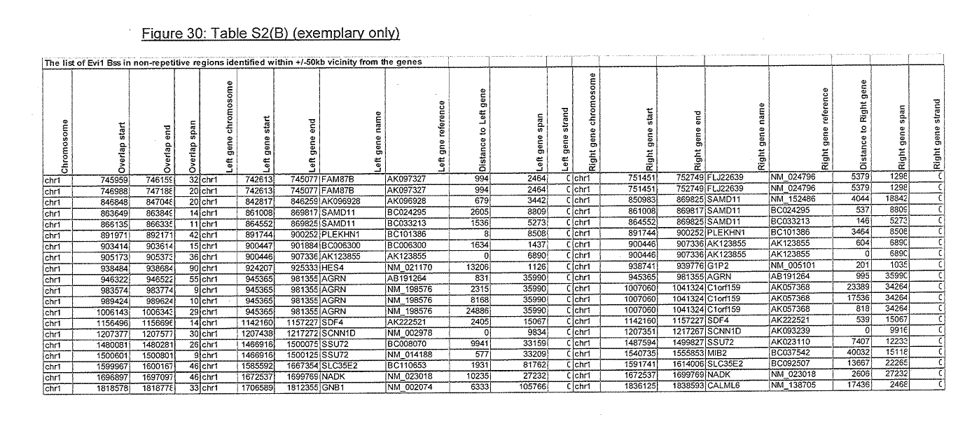

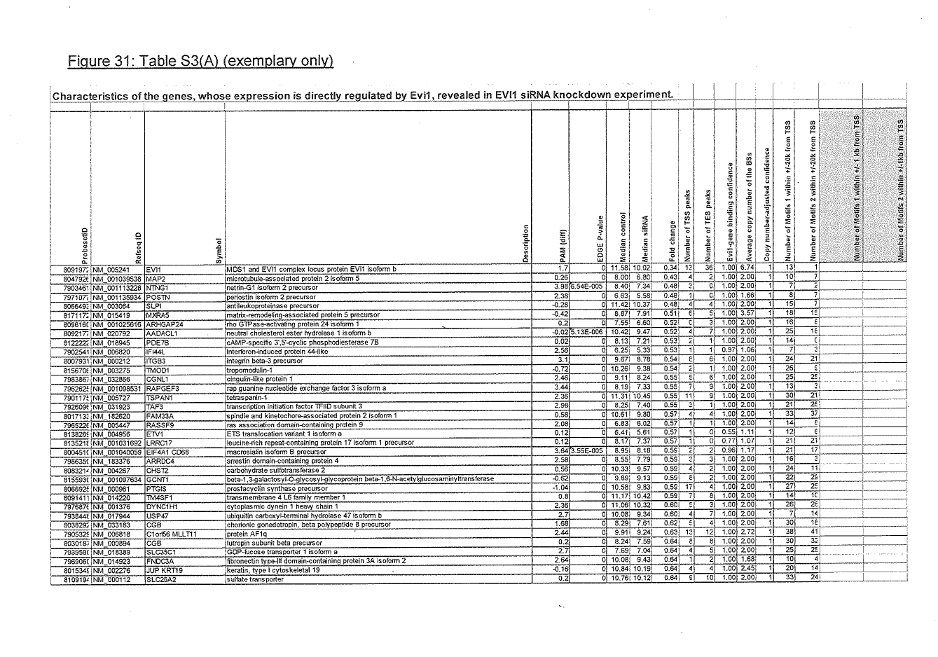

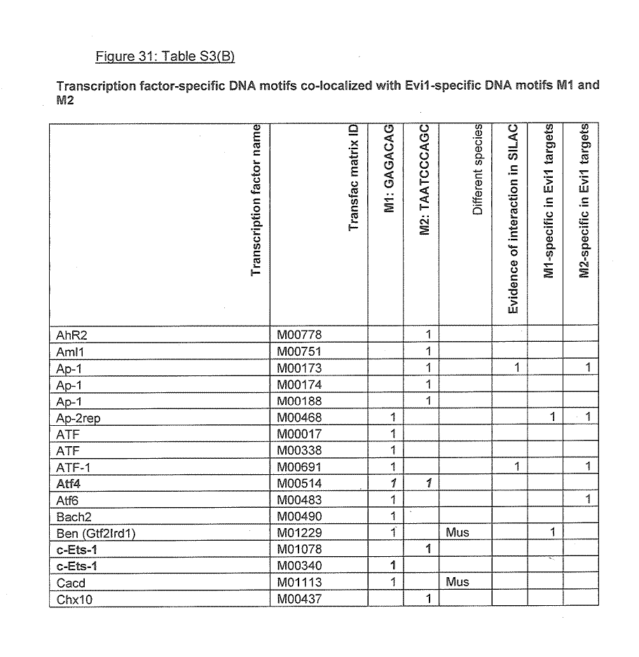

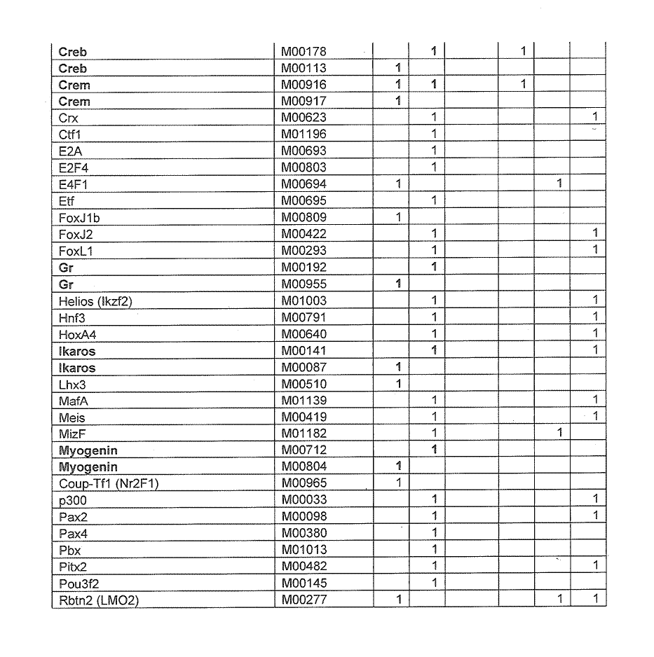

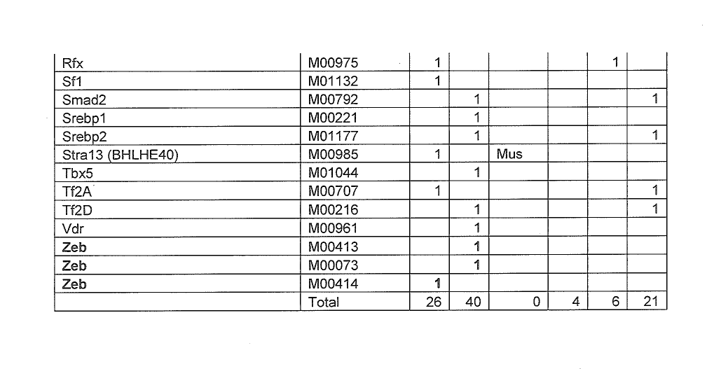

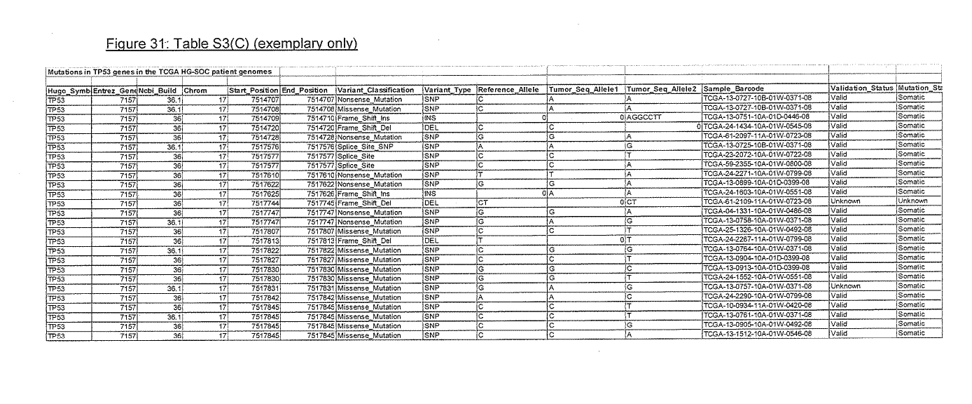

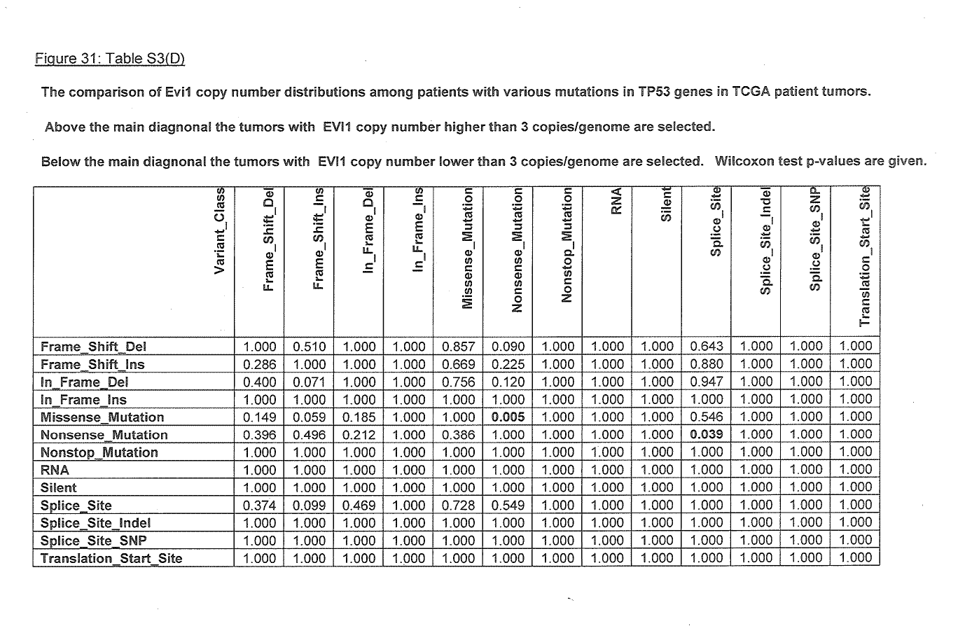

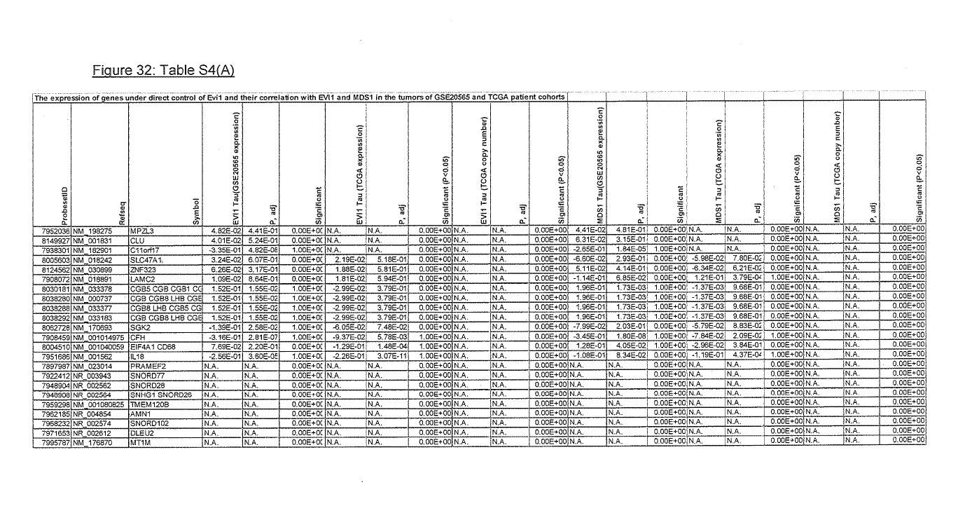

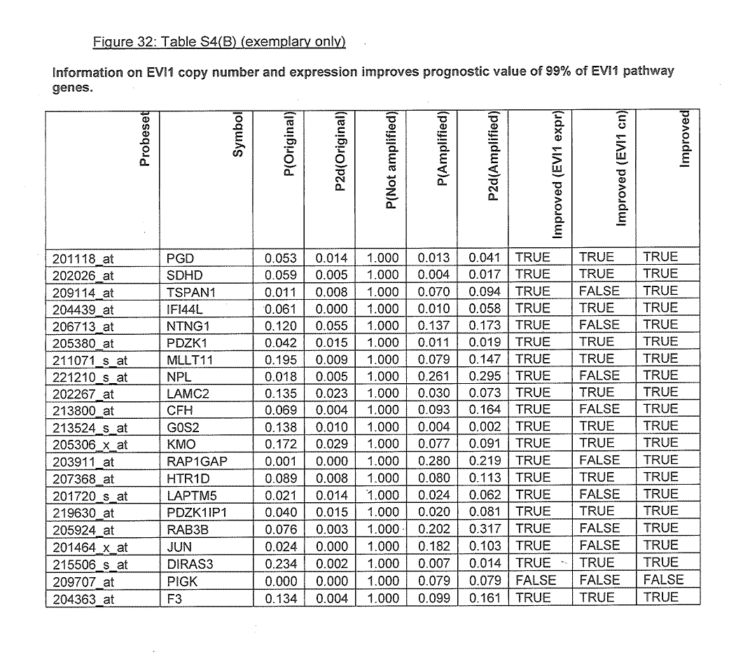

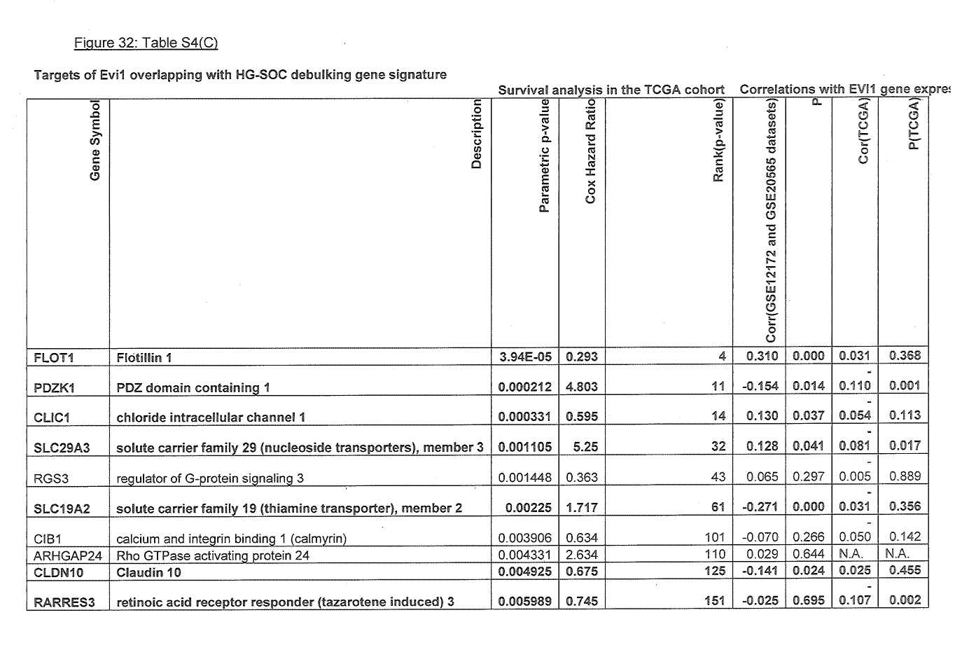

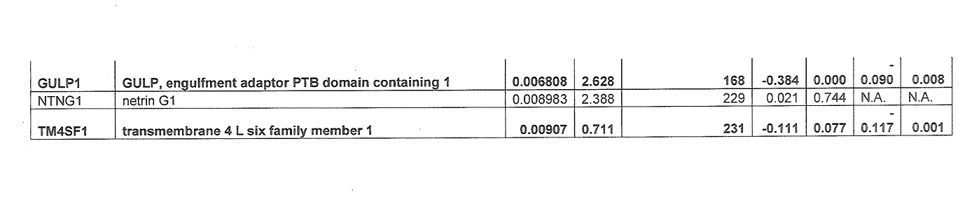

FIG. 30: Table S2: Novel Evi1-specific DNA motifs (M1 and M2), Evi1 BSs identified in the genome and their validation

(A) ChIP-seq peak clusters used for qPCR validation of Evi1 BS. The peak IDs correspond to their genomic location (hgl18) in the format: CHROMOSOME.CENTER, where CENTER denotes the location of the peak center on the given chromosome.

(B) The list of Evi1 Bss in non-repetitive regions identified within +/−50 kb vicinity from the genes Sheet C

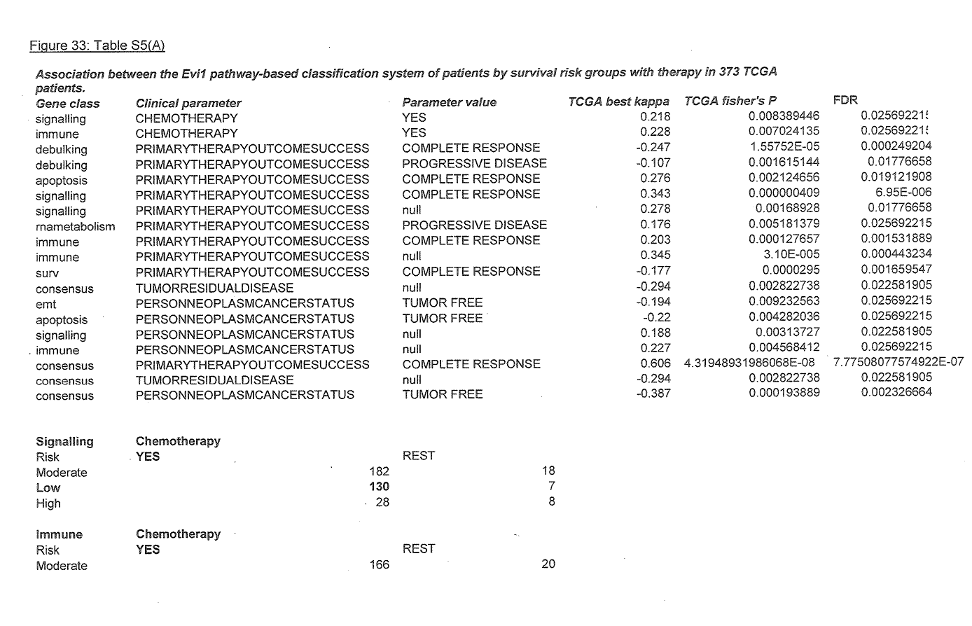

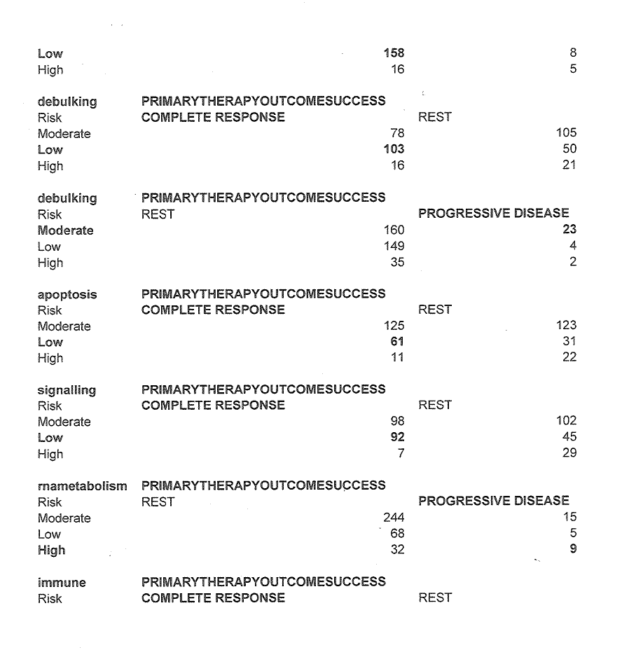

FIG. 31: Table S3:

(A) Evi1 direct target genes

(B) Transcription factor-specific DNA motifs co-localized with Evi1-specific DNA motifs M1 and M2

(C) List of mutations in the TP53 gene found in the TCGA patient cohort.

(D) Comparison of Evi1 copy number distributions among patients with various mutations in TP53 genes in TCGA patient tumors. Above the main diagnonal the tumors with EVI1 copy number higher than 3 copies/genome are selected. Below the main diagnonal the tumors with EVI1 copy number lower than 3 copies/genome are selected. Wilcoxon test p-values are given.

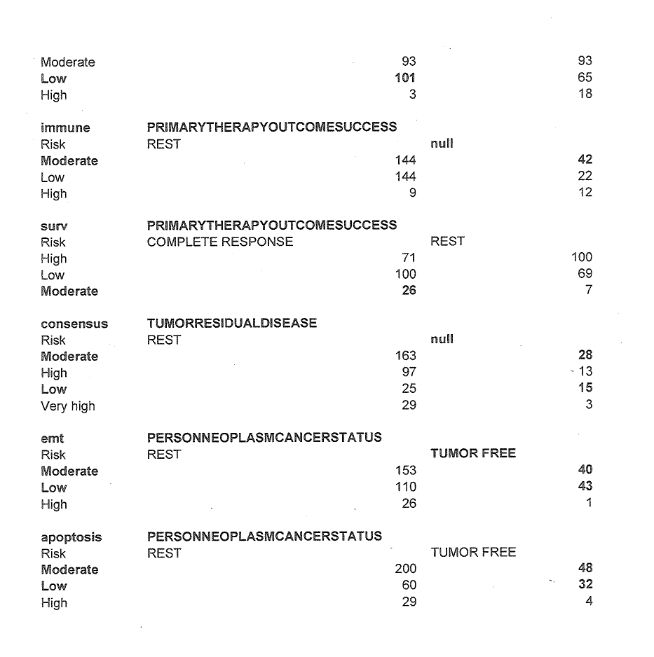

FIG. 32: Table S4: The expression of genes under direct control of Evi1 and their correlation with EVI1 and MDS1 in the tumors of GSE20565 and TCGA patient cohorts

(A) The list of genes with their corresponding values.

(B) Information on EVI1 copy number and expression improves prognostic value of 99% of EVI1 pathway genes.

(C) Targets of Evi1 overlapping with HG-SOC debulking gene signature.

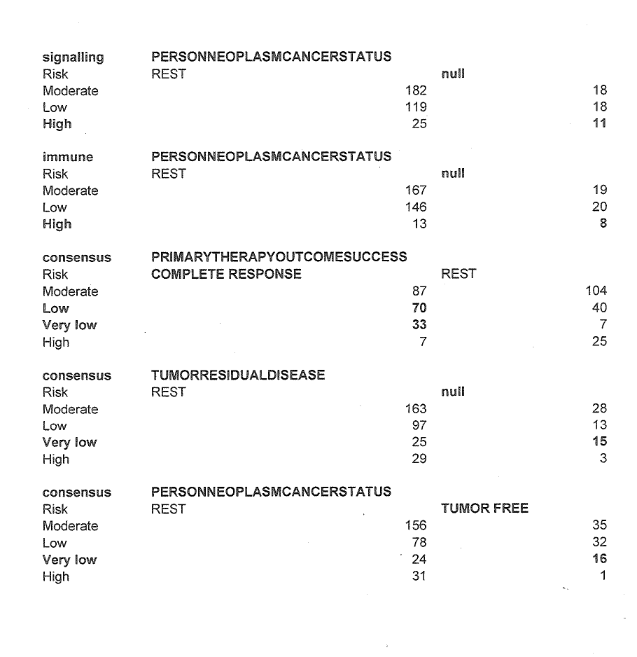

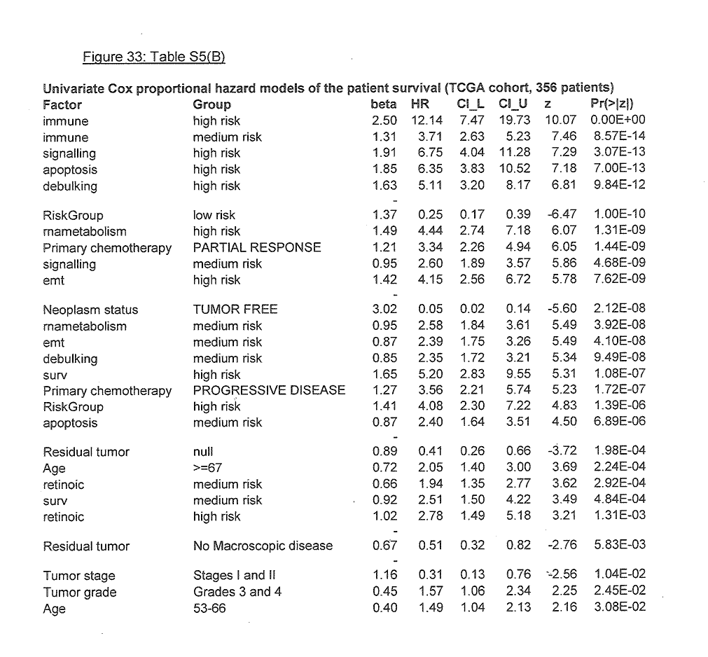

FIG. 33: Table S5: Association between Evi1 pathway-based patient stratification and other diagnostic classification systems

(A) Association between the Evi1 pathway-based classification system of patients by survival risk groups with therapy in 373 TCGA patients.

(B) Univariate Cox proportional hazard models of the patient survival (TCGA cohort, 356 patients)

(C) Multivariate Cox proportional hazard model of the patient survival, based on the diagnostic criteria available at the operation time (TCGA cohort, 356 patients)

(D) Multivariate Cox proportional hazard models of the survival of the patients with different response to the primary

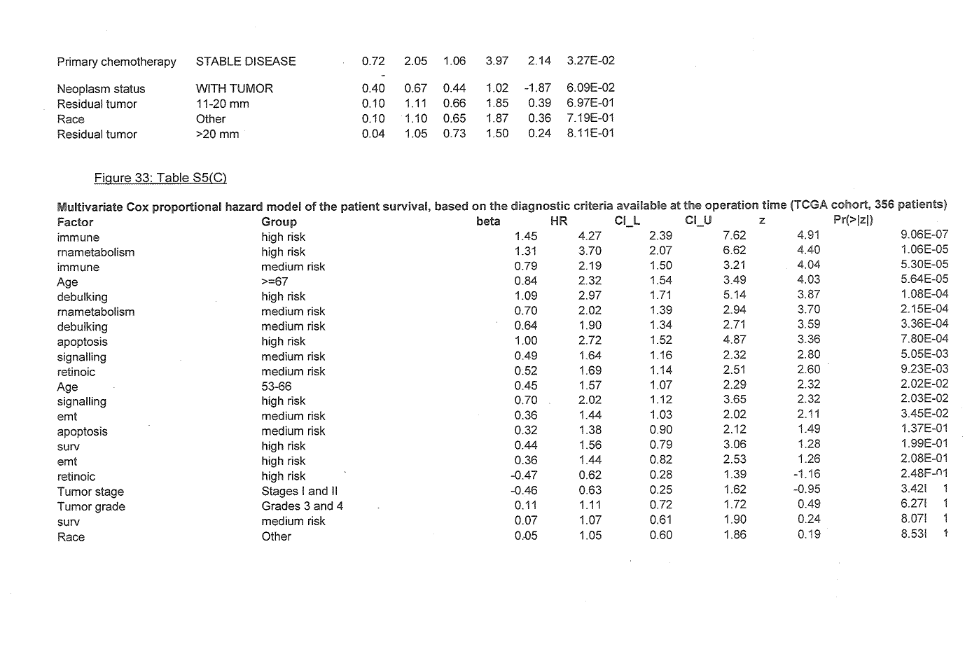

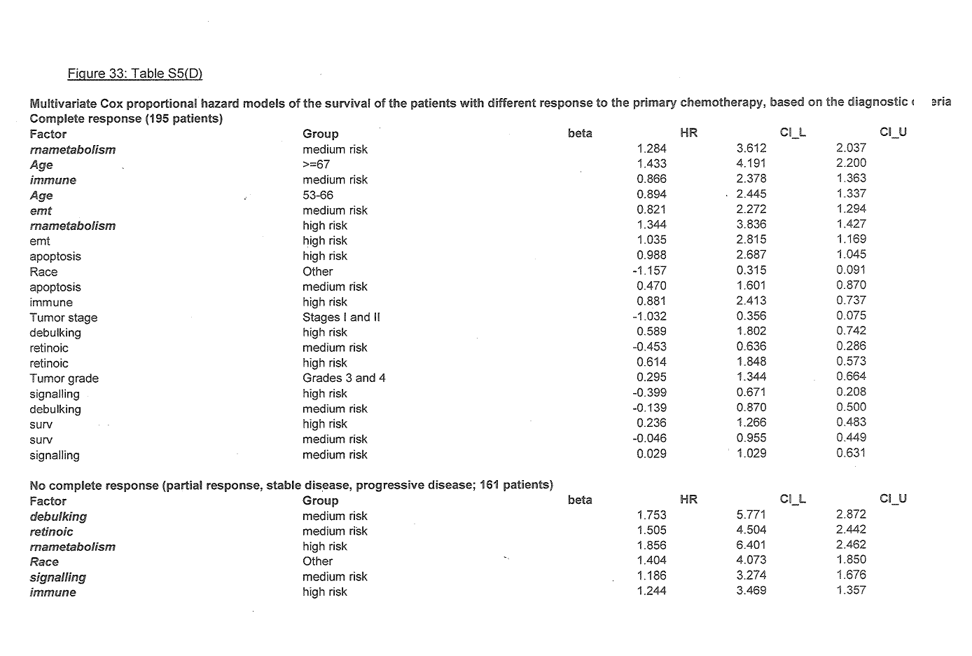

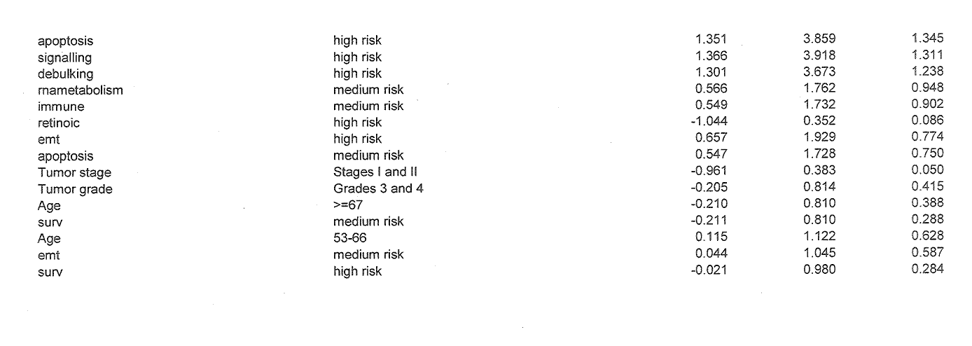

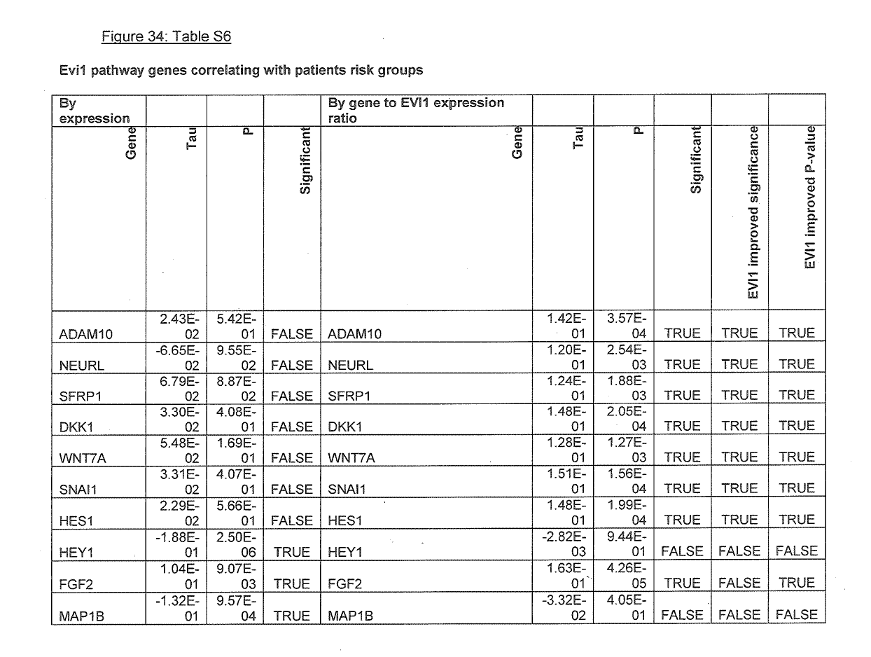

FIG. 34: Table S6: Evi1 pathway genes correlating with patients risk groups.

FIG. 35: Table S7: Characteristic TCGA HG-SOC genes (from Domcke et al., 2013) with CNA alterations in SKOV3.

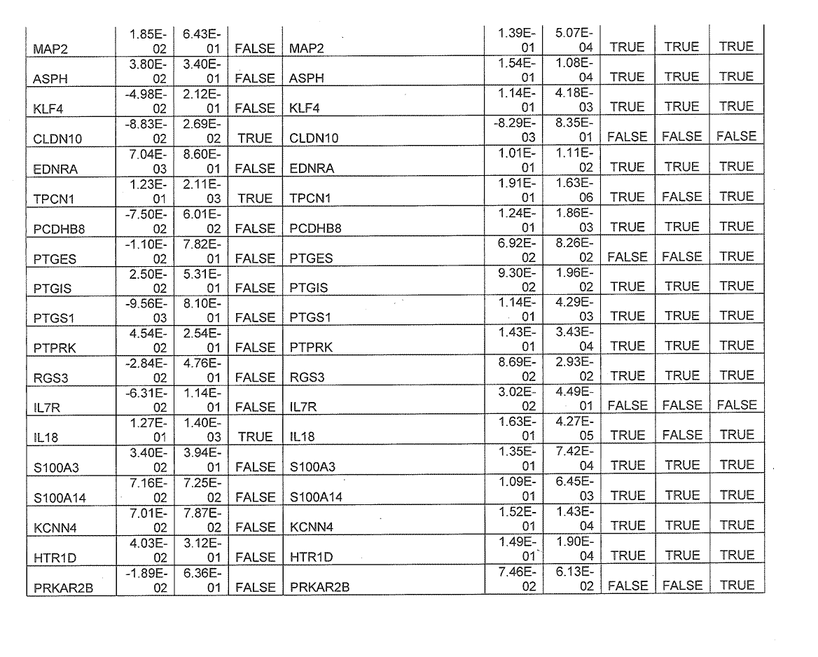

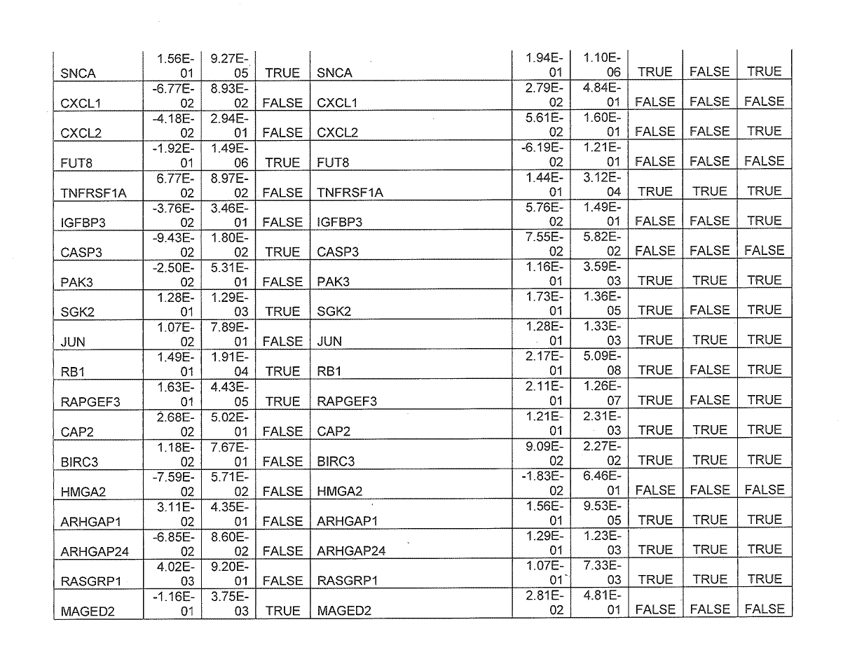

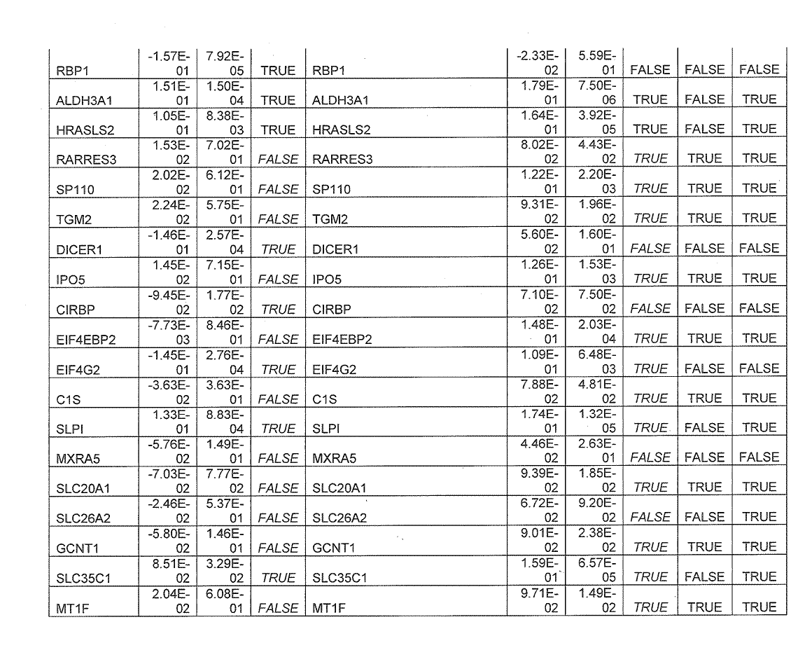

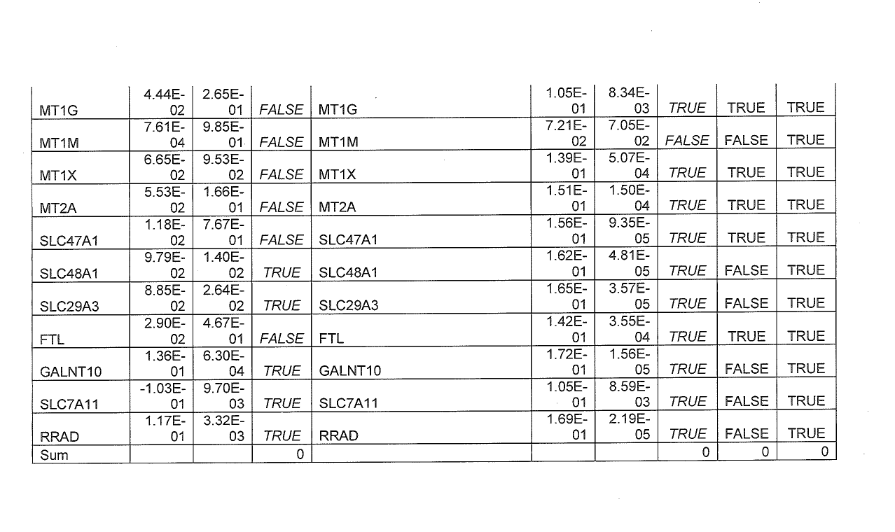

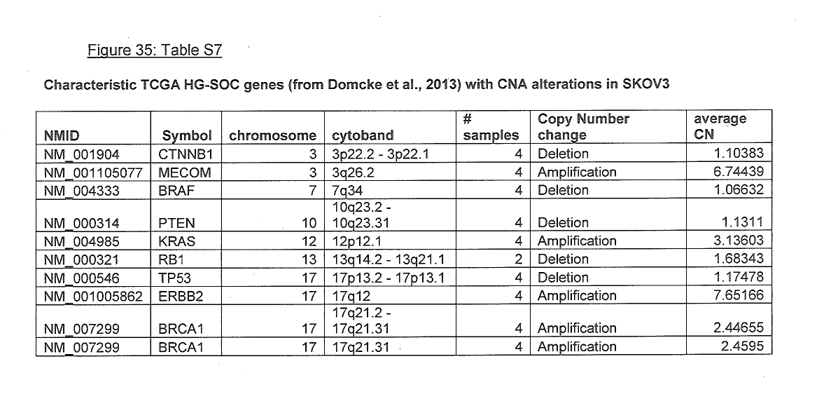

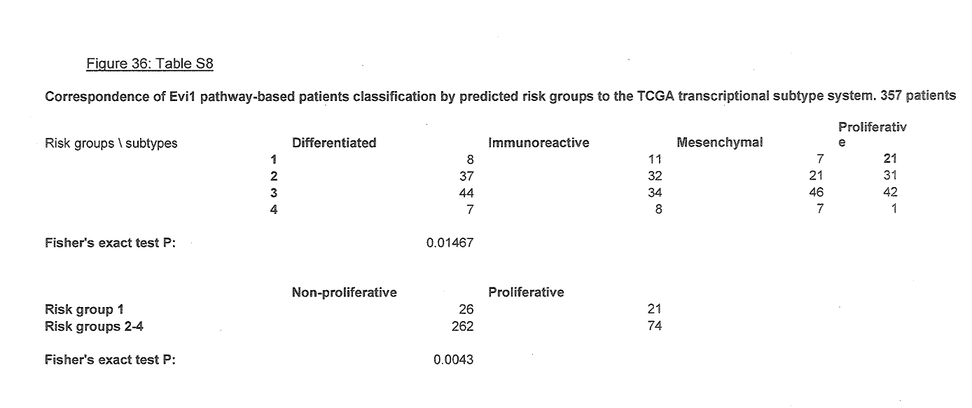

FIG. 36: Table S8: Correspondence of Evi1 pathway-based patients classification by predicted risk groups to the TCGA transcriptional subtype system. 357 patients.

### EXAMPLES

### Clinical Data Description

The National Institute of Health (NIH) Cancer Genome Atlas (TCGA) data set with 514 EOC patients was used for the analysis of CNV, gene expression and patient survival. The patients, which EOC tumors had EVI1 gene amplified (average EVI1 gene copy number not less than 2.5 per cell), defined here as ‘EVI1 amplified group, were analyzed separately. The 5-year survival for this group of patients was 36 per cent. The 5-year survival of the whole patient cohort was 28 per cent. The 2-year survival of the whole patient cohort was 74 per cent.

Gene Expression Ominibus (NIH) repository was used to obtain GSE9899 (accession number) data set containing 246 samples. From this set 16 patients were removed after a quality control assessement. The 5-year survival of the whole patient cohort was 44 per cent. The 2-year survival of the whole patient cohort was 57 percent.

### Statistical Methods

The methods described below extend the methods detailed in the Summary section. The methods below are specific to the Examples section.

For the analysis of co-expression of EVI1 and its potential interactors expression probe sets on the first stage only with strong Kendall correlations (|τ|0.3 and P_{FDR}(τ)0.01) with EVI1,were analyzed as potential survival group-separating variables as described. On the second stage, the paired combinations of each EVI1 probeset with each strongly correlating probe set were analyzed using the “2-D algorithm” [6]. In each survival group the probe sets correlating with EVI1 with significant impact on the survival of the patients by the FDR-corrected P-values were selected based on the criterion P_{FDR}(X²)<=0.05.

The gene signatures were formulated as follows. The branches of the Evi1 pathways were identified based on the literature data on the protein-protein interactions within the list of genes—direct targets of Evi1 regulation of transcription. The gene-targets of Evi1, which expression has been previously reported to be associated with suboptimal debulking [10], has been added to the list of the branches. For the genes of each branch the corresponding Affymetrix U133A probe sets were identified. In each list of probe sets (corresponding to each of the branches) for each probe set the best patient stratification strategy (and the corresponding threshold values) were found by the “1-D algorithm” (a gene expression voting procedure) [6]. Within each probe set list the probes were ranked by the P-values (smallest to largest) of their best patient stratification. Each signature (corresponding to each probe set list and each branch) was composed by evaluating the patient stratification resulting from a set of a given number of the top-ranked probe sets (genes) determined by independent voting procedure. The number of top-ranked probe sets in the voting was iteratively increased from two to the total number of the probe sets for each given probe set list, the performance measured by the P-value (Cox-proportional model) of the survival in the resulting patient strata. The list of top n probe sets for which the performance measure is less than 0.01 and the difference between the fraction of patients between these groups with the best and the worst prognosis is maximal, were selected as the gene signature characterizing the corresponding branch.

The clinical information obtained for each patient sample from tissue array qPCR was used for survival analysis. Out of 81 patient samples, 28 patients had clinical information with respect to survival (dead/alive with months of survival), metastatic status (yes/no), and relapse of tumor (yes/no). The survival plots were constructed using fold change values of various patient samples along with corresponding overall survival information.

Cohen's kappa correlation coefficient [11] was used for evaluation of correspondence between any two given classification systems. In the cases when any two compared classification systems contained different number of classes, all combinations.

Probabilities of the EOC patients overall survival were estimated with the Kaplan-Meier method. The Cox proportional models with single individual diagnostic and prognostic factors (univariate), as well as with multiple factors (multivariate), were applied to calculate the survival hazards in the in the patient cohorts. The Wald and log-rank tests were applied to estimate the survival significance. Pair-wise survival-based associations between the MECOM genes (EVI1 and MDS1) expression and their DNA copy numbers in the EOC tumors were obtained from evaluation of the individual genes expression survival significance in the patient strata defined by the genes' CN threshold. The individual CN threshold values were chosen in such way that maximized the difference between the survival rates of the resulting patient strata. The pair-wise survival associations between the expression of EVI1 and the individual Evi1 pathway genes were tested by the improvement of the survival significance of the patient stratification by an Evi1 pathway gene expression normalized by EVI1 expression, compared with the original gene expression values. The pair-wise survival associations between EVI1 expression and the individual pathway gene(s) expression was assessed by an increase of the survival significance of the patient stratification based on the combination of the pathway gene and EVI1 expression thresholds, compared with the stratification based on the pathway gene expression threshold alone.

### Example 1

The EMT signature expression can significantly discriminate 3 patient cohorts with different survival prognosis for the patients with tumors having EVI1 gene amplified, as well as for the whole patient cohort (FIG. 1).

The TCGA group of patients with EVI1 amplifications in the tumors, was stratified (P=1.1·10⁻⁸) by the signature into three prognostic subgroups: with ‘low-risk’, ‘medium-risk’, and ‘high-risk’ survival prognosis. The sizes of the groups were 75, 120 and 35 patients respectively, which corresponded to 33, 52 and 15 per cent of this patient group. The 5-year survival of the patients in each subgroup was 69, 39 and 0 per cent respectively. For the ‘low-risk’ and the ‘medium-risk’ subgroups the improvement in survival relative to the whole EVI1 amplifed patient group was 92 and 8 per cent respectively.

The whole TCGA patient cohort was stratified P=3.4·10⁻¹¹) by the signature into three prognostic subgroups: with ‘low-risk’, ‘medium-risk’, and ‘high-risk’ survival prognosis. The sizes of the groups were 157, 188 and 28 patients respectively, which corresponded to 42, 50 and 8 per cent of this patient group. The 5-year survival of the patients in each subgroup was 51, 15 and 6 per cent respectively. For the ‘low-risk’ subgroup the improvement in survival relative to the whole patient cohort was 82 per cent.

### Example 2

The Retinoic signature expression can significantly discriminate 3 patient cohorts with different survival prognosis for the patients with tumors having EVI1 gene amplified, as well as for the whole patient cohort (FIG. 2).

The TCGA group of patients with EVI1 amplifications in the tumors, was stratified (P=3.3·10⁻⁶) by the signature into three prognostic subgroups: with ‘low-risk’, ‘medium-risk’, and ‘high-risk’ survival prognosis. The sizes of the groups were 33, 149 and 48 patients respectively, which corresponded to 14, 65 and 21 per cent of this patient group. The 5-year survival of the patients in each subgroup was 84, 43 and 16 per cent respectively. For the ‘low-risk’ and the ‘medium-risk’ subgroups the improvement in survival relative to the whole EVI1 amplifed patient group was 133 and 19 per cent respectively.

The whole TCGA patient cohort was stratified P=7.4·10⁻⁶) by the signature into three prognostic subgroups: with ‘low-risk’, ‘medium-risk’, and ‘high-risk’ survival prognosis. The sizes of the groups were 57, 288 and 28 patients respectively, which corresponded to 42, 50 and 8 per cent of this patient group. The 5-year survival of the patients in each subgroup was 52, 26 and 5 per cent respectively. For the ‘low-risk’ subgroup the improvement in survival relative to the whole patient cohort was 86 per cent.

### Example 3

The Signalling signature expression can significantly discriminate 3 patient cohorts with different survival prognosis for the patients with tumors having EVI1 gene amplified, as well as for the whole patient cohort (FIG. 3).

The TCGA group of patients with EVI1 amplifications in the tumors, was stratified (P=3.8·10⁻¹³) by the signature into three prognostic subgroups: with ‘low-risk’, ‘medium-risk’, and ‘high-risk’ survival prognosis. The sizes of the groups were 94, 115 and 21 patients respectively, which corresponded to 41, 50 and 9 per cent of this patient group. The 5-year survival of the patients in each subgroup was 75, 27 and 13 per cent respectively. For the ‘low-risk’ subgroup the improvement in survival relative to the whole EVI1 amplifed patient group was 108 per cent.

The whole TCGA patient cohort was stratified P=2.3·10⁻¹⁵) by the signature into three prognostic subgroups: with ‘low-risk’, ‘medium-risk’, and ‘high-risk’ survival prognosis. The sizes of the groups were 112, 234 and 27 patients respectively, which corresponded to 30, 63 and 7 per cent of this patient group. The 5-year survival of the patients in each subgroup was 71, 44 and 19 per cent respectively. For the ‘low-risk’ and the ‘medium-risk’ subgroups the improvement in survival relative to the whole patient cohort was 154 and 57 per cent respectively.

### Example 4

The Apoptosis signature expression can significantly discriminate 3 patient cohorts with different survival prognosis for the patients with tumors having EVI1 gene amplified, as well as for the whole patient cohort (FIG. 4).

The TCGA group of patients with EVI1 amplifications in the tumors, was stratified (P=1.6·10⁻⁶) by the signature into three prognostic subgroups: with ‘low-risk’, ‘medium-risk’, and ‘high-risk’ survival prognosis. The sizes of the groups were 95, 101 and 34 patients respectively, which corresponded to 41, 44 and 15 per cent of this patient group. The 5-year survival of the patients in each subgroup was 61, 38 and 10 per cent respectively. For the ‘low-risk’ and the ‘medium-risk’ subgroups the improvement in survival relative to the whole EVI1 amplifed patient group was 69 and 6 per cent respectively.

The whole TCGA patient cohort was stratified P=6.5·10⁻¹²) by the signature into three prognostic subgroups: with ‘low-risk’, ‘medium-risk’, and ‘high-risk’ survival prognosis. The sizes of the groups were 92, 248 and 33 patients respectively, which corresponded to 25, 66 and 9 per cent of this patient group. The 5-year survival of the patients in each subgroup was 56, 22 and 0 per cent respectively. For the ‘low-risk’ subgroup the improvement in survival relative to the whole patient cohort was 100 per cent.

### Example 5

The Immune signature expression can significantly discriminate 3 patient cohorts with different survival prognosis for the patients with tumors having EVI1 gene amplified, as well as for the whole patient cohort (FIG. 5).

The TCGA group of patients with EVI1 amplifications in the tumors, was stratified (P=2.2·10⁻¹¹) by the signature into three prognostic subgroups: with ‘low-risk’, ‘medium-risk’, and ‘high-risk’ survival prognosis. The sizes of the groups were 78, 132 and 20 patients respectively, which corresponded to 34, 57 and 9 per cent of this patient group. The 5-year survival of the patients in each subgroup was 75, 31 and 9 per cent respectively. For the ‘low-risk’ subgroup the improvement in survival relative to the whole EVI1 amplifed patient group was 108 per cent.

The whole TCGA patient cohort was stratified P=3.9·10⁻¹⁵) by the signature into three prognostic subgroups: with ‘low-risk’, ‘medium-risk’, and ‘high-risk’ survival prognosis. The sizes of the groups were 100, 245 and 28 patients respectively, which corresponded to 27, 66 and 7 per cent of this patient group. The 5-year survival of the patients in each subgroup was 53, 21 and 0 per cent respectively. For the ‘low-risk’ subgroup the improvement in survival relative to the whole patient cohort was 89 per cent.

### Example 6

The RNA metabolism signature expression can significantly discriminate 3 patient cohorts with different survival prognosis for the patients with tumors having EVI1 gene amplified, as well as for the whole patient cohort (FIG. 6).

The TCGA group of patients with EVI1 amplifications in the tumors, was stratified (P=6.6·10⁻⁴) by the signature into three prognostic subgroups: with ‘low-risk’, ‘medium-risk’, and ‘high-risk’ survival prognosis. The sizes of the groups were 46, 155 and 29 patients respectively, which corresponded to 20, 67 and 13 per cent of this patient group. The 5-year survival of the patients in each subgroup was 66, 44 and 13 per cent respectively. For the ‘low-risk’ and the ‘medium-risk’ subgroups the improvement in survival relative to the whole EVI1 amplifed patient group was 83 and 22 per cent respectively.

The whole TCGA patient cohort was stratified P=3.4·10⁻⁸) by the signature into three prognostic subgroups: with ‘low-risk’, ‘medium-risk’, and ‘high-risk’ survival prognosis. The sizes of the groups were 51, 268 and 54 patients respectively, which corresponded to 14, 72 and 14 per cent of this patient group. The 5-year survival of the patients in each subgroup was 60, 28 and 3 per cent respectively. For the ‘low-risk’ subgroup the improvement in survival relative to the whole patient cohort was 114 per cent.

### Example 7

The survival signature expression can significantly discriminate 3 patient cohorts with different survival prognosis for the patients with tumors having EVI1 gene amplified, as well as for the whole patient cohort (FIG. 7).

The TCGA group of patients with EVI1 amplifications in the tumors, was stratified (P=4.6·10⁻⁷) by the signature into three prognostic subgroups: with ‘low-risk’, ‘medium-risk’, and ‘high-risk’ survival prognosis. The sizes of the groups were 33, 124 and 73 patients respectively, which corresponded to 14, 54 and 32 per cent of this patient group. The 5-year survival of the patients in each subgroup was 79, and 0 per cent respectively. For the ‘low-risk’ and the ‘medium-risk’ subgroups the improvement in survival relative to the whole EVI1 amplifed patient group was 119 and 31 per cent respectively.

The whole TCGA patient cohort was stratified P=2.0·10⁻⁷) by the signature into three prognostic subgroups: with ‘low-risk’, ‘medium-risk’, and ‘high-risk’ survival prognosis. The sizes of the groups were 44, 286 and 43 patients respectively, which corresponded to 12, 77 and 11 per cent of this patient group. The 5-year survival of the patients in each subgroup was 60, 27 and 9 per cent respectively. For the ‘low-risk’ subgroup the improvement in survival relative to the whole patient cohort was 114 per cent.

### Example 8

The debulking-significant signature expression can significantly discriminate 2 patient cohorts with different survival prognosis for the patients with tumors having EVI1 gene amplified, as well as for the whole patient cohort (FIG. 8).

The TCGA group of patients with EVI1 amplifications in the tumors, was stratified (P=9.5 10⁻⁶) by the signature into three prognostic subgroups: with low-riks', ‘medium-risk’, and ‘high-risk’ survival prognosis. The sizes of the groups were 68, 138, and 96 patients, respectively. The 5-year survival of the patients in each subgroup was 82, 55, and 29 per cent respectively.

The whole TCGA patient cohort was stratified P=8.0·10⁻¹¹) by the signature into two prognostic subgroups: with ‘low-risk’ ‘medium-risk’, and ‘high-risk’ survival prognosis. The sizes of the groups were 108, 222, and 43 patients, respectively, which corresponded to 74 and 26 per cent of this patient group. The 5-year survival of the patients in each subgroup was 48, 2, and 0 per cent respectively.

### Example 9

For the TCGA cohort, the best prognostic markers from each Evi1 pathway module signature were combined into an optimal 12 genes- signature(Figure 9AB). Similarly, for the cohort

GSE9899, the best Evi1 pathway module signatures were combined into an optimal 28 genes-signature(Figure 9CD). In their respective cohorts, both optimal signatures attained an unprecedented power to stratify the whole patient cohort by the survival prognosis into three groups (P<10⁻⁵⁰): with ‘low-risk’, ‘medium-risk’, and ‘high-risk’ survival prognoses.

### Example 10

The most significant prognostic stratification of the patients was obtained when the specific signatures of each module of Evi1 were combined in a single signature by a statistical procedure called voting (FIG. 10, Table 12). This signature provided a stratififcation EOC patients into four groups (P<1.0·10⁻⁵⁰): with ‘lowest-risk’, ‘low-risk’, ‘medium-risk’ and ‘high-risk’ survival prognoses. The sizes of the groups were 41, 111, 194 and 27 patients respectively, which corresponded to 11, 30, 52 and 7 per cent of this patient group. The 5-year survival of the patients in each subgroup was 84, 46, 11 and 0 per cent respectively. For the ‘lowest-risk’ and the ‘low-risk’ subgroups the improvements in survival relative to the whole patient cohort were 200 and 64 per cent respectively. The 2-year survival of the patients in each subgroup was 100, 94, 64 and 39 per cent respectively. For the ‘lowest-risk’ and the ‘low-risk’ subgroups the improvements in survival relative to the whole patient cohort were 35 and 27 per cent respectively.

### Example 11

The present theory states that fallopian tube epithelium (FTE) is the origin of EOC. It is consistent with the functions of Evi1 pathway. The expression of 43 genes of Evi1 pathway was significantly different (P<0.05) in FTE, in comparison with any stage of EOC (FIG. 11). Hierarchical clustering of these genes by their median expression in FTE and EOC of different stages discriminated four clusters: 1) high expression in FTE and low in EOC, 2) high expression in both FTE and EOC, 3) low expression in FTE and high in EOC, 4) low expression in both FTE and EOC.

The first cluster (genes repressed in FTE-EOC transformation) included cell contact proteins markers of EMT (CLDN10, LAMA4, fibronectin FNDC3A, TGM2); cytoskeleton and motility (crystallin AIM1, TPM2, ENPP2); apoptosis (GNG11, GULP1); phospholipid metabolism and prostaglandin biosynthesis (ENPP2, AKR1C3, PTG/S); cell membrane (TSPANI); Wnt-signalling pathway (SFRP1). Notably, a multi-functional enzyme transglutaminase (TGM2) is overexpressed in FTE, in comparison with EOC. TGM2 is involved in many cellular processes. It induces and is induced by RA signalling, activates apoptosis and phospholipase C and can be secreted . In relation to cell contacts, TGM2 can cross-link fibronectin thus increasing cell rigidity. Recent studies more directly suggest that TGM2 activity induces EMT [7].

The second cluster contained strongly expressed genes. Three of them were down-regulated in EOC (CLU, CIRBP and CAT) and one up-regulated (FTL).

The third cluster (genes actived in FTE-OC transformation) FHL2, a transciptional coactivator, was found to be activated by p53 and overexpressed in EOC [8]. Matrix modification proteins (MXRA5), metabolism (CALU, SLC20A1), cytoskeleton and motility (DBN1), metallothioneins (MTIG, MTIX).

The fourth cluster (low-expressed genes suppressed in FTE-OC tranformation) included importin IPO5 frequently mutated in cancer, KRAS-specific effector and tumor-supressor RASSF2, a cytosceletal protein TLN2, LRRC17 an inhibitor of phospholipase C signalling, PAK3 a downstream effector kinase of CDC42, thrombomodulin THBD, stem cell-associated TF KLF4, endothelin receptor EDNRA, genes regulating cellular metabolism (GNA14, RRAD, PPPIR3C, SLC47A1).

### Example 12

The signatures of the Evi1 pathway modules demonstrate strongly significant prognostic properties not only individually, but also in a combination as independent prognostic factors (FIGS. 10 and 11). The genes of Evi1 pathway can be used as sources of prognostic features not only as parts of the modules, but also individually. For the majority of Evi1 pathway genes its is possible to find such expression threshold that would classify the patients in a given group into two subgroups with significantly different overall survival times (Table 12). Gene expression is not the only feature that can be used for survival prognosis, based on these genes. Similarly to the gene expression, the average number of copies of a given gene per cell in a given tumor sample can be compared to a threshold value classifying the patients into two groups significantly different by overall survival times (Table 13). It is possible to provide a combined survival prognosis, based on both expression and copy number measurements of a given gene or a set of genes (FIG. 15).

The primary data to derive the gene expression or copy number threshold values as prognostic, diagnostic or predictive features can be obtained from the samples taken from a patient cohort defined here as the training cohort. The threshold of the feature is found as as such value that would classify all the patients of the training cohort into two subgroups, such that the maximal statistical significance of the difference (measured as overall survival time, diagnosis, or treatment outome) is obtained. Then, the survival prognosis (diagnosis, or outcome prediction) of the patients from another patient cohort, here defined as the testing cohort, can be determined by comparision the feature value in the respective sample, as outlined above (FIG. 15). If two or more patient cohorts are available for the role of the training cohorts, the threshold values can be obtained from each of the cohorts independently and then combined into consensus thresholds (FIG. 14). The consensus threshold of a feature can be used in the way as the usual threshold value, but is considered more robust, since it is ensured that the classification rule the threshold provides is significant in two independent cohorts.

### Example 13

Genomic sequences centered at the locations of 17746 Evi1 ChIP peaks were used for the the search of overrepresented DNA motifs (FIG. 13A,). The computational data analysis used motif finding method revealed two novel Evi1-specific DNA motifs - GAGACAG (M1) and TAATCCCAGC (M2)—the families of the relatively short DNA sequences over-represented within +/−100bp and +/−1 kb distances from the peaks respectively, assigned by our method with specific binding site of the EVI1 protein, acting as transcription factor (TF). The motifs occurred in the 20-kb vicinity of the experimentally defined EVI1 BSs 3.3 times more often than all the previously reported Evi1 motifs taken together [9]. Occurrence of at least one of the aboved meantioned motifs and experimentally defined EVI1 TF BS locus (Evi1 ChIP peaks) located in a vicinity of expressed gene, assigned experimentally for EVI1 TF BS, is used as essential characteristic of the of EVI1 pathway gene.

We experimentally confirmed that Evi1 protein can specifically bind M1 and M2 (FIG. 1). We found that, if taken in the local sequence context (shown on FIG. 3F), M1 has affinity to both isoforms, while M2 has affinity to the 8-Zn-finger isoform (Evi1Δ324). Point mutations in the motif sequences, decreased the affinities of M1 and M2 to 10-Zn-finger and 8-Zn-finger isoforms respectively. The affinity of both motifs to both isoforms disappeared after the motifs were isolated from their sequence context conservative in human genome. Remakrably, M1 and M2 are co-localized in 20 kb vicinity of Evi1 BSs more often than any previously reported motifs specific to Evi1 (FIG. 13C). M1 and M2 were co-localized with 99.9% (17726/17746) 97% (17185/17746) of the peaks respectively. Among the previously reported motifs the most frequent was GAGAAAGAG motif, co-localized with only 48%(8568/17746) of the BSs. It allowed us to consider M1 and M2 as the most general Evi1-specific and the only truly representative motifs reported so far.

### Example 14

To demonstrate the individual contributions of the pathway branches into the patient survival, we used univariate and multivariate proportional hazard survival models integrating the diagnostic parameters of the patients known at the time of the surgery. The univariate analysis (Table 14) demonstrated that the significant (P<0.01) diagnostic factors predicting the patient's survival included the pathway consensus signature, all the individual pathway branch signatures, and the advanced patient age (67 y.o. or more). The high-risk patient group predicted by the immune pathway branch was characterized with the highest hazard ratio of all individual predictors. When the multifactorial survival model (that excluded the consensus signature) was considered (Table 15), all the individual branches again were significant contributors to the overall hazard, except for the EMT branch (with P=0.03). Classification of the patients into two groups by the observation of the response to the primary chemotherapy revealed that in the respective multivariate models (Table 15) only two variables (corresponding to the immune response and the RNA metabolism signatures) were common among the significant predictors in both patient groups. The EMT branch signature and advanced patient age (53 y.o. or more) were the significant (P<0.01) prognostic factors among the patients with the complete response. The EMT branch signature was a significant hazard contributor among the patients with complete response, while three other branch signatures (the sub-optimal debulking, retinoic acid and signaling branches), along with the non-white race, were the unique significant survival factors for the patients with non-complete response types.

These result demonstrate that: i) Evi1 pathway is an independent survival factor; ii) at the time of the surgery, Evi1 the pathway signatures perform better as survival predictors, compared to any clinical parameters available for our study.

The Role of MECOM Complex Locus and Its Products in High-Grade Ovarian Carcinoma Pathogenesis, Early Diagnosis and Clinical Outcomes

Epithelial ovarian cancer (EOC) is a very heterogeneous and the most lethal gynecologic malignancy, ranked fifth in mortality among female cancers. For the past 30 years, mortality rate of EOC has remained high, despite considerable efforts directed to improvement of diagnosis and treatment of this disease. Most of the EOC patients are diagnosed as high-grade serous ovarian carcinoma (HG-SOC) histological type and defined clinically at stages III-IV when the tumor is heterogeneous and almost incurable. Therefore, understanding the early molecular pathogenesis of HG-SOC and discovery of novel prognostic and predictive biomarkers of HG-SOC is crucially important. Combining computational systems biology, genome-wide and validating experimental studies, we found that in HG-SOC cells the MECOM complex locus and its protein product Evi1 together promote genomic instability, alteration of damaged DNA repair system and control expression of specific Evi1 oncogene pathway integrating hundreds genes involving in embryogenesis, epithelial-mesenchymal transition, RNA metabolism, cancer cell survival and proliferation, and retinoic acid, anti-viral and therapeutic responses. Our results suggest that MECOM/Evi1 and the Evi1 pathway affect non-homologous end-joining, genome instability and could promote clonal variation and heterogeneity of the tumors. Using independent HG-SOC cohorts, we demonstrated that MECOM/Evi1 and the Evi1 pathway provide novel mechanistic means and high confidence and reproducible signatures for early detection and survival significant sub-classification of HG-SOC and proposed the Evi1 pathway targets for personalized therapeutics of HG-SOC patients.

Proto-oncogenes can mutate to oncogenes via point mutations, genomic translocations and amplifications. These mutations may drive the cells to malignancy, accelerate cancer progression and complicate treatment, subsequently leading to shorter patient survival times. The ecotropic virus integration site 1 gene (EVI1) together with myelodysplastic syndrome 1 gene (MDS1) and EVI1-MDS1 fusion gene are located in the MECOM complex locus. The human EVI1 encodes at least 7 zinc-finger domain-containing isoforms [24-25]. The Evi1 protein is an evolutionary conserved transcription factor (TF) with a 94% amino acid sequence similarity between humans and mice. EVI1 is highly expressed in the urinary system, lungs and heart. Its activity is vital for embryonic development [26-27]. EVI1 expression is essential for hematopoietic stem cell self-renewal and repopulation capacity of differentiating blood cells. Its mis-regulation leads to several myeloid malignancies (e. g., acute myeloid leukemia (AML), myelodysplasia) [65]. Elevated expression of EVI1 was also observed in breast carcinoma basal subtype and estrogen receptor-negative breast cancer patients [10], suggesting epithelial to mesenchymal transition (EMT) cell phenotype of cancer cells, often observed in epithelial ovarian carcinomas (EOC). MECOM complex locus can be amplified in malignant tissues in various organs including the ovary, cervix, lung, esophagus, colon, head and neck, prostate, and could contribute to the patho-biology of these neoplasms [28].

EOC is very heterogeneous and the most lethal gynecologic malignancy, ranked fifth in mortality among female cancers [16-18]. High-grade serous ovarian carcinoma (HG-SOC) represents the most common and aggressive EOC histologic form. Studies have indicated that most HG-SOC tumors originate from the fallopian tubes. However, malignant cells from other tissues (e.g., breast, colon, or endometrium) can also metastasize to the ovaries [20-23]. Currently HG-SOC is one of the most poorly understood cancers, with a lack of biomarkers identified for clinical use including early detection and personalized treatment. The overall 5-year survival rate of HG-SOC is only 28% [17]. The vast majority of deaths from HG-SOC are explained by a late diagnosis, the low sensitive prognostic factors and a widespread acquired resistance to chemotherapy. Therefore, understanding the molecular pathogenesis of HG-SOC at early stages and discovery of novel prognostic and predictive biomarkers of HG-SOC is crucially important.

Recent studies have demonstrated that the mutation events observed in HG-SOC are highly diverse and heterogeneous [18, 81]. The mutations of TP53 are the most frequent somatic point mutations detected in 87-93% HG-SOC [18, 19]. However, TP53 mutations are not clinical prognostic markers of HG-SOC, because the survival rates of the patients with TP53-mutation negative and TP53-mutation positive HG-SOCs are very similar [78]. In less than 17% of HG-SOC patients inherited loss-of-function mutations in BRCA1/2, CHECK2, RPS6KA2 or MLL4 are observed[81] Specific mutations in CHECK2, RPS6KA2 or MLL4 were associated with a very low overall survival rate and a high probability of post-surgery drug treatment resistance [66], however mutations in BECA1/2 are associated with lets aggressive disease[66]. Other prognosis-significant point mutations, mostly of somatic origin, are also relatively rare, but at least one of these mutations occurs in about 12% of HG-SOC patients and associates with very poor survival rates [81]. Thus, about 70% of HG-SOC cases might be related to alternative etiological and patho-biological mechanisms. Currently, at least fifteen oncogenes (RAB25, MECOM, EIF5A2, PKRCI, PIK3CA, FGF1, MYC, EGFR, NOTCH3, KRAS, ERBB2, PIK3R1, CCNE1, AKT2, and AURKA), two of which are transcription factors (MYC and EVI1), are considered important for EOCs [16,18,20]. Eleven of them, including MECOM, are amplified in cancer cell genomes [20]. MECOM is highly amplified in most HG-SOC cases; however, possible roles of this amplification in HG-SOC progression have been poorly studied. Particularly, MECOM locus amplification and the activity of its encoded protein Evi1 have never been studied as a single composite system affecting HG-SOC pathogenesis. It may be a reason why reports on Evi1 oncogenic or tumor suppressing functions, as well as on its clinical diagnostic and prognostic value, have been contradictory [29,30]. Moreover, despite Evi1 being a transcriptional regulator, its direct target genes in HG-SOC cells remain incompletely defined (see below) and/or poorly functionally characterized, except for a minority, including PTEN, MIR-9, BCL-xL [82-84].

To fill these gaps, using novel computational approaches, we integrated our experimental studies and clinical meta-data to elucidate the patho-biological functions of MECOM complex locus together with its products and EV11-controled gene targets in HG-SOC. Specifically, we carried out high-throughput genomic, transcriptomic and proteomic experiments using cultured HG-SOC cells and integrated the results with the genomic, transcriptomic and clinical data obtained in massive studies of HG-SOC patient tumors. This strategy allowed us to identify the major, previously unknown, DNA motifs binding Evi1 and to specify hundreds of genes directly or indirectly targeted by Evi1 via these motifs. Our findings suggested that Evi1 can control a distinct regulatory pathway that drive the cancer progression and could be organized into seven functional modules. These modules collectively stratified HG-SOC patients into four post-surgery survival groups at P<1.0×10⁻⁵⁰. As a prognostic indicator, Evi1 pathway outperforms the clinico-pathological parameters of all previous HG-SOC predictors and correlates with therapeutic treatment outcomes. We observed that the protein-protein interactions (PPIs) of Evi1 with other DNA-binding proteins could promote double-strand DNA break (DSB)—mediated recombination, where Evi1 itself controls the non-homologous end joining (NHEJ) of the DSBs. Our results suggested that experimentally and clinically observed MECOM/Evi1-dependent genomic instability might be an essential factor of HG-SOC diversity and progression. We found that the MECOM locus, Evi1 protein and Evi1 pathway altogether integrate crucial biological processes that are common to embryogenesis, cancer initiation, progression and metastasis. It allowed us to select perspective Evi1 pathway genes and gene subsets which exhibited high-confidence and robust features as biomarkers for the ovarian cancers early detection, differential diagnosis, prognosis and treatment prediction. Finally, we firstly developed prototype of PCR-based assay for detection of the RNA products of MECOM and discussed how our findings could provide insights into improving the clinical practice.

Results

Alternative transcripts of EVI1 and MDS1 genes encoded in MECOM complex locus could be highly specific and sensitive markers of HG-SOC. MECOM complex locus includes two genes EVI1 and MDS1. To clarify the role of these genes in HG-SOC, its expression in the tumors was studied using publicly available microarray datasets. The analysis demonstrated an overexpression of MDS1 and EVI1 transcripts in HG-SOC tumors, compared with normal ovarian surface epithelium samples (FIG. 18A). The expression levels of MDS1 and EVI1 transcripts in EOC positively correlated with each other (FIG. 18B). To evaluate the diagnostic potential of EVI1 expression observed in the microarray data, we designed PCR primers specific to the exonic region common to five of the seven EVI1 transcription variants [44] and applied them to stratify 76 HG-SOC tumor samples and 14 normal ovary control samples (FIGS. 18C and 18D). In 100% of the cases, EVI1 expression discriminated between the normal ovarian surface epithelium (OSE) and the HG-SOC tumors, with the average expression being up to 20 times higher in the tumors (at stage IV of HG-SOC), compared with the OSE. Interestingly, EVI1 expression at early HG-SOC stages (I and II) was significantly higher than at late stages (III and IV). We also observed that EVI1 and MDS1 expression discriminated (P=0.048 for EVI1; P=0.039 for MDS1) between the HG-SOC stage I tumors and the fallopian tube epithelium (FTE; FIG. 18E, Table S1). Significant differences were observed between the EVI1 expression levels in the stage I and stage IV HG-SOC tumors (P=0.025) and between the tumors of all HG-SOC stages and the FTE.

The DNA copy number variation (CNV) analysis revealed that the fraction of tumors with amplified MECOM (on average, more than 2.5 copies of MECOM complex locus per tumor genome) increased from 58% at OC stage I to 74% at stage IV (FIGS. 18F and SIC). By contrast, the average MECOM copy number (CN) was normal in all of the control FTE samples.

Thus, these results suggest that in the HG-SOC, MECOM amplification and EVI overexpression are as common as point mutations in TP53. Therefore, we analyzed the associations between TP53 mutation types and EVI1 gene CNV in the patient tumor data, available from the TCGA. We classified the 336 TP53 point mutations observed in 316 patients by their structural and functional effects on the TP53 transcripts and p53 protein into 13 classes [18]. A pair-wise comparison between the frequencies of different TP53 mutation classes (Tables S3D-E) revealed that in HG-SOC tumors with strongly amplified MECOM (3 or more copies per genome) the missense mutations in TP53 were characterized with a significantly (P=0.0047) higher MECOM CN (mean 4.3) compared with the tumors carrying the nonsense mutations (mean 3.55). Similarly, a significant difference (P=0.039) between the frequency of TP53 splicing sites altering mutations (at the average MECOM CN per tumor genome 4.0) and nonsense mutations was observed. In the tumors, where the average MECOM CN per genome was lower than 3, no significant differences between the frequencies of the TP53 mutation classes were revealed. Thus, it could be concluded that the HG-SOC tumors, where p53 is not inactivated by mutations, are more likely to survive if their genomes contain several copies of the amplified MECOM locus.

We observed that alterations of the MECOM locus genes expression reliably discriminated HG-SOC from a wide range of other secondary tumors found in ovaries. The expression levels of both MECOM genes, EVI1 and MDS1, could discriminate between EOC of the low malignancy potential tumor (LMP) and HG-SOC samples (at P(EVI1)=0.02 and P(MDS1)=2.1·10⁻⁴; FIG. 18G). High expression of the EVI1 and MDS1 could discriminate between HG-SOC and breast cancer metastases in the ovaries with a high accuracy (at P(EVI1)=5.8·10⁻¹⁸ and P(MDS1)=5.7·10⁻¹²; FIG. 1H). We also found a significant over-expression of the MECOM genes in HG-SOC compared to ovarian endometriosis lesions ovaries (at P(EVI1)=7.6·10⁻⁵ and P(MDS1)=9.8·10⁻⁵; FIG. 181).

Thus, MECOM amplification and high expression of its genes EVI1 and MDS1 are essential characteristics of HG-SOC, and can be considered as highly specific and sensitive perspective over-expressed biomarkers for early detection and differential diagnosis of the disease. To determine whether the specificity of MECOM and EVI1 is linked to their prognostic significance, we evaluated the genome-wide CN and expression data in the TCGA HG-SOC patient cohort [18].

The prognostic significance of EVI1 and MDS1 genes expression could be predicted by the number of their genomic copies in the HG-SOC tumors. Proto-oncogenes can mutate to oncogenes via point mutations and genomic aberrations. These mutations may accelerate cancer progression and complicate treatment, subsequently leading to shorter patient survival times. We observed that in the TCGA patient cohort, MECOM, along with TP53, was among the most frequently mutated loci. However, unlike the point mutations in TP53 [18], the primary mechanism of MECOM mutation was chromosomal aberration. The MECOM CN averaged at 2.5 copies per tumor genome in 84% of the OC samples; the 3′-end of the EVI1 gene was the most strongly amplified region on chromosome 3, reaching a maximal amplification frequency at a location 150 kb downstream of EVI1 (FIG. 18J). Across TCGA OC patients, the EVI1 region was characterized by a mean of 3.52 and a median of 3.19 copies per tumor genome and the MDS1 region averaged a mean of 3.39 and a median of 3.18 copies, respectively. These values were higher than the previously reported values [45]. The survival analysis of advanced HG-SOC (stages III and IV) demonstrated that EVI1 gene expression significantly affected patient survival (P<0.01) in these tumors. The survival significance of MDS1 expression was marginal (P=0.049). Next, we separated these patients into four groups: the patients with tumors containing a high CN of EVI1 or MDS1 and the two respective control patient groups with low EVI1 and MDS1 CN values (FIGS. 18K-N). In the group of patients (64% of the patient population), whose tumors were characterized with the MDS1 CN higher than averaged 3.6 copies per tumor genome (FIG. 18M) MDS1 expression was a strong prognostic factor that separated patients by survival (P=0.0028), showing a remarkable improvement of the P-value by a factor of 17 compared with the whole population (P=0.049). In the sub-population with the tumors, where MDS1 was not amplified (FIG. 1N), no survival significance of its expression was observed (P=0.39). Thus, MDS1 expression was a significant prognostic factor only when the tumor MDS1 gene CN was on average higher than 3.6 copies per tumor genome. At the same time, using our prediction model (Methods), EVI1 expression was characterized with two distinct cutoff levels separating the patients into the groups with favorable and unfavorable prognoses: a lower cutoff for the group of patients where EVI1 gene CN was higher than 3.5 (39% of the population; the survival significance at P=1.3·10⁻⁴; FIG. 18K) and a higher cutoff where its CN was lower than 3.5 (61%; the survival significance at P=0.0043; FIG. 18L). The existence of at least two combinations of survival-significant expression and CN thresholds for the MECOM locus genes EVI1 and MDS1 suggests that Evi1 may participate in several alternative survival-related molecular mechanisms of carcinogenesis that are specific to tumors with different CN values of this locus.

After we found that EVI1 expression and gene CN were strong pro-oncogenic prognostic markers of HG-SOC on a genomic scale, we studied the relationship between the Evi1-dependent mechanisms and HG-SOC progression via analysis of genes whose expression is transcriptionally regulated by Evi1 binding, designated as Evi1 targets.

The novel sequence motifs in the DNA mediate Evi1 cooperation with TFs to regulate target gene expression. Genomic sequences centered at the locations of 17,746 Evi1 ChIP-seq peaks were used to search for overrepresented DNA motifs (Figure S2A, Table S2). Evi1 BSs revealed a high diversity in their localization relative to the neighboring genes. For example, in the MECOM gene itself, strong BSs were localized in the exons (FIG. 19A). In DICER1, which is known to regulate RNA metabolism [35], the BS was localized 543 bp downstream from the promoter, in the first intron (FIG. 19B). In SNAI1, a central EMT regulator [51], it localized 10.6 kb downstream from the transcription end site (FIG. 19C). At the same time, in TDG, the gene of a key enzyme in DNA demethylation and repair [52], an Evi1 BS was localized in a canonical promoter region, 96 bp downstream from the transcription start site (FIG. 19D).

The motif prediction analysis (Detailed in Supplementary Methods) revealed two novel Evi1-specific DNA motifs, GAGACAG (M1) and TAATCCCAGC (M2), that were over-represented within +/−100 bp and +/−1 kb distance from the ChIP-seq peaks, respectively. We experimentally confirmed that the Evi1 protein specifically bound M1 and M2 (FIG. 19E). We observed that within its local genomic sequence context (shown in FIG. 19F), M1 had an affinity to both major Evi1 isoforms, while M2 had an affinity to the 8-Zn-finger isoform (Evi1Δ324). In contrast with all previous studies, M1 and M2 co-localized in the 20-kb vicinity of 99.9% (17726/17746) and 97% (17185/17746) Evi1 BSs, respectively. The co-localization occurred more frequently than for any previously reported motifs specific to Evi1 combined (Figure S2C) [35, 44]. Among the previously reported motifs, the most frequent was GAGAAAGAG, which co-localized with only 48% (8568/17746) of the BSs. These data allowed us to consider M1 and M2 to be novel and the most general Evi1-specific motifs reported to date. We analyzed the presence of DNA motifs in the vicinity of genes differentially regulated by Evi1 using two experiments: a) siRNA knock-down of the EVI1 transcript in SKOV3 cells (Table S3A) and b) EVI1 gene transient overexpression (Table S3B). Our results suggest that 309 genes containing high confidence Evi1 BSs were under its direct control, with 204 being activated by Evi1 and 105 being repressed (Tables 53A and B). Approximately 98% of the genes (303/309) contained Evi1 BSs within +/−20 kb from the target gene TSSs, and 90% (280/309) were around their TESs. Interestingly, the lists of Evi1 direct targets obtained with both experiments overlapped only in four genes (EVI1, BST2, IFI30, and IFI44L). Evi1 BS distribution relative to the targeted genes was unusual for a TF. Located within a +/−10 kb distance from the gene flanking regions (TSS and TES), Evi1 BSs cover 29% (10349/35897, RefSeq) of human genes. A total of 43% (4495/10349) of the genes contained Evi1 BSs at both TSS and TES (FIG. 20D). Such cases were significantly more frequent than expected by chance (P=2.2·10⁻¹⁶). It may suggest a cooperatively and/or a distant binding-mediating Evi1-DNA interaction. At the flanks of the target genes, Evi1 BSs were, on average, most densely located around 17 bp upstream of the TSS and 221 bp downstream of the TES (Figure S4A).

To test whether the presence of M1 and M2 in the TSS vicinity of the target genes could affect their Evi1-dependent expression, we analyzed the correlation between the EVI1 fold change obtained in the Evi1 knock-down experiment and the motifs (M1 and M2) occurrence frequencies in the region within +/−20 kb upstream of the Evi1 targets (Table S3A). We found that the occurrence of M1 and M2 at the TSSs of the Evi1-activated genes positively correlated (τ=0.26, P=0.04 for M1, τ=0.35, P=0.004 for M2 for genes with an Evi1-induced change of 1.7-fold or greater) with their fold activation (Figures S2D). In the Evi1 overexpression experiment (Table S3B), unlike the M2 motif, M1 occurrence near the TSSs revealed a tendency for a positive correlation (τ=0.37, P=0.098, for genes with a fold change difference of 1.8 or greater; Figure S2E). At the same time, a positive correlation was observed between the occurrence of M1 and M2 and the Evi1 BS occurrence within a +/−20 kb region of the TSS of Evi1 target genes, whose expression changed at least 1.7 times (τ=0.28, P=0.003 for M1, τ=0.26, P=0.006 for M2; Figure S2F). Thus, the identified genes targeted by Evi1 simultaneously satisfied three criteria: 1) the presence of Evi1-specific motifs; 2) the presence of ChIP-seq-detected Evi1 BSs; and 3) their differential expression upon experimentally altering the Evi1 expression levels. We defined such genes (listed in Tables S3A-B) as the direct targets of Evi1 transcriptional control. Among them, we identified a subset of genes, whose protein products share the same regulatory pathways (see below).

The analysis of M1 and M2 genomic locations revealed that these motifs co-localized (at the above conditions) both with each other and with motifs specific to other transcription factors (FIG. 19G-I). It allowed us to propose a list of 53 TFs potentially interacting with Evi1 via M1 and M2 (Table S3C). The motifs of 26 of these TFs (including 21 specific for M2 of Evi1 and 6 specific for M1) were enriched in the regions flanking Evi1-regulated genes. It is likely that at least one of these TFs, myeloid ectopic viral integration site 1 (Meis1), could be an isoform-specific protein that interacts with Evi1 in HG-SOC tumors. Indeed, Meis1 is a homeobox cofactor that plays a pivotal role in the induction AML, cooperates with Evi1 during myeloid proliferation and accelerates AML progression. Both the self-renewal and proliferative activities of Hoxa9/Meis1-expressing myeloid progenitors are enhanced by Evi1 [53]. Gene expression analysis of the HG-SOC tumors of the TCGA patient cohort (Figure S3A) revealed 238 genes whose expression positively correlated (τ≧0.30) with the MDS1 transcript. Surprisingly, 50% of these genes contained Meis1 transcription factor BSs.

Evi1 genome-wide binding to DNA promotes local genomic rearrangements. Previously, it was demonstrated that Evi1 physically interacts with Yyl [35, 54]. Here we identified Yy1 among the 53 TFs interacting with Evi1 (see above). This TF is involved in both DNA repair and transcription regulation [55]. Thus, it is possible that Evi1 interacts with the DNA recombination/repair complex. To test this hypothesis, we isolated Evi1-enriched protein complexes from the nuclei of SKOV3 cells and analyzed their composition using the SILAC mass spectrometry method. We identified twelve proteins that participate in DNA recombination and repair (FIGS. 20A and 20B). They formed three PPI clusters: 1) response to DNA damage stimulus (Tp52bp1, Smc4, Smc3, SMclA, Trim28, and RuvBL1); 2) homologous recombination, HR (Rad50, Msh2, Tp53bp1, and Yy1); and 3) non-homologous end joining, NHEJ (Rad50, Ku70, Ku80, and Prkdc). Among them, Rad50 revealed the strongest Evi1 enrichment, ranging from 7.11 to 47.8 (H/L ratio). Along with the peptides belonging to Rad50 isoform 1, a peptide specific to a poorly characterized isoform of this protein was identified in the mass spectra (denoted as Rad50-E7EN38 in FIG. 20A). Described in Release 8 of Ensemb1 gene annotation, its protein sequence differs from that of Rad50 Isoform 1 in exon 10, which is skipped, and exhibits a truncation of all exons beyond the first twelve (Figure S4B). If Evi1, as a part of the above protein complexes, is indeed involved in DNA recombination events causing CNA in HG-SOC genomes, its genomic BSs are expected to coincide with CNA sites. To test this hypothesis, we integrated our CNA data with the Evi1 ChIP-seq data. In SKOV3 cells, we observed that Evi1 BSs non-randomly co-localized with CNA regions (FIG. 20D-F). The frequency of gene amplification in SKOV3 cells was 2.4 times larger (P=1.2·10⁻²⁷) for the genes flanked by Evi1 binding sites on both ends (TSS and TES) compared with those with Evi1 BSs on either end (TSS or TES). Remarkably, Evi1 BSs with the strongest affinity co-localized with the region of the most frequent CNA in the genome, MECOM. Moreover, for direct targets of Evi1, the fraction of genes with symmetric translocations was even higher, with the ratio reaching 3.7 (P=7.5·10⁻⁵). To investigate whether the observed relationship between Evi1 binding and CNA was recombination-specific, we analyzed the data on the gene fusion transcripts observed in cancers [56]. Compared with gene amplifications (symmetric translocations), gene fusions (asymmetric translocations) in cancer cells were characterized with a 1.72 times larger fraction in Evi1 binding sites at either TSS or TES regions of the genes compared with the genes that have Evi1 binding sites in both regions (P=3.2·10⁻⁴). Finally, we evaluated a set of genes with fragile regions induced by chemical mutagenesis. In this case, no significant enrichment in symmetric vs. asymmetric Evi1 binding events was observed. Interestingly, the genome-wide frequency of CNA regions overlapping with genes was higher if the genes contained Evi1 BSs in close proximity (5 kb) of their CpG islands (P=9.4·10⁻⁴; FIG. 20C). Thus, our results indicated that in the HG-SOC genome, the (a)symmetry of Evi1 BSs location relative to the genes flanking regions, specifically correlated with the (a)symmetry of CNV of these genes. This finding supports the hypothesis that the above mentioned Evi1-containing protein complexes are involved in homologous recombination and NHEJ. DNA-methyltransferase-dependent double strand break repair mechanisms may be involved in this process as well [57]. As NHEJ, apparently, plays the largest role in double strand DNA breaks repair in humans [86], we studied the role of Evi1 in this mechanism. We carried out an experiment, where the in vitro NHEJ activity of nuclear protein extracts was quantified using a qPCR assay [87]. We observed that upon siRNA knock-down of EVI1 transcript level to 30% of its control value, the NHEJ activity of the nuclear proteins reduced to less than 20% of its original level (FIGS. 20G and 20H). Thus, for the first time, we demonstrated induction of genomic aberrations as a novel function of Evi1 via NHEJ mechanism.

NHEJ activity, specifically enhanced by Evi1, may induce CNV of its target loci, including its own locus MECOM. Therefore, a correlation between the MECOM CN and gene Evi1 target gene expression may be expected, since Evi1 binding is a common regulator for both. Otherwise, if MECOM CN is an active factor of HG-SOC progression, its effects could potentially be revealed as independent of Evi1 activity and expression. To test this hypothesis, we compared Evi1 target genes expression upon perturbations in the MECOM locus CN vs. EVI1 transcript expression. We analyzed the TCGA patient HG-SOCs and classified these tumors into three groups (designated as low medium, and high, respectively by EVI1 expression level relative to the threshold values 9.5 and 10.2. Correlations between MECOM CN and Evi1 target genes expression were studied separately in each group. The Evi1 target genes obtained in the two types of Evi1 perturbation experiments were grouped into two respective classes: class 1 targets revealed by the siRNA-mediated EVI1 transcript knock-down and class 2 targets revealed by the plasmid-mediated EVI1 overexpression. We found that in the tumors with low EVI1 expression, the expression of the class 2 targets correlated (Kendall's correlation, P<0.05) with MECOM CN 1.9 times more frequently than the class 1 targets (8/21 for the class 2 vs. 48/245 for the class 1, P=0.05; Fisher's exact test). The incidence of significant correlation between the class 2 targets expression and MECOM CN was even more frequent compared with the genes not reported as direct Evi1 targets in the present study (8/21 for the class 2 targets vs. 2304/13010 for the non-target genes; frequency ratio 2.2, P=0.01). Interestingly, in the tumors with high EVI1 expression, the fraction of significantly correlating targets was less in class 2 (2/21 targets, P=0.06), while was the same in class 1 (45/245, P=1.0). These results suggest that in the HG-SOC tumors with low EVI1 expression, the correlation between the MECOM CN and Evi1 target genes expression is significant, and can be attributed to the changes in the number of MECOM locus genomic copies, rather than to the changes in cellular EVI1 transcript levels.

Evi1 Controls the Transcription of a Specific Regulatory Pathway Promoting HG-SOC Tumor Progression.

The 235 genes, whose expression was positively correlated with EVI1 in the HG-SOC tumors, were strongly enriched in epithelial cell proteins (108/235, Enrichment 3.4, P=2.3·10⁻³¹. About 60% (140/235) of these proteins were localized in the nucleus, and 21 of them participate in chromatin binding (Ctcf, Dnmt3A, PatZ1, SmarcA4, SmarcCl, HupF1, Adnp1, CenpF, Cbx5, Chd3, Chd4, Chd7, Hells, Crsp1, NfatC3, HoxA7 (Hox 1.1), Pbrm1, Pbx2 (Hox12), Meisl, Sf3B1, and Top2B). Three of them (Meisl, HoxA7 and Pbx2), predicted in the present work as Evi1 interactors, have been previously reported to directly interact with each other in myeloid cells [43, 44].

Analysis of the genes that are differentially expressed in Evi1 knock-down SKOV3 cells (Table S2B), revealed 364 genes controlled by Evi1 (see above). Integrating this information with the ChIP-seq data showed that 285 of these genes (78%) were controlled by Evi1 direct binding in their genomic vicinity (Table S3A-B). In the experiment when Evi1 was overexpressed, only 31, mostly up-regulated (29/31) genes, were identified. Strikingly, 15 of them act in cells in response to viral infection, including seven induced by interferon-y (P=2.36·10⁻⁶). In total, 13 genes (IF144L, IF144, 1F130, IF16, IFIT2, IFIT3, IFIT1, IFITM1, IF127, IF135, IFIH1, MX2, and MX1) involved in the interferon-y pathway could be considered as Evi1 direct targets. Overall, more than 50 of the proteins encoded by the Evi1 direct targets are connected with PPIs in a single regulatory pathway termed here as the Evi1 pathway (Table S3F). The pathway consists of seven major parts, referred to as the Evi1 pathway branches: EMT; apoptosis; the immune response; signaling; retinoic acid metabolism; RNA metabolism; and tumor survival mechanisms (FIGS. 21 and S5).

The annotation of Evi1 direct targets indicates that Evi1 can induce EMT via three regulatory pathways: Wnt—Frizzled—Snail; Notch-Hes/Hey-Snail; and autocrine Fgf2—Mek/Erk—Snail. Remarkably, the Evi1 pathway overlaps with nine apoptotic pathways. Among them, two contain proteins encoded by the Evi1-activated genes: 1) Tnfr—p38—Ca²⁺—NFkB and 2) Tnfr/Igfbp—Casp3. On the other hand, the activation of the genes of following five apoptotic pathways leads to the repression of p53: 1) Gna14/Gng11—Phospholipase C—Ca²⁺—Protein kinase C—Mek/Erk—p53; 2) Gna14/Gng11—Phospholipase C—Sgk2—p53; 3) Tnfr—Casp3—Pak3—p53; 4) Mub1—p53; and 5) Fgf2/Rapgef3/Cap2/Grb2—Map2—p53. In addition, the genes of three p53 activators are repressed by Evi1: JUN, PERP and KLF4. Thus, the functions of the direct Evi1 targets forming the regulatory pathway suggest a mechanism for potentially mediating HG-SOC progression. Importantly, the expression of 43 Evi1 direct target genes differed (P<0.05) in FTE compared with any stage of HG-SOC (Figure S5J). Their median expression levels by HG-SOC stage formed four clusters: 1) high expression in FTE and low in HG-SOC; 2) high expression in both FTE and HG-SOC; 3) low expression in FTE and high in HG-SOC; and 4) low expression in both FTE and HG-SOC (Figure S5J). These results support the widely accepted opinion that HG-SOC tumors originate in the FTE cells carrying TP53 mutations, whose apoptosis is blocked [21].

Evi1 pathway is active in HG-SOC tumors and is significant for HG-SOC patient survival. We tested whether the Evi1 pathway, suggested by our studies of SKOV3 cells, is active in the tumors of HG-SOC patients. We investigated a possible relationship between the expression of the Evi1-controlled genes and the MECOM transcripts using data from two patient cohorts, TCGA and GSE20565 (Table 54A). While the expression levels of EVI1 and MDS1 genes were strongly correlated (τ=0.38), EVI1 expression was also correlated (P<0.05) with the expression of 53% of the direct Evi1 targets, 86 of which correlated in the two cohorts. The concordance between the cohorts was observed with respect to gene expression (Figure S3B) and CN (Figure S3C). These results suggest that the Evi1 pathway is indeed active in the tumors of diverse groups of patients with HG-SOC. Surprisingly, the expression of 85% (56/66) direct Evi1 targets, whose CN significantly correlated with EVI1 CN, was also significantly correlated (P<0.05) with EVI1 expression. This result could be explained by the positive clonal selection of HG-SOC cells for the Evi1 pathway as a single regulatory unit active in the tumors. Interestingly, the Evi1 pathway genes most frequently co-amplified with EVI1, are implicated in lipid metabolism and signaling (AADACLI, ETV5, LIPH), cancer, and development (HES1, TM4SF18, TM4SF1, TM4SF4).

Next, we tested whether the Evi1 pathway activity in HG-SOC tumors was associated with an increased risk for the patients. Among the 289 Evi1-controlled genes (whose expressions were available for study in TCGA tumors), 99% (285/289) demonstrated a synergy with EVI1 expression in the 5-year survival prognosis; the prognostic significance of 44% (127/289) depended on the EVI2 CN (Table S4B). Moreover, the prognostic status of 43% (124/289) Evi1 pathway genes became significant after considering their expression values in the context of EVI1 expression and CN. In addition, we observed that 14 of the 238 Evi1 target genes (with enrichment 3.1, P=5.2·10⁻⁵; Table S4C) were significant for HG-SOC patient survival after suboptimal debulking [60]. These data further suggest the relations between Evi1 activity, response tumor to therapy and the progression of HG-SOC clones in the peritoneal cavity. Each branch of the Evi1 pathway, plus the debulking-significant genes described above, was independently assessed as a potential prognostic gene signature (FIGS. 21 and S5). The genes reliably identified a group of patients who had a 5-year survival rate (FIGS. 21A and S5) that was approximately two times higher than the average for HG-SOC [17].

The Evi1 pathway branches, both individually and within complex gene signatures, are strongly significant for patient survival prognosis. To investigate whether the Evi1 pathway branches contribute to SOC patient survival, we used the Kaplan-Meier models resulting from the segregation of the patients by gene expression levels in each individual branch of the Evi1 pathway. For each branch, the top survival significant genes were combined into a single prognostic gene signature according to [61]. The patient stratification into survival groups, using the signatures, was performed as follows (FIGS. 21A and S5K). Our stratification method can be viewed as a two-step voting procedure on the prognostic variables (e.g., gene expression levels) predicting the overall patient survival. The first set of categorizing variables included individual genes, while the second consisted of individual branches of the Evi1 pathway (FIG. 21A).

The first voting was conducted with a simple majority rule providing the survival prognosis corresponding to the most frequent outcome predicted by the individual branches. The second voting included advanced rules for defining the prognostic groups (see FIGS. 21A-C): 1) a high-risk group if the patient was classified as high-risk by more than two branch-specific signatures; 2) a low-risk group if the patient was classified as low-risk by more than two low-risk and, at the same time, fewer than two high-risk signatures; 3) otherwise, the patient was classified as medium risk. This method allowed us to classify the patients into four distinct survival groups with a strong confidence (P<10⁻⁵⁰; FIGS. 21B-C).

Indeed, for 82% (64/78) of the genes constituting the Evi1 pathway gene signature, the ratio of their expression values to EVI1 expression correlated with patient survival prognosis stronger than did gene expression alone (Table S6). For 54% (42/78) of these genes, such normalization resulted in their correlation with prognosis changing from insignificant to significant. We validated the robustness of the Evi1 pathway signatures and our method of consensus prognoses, by obtaining similarly strong survival significance in another patient cohort [79] using the same grouping rules and gene expression threshold values (FIG. 21C).

In addition, we modified the patient grouping rules, and repeated the analysis of the TCGA patients while stratifying them into four post-surgery disease development risk groups based on the expression of the same branch-specific gene signatures (FIG. 55L),: 1) a lowest-risk group if the patient was classified as low-risk by more than four branch-specific signatures; 2) a low-risk group if the patient was classified as low-risk by two to four signatures; 3) a high-risk group if the patient was classified as high-risk by more than two branch-specific signatures; 4) otherwise, the patient was classified as medium risk. Application of this classification method provided HG-SOC patient stratification at a strongly survival-significant level (P<10⁻⁵⁰) reproducible across two patient cohorts.

Among the patients with the most favorable prognoses (FIG. 55L), 85% exhibited a complete (therapeutic) response to post-surgery primary therapy (P=8.3·10⁻⁶). These patients represented 11% of the population and were characterized by an 84% 5-year survival rate. The group with less favorable prognoses (a 46% 5-year survival rate) constituted 30% of the total population. For the rest of the patients (59% of the whole cohort), the 5-year survival rate did not exceed 11%. At the same time, the risk level predicted by three branches (signaling, immune response, survival) of Evi1 pathway was associated with primary chemotherapy outcomes in the TCGA cohort (Table S5A). As we found, EVI1 expression decreased as a result of chemotherapy (P=0.0032). It suggests that the treatment may also reduce the expression of the Evi1 pathway.

To study the individual contributions of the pathway branches into the patient survival, we used univariate and multivariate proportional hazard survival models integrating the clinical parameters of the patients known at the time of the surgery. The univariate analysis (Table S5B) demonstrated that the significant (P<0.01) diagnostic factors predicting the patient's survival included the Evi1 pathway consensus signature (P=1·10⁻¹⁰, HR=0.25), all the individual pathway branch signatures (P<3·10⁴, HR from 1.94 to 12.14), and the advanced patient age (67 y.o. or more; P=2.2.10⁴, HR=2.05). In the high-risk patient group predicted by the immune pathway branch, the disease development was characterized with the highest hazard ratio of all individual predictors (P<1·10⁻¹⁵, HR=12.14). In the patient risk groups associated with negative prognoses, as defined by the individual Evi1 pathway branches, the hazard ratios were typically high (the lower bounds of the HR respective confidence intervals exceeding the value of 2). At the same time, the only patient group characterized with a low hazard ratio was the one predicted by the Evi1 pathway consensus signature (P=1·10⁻¹⁰; HR=0.25, with the upper bound of the hazard ratio 0.39). There were several clinical variables providing survival-significant univariate hazard models, e.g. primary chemotherapy outcome (P=1.4·10⁻⁹, HR=3.34) and neoplasm status (P=2.1·10⁻⁸, HR=0.05). However, unlike the Evi1 pathway signatures expression and the patient's age, these variables could not be measured at the time of initial diagnostics or/and the surgery treatment.

When the multifactorial survival model (that excluded the consensus signature) was considered (Table S5C), all the individual Evi1 pathway branches again were strong significant contributors to the overall hazard (P<0.01; HR from 1.64 to 4.27), except for the EMT (with P=0.03; HR=1.44) and the tumor survival (P=0.20; HR=1.56) branches. Classification of the patients into two groups by the observation of the response to the primary chemotherapy revealed that in the respective multivariate models (Table S5D) only two variables were common among the significant predictors in both patient groups. They were: 1) the immune response signature (with P=2.3·10⁻³, HR=2.4 for the patients with the complete response and P=9.4·10⁻³, HR=3.5 for the patients with other response types), and 2) the RNA metabolism signature (with P=1.1·10⁻⁵, HR=3.6 for the patients with the complete response and P=1.4·10⁻⁴, HR=6.4 for the patients with other response types). The EMT branch signature (P=4.3·10⁻³, HR=2.3) and advanced patient age (53 y.o. or more; P=1.3·10⁻⁵, HR=4.2) were the significant (P<0.01) prognostic factors among the patients with the complete response. Three other branch signatures, the sub-optimal debulking (P=8.5·10⁻⁷, HR=5.8), retinoic acid (P=1.5·10⁻⁶, HR=4.5) and signaling branches (P=5.1·10⁻⁴, HR=3.3), along with the non-white race (P=4.9·10⁻⁴, HR=4.1), were the unique significant survival factors for the patients with non-complete responses. Thus, it was observed that the individual branches of Evi1 pathway both separately and as synergistic factors of a multifactorial proportional hazard model, stratify the patients into survival groups with significantly different hazards. At the same time, two multifactorial models, each combining different individual Evi1 pathway branches, were specific to the type of the patients' response to the primary chemotherapy.

These results indicate, for the first time, that in HG-SOC tumors, the amplification of MECOM complex locus encoding over-expressed EVI1 and MDS1 genes, together with activation of the Evi1 pathway act as co-amplified copy number variation and co-expressed gene network. This pathophysiological network affects both patient survival and HG-SOC response to primary chemotherapy.

Discussion

In this study, we observed that a large-scale CNV was present in all HG-SOC tumor genomes at the earliest stages of the disease and remained high during disease progression (FIG. 51B).

At the same time, the CN of the most frequently amplified locus MECOM progressively increased with stage (Figure S1C). These observations suggest that MECOM might have a clinical role as a biomarker reflecting early HG-SOC progression and led us to an in-depth study of this locus and its genes (EVI1 and MDS1), with a strong focus on the diagnostic and prognostic indicators of the disease.

In HG-SOC, we determined the expression and CN of EVI1 and MDS1 genes of MECOM locus. Our qRT-PCR analyzes showed that for all cancer samples, EVI1 expression discriminated between normal ovarian surface epithelium (OSE) and HG-SOC(stage IV), with the average expression being up to 20 times higher in HG-SOC compared with normal ovarian epithelium. For the first time, we have proposed an HG-SOC biomarker that has the potential to be simultaneously used for diagnostics (FIGS. 18A-J), prognosis (FIGS. 18K-M, 4, and S5) and treatment outcome prediction (Table S5A). Thus, the MECOM locus, its transcripts (EVI1 and MDS1), and the Evi1 protein itself could be used to derive a basis for a robust survival-related HG-SOC classification with a confidence higher than that for other molecular markers previously discussed in the above clinical contexts.

Previously, the TCGA consortium classified patients into four molecular subtypes (differentiated, immunoreactive, mesenchymal, and proliferative) based on gene expression [18]. Interestingly, this classification was insignificant for overall patient survival [63]. As expected, we determined borderline- significant correlation (P=0.015) between our and the TCGA HG-SOC classifications of the same patient cohort (Table S8B). However, the patients in our relatively low-risk survival group were significantly associated with the proliferative TCGA molecular subtype (P=0.0043).

To elucidate the mechanisms underlying the role of Evi1 in clinical cases of HG-SOC, we performed a series of experiments using the SKOV3 cell line. First, using CGH- and gene expression microarrays, we demonstrated that our model, on the genomic scale, can adequately reproduce the important CN and gene expression features observed in the patient tumors (Figures S7 and S8). Curiously, a recent study on ovarian cancer cell lines reported SKOV3 as having a flat CN profile and lacking TP53 mutations, thus not resembling TCGA HG-SOC samples [62]. In fact, that report contradicts with the characteristics of this cell line provided by the source tissue culture collection (ATCC). By contrast, we here demonstrate that the SKOV3 genome contains numerous CNAs covering nine key genes with altered CN values in the clinical HG-SOC tumors (Table S7, Figure S7G). To account for the frequent CNAs introducing systematic biases in the genome-wide assays, we implemented a novel algorithm that we applied to the analysis of the Evi1 binding sites in the SKOV3 cell genome, integrated with microarray gene expression data (Table S3, Figure S5E). This analysis allowed us to the discover two major DNA motifs that could explain the specific binding of Evi1 in over 90% of the cases and to determine for the first time the direct targets of Evi1 in the HG-SOC genome (FIGS. 19, 20D-F, and S2; Table S3). We increased the number of Evi1-bound genes from 5753 [35] to 13501, and the number of direct Evi1 targets from 165 to 310, 254 of which being reported here for the first time (Table 58A). The discovery of these direct targets encoding a set of interconnected proteins led us to a model of a novel Evi1-controlled regulatory pathway (FIGS. 21 and S5). Among the Evi1 pathway genes, several are involved in the regulation of RNA metabolism and translation, including DICER1, which is known to regulate the metabolism of snoRNAs and miRNAs [50]. Interestingly, this gene was prognosis-significant in several cancers including HG-SOC [63-65]. In addition, small nucleolar RNA genes (snorD28, snorA2) and long ncRNA genes (DLEU2, MT1DP, CIRBP, C11orf17, and RGS3) were observed among the direct gene targets of Evi1 (Table S3A). As the evidence grows, the (de)regulation of RNA metabolism may emerge as a new hallmark of cancer in the future. This mechanism appears to be tightly coupled [66-68] with the system of Evi1-controlled genes mediating interferon-y-dependent antiviral RNA response, as reported here for the first time. Interestingly, one of these genes (IFITMI), which is implicated in glioma [69], colorectal [70] and gastric cancer [71], was recently found among the Evi1-regulated genes in murine myeloid leukemia cell lines [72]. Unlike the case of interferon-a [73], we might expect a synergy between the Evi1 and interferon-γ pathways, which might be interesting in context of mechanistic studies and therapeutic applications.

Integrating the genomic data with the results of our proteomics studies revealed that in the HG-SOC tumors, Evi1-containing protein complexes may be directly involved in CNV induction via NHEJ and HR mechanisms (FIGS. 20A and 20B). Since NHEJ is considered as the dominant factor of double-strand DNA breaks-mediated genome instability [87], we experimentally studied the role of Evi1 in this process. We found that Evi1 suppression strongly reduces NHEJ activity of nuclear proteins in OC cells (FIGS. 20G and 20H). Moreover, we observed that the Evi1 binding sites on DNA co-localized with DNA recombination sites (FIG. 21D-F). Another example of a TF involved in DNA recombination is Yy1[40], previously reported to interact with Evi1 [35]. Thus, the unique oncogenic role of the Evi1 protein may be dual; Evi1 assists recombination-mediated mutagenesis in cancer cells, and provides aberrant control of hundreds of its target genes expression.

Discovered in the present study, a specific correlation between the expression of certain Evi1 target genes with the number of genomic copies of the locus MECOM/EVI1, rather than with the EVI1 expression level, suggests a gene expression control mechanism different from a direct transcription factor-mediated regulation. This effect could possibly be explained by a clonal selection in the HG-SOC cells population, where the cells with high MECOM CN expressing a specific subset of Evi1 targets acquire a competitive. We suggest that high-level chromosome instability, which is an intrinsic feature of the HG-SOC, may provide a genetic basis for positive clonal selection of the cells with high copy number of MECOM complex locus that ‘drives’ abnormal variation of the Evi1 pathway. In its turn, the Evi1 pathway activation leads the cells to de-differentiation and progression of the tumor clones with changes in numerous genomic regions, altering expression of the genes located in these regions. Acquisition of such genotypic and phenotypic variations can also be beneficial for the tumor cell as an adaptation to stressful environmental conditions and drug therapy. More in-depth studies at the single cell genome level would clarify whether such type of clonal selection takes place in the HG-SOC tumors.

The “parallel progression model” of metastatic tumors has recently attracted attention from the research community [75]. This model describes cancers that exhibit a massive generation of distinct clones at the early stages of tumor progression. The clones disseminate and evolve into slow-growing metastases. In this model, point mutations and CNV play a central role in the genetic divergence of the clones, providing a permissible genomic landscape for selection, which enables the clones to survive [76]. Although the parallel progression model has never been considered in the context of OC, the characteristics of HGS-OC match it. By re-analyzing the TCGA data [18], we discovered that the fraction of genes with genomic aberrations across 12 tumors at the earliest detected HG-SOC stage (stage I) is 93%, which is 99 times higher than that in the precursor tissue, fallopian tube epithelium (FIG. 51B). These data indirectly indicate the existence of numerous clones with CNV developing in parallel within a single tumor.

We report that MECOM is a target of DNA recombination activity in HG-SOC cells and could also be an important independent factor of this activity, along with Evi1 protein itself. These results allowed us to consider the amplified MECOM locus and overexpressed Evi1 protein as a composite factor, which may be an essential to inducing CNV in the tumor cells via direct and indirect mechanisms, while the Evi1 pathway, active in the same cells, may further accelerate these processes. Thus, the patho-biological model of HG-SOC proposed here suggests that the MECOM locus, its transcripts EVI1 and MDS1, their product protein Evi1, and the pathway controlled by Evi1 may act as a single co-expressed and co-amplified functional unit. The functions of Evi1, established in the present work, are related to the development of all six cancer hallmark capabilities (resisting cell death, inducing angiogenesis, sustaining proliferative signaling, activating invasion and metastasis, evading growth suppressors, and enabling replicative immortality), one emerging hallmark (avoiding immune destruction), and one enabling characteristic (genome instability) [77]. To obtain a strongly specific and balanced patient stratification, we combined the classification results of eight individual functional gene expression signatures playing essential roles in malignancy and cancer progression, as well as response to therapy (FIG. 21A), which were derived from Evi1 pathway branches (Figure S5), and calculated the probability of each patient belonging to one of the four prognostic subgroups based on the voting of these functional signatures. The resulting patient classification systems (FIG. 21) are characterized by a strong survival significance (P<10⁻⁵⁰) and a clear biological meaning, which is determined by their correspondence to the expression of seven functional modules of the Evi1 pathway. Importantly, Evi1 is a target for therapeutic intervention with exiting drugs (arsenic trioxide, rapamycin and pyrrole-imidazole-polyamide, retinoic acid Figure S6). In addition, each Evi1 pathway module contains proteins targeted drugs. For instance, the EMT module is targeted by batimastat (ADAM10); ATRA (Snail, Hesl), norethisterone (Wnt7A); dehydroepiandrosterone (Map2); prostaglandins, suramin and etodolac (Fgf2); estradiol (Snai1, Hes1, Wnt7A). We expect that this knowledge could be used in clinical studies to optimize combined chemotherapies.

Future studies of the cross-talks between the Evi1 pathway and embryonic morphogens (e.g. response to retinoic acid) may provide novel approaches to improving HG-SOC classification, early diagnostics, survival stratification and specific anti-OC therapies. The Evi1-mediated recombination mechanism of CNV in HG-SOC, revealed here, has yet to be studied in detail. For instance, it is unclear whether this mechanism is specific to HG-SOC cells or whether it is active in healthy epithelial cells and how it relates to the non-pathological functions of Evi1. The lack of progress in HG-SOC diagnostics and treatment over the past 30 years has resulted in a lack of confidence in the present HG-SOC classification system. The traditional classification of HG-SOC tumors fails to aid in diagnosis and patient survival prognosis. At the same time, no existing clinical biomarkers can efficiently stratify the tumors. These facts call for the formulation of a new classification system for ovarian cancer that can adequately reflect EOC diagnostics, prognosis and treatment outcome prediction [22]. The present work proposes 1) a novel combined diagnostic and prognostic biomarker including the MECOM CN and its products (EVI1, MDS1), 2) a novel classification of HG-SOCs based on an Evi1 pathway-centric model of survival stratification, establishing a hypothesis of a parallel progression of this cancer in which the MECOM/EVI1 locus is critical for HG-SOC initiation and progression. This model could direct future studies focusing on the discoveries of high-confidence early detection biomarkers and new therapeutic targets for personalized ovarian cancer therapy.

Methods

Microarray comparative genomic hybridization. SKOV3 (HTB-77; ATCC, Manassas, Va.) cells were grown in RPMI with 10% FBS. The cell culture was maintained in 5% CO2 at 37° C. Four biological replicates were used for CN analysis. The DNA was extracted using Puregene Cell and Tissue Kit and hybridized to Affymetrix Genome-Wide Human SNP 6.0 Arrays. The data can be accessed at Gene Expression Omnibus (GEO) with accession numbers GSE53121. Microarray gene expression analysis. SKOV3 cells were transfected with EVI1-Flag plasmid or, as a control, Flag plasmid in four biological replicates each. The cells were harvested 60 h later for RNA extraction and subsequent reverse transcription. Transcriptome-wide RNA expression was measured using Affymetrix Human Gene 1.0 ST arrays. Direct targets genes of Evi1 were identified as significantly (EDGE and PAM statistical criteria) overexpressed with a fold change 1.3 and containing Evi1 BSs within 50 kb of their TSS or TES, as identified in the ChIP-seq analysis. The data can be accessed at GEO as GSE53120.

Mass spectrometry. Stable isotope labeling with amino-acids in cell culture (SILAC) was performed as previously described [31]. SKOV3 cells transfected with the EV11-Flag construct were saturated with heavy arginine and lysine, and SKOV3 cells transfected with the Flag construct were used as a control. Intracellular proteins were extracted and immunoprecipitated with Flag antibodies. Proteins were eluted from the beads with the Flag peptide, combined, and digested with trypsin. Tryptic peptides were sequenced and quantified via LC-MS/MS. The peptide peaks were obtained from the MS using the MaxQuant software [32]. Peptides of at least 6 amino acids in length [85] were identified in the list of SILAC tryptic peptides, as resolved using MaxQuant. Proteins identified by at least 2 peptides with similar H/L ratios higher than 2 were considered candidate Evi1 interactors. Candidate Evi1-interacting proteins belonging to DNA recombination and repair gene ontologies GO:0006310 and GO:0006281, obtained from AmiGO [33], were filtered through THE STRING database [34] to obtain the DNA recombination related interactome of Evi1.

ChIP-seq data analysis. Primary ChIP-seq data were obtained from [35]. The sequencing data were mapped to the human genome (NCBI v.36), and the peaks of immunoprecipitation in the genome of EVI1-Flag vs. Flag control were identified as described previously [36]. The Chip-Seq peaks mapping to the repeat and low-complexity regions masked according to the RepeatMasker software [37] or located farther than 50 kb from the nearest RefSeq gene were excluded from the analysis. The FDR of ChIP-seq BS identification was estimated with qPCR [35] for 144 BSs.

Ovarian cancer qPCR tissue array. Two panels of actin-normalized commercial ovarian cancer tissue array qPCR plates HORT01, HORT02 (Origene technologies) were used. The PCR reactions were performed using Taqman universal master mix (cat. no: 4304437). CT values were obtained, and relative quantification was estimated using the ddCT method [38]. The obtained fold change values were used for further analysis. Survival analysis was performed on the fold change value for the 28 patients for whom clinical data were available.

Non-homologous end joining assay. Three biological replicates (of each sample) of SKOV3 cells were grown for four days in 6-well microwell plates, harvested into BSA medium at the density of 106 cells per plate. After centrifugation at 2000 g for 5 minutes, the cells were resuspended in buffer I (10 mM HEPES, 10 mM KCl, 1.5 mM MgCl2, 500 mM PMSF, 1 mM DTT and protease inhibitor mixture (Cat # p-8340, Sigma, St. Louis, Mo.)). After 15 min incubation on ice, 6 ml of 10% Nonidet P-40 were added to the cell lysates and vortexed. Nucei were isolated by centrifugation at 6000g for 5 minutes. The nuclear pellet was re-suspended in 50 ml of buffer 11 (20 mM HEPES, 420 mM NaCl, 1.5 mM MgCl2, 0.2 mM EDTA and 25% v/v Glycerol) and incubated for 40 minutes at 37° C. The nuclear protein extracts were collected after centrifugation at 13000 g for 10 min at 4° C., and stored at -80° C. The protein concentration in the extracts was determined by Bradford assay (Bio-Rad, Hercules, CA). 150 ng of the linearized pDsRed2ER (Clontech, Mountain View, CA) plasmid was incubated with various concentrations of the nuclear protein extracts in the end joining reaction buffer (1 mM ATP, 0.25 mM dNTP, 25 mM Tris acetate, 100 mM potassium acetate, 10 mM magnesium acetate, 1 mM DTT at pH 7.5) for 1 h at 37° C. The reaction mixture was treated with 1 mg/ml proteinase K at 65° C. for 30 min. For visual detection of the reaction products (FIG. 21E) they were separated by electrophoresis on a 0.7% agarose gel. DNA was detected using a gel imaging system (Alpha Innotech Corp. Santa Clara, Calif.) after ethidium bromide staining). For the products quantification (FIG. 21F), the resulting ligated DNA was diluted with 10 mM pH 8.5 buffer (1:10,000). One ml of the diluted DNAs was used in the qPCR reaction using three pairs of primers (Ds-F1 and Ds-R2, Ds-F1 and Ds-R3, and Ds-F2 and Ds-R2) , according to the protocol described previously [71]. Amplifications from Ds-F1 and Ds-R1 primers were used as an internal control.

Clinical data. The clinical data on 876 patients in six cohorts were retrieved from public databases. The National Institute of Health (NIH) Cancer Genome Atlas (TCGA) dataset with 514 EOC patients was used for the analysis of CNV, gene expression and patient survival. Gene expression data in the following publicly available GEO datasets were used for the analysis of gene diagnostics potential: GSE12172 with 90 patients [39], GSE20565 with 172 patients [40], GSE7463 with 43 patients [41], GSE14407 with 24 patients [42], GSE9899 with 270 patients [79], GSE7305 with 20 patients [88]. In addition 79 HG-SOC (stages 1 and II) samples were selected from the online database [43].

Univariate analysis of survival hazards. Probabilities of the HG-SOC patients overall survival (OS) were estimated with the Kaplan-Meier survival curve method. The Cox proportional models were constructed based on single individual diagnostic and prognostic factors, whose measurements were available at the time of the surgical treatment of the patient (the expression signatures of individual Evi1 pathway branches, their consensus signature, the patient's age and race, the HG-SOC stage and grade), as well as the ones assessable after the full course of the therapy (the type of response to the primary chemotherapy, tumor neoplasm status, and the residual tumor size). The models were applied to calculate the survival hazards. The Wald test was applied to estimate the significance of the difference between the hazards models of the patient survival groups defined by each individual factor listed above. For other types of survival analysis, where the survival significance of patient stratification was assessed, the log-rank test was applied to identify the factors (the individual gene expression signatures and clinical factors) significant for such stratification.

Multivariate survival analyses. To evaluate the relative contributions of individual factors, whose measurements were known at the surgery time, to the overall survival hazard, the multivariate Cox proportional models with multiple factors were applied. The Wald test was applied to estimate the survival significance of the compared survival curves.

Pair-wise survival-based associations between the MECOM genes (EVI1 and MDS1) expression and their DNA copy number (CN) values in the tumor samples were obtained from evaluation of the survival significance of the patient stratification by a combination of two threshold values: one of expression of the MECOM genes and the other of the CN values. The individual threshold values were chosen in such way that maximized the difference between the overall survival rates of the resulting patient strata via minimization of the log-rank statistics, as described earlier [61, 78]. The pair-wise survival associations between EVI1 expression values and the expression of individual pathway genes were assessed by an increase of the survival significance of the patient stratification, when EVI1 expression threshold was considered (additionally to the pathway genes thresholds) and compared with the stratification based on the individual pathway gene expression threshold values alone.

Additional Methods

Ovarian Cancer qPCR Tissue Array

Two panels of actin normalized commercial ovarian cancer tissue array qPCR plates (96 wells) HORT01, HORT02 (Origene technologies) were used for current studies. Each panel contains 48 patient samples of cDNA (normalized with actin) that include normal and stage specific ovarian patients for relative comparison of gene expression of various potential biomarkers. Both panels have unique patient IDs and a total of 15 normal and 81 ovarian patient sample cDNA with various stages enlisted as follows: stage IA=11, stage IB=6, stage IC=7, stage IIA=3, stage IIB=6, stage IIC=3, stage IIIA=11, stage IIIB=11, stage IIIC=14, stage IV=9. Primer3 open source software was used to design forward and reverse primers along with fluorescent probes for qPCR studies. A forward and reverse primer along with fluorescent probe was designed targeting EVI1 isoform (For- GGTTCCTTGCAGCATGCAAGACC, Rev-GTTCTCTGATCAGGCAGTTGG, probe-FAME-TACTTGAGGCCTTCTCCAGG-TAMRA). Primers were designed for endogenous control beta actin (For-CAGCCATGTACGTTGCTATCCAGG, Rev-AGGTCCAGACGCAGGATGGCATG and probe-FAM-actggcatcgtgatggactc-TAMRA) for relative quantification studies. Each primer concentration was optimized and subsequently used on tissue array qPCR panel I and II for gene expression studies. PCR reaction was run on 7500 ABI machine using Taqman universal master mix (cat.no: 4304437) and CT values were obtained and relative quantification was estimated using ddCT method [89]. The obtained fold change values were used for further analysis.

Microarrays

Clinical microarray data was acquired with Affymetrix U133-A (TCGA cohort) and Affymetrix U133-Plus-2.0 (In the microarray data the expression of EVI1 was represented by probesets 226420_at for MDS1 (RefSeq MECOM, transcript variant 4, NM_004991.3) and 208434_at for EVI1 (RefSeq MECOM, transcript variant 2, NM_005241.3) transcripts respectively. The expression of other markers were measured by the corresponding probesets as follows: KRAS—204009_at, ERBB2—216836_at, MUC16 (CA125)—220196_at, WFDC2 (HE4)—203892_at, P53—201746_at, MYC—202431_s_at.

To normalize gene expression data in clinical samples MBEI normalization method was used in each dataset. ANOVA method was utilized to adjust the batch effect among three datasets. All procedures described for TCGA expression data analysis were performed in a similar manner in the GEO expression datasets.

To study the effect of Evi1 on gene expression in siRNA knock-down and overexpression knock-in experiments on this gene in SKOV3 cells were used. The knock-down experiment has previously been reported in [90]. Four samples of the cells treated with control siRNA were compared with four samples of the cells treated with siRNA against Evi1. Raw data (obtained with Affymetrix Human Gene 1.0 ST microarrays) were normalized with RMA algorithm [91]. Differential expression of genes in the (sample vs. control) were evaluated with “nearest shrunken centroids” method score [92] (“PAM score”) and the the q-value of the “optimal discovery procedure” method [93, 94] (“EDGE P-value”). Genes located not further than 50 kb from Evi1 peaks, differentially expressed with: fold change≧1.3, EDGE 0.01, PAM≦−1 if the gene is overexpressed (by fold change) or PAM≧1 if the gene is underexpressed (by fold change).

For the knock-in experiment the SKOV3 cells were transiently transfected with Flag-EVI1 construct, and compared with the cells transfected with Flag construct. Total RNA was extracted with TRIzol and submitted to Origen Labs for analysis. For each sample 200 ng of RNA was reverse-transcribed to produce cDNA, which was then used as a template for cRNA. The cRNA was converted to single-stranded DNA (ssDNA). The ssDNA was labeled with biotin, fragmented and hybridized to Affymetrix Human Gene 1.0 ST arrays for 16 hours at 45 C with 60 rpm rotation. The RNA samples were of high quality with OD₂₆₀/OD₂₈₀ ratios ranging between 1.56 and 1.81, concentrations ranging between 128 and 213 ng/μL, and RNA Integrity number (Agilent Bioanalyzer) values ranging from 9.6 to 10. High quality of microarray hybridization was asserted with polyA and bacterial spike controls. Raw expression data were normalized with RMA algorithm. Genes located not further than 50 kb from Evi1 peaks, differentially expressed with: fold change≧1.3, EDGE≧0.05, PAM≦−1 if the gene is overexpressed (by fold change) or PAM≧1 if the gene is underexpressed (by fold change).

All expression values are given in log₂-transformed form.

ChIP-seq

The ChIP-seq data resulting from the processing by T2G software [95] containing the predictions of ChIP-seq peaks with the length of sequence reads overlaps 7 to 402 bp (38649 ChIP-seq peaks) were used. The data contained the results of the sequence alignment to the human genome (hg18), with the best aligned location corresponding to the sequence cluster of each of the 38649 ChIP-seq peaks. Chromosomal location of the T2G genomic alignments was used to identify the peaks localized in the low-complexity regions masked with RepeatMasker [96]. The 563 peaks localized in the following types of low-complexity regions (RepeatMasker terms) were excluded from the further analysis: “low complexity”, “simple repeats”, “centromeric regions”, “telomeric regions”. The 13128 peaks localized further than 50 kb away from the closest RefSeq-annotated genes were excluded as well. For further analysis, the 17746 peaks with the overlap lengths 9 to 100 bp were chosen (Table S3A).

Kolmogorov-Waring model was used to parametrize the avidity of the BSs [97]. To exclude the BS frequency bias observed at overlap tag number the lower boundary of the fit range was chosen to be 10 overlapping tags/peak. To estimate the robustness of the parameters bootstrapping was performed as follows. At the initial step the parameters of the best fit model for the observed data were obtained. At each of 300 bootstrap iterations normally distributed noise N(μ=0, a=0.01Yₖ) was added to each k-th tag overlap data point Yₖ.

The FDR of ChIP-seq BS identification was estimated with qPCR [90] for 144 BSs. True positive peaks were considered to have qPCR fold change not less than 2 based on the qPCR results in the following lists. For BSs with ChIP peak overlap count 0 (control), 7, 8, 9, 10, 11, 12-20 and over 20 the total number of genes (with a single of a given overlap count) analyzed was 6, 31, 30, 30, 22, 5, 7 and 13 respectively; the number qPCR-validated genes was 0, 17, 23,25, 20, 5, 7 and 13 respectively; the resulting validation rate per cent was 0, 54.8, 76.7, 83.3, 90.9, 100, 100 and 100 respectively. The detailed list of validated peaks is shown in Table S3C.

Based on this data confidence binding to each gene was calculated as a complement of the product of the false positive rate for every peak in 50 kb vicinity of the gene TSS and TES. The ChIP-seq peak overlap height and the confidence were additionally adjusted for the number of copies of the genomic segments containing the peak.

Copy Number Variation-Adjusted ChIP-seq Analysis of Evi1 Binding Sites

After filtering out Evi1 BSs in the repetitive regions, we performed with qPCR validation studies of 144 BSs (Table S3A) in genic regions with ChIP-seq signal values representing its full range and combined into seven intervals Sᵢ, with their index i increasing from 1 to 7 in the sequence: 1) the ChIP-seq signal is 6 and lower, 2) 6 to 7, 3) 7 to 8, 4) 8 to 9, 5) 9 to 10, 6) 10 to 11, and 7) 12 and higher.

Since the ChIP-seq signal was measured in discrete values (number of overlapping sequences in a given ChIP-seq peak), the qPCR validation rates v could be directly measured only at the signal interval upper (Ŝᵢ) and lower (\check{S}ᵢ) boundaries of each i-th signal interval:

$$v ( {\hat{S}}_{i} ) = \frac{\vert ChIP ( {\hat{S}}_{i} ) ⋂ qPCR ( {\hat{S}}_{i} ) \vert}{ChIP ( {\hat{S}}_{i} )} , i = 1 … 7$$

For the discrete ChIP-seq signal values 6 to 11, representing the interval boundaries, we experimentally obtained the average signal validation rates, respectively: 1) 0.568, 2) 0.767, 3) 0.833, 4) 0.909, 5) 1.0. For ChIP-seq peaks with signal values 11 and higher the validation rate was 1.0. Peaks with magnitude lower than 6 were not considered reliable, and the validation rate was set to 0.

Next, we used these estimates to obtain the expected Evi1 binding probability (P) in a given genomic interval (d) that contained a set of (n) ChIP-seq peaks with their validation rates v(sₖ) defined for each k-th ChIP-seq peak:

$$P ( d ) = 1 - {\prod }_{k = 1}^{n} ( 1 - v ( {s}_{k} ) )$$

We applied this procedure to analyze Evi1 binding sites in the +/−50k genomic intervals flanking the RefSeq genes (Table S3B). We identified 305 genes with high probability of Evi1 binding (P>0.95). To account for the high CNV in the SKOV3 genome, the ChIP-seq signal has to be normalized by the copy number (CN) of the respective genomic intervals. The scaling results in the adjusted ChIP-seq signal values taking continuous, rather than discrete values. Therefore, the formula for the qPCR validation rate v estimate has to be adjusted accordingly. Using the average validation rate for the discrete values of the seven signal interval boundaries, were obtained the interval-wise linear interpolation (Lerp) to estimate the validation rate Lv(s) for the continuous signal values s:

Lv(s)=Lerp(s, Sᵢ, v(\check{S}ᵢ), v(Ŝᵢ))|s∈Sᵢ

Thus we obtained the final formula for CNV-adjusted of Evi1 binding probability C, reflecting the confidence of Evi1 binding in a given region of the genome, given that n Evi1 BSs are observed with ChIP-seq in this region:

$$C ( d ) = 1 - {\prod }_{k = 1}^{n} [ 1 - Lv ( {s}_{k} \cdot CN ( d ) ) ]$$

The CNV adjustment resulted in an increase of the total number of Refseq genes, identified as directly controlled by Evi1, to 313 (Table S4A). Among them nine (ETV1, SLC47A1, PDE1C, LRRC17, CIS, IPO5, STK17A, CLDN10, and RB1) were bound by this protein. Evi1 binding probability near these genes increased from P<0.9 to C≧0.95. At the same time, the binding probability near five more genes (TMTC1, SGK1, L1CAM, MAP7D2, PKP2) was reduced from P>0.95 to C<0.3.

Evi1 Binding Motif Search in the ChIP-seq Data.

BioProspector r.2004 [98] software was used to identify motifs over-represented in the genomic sequences centered at the ChIP-seq cluster overlap regions identified with T2G algorithm [95]. Two sets of sequences were used in the analysis: 1) 200 bp long (+/−100 bp from the cluster overlap centers), 2) 2000 bp long (+/−1000 bp from cluster overlap centers). The following set of parameters was used: single block motif length 10, background information extracted from the input sequence number of iterations 100 report the results of all the iterations Bayesian motif scoring function. The motif matrices were analyzed and the consensus motifs were derived as the longest common subsequence of 10 top scoring motifs. GAGACAG and TAATCCCAGC motifs were identified as the most representative for sequence sets 1 and 2, respectively.

Validation the Novel Evi1-Specific DNA Motifs

To validate if the motifs over-represented around Evi1 BSs can specifically bind Evi1, two series of experiments with DNA probes (pairs of forward F reverse complementary R oligonucleotides) were carried out. In the first series the motifs affinity were analyzed with the following DNA probe pairs surrounded with the genomic consensus of the motifs:

probeGAGACAGF:
5′-CAAAATCTCTGTTTCAGTTTGAGACAGAGTTTCCCTCTTTTCGTCA
GG-3′,
probeGAGACAGR:
5′-CCTGACGAAAAGAGGGAAACTCTGTCTCAAACTGAAACAGAGATTT
TG-3′;
probeGAGACAGmtF:
5′-CAAAATCTCTGTTTCAGTTTGAAACAGAGTTTCCCTCTTTTCGTCA
GG-3′,
probeGAGACAGmtR:
5′-CCTGACGAAAAGAGGGAAACTCTGTTTCAAACTGAAACAGAGATTT
TG-3′;
probeGAGACAGFmt1:
5′-CAAAATCTCTGTTTCAGTTTCCCTTTTAGTTTCCCTCTTTTCGTCA
GG-3′,
probeGAGACAGRmt1:
5′-CCTGACGAAAAGAGGGAAACTAAAAGGGAAACTGAAACAGAGATTT
TG-3′;
probeGAGACAGFmt2:
5′-CAAAATCTCTGTTTCAGTTTCCCCCAGAGTTTCCCTCTTTTCGTCA
GG-3′,
probeGAGACAGRmt2:
5′-CCTGACGAAAAGAGGGAAACTCTGGGGGAAACTGAAACAGAGATTT
TG-3′;
probeGAGACAGFmt3:
5′-CAAAATCTCTGTTTCAGTTTGAAACGACCCTTCCCTCTTTTCGTCA
GG-3′,
probeGAGACAGRmt3:
5′-CCTGACGAAAAGAGGGAAGGGTCGTTTCAAACTGAAACAGAGATTT
TG-3′;
probeGAGACAGFmt4:
5′-CAAAATCGTCCATACAGTTTCCCTTTTAGTTTCCCTCTTTTCGTCA
GG-3′,
probeGAGACAGRmt4:
5′-CCTGACGAAAAGAGGGAAACTAAAAGGGAAACTGTATGGACGATTT
TG-3′;
TAATCCCAGCF:
5′-CAAAAGTGGCTCACGCCTGTAATCCCAGCACTTTGGGAGGCTGAGG
CAG-3′,
TAATCCCAGCR:
5′-CTGCCTCAGCCTCCCAAAGTGCTGGGATTACAGGCGTGAGCCACTT
TTG-3′;
pureTAATsurrF:
5′-CAAAGTGGCTCACGCCTGTAATCCCAGCACTTTGGGAGGTAG-3′,
pureTAATsurrR:
5′-CTACCTCCCAAAGTGCTGGGATTACAGGCGTGAGCCACTTTG-3′;
TAATCCCAGCmtF:
5′-CAAAAGTGGCTCACGCCTGTAAGTACAGCACTTTGGGAGGCTGAGG
CAG-3′,
TAATCCCAGCmtR:
5′-CTGCCTCAGCCTCCCAAAGTGCTGTACTTACAGGCGTGAGCCACTT
TTG-3′;
TAATCCCAGCmt2F:
5′-CAAAGTGGCTCACGCCTGGCTGTACCTGACTTTGGGAGGCTGAGGC
AG-3′;
TAATCCCAGCmt2R:
5′-CTGCCTCAGCCTCCCAAAGTCAGGTACAGCCAGGCGTGAGCCACTT
TTG-3′.

In the second series the motifs affinity were analyzed with the following DNA probes surrounded with a repetitive sequence ((AGCT)ₙ-M-(AGCT)ₙ, where M stands for a specific motif sequence):

Cells were harvested by trypsinization washed with cold PBS, centrifuged for 5 mins at 250 g at 4° C., re-suspended in 4 volumes of cold buffer A (10 mM HEPES, pH 7.9, 10 mM KCl, 0.1 mM EDTA, pH 8.0, 0.1 mM EGTA, pH 8.0) and incubated for 15 min on ice. The cells were lysed by 10% Nonidet P-40 ( 1/16th volume) and vortexing for 10 s. The nuclei were spun-down and washed with buffer A. After estimation of the volume of the nuclei, two volumes of cold buffer B (150 mM NaCl, 0.1% Tween-20, 50 mM Hepes, pH 7.3) were added. The nuclei were sonicated and the debris was removed by centrifugation at 20000 g for 20 min.

The probes in pairs were ordered from First BASE Laboratories. One of the probes was biotin-TEG modified at the 5′-end. MyOne Dynabeads T1 streptavidin beads (10 μg) were prepared by washing with TE buffer (10 mM Tris-.HCl, pH 8, 1 mM EDTA) followed by two washes with DW buffer (20 mM Tris HCl, pH 8, 2 M NaCl, 0.5 mM EDTA, 0.03% Nonidet P-40) followed by re-suspension in 50 μL DW. After reconstituting the DNA probes to concentration 100 nmol/mL in annealing buffer (20 mM Tris-.HCl, pH 8, 10 mM MgCl2, 0.1 M KCl), the probes were annealed. The annealed probes were captured on the beads by 2-h rotating incubation at room temperature. 2 μL of the annealed probes were captured with 50 μL of the beads suspension in DW. The beads were washed with DW and incubated with blocking buffer (20 mM HEPES, pH 7.9, 0.05 mg/mL BSA, 0.05 mg/mL glycogen, 0.3 M KCl, 0.02% Nonidet P-40, 2.5 mM DTT, 5 mg/mL poly-vinylpyrrolidone) for 30 minutes at room temperature followed by three washes with buffer G (20 mM Tris-.HCl, pH 7.3, 10% (vol/vol) glycerol, 0.1 M KCl, 0.2 mM EDTA, 10 mM potassium glutamate, 0.04% Nonidet P-40, fresh, 2 mM DTT, complete EDTA-free protease inhibitor from Roche, 1 mM NaVO₃). 15 μL 8 mg/mL nuclear lysate was adjusted to 10 mM potassium glutamate and diluted with 3 volumes of buffer G containing 0.2 mg/mL poly dldC and 0.2 mg/mL poly dAdT. After 20 min spin-down at 20000g, the supernatant was collected, mixed with the beads and incubated overnight at +4° C. with rotation. For elution, after washing with buffer G twice, the beads were re-suspended in Nupage sample buffer (Invitrogen) and incubated for 10 min at 70° C. The eluate was used for immunoblotting with Evi1 antibodies. The Evi1 antibodies were captured on Dynabeads protein G beads (2 μL antibody and 12.5 μL beads per immunoprecipitation; Invitrogen) as indicated by the manufacturer before incubation with the lysate for 2 h at 4° C. The beads were then washed with three different washing buffers [(i) 0.1% Nonidet P-40, 1% Triton X-100, 2 mM EDTA, pH 8.0, 20 mM Tris-.HCl, pH 8.0, 150 mM NaCl; (ii) 0.5% Tween-20, 1% Triton X-100, 2 mM EDTA, pH 8.0, 20 mM Tris-.HCl, pH 8.0, 500 mM NaCl; and (iii) 0.25 M LiCl, 1% Nonidet P-40, 1% Tween-20,1 mM EDTA, pH 8.0, 10 mM Tris-.HCl, pH 8.0). The beads were washed before elution with SDS. The beads were re suspended in Nupage sample buffer (Invitrogen) for elution and incubated at 70 -° C. for 10 min. Western blot analysis was carried out to test the presence Evi1. After pull down, immunoblotting was performed to identify which TFs were attached to the DNA probes.

All buffers were supplemented with Complete EDTA-free protease inhibitor (Roche) and 1 mM NaVO3 phosphatase inhibitor.

Integration of RNA Expression Microarray Data with ChIP-seq Data.

For the expression data obtained in the siRNA knock-down expression experiment the list of differentially expressed probesets was selected based on simultaneously meeting the criteria: 1) the gene, corresponding to each given probeset, has a reliable Evi1 binding site 2) PAM1 -PAM2<1, 3) EDGE P<=0.008. Probeset to gene mapping was performed automatically with a Python script using RefSeq gene annotations stored in APMA database [99].

Copy Number Data Analysis.

Partek software was used to analyze the data. The original data (in Affymmetrix .cel format) from each of the tumor/negative control (blood cells) samples was imported into Partek. The dsDNA copy number in tumor samples were estimated by pairing them with their corresponding control sample data and subsequently performing paired data analysis (CNV in blood cells were used as the negative control). To identify significant segments, the Partek segmentation algorithm was run with segmentation p-value threshold 0.001, minimal number of markers 10, and signal/noise ratio equal 0.5. The segment was considered amplified if its average copy number per cell was, at least, 2.5. The segment was considered deleted if its average copy number per cell did not exceed 1.5. The gene was considered amplified or deleted if any of its segments was amplified or deleted in, at least, 20% of the sample population. Partek Chromosome View was applied for data visualization.

CNV of EVI1 and MDS1 loci were analyzed separately. The CNV value for each locus was estimated by the median CNV of all amplification segments overlapping with the corresponding probesets (226420_at for EVI1 and 208434_at for MDS1). The median values of the CNV across all the patients were calculated for the each locus separately. For other loci the analysis was performed in a similar way. The analysis of SKOV3 cell line was performed similarly to the one for the patient data. The median values of SKOV3 cell line amplifications of top 100 amplified loci were compared with the CNV of these loci in the patients by Kendall's tau correlation analysis. Two types of correlations were calculated: 1) between the CNV ranks of the loci in the SKOV3 dataset and the ranks in the clinical dataset, 2) between the median copy number of the loci in the SKOV3 dataset and the median in the clinical dataset.

Statistical Analysis.

Comparisons of expression values distributions across the groups defined by the clinical qualitative variables (such as tumor type - ‘primary’ or ‘metastasis’) were performed with Kruskal-Wallis test. Correlations between quantitative variables (such as expression values) were assessed with Kendall correlation coefficient. To assess the differences between the survival functions of the patient groups the Wald metric of the Cox-proportional hazard model was used in the implementation of the standard library survival of R statistical environment. Kaplan-Meier model was used to assess the survival curves and calculate the survival predictions. In the cases where expression value was tested for survival significance, the patients were separated by the best cutoff of the expression value found as a result of a P-value minimization with an exhaustive search for the best patient stratification, as described in [100]. For all the Evi1 gene signatures the patients were stratified into three risk groups (low-medium- and high-risk). Each of the three risk groups could not be reduced to two, since the each possible pair of the groups demonstrates a significant difference. The null-hypothesis that any two of the three proposed strata belong to the same survival group was rejected at significance level α=0.05 for every possible combination of the paired group for each gene signature (Table S5E). For the analysis of co-expression of EVI1 and its potential interactors expression probesets on the first stage only with strong Kendall correlations (|τ|0.3 and P_{FDR}(τ)≦0.01) with EVI1,were analyzed as potential survival group-separating variables as described above. On the second stage, the paired combinations of each EVI1 probeset with each strongly correlating probeset were analyzed using the “2-D algorithm” [101]. In each survival group the probesets correlating with EVI1 with significant impact on the survival of the patients by the FDR-corrected P-values were selected based on the criterion P_{FDR}(X²)<=0.05.

The gene signatures were formulated as follows. The branches of the Evi1 pathways were identified based on the literature data on the protein-protein interactions within the list of genes—direct targets of Evi1 regulation of transcription. The genes-targets of Evi1, which expression has been previously reported to be associated with suboptimal debulking [101], has been added to the list of the branches. For the genes of each branch the corresponding Affymetrix U133A probesets were identified. In each list of probesets (corresponding to each of the branches) for each probeset the best patient stratification strategy (and the corresponding cutoff values) were found by the “1-D algorithm” [100]. Within each probeset list the probes were ranked by the P-values (smallest to largest) of their best patient stratification. Each signature (corresponding to each probeset list and each branch) was composed by evaluating the patient stratification resulted from a set of a given number of the top-ranked probesets (genes) determined by independent voting procedure [102]. The number of top-ranked probesets in the voting was iteratively increased from two to the total number of the probesets for each given probeset list, the performance measured by the P-value (Cox-proportional model) of the survival in the resulting patient strata. The list of top n probesets for which the performance measure is less than 0.01 and the difference between the fraction of patients between these groups with the best and the worst prognosis is maximal, were selected as the gene signature characterizing the corresponding branch.

The clinical information obtained for each patient sample from tissue array qPCR was used for survival analysis. Out of 81 patient samples, 28 patients had clinical information with respect to survival (dead/alive with months of survival), metastatic status (yes/no), and relapse of tumor (yes/no). The survival plots were constructed using fold change values of various patient samples along with corresponding overall survival information.

Cohen's kappa correlation coefficient was used for evaluation of correspondence between any two given classification systems. In the cases when any two compared classification systems contained different number of classes, all combinations.

Evi1 Pathway Genes

Literature analysis allowed us to discriminate the following regulatory pathways integrating the genes under direct control of Evi1 into a single regulatory network. Defense against the host immune system is supported in four ways: 1) Direct deactivation of complement system (CIS) and lymphocyte peptidases (SLPI), 2) activation of synthesis of intracellular matrix (MXRA5), promotion of its calcification (SLC20A1) and sulfation (SLC26A2), 3) support of protein glycosylation, including fucosylation (FUT8), 0-glycosilation (GCNT1) and N-glycosilation (SLC35C1), 4) direct repression of cytokine and chemokine synthesis (CXCL1, CXCL2, IL7A, IL18).

Paracrine signaling can be affected by Evi1 via regulation of secretion and signal transduction of the following signals: endothelin, FGF2, TNFα,VEGF, prostaglandins, chemokines, interleukins, IGF.

Gene clusters of Evi1 patway discriminating between fallopian tubes and HG-SOC are given below.

The first cluster (genes repressed in FTE-OC transformation) included cell contact proteins markers of EMT (CLDN10, LAMA4, fibronectin FNDC3A, TGM2); cytoskeleton and motility (crystallin AIM1, TPM2, ENPP2); apoptosis (GNG11, GULP1); phospholipid metabolism and prostaglandin biosynthesis (ENPP2,AKRIC3,PTG/S); cell membrane (TSPAN1); Wnt-signaling pathway (SFRP1). Notably, a multi-functional enzyme transglutaminase (TGM2) is overexpressed in FTE, in comparison with HG-SOC. TGM2 is involved in many cellular processes. It induces and is induced by RA signaling, activates apoptosis and Phospholipase C and can be secreted . In relation to cell contacts, TGM2 can cross-link fibronectin thus increasing cell rigidity. Recent studies more directly suggest that TGM2 activity induces EMT [103]. The second cluster contained strongly expressed genes. Three of them were down-regulated in HG-SOC (CLU,CIRBP and CAT) and one up-regulated (FTL).

The third cluster (genes activated in FTE-OC transformation) FHL2, a transcriptional co-activator, was found to be activated by p53 and overexpressed in HG-SOC [104]. Matrix modification proteins (MXRA5), metabolism (CALU, SLC20A1), cytoskeleton and motility (DBN1), metallothioneins (MTIG,MTIX).

The fourth cluster (low-expressed genes suppressed in FTE-OC transformation) included importin IPO5 frequently mutated in cancer, KRAS-specific effector and tumor-suppressor RASSF2, a cytoskeletal protein TLN2, LRRC17 an inhibitor of Phospholipase C signaling, PAK3 a downstream effector kinase of CDC42, thrombomodulin THBD, stem cell-associated TF KLF4, endothelin receptor EDNRA, genes regulating cellular metabolism (GNA14, RRAD, PPPIR3C, SLC47A1).

RNA metabolism is represented by the following targets of Evi1: DICER, IPO5, INT2, SNORD116-12, SNORD28, CIRBP. Internal ribosomal entry site (IRES) translation is affected by Evi1 via up-regulation of transcripts of the following proteins: p38, CASP3, EIF4-a and y. In addition, a separate group of Evi1 targets could be associated with tumor survival and resistance to therapeutic treatment. The action of EVI1 includes suppression of metallothioneins (MTIB, MTIDP, MTIF, MTIG, MTIM, MTIX, MT2A), activation of nutrient metabolism (GALNT4, GALNTIO, SLC7A11, RRAD), induction drug transporters (SLC47A1, SLC48A1, SLC29A3) and up-regulation of ferritin (FTL).

Evi1 Pathway Gene Signatures

The following genes and respective probesets were used as a gene signature for apoptosis branch of Evi1 pathway:
SGK2—Homo sapiens serum/glucocorticoid regulated kinase 2 (SGK2): NM_170693 , 220357_s_at
JUN—Homo sapiens jun proto-oncogene (JUN): NM_002228 , 201464_x_at
CAP2—Homo sapiens CAP : NM_006366 , 212551_at
HMGA2—Homo sapiens high mobility group AT-hook 2 (HMGA2): NM_003483 , 208025_s_at
IGFBP3—Homo sapiens insulin-like growth factor binding protein 3 (IGFBP3): NM_000598 , 210095_s_at
RAPGEF3—Homo sapiens Rap guanine nucleotide exchange factor (GEF) 3 (RAPGEF3): NM_001098531, 210051_at
BIRC3—Homo sapiens baculoviral IAP repeat containing 3 (BIRC3): NM_001165, 210538_s_at
ARHGAP1—Homo sapiens Rho GTPase activating protein 1 (ARHGAP1): NM_004308, 202117_at
MAP2—Homo sapiens microtubule-associated protein 2 (MAP2): NM_001039538, 210015_s_at
MAP1B—Homo sapiens microtubule-associated protein 1B (MAP1B): NM_005909, 212233_at
RBI—Homo sapiens retinoblastoma 1 (RBI): NM_000321 , 203132_at
CASP3—Homo sapiens caspase 3: NM_032991, 202763_at
TNFRSFIA—Homo sapiens tumor necrosis factor receptor superfamily: NM_001065, 207643_s_at
ARHGAP24—Homo sapiens Rho GTPase activating protein 24 (ARHGAP24): NM_001025616 , 221030_s_at
RASGRP1—Homo sapiens RAS guanyl releasing protein 1 (calcium and DAG-regulated) (RASGRP1): NM_005739, 205590_at

The following genes and respective probesets were used as a gene signature for the immune response branch of Evi1 pathway:
IL18—Homo sapiens interleukin 18 (interferon-gamma-inducing factor) (IL18): NM_001562 , 206295_at
SLPI—Homo sapiens secretory leukocyte peptidase inhibitor (SLPI): NM_003064, 203021_at
SLC20A1—Homo sapiens solute carrier family 20 (phosphate transporter): NM_005415, 201920_at
CXCL1—Homo sapiens chemokine (C—X—C motif) ligand 1 (melanoma growth stimulating activity: NM_001511, 204470_at
GALNT10—Homo sapiens UDP-N-acetyl-alpha-D-galactosamine:polypeptide N-acetylgalactosaminyltransferase 10 (GalNAc-T10) (GALNT10): NM_198321, 207357_s_at
CXCL2—Homo sapiens chemokine (C—X—C motif) ligand 2 (CXCL2): NM_002089, 209774_x_at
SLC35C1—Homo sapiens solute carrier family 35: NM_018389, 218485_s_at
SLC26A2—Homo sapiens solute carrier family 26 (sulfate transporter): NM_000112, 205097_at
FUT8—Homo sapiens fucosyltransferase 8 (alpha (1: NM_004480, 203988_s_at
CIS —Homo sapiens complement component 1: NM_201442 , 208747_s_at
GCNT1—Homo sapiens glucosaminyl (N-acetyl) transferase 1: NM_001097634, 205505_at
MXRAS —Homo sapiens matrix-remodelling associated 5 (MXRAS): NM_015419, 209596_at

The following genes and respective probesets were used as a gene signature for the cell survival branch of Evi1 pathway:
SLC47A1—Homo sapiens solute carrier family 47: NM_018242, 219525_at
MT1M—Homo sapiens metallothionein 1M (MT1M): NM_176870, 217546_at
SLC48A1—Homo sapiens solute carrier family 48 (heme transporter): NM_017842, 218416_s_at
FTL—Homo sapiens ferritin: NM_000146, 212788_x_at
RRAD—Homo sapiens Ras-related associated with diabetes (RRAD): NM_001128850, 204802_at
MT1G—Homo sapiens metallothionein 1G (MT1G): NM_005950, 204745_x_at
MT1X—Homo sapiens metallothionein 1X (MT1X): NM_005952, 204326_x_at
SLC7A11—Homo sapiens solute carrier family 7 (anionic amino acid transporter light chain: NM_014331 , 207528_s_at
MT2A—Homo sapiens metallothionein 2A (MT2A): NM_005953, 212185_x_at
MT1F—Homo sapiens metallothionein 1F (MT1F): NM_005949, 213629_x_at
SLC29A3—Homo sapiens solute carrier family 29 (nucleoside transporters): NM_018344 , 219344_at

The following genes and respective probesets were used as a gene signature for the retinoic acid branch of Evi1 pathway:
TGM2—Homo sapiens transglutaminase 2 (C polypeptide: NM_004613, 201042_at
HRASLS2—Homo sapiens HRAS-like suppressor 2 (HRASLS2): NM_017878, 216759_at
RARRES3—Homo sapiens retinoic acid receptor responder (tazarotene induced) 3 (RARRES3): NM_004585, 204070_at
ALDH3A1—Homo sapiens aldehyde dehydrogenase 3 family: NM_001135167, 205623_at
FGF2—Homo sapiens fibroblast growth factor 2 (basic) (FGF2): NM_002006, 204421_s_at
SP110—Homo sapiens SP110 nuclear body protein (SP110): NM_004510, 208012_x_at

The following genes and respective probesets were used as a gene signature for the signaling branch of Evi1 pathway:
IL18—Homo sapiens interleukin 18 (interferon-gamma-inducing factor) (IL18): NM_001562 , 206295_at
HTR1D—Homo sapiens 5-hydroxytryptamine (serotonin) receptor 1D: NM_000864, 207368_at
PTPRK—Homo sapiens protein tyrosine phosphatase: NM_001135648, 203038_at
PTGIS—Homo sapiens prostaglandin 12 (prostacyclin) synthase (PTGIS): NM_000961, 208131_s_at
KCNN4—Homo sapiens potassium intermediate/small conductance calcium-activated channel: NM_002250, 204401_at
S100A3—Homo sapiens S100 calcium binding protein A3 (S100A3): NM_002960, 206027_at
PRKAR2B—Homo sapiens protein kinase: NM_002736 , 203680_at
S100A14—Homo sapiens S100 calcium binding protein A14 (S100A14): NM_020672, 218677_at
CXCL1—Homo sapiens chemokine (C—X—C motif) ligand 1 (melanoma growth stimulating activity: NM_001511, 204470_at
PCDHB8—Homo sapiens protocadherin beta 8 (PCDHB8): NM_019120, 221319_at
CXCL2—Homo sapiens chemokine (C—X—C motif) ligand 2 (CXCL2): NM_002089, 209774_x_at
TPCN1—Homo sapiens two pore segment channel 1 (TPCN1): NM_017901, 217914_at
PTGS1—Homo sapiens prostaglandin-endoperoxide synthase 1 (prostaglandin G/H synthase and cyclooxygenase) (PTGS1): NM_000962, 205127_at
EDNRA—Homo sapiens endothelin receptor type A (EDNRA): NM_001957, 204463_s_at
PTGES—Homo sapiens prostaglandin E synthase (PTGES): NM_004878, 207388_s_at
FUT8—Homo sapiens fucosyltransferase 8 (alpha (1: NM_004480, 203988_s_at
SNCA—Homo sapiens synuclein: NM_000345, 204466_s_at
RGS3—Homo sapiens regulator of G-protein signaling 3 (RGS3): NM_021106, 203823_at
FGF2—Homo sapiens fibroblast growth factor 2 (basic) (FGF2): NM_002006, 204421_s_at

The following genes and respective probesets were used as a gene signature for the RNA metabolism branch of Evi1 pathway:
IP05—Homo sapiens importin 5 (IP05): NM_002271, 211952_at
CIRBP—Homo sapiens cold inducible RNA binding protein (CIRBP): NR_023313, 200810_s_at
CASP3—Homo sapiens caspase 3: NM_032991, 202763_at
FGF2—Homo sapiens fibroblast growth factor 2 (basic) (FGF2): NM_002006, 204421_s_at
EIF4G2—Homo sapiens eukaryotic translation initiation factor 4 gamma: NM_001042559, 200004_at
EIF4EBP2—Homo sapiens eukaryotic translation initiation factor 4E binding protein 2 (EIF4EBP2): NM_004096, 208769_at
DICERI—Homo sapiens dicer 1: NM_030621, 206061_s_at

The following genes and respective probesets were used as a gene signature for the genes of Evi1 pathway siginificant for suboptimal debulking:
TM4SF1—Homo sapiens transmembrane 4 L six family member 1 (TM4SF1): NM_014220, 209386_at
CLDN10—Homo sapiens claudin 10 (CLDN10): NM_182848, 205328_at
NTNG1—Homo sapiens netrin G1 (NTNG1): NM_001113228, 206713_at
CLIC1—Homo sapiens chloride intracellular channel 1 (CLIC1): NM_001288, 208659_at
FLOT1—Homo sapiens flotillin 1 (FLOT1): NM_005803, 208748_s_at
RARRES3—Homo sapiens retinoic acid receptor responder (tazarotene induced) 3 (RARRES3): NM_004585, 204070_at
SLC19A2—Homo sapiens solute carrier family 19 (thiamine transporter): NM_006996, 209681_at
ARHGAP24—Homo sapiens Rho GTPase activating protein 24 (ARHGAP24): NM_001025616 , 221030_s_at
RGS3—Homo sapiens regulator of G-protein signaling 3 (RGS3): NM_021106, 203823_at
SLC29A3—Homo sapiens solute carrier family 29 (nucleoside transporters): NM_018344, 219344_at
CIB1—Homo sapiens calcium and integrin binding 1 (calmyrin) (CIB1): NM_006384, 201953_at

The following genes and respective probesets were used as a gene signature for the genes of Evi1 pathway siginificant for antiviral response:
IF144L—Homo sapiens interferon-induced protein 44-like (IF144L): NM_006820, 204439_at
IF130—Homo sapiens interferon: NM_006332 , 201422_at
ISG15—Homo sapiens ISG15 ubiquitin-like modifier (ISG15): NM_005101, 205483_s_at
IF144L—Homo sapiens interferon-induced protein 44-like (IF144L): NM_006820, 204439_at
IF144—Homo sapiens interferon-induced protein 44 (IF144): NM_006417, 214059_at
IF16—Homo sapiens interferon: NM_022872, 204415_at
IFIT1—Homo sapiens interferon-induced protein with tetratricopeptide repeats 1 (IFIT1): NM_001548, 203153_at
IFITM1—Homo sapiens interferon induced transmembrane protein 1 (IFITM1): NM_003641, 201601_x_at
OAS1—Homo sapiens 2′-5′-oligoadenylate synthetase 1: NM_001032409, 202869_at
OAS3—Homo sapiens 2′-5′-oligoadenylate synthetase 3: NM_006187 , 218400_at
OAS2—Homo sapiens 2′-5′-oligoadenylate synthetase 2: NM_002535 , 204972_at
IF135—Homo sapiens interferon-induced protein 35 (IF135): NM_005533, 209417_s_at
IF130—Homo sapiens interferon: NM_006332, 201422_at
IFIH1—Homo sapiens interferon induced with helicase C domain 1 (IFIH1): NM_022168, 216020_at
MX2—Homo sapiens myxovirus (influenza virus) resistance 2 (mouse) (MX2): NM_002463, 204994_at
MX1—Homo sapiens myxovirus (influenza virus) resistance 1 : NM_002462, 202086_at

Motif Co-Localization Analysis

TRANSFAC database v.2009.2 [105] containing information on TFs and their corresponding binding matrices was used. To achieve the efficiency of motif searches, the binding matrices were converted the corresponding sequences of the 16 IUPAC nucleotide symbols, according to the data in TRANSFAC. Each IUPAC motif string was stripped from the leading and trailing ‘N’ symbols and converted to the regular expression code. The automatic searches of the binding sites in the genomic sequences in the sense and anti-sense orientations were performed with a Python script based upon Python regular expression engine.

As a statistical characteristic of pairs of motifs the distributions of the distances between the motifs were studied with a set of algorithms implemented as Python scripts extended with a set of C functions. For each motif of a given pair all its localizations in a given sequence were determined. From the results of this step, the number of distances between each location (in the given sequence) of one motif and each location of another motif was calculated. If this number was large enough to exceed the amount of available RAM of the computer to store them, 4000 random instances of the locations were chosen for each of the motifs, otherwise, the two complete sets of locations were chosen. The distribution of all the distances between the chosen sets was calculated. A histogram with 2000 bins was constructed for the distribution. To characterize the distance distribution function, the given sample distribution was used to calculate the best estimation of the parameters of extreme cases of distance distributions: 1) uniform, 2) normal , 3) Cauchy , 4) triangular . All of the four parametrized extreme distributions were constructed and compared with the sample distribution by RMS difference between their cumulative functions and the corresponding Kolmogorov-Smirnov statistic. Histograms of the sample distance distributions not fitting to any of the four distribution classes were visually studied and only the ones classified as ‘Spike’ distribution were chosen as the cases where the distance between the two motifs was non-randomly biased to a narrow range.

Genome sequence analysis demonstrated that in the vicinity of +1-10 kb from gene promoters motifs M1 and M2 co-localize with each other, as well as with many DNA motifs belonging to other TFs (Table S3). M1 and M2 co-localize with, at least, 31 and 45 (respectively) DNA motifs belonging to 42 DNA-binding proteins, including TFs involved in development (HoxA4, Pax2, Pax4, FoxJ2, FoxL1, Tbx5), leukemogenesis (Meis1, Vdr, Atf4, Ikaros, Helios, Aml1) and cancer (Smad2, Zeb, Ap-1, Ap-2, Srebp2, Etsl (p54)). Motifs of TFs with homeodomains are abundant in this list (Zeb, Crx, Chx10, HoxA4, Pax2, Pax4, Meis, Pou3F2, Pitx2).

Some of these proteins were previously reported to interact with Evi1, e.g. Ap-1 and Pax2 [90], but for most of them association with Evi1 is demonstrated for the first time.

Clinical Data

The National Institute of Health (NIH) Cancer Genome Atlas (TCGA) database [18] was used for copy number analysis of patients with ovarian cancer. The data on 514 HG-SOC patients were downloaded from the TCGA website. Among them 449 (87%) patients were initially classified to stages 3 and 4 of HG-SOC; 448 (87%) patients received chemotherapy; 44 (8.5%) patients were younger than 45 years old, 21 (4%) between 45 and 65 years old and 290 (56%) older than 65. For 484 (94%) patients the time of the last follow-up and for 264 (51%) the time of death were defined. For copy number variation analysis the data on 504 patients (of 514 in total) in TCGA set were available. Among them for 337 samples gene expression data was also available. This group of samples was used for survival analysis.

For gene expression analysis the following publicly available datasets were obtained from Gene Expression Omnibus (GEO) website [107]: GSE12172 including 90 samples [108], GSE20656 including 172 samples [109], and GSE9899 incuding 246 samples [119].

Among the 72 patients of CSE12172 dataset, which passed the quality assessment, all the tumors were characterized with serous phenotype, 56 (77%) were classified to stages 3 and 4 of ovarian cancer, 50 (72%) tumors were characterized as malignant, 22 (28%) were characterized as LMP (low malignancy potential).

Among the 116 patients of CSE20656 dataset, which passed the quality assessment, 59 tumors (78% of of the 76 tumors with available information) were characterized with serous phenotype, 55 (68% of the 81 tumors with available information) were classified to stages 3 and 4 of HG-SOC, 83 (72%) tumors were identified as primary HG-SOC, 28 (72%) were identified as breast cancer metastases in the ovaries.

The GSE9899 (accession number) data set containing 246 samples. From this set 16 patients were removed after a quality control assessement. The 5-year survival of the whole patient cohort was 44 per cent. The 2-year survival of the whole patient cohort was 57 per cent. In addition 79 HG-SOC samples were selected from the online database [110]. All these patients were diagnosed with stages I and II.

Supplementary Discussion

Previous studies have reported conflicting evidence regarding the effect of MECOM transcripts on HG-SOC patient survival prognosis [111], which resulted in neglecting EVI1 as an HG-SOC oncogene, despite its wide acceptance as one for myeloid leukemias. In the present work, we resolved this contradiction by demonstrating that in a population of HG-SOC patients, at least two tumor classes were found: those with high and with low MECOM copy numbers. Each tumor class is characterized by a unique combination of expression values of MDS1 and EVI1 transcripts that separate the patients into groups of favorable and unfavorable survival prognoses (FIG. 2A2). We observed that amplification of the MECOM locus occurs at the earliest stages of HG-SOC development. These data indicate that MECOM and its transcripts, Evi1 and secreted Evi1-dependent proteins, are promising candidate biomarkers for early HG-SOC diagnosis. In the largest existing HG-SOC dataset [110], we found that MECOM transcripts could reliably predict the survival of patients at the earliest stages of HG-SOC (Figure S6C and D). At the same time, high EVI1 and MDS1 expression was specific to HG-SOC tumors compared with fallopian tube and normal ovarian epithelia and reaches its maximal levels in early stage tumors (FIG. 18). The proposed MECOM-based system of HG-SOC classification allows us to robustly identify fallopian tube-derived highly malignant tumors in ovaries, the development of which leads to significantly shorter patient survival times (FIGS. 19, 5, S5 and 6).

The results reported by the TCGA consortium [106] ssuggested the existence of four subgroups based on the signature containing ˜1500 genes. SSurprisingly, in our study, three Evi1 pathway branches (apoptosis, RNA metabolism and tumor survival) combining only 35 genes were sufficient to classify the patients into three of the four groups proposed by TCGA (differentiated, proliferative and mesenchymal subtypes). The P-values of the correspondence between the

TCGA and our classification systems ranged from 0.018 to 3.8·10⁻⁴ (Table S6). We observed that the recombination events, which are associated with Evi1 binding, co-localize with CpG regions actively de-methylated in cancer. Recently, it was discovered that a direct interaction between Evi1 and the DNA methyltransferase Dnmt3 causes DNA methylation in the target regions [112]. TTherefore, recombinational activity associated with Evi1 binding may also be related to its interaction with Dnmt3 or other DNA methyltransferases. A recent report suggests that Evi1 may induce CNV by acting via members of the Evi1 pathway, cyclins and those that interact with p53 [113]

The effects of EVI1 overexpression during tumor progression are likely to be related to its functions in embryogenesis. As we observed, genes downstream of the FGF2 signaling branch are activated by Evi1, while genes of the retinoic acid pathway, antagonistic to FGF2, are repressed. The cells expressing EVI1 undergo EMT that is induced via the Wnt pathway. Cell proliferation and further differentiation from epithelial towards mesenchymal and mesodermal phenotypes may proceed under the control of homeotic TFs that are partners of Evi1 (such as Meisl and Pbxl). Thus, our findings suggest a novel link between late embryogenesis and tumor development, the discussion of which has recently begun [114].

The initial inducer of MECOM amplification is yet to be named. It is possible that this role can be attributed to somatic mutations in the DNA-binding domain of TP53 present in over 95% of HG-SOC tumors [106]. We noticed that TP53 mutations of this type represent 85% of somatic missense mutations in HG-SOC (TCGA). Interestingly, Li-Fraumeni syndrome patients carrying germ-line mutations of a similar type are characterized by an increased CNA frequency in blood cells. These CNAs include frequent translocations of the MECOM locus and adjacent regions on chromosome 3 [115]. Therefore, it is possible that in FTE cells, such TP53 mutations block the activation of DNA repair mechanisms, thus allowing sporadic copy number variations to be tolerated. If this process involves MECOM amplification, its feed-forward loop hypothesized here would unleash genome-wide CNV. It would inevitably lead to generation of numerous HG-SOC clones, their shedding from the fallopian tubes and invasion into neighboring organs (ovaries) and distant tissues within the peritoneal cavity. A similar picture is observed in clinical cases. We propose that certain known drugs targeting Evi1/Mds1 directly [116] and its products and interaction partners [117, 118] could be tested for HG-SOC treatment. In addition, discovery of the Evi1 pathway consisting of six distinct branches opens possibilities for novel therapeutic strategies. The MECOM/Evi1-dependent model of HG-SOC parallel progression suggests that treatment efficienty may be increased, Evi1 itself and all the seven branches under its control need to be treated simultaneously, with the predicted 5-year survival rate rising to 90%. For this purpose, we suggest considering a specific combination of existing FDA-approved drugs for simultaneous administration to HG-SOC patients, as presented in Figure S7.

### REFERENCES

References
[1] Hanahan D, Weinberg R A (2011) Hallmarks of cancer: the next generation. Cell 144: 646-74.
[2] Klein C A (2009) Parallel progression of primary tumours and metastases. Nat Rev Cancer 9: 302-12.
[3] Wang T, Li Q H, Hao G P, Zhai J (2010) Antitumor activity of decoy oligodeoxynucleotides targeted to nf-kappab in vitro and in vivo. Asian Pac J Cancer Prey 11: 193-200.
[4] Govan J M, Lively M O, Deiters A (2011) Photochemical control of dna decoy function enables precise regulation of nuclear factor kappa b activity. J Am Chem Soc 133: 13176-82.
[5] Wang Y, Wu L, Wang P, Lv C, Yang Z, et al. (2012) Manipulation of gene expression in zebrafish using caged circular morpholino oligomers. Nucleic Acids Res 40: 11155-62.
[6] Motakis E, Ivshina A V, Kuznetsov V A (2009) Data-driven approach to predict survival of cancer patients: estimation of microarray genes' prediction significance by cox proportional hazard regression model. IEEE Eng Med Biol Mag 28: 58-66.
[7] Lin C Y, Tsai P H, Kandaswami C C, Chang G D, Cheng C H, et al. (2011) Role of tissue transglutaminase 2 in the acquisition of a mesenchymal-like phenotype in highly invasive a431 tumor cells. Mol Cancer 10: 87.
[8] Kleiber K, Strebhardt K, Martin B T The biological relevance of fhl2 in tumour cells and its role as a putative cancer target. Anticancer Res 27: 55-61.
[9] Wieser R (2007) The oncogene and developmental regulator evi1: expression, biochemical properties, and biological functions. Gene 396: 346-57.
[10] Bonome T, Levine D A, Shih J, Randonovich M, Pise-Masison C A, et al. (2008) A gene signature predicting for survival in suboptimally debulked patients with ovarian cancer. Cancer Res 68: 5478-86
[11] Cohen J (1960). A coefficient of agreement for nominal scales. Educational and Psychological Measurement 20 (1): 37-46.
[12] Kruskal W H, Wallis W A (1952). Use of ranks in one-criterion variance analysis. Journal of the American Statistical Association 47 (260): 583-621.
[13] Kendall M. (1938). “A New Measure of Rank Correlation”. Biometrika 30 (1-2): 81-89.
[14] Andersen P., Gill R. (1982). Cox's regression model for counting processes, a large sample study. Annals of Statistics 10, 1100-1120.
[15] Kaplan E L, Meier P (1958). Nonparametric estimation from incomplete observations. J. Amer. Statist. Assn. 53 (282): 457-481.
[16] Despierre E, Lambrechts D, Neven P, Amant F, Lambrechts S, et al. (2010) The molecular genetic basis of ovarian cancer and its roadmap towards a better treatment. Gynecol Oncol 117: 358-65
[17] Jemal A, Siegel R, Xu J, Ward E (2010) Cancer statistics, 2010. CA Cancer J Clin 60: 277-300
[18] Bell D, Berchuck A, Birrer M, Chien J, Cramer D, et al. (2011) Integrated genomic analyses of ovarian carcinoma. Nature 474: 609-15
[19] Hoogstraat M, de Pagter M S, Cirkel G A, van Roosmalen M J, Harkins T T, et al. (2013) Genomic and transcriptomic plasticity in treatment-naive ovarian cancer. Genome Res. Nov. 12, 2013, doi: 10.1101/gr.161026.113
[20] Bast R C, Hennessy B, Mills G B (2009) The biology of ovarian cancer: new opportunities for translation. Nat Rev Cancer 9: 415-28
[21] Karst A M, Levanon K, Drapkin R (2011) Modeling high-grade serous ovarian carcinogenesis from the fallopian tube. Proc Natl Acad Sci USA 108: 7547-52
[22] Kurman R J, Shih I M (2011) Molecular pathogenesis and extraovarian origin of epithelial ovarian cancer—shifting the paradigm. Hum Pathol 42: 918-31
[23] Hillier S G (2012) Nonovarian origins of ovarian cancer. Proc Natl Acad Sci USA 109: 3608-9
[24] Morishita K, Parker D S, Mucenski M L, Jenkins N A, Copeland N G, et al. (1988) Retroviral activation of a novel gene encoding a zinc finger protein in it-3-dependent myeloid leukemia cell lines. Cell 54: 831-40
[25] Mucenski M L, Taylor B A, Ihle J N, Hartley J W, Morse H C, et al. (1988) Identification of a common ecotropic viral integration site, evi-1, in the dna of akxd murine myeloid tumors. Mol Cell Biol 8: 301-8
[26] Perkins A S, Mercer J A, Jenkins N A, Copeland N G (1991) Patterns of evi-1 expression in embryonic and adult tissues suggest that evi-1 plays an important regulatory role in mouse development. Development 111: 479-87
[27] Buonamici S, Chakraborty S, Senyuk V, Nucifora G (2003) The role of evi1 in normal and leukemic cells. Blood Cells Mol Dis 31: 206-12
[28] Osterberg L, Levan K, Partheen K, Delle U, Olsson B, et al. (2009) Potential predictive markers of chemotherapy resistance in stage iii ovarian serous carcinomas. BMC Cancer 9: 368
[29] Nanjundan M, Nakayama Y, Cheng K W, Lahad J, Liu J, et al. (2007) Amplification of mdsl/evi1 and evi1, located in the 3q26.2 amplicon, is associated with favorable patient prognosis in ovarian cancer. Cancer Res 67: 3074-84
[30] Jazaeri A A, Ferriss J S, Bryant J L, Dalton M S, Dutta A (2010) Evaluation of evi1 and evi1s (delta324) as potential therapeutic targets in ovarian cancer. Gynecol Oncol 118: 189-95
[31] Trinkle-Mulcahy L, Boulon S, Lam Y W, Urcia R, Boisvert F M, et al. (2008) Identifying specific protein interaction partners using quantitative mass spectrometry and bead proteomes. J Cell Biol 183: 223-39
[32] Cox J, Mann M (2008) Maxquant enables high peptide identification rates, individualized p.p.b.-range mass accuracies and proteome-wide protein quantification. Nat Biotechnol 26: 1367-72
[33] Carbon S, Ireland A, Mungall C J, Shu S, Marshall B, et al. (2009) Amigo: online access to ontology and annotation data. Bioinformatics 25: 288-9
[34] Jensen L J, Kuhn M, Stark M, Chaffron S, Creevey C, et al. (2009) String 8—a global view on proteins and their functional interactions in 630 organisms. Nucleic Acids Res 37: D412-6
[35] Bard-Chapeau E A, Jeyakani J, Kok C H, Muller J, Chua B Q, et al. (2012) Ecotopic viral integration site 1 (evi1) regulates multiple cellular processes important for cancer and is a synergistic partner for fos protein in invasive tumors. Proceedings of the National Academy of Sciences 109(6):2168-73
[36] Chen X, Xu H, Yuan P, Fang F, Huss M, et al. (2008) Integration of external signaling pathways with the core transcriptional network in embryonic stem cells. Cell 133: 1106-17
[37] Chen N (2004) Using repeatmasker to identify repetitive elements in genomic sequences. Curr Protoc Bioinformatics Chapter 4: Unit 4.10
[38] Livak K J, Schmittgen T D (2001) Analysis of relative gene expression data using real-time quantitative per and the 2(-delta delta c(t)) method. Methods 25: 402-8
[39] Anglesio M S, Arnold J M, George J, Tinker A V, Tothill R, et al. (2008) Mutation of erbb2 provides a novel alternative mechanism for the ubiquitous activation of ras-mapk in ovarian serous low malignant potential tumors. Mol Cancer Res 6: 1678-90
[40] Meyniel J P, Cottu P H, Decraene C, Stern M H, Couturier J, et al. (2010) A genomic and transcriptomic approach for a differential diagnosis between primary and secondary ovarian carcinomas in patients with a previous history of breast cancer. BMC Cancer 10: 222
[41] Moreno C S, Matyunina L, Dickerson E B, Schubert N, Bowen N J, et al. (2007) Evidence that p53-mediated cell-cycle-arrest inhibits chemotherapeutic treatment of ovarian carcinomas. PLoS ONE 2: e441
[42] Bowen N J, Walker L D, Matyunina L V, Logani S, Totten K A, et al. (2009) Gene expression profiling supports the hypothesis that human ovarian surface epithelia are multipotent and capable of serving as ovarian cancer initiating cells. BMC Med Genomics 2: 71
[43] Gyorffy B, Lanczky A, Szallasi Z. (2012) Implementing an online tool for genome-wide validation of survival-associated biomarkers in ovarian-cancer using microarray data from 1287 patients. Endocr Relat Cancer 19: 197-208
[44] Wieser R (2007) The oncogene and developmental regulator evi1: expression, biochemical properties, and biological functions. Gene 396: 346-57
[45] Etemadmoghadam D, deFazio A, Beroukhim R, Mermel C, George J, et al. (2009) Integrated genome-wide dna copy number and expression analysis identifies distinct mechanisms of primary chemoresistance in ovarian carcinomas. Clin Cancer Res 15: 1417-27
[46] Faratian D, Zweemer A J M, Nagumo Y, Sims A H, Muir M, et al. (2011) Trastuzumab and pertuzumab produce changes in morphology and estrogen receptor signaling in ovarian cancer xenografts revealing new treatment strategies. Clin Cancer Res 17: 4451-61
[47] Shaw T J, Senterman M K, Dawson K, Crane C A, Vanderhyden B C (2004) Characterization of intraperitoneal, orthotopic, and metastatic xenograft models of human ovarian cancer. Mol Ther 10: 1032-42
[48] Haverty P M, Hon L S, Kaminker J S, Chant J, Zhang Z (2009) High-resolution analysis of copy number alterations and associated expression changes in ovarian tumors. BMC Med Genomics 2: 21
[49] Engler D A, Gupta S, Growdon W B, Drapkin R I, Nitta M, et al. (2012) Genome wide dna copy number analysis of serous type ovarian carcinomas identifies genetic markers predictive of clinical outcome. PLoS ONE 7: e30996
[50] Li Z, Ender C, Meister G, Moore P S, Chang Y, et al. (2012) Extensive terminal and asymmetric processing of small rnas from rrnas, snornas, snrnas, and trnas. Nucleic Acids Res 40: 6787-99
[51] Wu Y, Zhou B P (2010) Tnf-alpha/nf-kappab/snail pathway in cancer cell migration and invasion. Br J Cancer 102: 639-44
[52] Kohli R M, Zhang Y (2013) Tet enzymes, tdg and the dynamics of dna demethylation. Nature 502: 472-9
[53] Jin G, Yamazaki Y, Takuwa M, Takahara T, Kaneko K, et al. (2007) Trib1 and evi1 cooperate with hoxa and meisl in myeloid leukemogenesis. Blood 109: 3998-4005
[54] Bard-Chapeau E A, Gunaratne J, Kumar P, Chua B Q, Muller J, et al. (2013) Evi1 oncoprotein interacts with a large and complex network of proteins and integrates signals through protein phosphorylation. Proceedings of the National Academy of Sciences. 110(31):E2885-94.
[55] Wu S, Shi Y, Mulligan P, Gay F, Landry J, et al. (2007) A yyl-ino80 complex regulates genomic stability through homologous recombination-based repair. Nat Struct Mol Biol 14: 1165-72
[56] Novo F J, de Mendibil I O, Vizmanos J L (2007) Ticdb: a collection of gene-mapped translocation breakpoints in cancer. BMC Genomics 8: 33
[57] Niehrs C (2009 January) Active dna demethylation and dna repair. Differentiation; research in biological diversity 77: 1-11
[58] Nakamura T, Jenkins N A, Copeland N G (1996) Identification of a new family of pbx-related homeobox genes. Oncogene 13: 2235-42
[59] Shen W F, Rozenfeld S, Kwong A, Kom ves L G, Lawrence H J, et al. (1999) Hoxa9 forms triple complexes with pbx2 and meisl in myeloid cells. Mol Cell Biol 19: 3051-61
[60] Bonome T, Levine D A, Shih J, Randonovich M, Pise-Masison C A, et al. (2008) A gene signature predicting for survival in suboptimally debulked patients with ovarian cancer. Cancer Res 68: 5478-86
[61] Motakis E, Ivshina A V, Kuznetsov V A (2009) Data-driven approach to predict survival of cancer patients: estimation of microarray genes' prediction significance by cox proportional hazard regression model. IEEE Eng Med Biol Mag 28: 58-66
[62] Domcke S, Sinha R, Levine D A, Sander C, Schultz N (2013) Evaluating cell lines as tumour models by comparison of genomic profiles. Nat Commun 4: 2126
[63] Kim J, Coffey D M, Creighton C J, Yu Z, Hawkins S M, et al. (2012) High-grade serous ovarian cancer arises from fallopian tube in a mouse model. Proc Natl Acad Sci USA 109: 3921-6
[64] Khoshnaw S M, Rakha E A, Abdel-Fatah T M, Nolan C C, Hodi Z, et al. (2012) Loss of dicer expression is associated with breast cancer progression and recurrence. Breast cancer research and treatment. September;135(2):403-13.
[65] Shu G S, Yang Z L, Liu D C (2012) Immunohistochemical study of dicer and drosha expression in the benign and malignant lesions of gallbladder and their clinicopathological significances. Pathol Res Pract 208: 392-7
[66] Witwer K W, Sisk J M, Gama L, Clements J E (2010) Microrna regulation of ifn-beta protein expression: rapid and sensitive modulation of the innate immune response. J Immunol 184: 2369-76
[67] Nazarov P V, Reinsbach S E, Muller A, Nicot N, Philippidou D, et al. (2013) Interplay of micrornas, transcription factors and target genes: linking dynamic expression changes to function. Nucleic Acids Res 41: 2817-31
[68] Gupta A, Swaminathan G, Martin-Garcia J, Navas-Martin S (2012) Micrornas, hepatitis c virus, and hcv/hiv-1 co-infection: new insights in pathogenesis and therapy. Viruses 4: 2485-513
[69] Yu F, Ng S S M, Chow B K C, Sze J, Lu G, et al. (2011) Knockdown of interferon-induced transmembrane protein 1 (ifitml) inhibits proliferation, migration, and invasion of glioma cells. J Neurooncol 103: 187-95
[70] Andreu P, Colnot S, Godard C, Laurent-Puig P, Lamarque D, et al. (2006) Identification of the ifitm family as a new molecular marker in human colorectal tumors. Cancer Res 66: 1949-55
[71] Lee J, Goh S H, Song N, Hwang J A, Nam S, et al. (2012) Overexpression of ifitml has clinicopathologic effects on gastric cancer and is regulated by an epigenetic mechanism. Am J Pathol 181: 43-52
[72] Glass C, Wuertzer C, Cui X, Bi Y, Davuluri R, et al. (2013) Global identification of evi1 target genes in acute myeloid leukemia. PLoS ONE 8: e67134
[73] Buonamici S, Li D, Mikhail F M, Sassano A, Platanias L C, et al. (2005) Evi1 abrogates interferon-alpha response by selectively blocking pml induction. J Biol Chem 280: 428-36
[74] Stein S, Ott M G, Schultze-Strasser S, Jauch A, Burwinkel B, et al. (2010) Genomic instability and myelodysplasia with monosomy 7 consequent to evi1 activation after gene therapy for chronic granulomatous disease. Nat Med 16: 198-204
[75] Klein C A (2009) Parallel progression of primary tumours and metastases. Nat Rev Cancer 9: 302-12
[76] Stoecklein N H, Klein C A (2010) Genetic disparity between primary tumours, disseminated tumour cells, and manifest metastasis. Int J Cancer 126: 589-98
[77] Hanahan D, Weinberg R A (2011) Hallmarks of cancer: the next generation. Cell 144: 646-74
[78] Tang Z, Ow G S, Thiery J P, Ivshina A V, Kuznetsov V A (2013) Meta-analysis of transcriptome reveals let-7b as an unfavorable prognostic biomarker and predicts molecular and clinical subclasses in high-grade serous ovarian carcinoma. Int J Cancer. 134(2): 306-18
[79] Tothill R W, Tinker A V, George J, Brown R, Fox S B, et al. (2008) Novel molecular subtypes of serous and endometrioid ovarian cancer linked to clinical outcome. Clin Cancer Res. 14(16): 5198-20865.
[80] Kataoka K, Sato T, Yoshimi A, Goyama S, Tsuruta T, et al. (2011). Evi1 is essential for hematopoietic stem cell self-renewal, and its expression marks ematopoietic cells with long-term multilineage repopulating activity. J Exp Med. 208: 2403-1
[81] Ow G S, Ivshina A V, Fuentes G, Kuznetsov V A (2014) Identification of two poorly prognosed ovarian carcinoma subtypes associated with CHEK2 germ-line mutation and non-CHEK2 somatic mutation gene signatures. Cell Cycle. 13: 2262-2280
[82] Yoshimi A, Kurokawa M (2011) Evi1 forms a bridge between the epigenetic machinery and signaling pathways. Oncotarget. 2: 575-86
[83] Senyuk V1, Zhang Y, Liu Y, Ming M, Premanand K, et al. (2013) Critical role of miR-9 in myelopoiesis and EVI1-induced leukemogenesis. Proc Natl Acad Sci U S A. 110: 5594-9.
[84] Pradhan A K, Halder A, Chakraborty S (2014) Physical and functional interaction of the proto-oncogene EVI1 and tumor suppressor gene HIC1 deregulates Bcl-xL mediated block in apoptosis. Int J Biochem Cell Biol. 53: 320-328
[85] Sirota F L, Batagov A, Schneider G, Eisenhaber B, Eisenhaber F, Maurer-Stroh S (2102) Beware of moving targets: reference proteome content fluctuates substantially over the years. J Bioinform Comput Biol. 10: 1250020
[86] Davis A J, Chen D J (2013) DNA double strand break repair via non-homologous end-joining. Transl Cancer Res. 2: 130-143.
[87] Shao L, Feng W, Lee KJ, Chen BP, Zhou D (2012) A sensitive and quantitative polymerase chain reaction-based cell free in vitro non-homologous end joining assay for hematopoietic stem cells. PLoS One 7: e33499
[88] Hever A1, Roth R B, Hevezi P, Marin M E, Acosta J A, Acosta H, Rojas J, Herrera R, Grigoriadis D, White E, Conlon P J, Maki R A, Zlotnik A. (2007). Human endometriosis is associated with plasma cells and overexpression of B lymphocyte stimulator. Proc Natl Acad Sci U S A. 104:12451-6.
[89] Livak K J, Schmittgen T D (2001) Analysis of relative gene expression data using real-time quantitative per and the 2(-delta delta c(t)) method. Methods 25: 402-8
[90] Bard-Chapeau E A, Jeyakani J, Kok C H, Muller J, Chua B Q, et al. (2012) Ecotopic viral integration site 1 (evi1) regulates multiple cellular processes important for cancer and is a synergistic partner for fos protein in invasive tumors. Proceedings of the National Academy of Sciences 109(6):2168-73
[91] Irizarry R A, Bolstad B M, Collin F, Cope L M, Hobbs B, et al. (2003) Summaries of affymetrix genechip probe level data. Nucleic Acids Res 31: e15
[92] Tibshirani R, Hastie T, Narasimhan B, Chu G (2002) Diagnosis of multiple cancer types by shrunken centroids of gene expression. Proc Natl Acad Sci USA 99: 6567-72
[93] Leek J T, Monsen E, Dabney A R, Storey J D (2006) Edge: extraction and analysis of differential gene expression. Bioinformatics 22: 507-8
[94] Storey J D, Dai J Y, Leek J T (2007) The optimal discovery procedure for large-scale significance testing, with applications to comparative microarray experiments. Biostatistics 8: 414-32
[95] Chen X, Xu H, Yuan P, Fang F, Huss M, et al. (2008) Integration of external signaling pathways with the core transcriptional network in embryonic stem cells. Cell 133: 1106-17
[96] Chen N (2004) Using repeatmasker to identify repetitive elements in genomic sequences. Curr Protoc Bioinformatics Chapter 4: Unit 4.10
[97] Kuznetsov V A (2009) Relative avidity, specificity, and sensitivity of transcription factor-dna binding in genome-scale experiments. Methods in molecular biology (Clifton, N.J.) 563: 15-50
[98] Liu X, Brutlag D L, Liu J S (2001) Bioprospector: discovering conserved dna motifs in upstream regulatory regions of co-expressed genes. Pac Symp Biocomput: 127-38
[99] Orlov Y L, Zhou J, Lipovich L, Shahab A, Kuznetsov V A (2007) Quality assessment of the affymetrix u133a&b probesets by target sequence mapping and expression data analysis. In Silico Biol (Gedrukt) 7: 241-60
[100] Motakis E, Ivshina A V, Kuznetsov V A (2009) Data-driven approach to predict survival of cancer patients: estimation of microarray genes' prediction significance by cox proportional hazard regression model. IEEE Eng Med Biol Mag 28: 58-66
[101] Bonome T, Levine D A, Shih J, Randonovich M, Pise-Masison C A, et al. (2008) A gene signature predicting for survival in suboptimally debulked patients with ovarian cancer. Cancer Res 68: 5478-86
[102] Liu E T, Kuznetsov V A, Miller L D (2006) In the pursuit of complexity: systems medicine in cancer biology. Cancer Cell 9: 245-7
[103] Lin C Y, Tsai P H, Kandaswami C C, Chang G D, Cheng C H, et al. (2011) Role of tissue transglutaminase 2 in the acquisition of a mesenchymal-like phenotype in highly invasive a431 tumor cells. Mol Cancer 10: 87
[104] Kleiber K, Strebhardt K, Martin B T The biological relevance of fhl2 in tumour cells and its role as a putative cancer target. Anticancer Res 27: 55-61
[105 ] Matys V, Fricke E, Geffers R, Gossling E, Haubrock M, et al. (2003) Transfac: transcriptional regulation, from patterns to profiles. Nucleic Acids Res 31: 374-8
[106] Bell D, Berchuck A, Birrer M, Chien J, Cramer D, et al. (2011) Integrated genomic analyses of ovarian carcinoma. Nature 474: 609-15
[107] Edgar R (2002) Gene expression omnibus: Ncbi gene expression and hybridization array data repository. Nucleic Acids Research 30: 207
[108] Anglesio M S, Arnold J M, George J, Tinker A V, Tothill R, et al. (2008) Mutation of erbb2 provides a novel alternative mechanism for the ubiquitous activation of ras-mapk in ovarian serous low malignant potential tumors. Mol Cancer Res 6: 1678-90
[109] Meyniel J P, Cottu P H, Decraene C, Stern M H, Couturier J, et al. (2010) A genomic and transcriptomic approach for a differential diagnosis between primary and secondary ovarian carcinomas in patients with a previous history of breast cancer. BMC Cancer 10: 222
[110] Gyorffy B, Lanczky A, Szallasi Z (2012) Implementing an online tool for genome-wide validation of survival-associated biomarkers in ovarian-cancer using microarray data from 1287 patients. Endocr Relat Cancer 19: 197-208
[111] Nanjundan M, Nakayama Y, Cheng K W, Lahad J, Liu J, et al. (2007) Amplification of mds1/evi1 and evi1, located in the 3q26.2 amplicon, is associated with favorable patient prognosis in ovarian cancer. Cancer Res 67: 3074-84
[112] Senyuk V, Premanand K, Xu P, Qian Z, Nucifora G (2011) The oncoprotein evi1 and the dna methyltransferase dnmt3 co-operate in binding and de novo methylation of target dna. PLoS ONE 6: e20793
[113] Karakaya K, Herbst F, Ball C, Glimm H, Kramer A, et al. (2012) Overexpression of evi1 interferes with cytokinesis and leads to accumulation of cells with supernumerary centrosomes in g0/1 phase. Cell Cycle 11: 3492-503
[114] Micalizzi D S, Farabaugh S M, Ford H L (2010) Epithelial-mesenchymal transition in cancer: parallels between normal development and tumor progression. J Mammary Gland Biol Neoplasia 15: 117-34
[115] Shlien A, Tabori U, Marshall C R, Pienkowska M, Feuk L, et al. (2008) Excessive genomic dna copy number variation in the li-fraumeni cancer predisposition syndrome. Proc Natl Acad Sci USA 105: 11264-9
[116] Raza A, Buonamici S, Lisak L, Tahir S, Li D, et al. (2004) Arsenic trioxide and thalidomide combination produces multi-lineage hematological responses in myelodysplastic syndromes patients, particularly in those with high pre-therapy evi1 expression. Leuk Res 28: 791-803
[117] Yoshimi A, Kurokawa M (2011) Evi1 forms a bridge between the epigenetic machinery and signaling pathways. Oncotarget 2: 575-86
[118] Zhang Y, Sicot G, Cui X, Vogel M, Wuertzer C A, et al. (2011) Targeting a dna binding motif of the evi1 protein by a pyrrole-imidazole polyamide. Biochemistry 50: 10431-41
[119] Tothill RW1, Tinker A V, George J, Brown R, Fox S B, et al. (2008) Novel molecular subtypes of serous and endometrioid ovarian cancer linked to clinical outcome. Clin Cancer Res. 14(16): 5198-208

1-45. (canceled)

46. A method for a) obtaining information in relation to a medical condition of a subject, the method comprising:
determining in a sample of the subject an expression level of at least one Evi1 pathway signature gene or protein, said at least one gene having within the gene start vicinity at least one Evi1 binding motif with a nucleic acid sequence selected from GAGACAG (SEQ ID NO:1) and TAATCCCAGC (SEQ ID NO: 2);
wherein the expression level of the, or each, gene/protein, when compared to a respective expression threshold value, is indicative of said information, and wherein the information is selected from the group consisting of:
whether the subject has epithelial EOC and/or a predisposition to epithelial EOC;
survival prognosis of the subject when the subject has EOC; and
effectiveness of treatment of EOC in the subject; or
b) determining effectiveness of treatment of EOC in a subject, the method comprising:
determining in a sample of the subject the expression level of at least one Evi1 pathway signature gene or protein, said at least one gene having having within the gene start vicinity at least one Evi1 binding motif with a nucleic acid sequence selected from GAGACAG (SEQ ID NO:1) and TAATCCCAGC (SEQ ID NO: 2);
wherein the expression level of the, or each, gene/protein, when compared to a respective expression threshold value, is indicative of the effectiveness of the treatment; or
c) determining survival prognosis of a subject with EOC, the method comprising:
determining in a sample of the subject a (such as DNA) copy number of at least one Evi1 pathway signature gene locus, said at least one gene locus having within the gene start vicinity at least one Evi1 binding motif with a nucleic acid sequence selected from GAGACAG (SEQ ID NO:1) and TAATCCCAGC (SEQ ID NO: 2);
wherein the (DNA) copy number of the, or each, locus, when compared to a respective copy number threshold value, is indicative of survival prognosis of the subject with EOC; or
d) determining effectiveness of treatment of EOC in a subject, the method comprising:
determining in a sample of the subject the (such as DNA) copy number of at least one Evi1 pathway signature gene locus, said at least one gene locus having within the gene start vicinity at least one Evi1 binding motif with a nucleic acid sequence selected from GAGACAG (SEQ ID NO:1) and TAATCCCAGC (SEQ ID NO: 2);
wherein the (DNA) copy number of the, or each, locus, when compared to a respective copy number threshold value, is indicative of the effectiveness of the treatment.

47. The method according to claim 46, wherein the method further comprises determining the copy number of at least one MECUM locus gene in the subject, and wherein the copy number, when compared to a respective MECOM locus copy number threshold value, is further indicative of survival prognosis or effectiveness of treatment in the subject.

48. The method according to claim 47, wherein determining survival prognosis and/or determining effectiveness of treatment further comprises the following steps for each said Evi1 pathway signature gene:
comparing the copy number of the at least one MECOM locus gene in the subject against the MECOM locus copy number threshold value;
if the copy number of the at least one MECOM locus gene in the subject exceeds the MECOM locus copy number threshold, further comparing the gene/protein expression level of the Evi1 pathway signature gene/protein in the subject against a gene/protein expression threshold value determined for a first cohort of reference subjects, the first cohort of reference subjects having copy numbers of the at least one MECOM locus gene which are above the MECUM locus copy number threshold value;
otherwise, comparing the gene expression level of the Evi1 pathway signature gene/protein in the subject against a gene/protein expression threshold value determined for a second cohort of reference subjects, the second cohort of reference subjects having copy numbers of the at least one MECOM locus gene which are equal to or below the copy number MECOM locus copy number threshold value; and
determining the survival prognosis and/or determining effectiveness of treatment based on the comparison between the respective expression levels of the Evi1 pathway signature gene(s)/protein(s) in the subject and the respective gene expression threshold values.

49. The method according to claim 46, wherein the Evi1 pathway signature gene(s)/protein(s) is or are selected from the genes listed in and of Tables 1-11, singly or in combination.

50. The method according to claim 46, wherein survival prognosis and/or effectiveness of treatment is determined by signature voting on a combination of two or more signatures selected from the signatures in Tables 1 to 11; or is determined by using a voting rule, a consensus rule, or a majority rule.

51. The method according to claim 46, wherein the EOC is a high grade adenocarcinoma in the ovary, in ascites or in metastases or wherein the EOC is primary EOC or serous tubal intraepithelial carcinoma.

52. The method according to claim 46, wherein the gene expression level is determined by measuring the mRNA or protein expression of the or each Evi1 pathway signature gene in the sample.

53. The method according to claim 46, wherein the method further comprises determining the gene expression level of at least one MECOM locus gene in the sample.

54. The method according to claim 46, further comprising a training stage prior to determining survival prognosis and/or determining effectiveness of treatment, the training stage comprising the following steps for each gene of the one or more Evi1 pathway signature genes:
for each of a plurality of training subjects with known outcome relating to EOC, determining a gene/protein expression level of the gene/protein in the training subject; and
determining an expression threshold value which divides the training subjects into two or more patient groups according to whether the gene/protein expression level of the gene/protein in each training subject exceeds the expression threshold value, and/or a copy number threshold value which divides the training subjects into two or more patient groups according to whether the copy number of the gene/protein in each training subject exceeds the copy number threshold value;
wherein the determined expression threshold value or copy number threshold value maximizes a measure of difference between the training subjects into the said two groups.

55. The method according to claim 46, further comprising a training stage prior to determining survival prognosis and/or determining effectiveness of treatment, the training stage comprising the following steps for one or more Evi1 pathway signature genes:
(i) for each of a plurality of training subjects with known outcome relating to EOC, determining copy number of at least one MECOM locus gene in the training subject;
(ii) estimating a sample copy number of at least one MECOM locus gene and dividing the training subjects into two cohorts according to whether the copy number of a MECOM locus gene in each training subject is above or below the sample copy number;
(iii) for each said cohort, determining a sample expression value which divides the training subjects in the cohort into two or more groups according to whether the gene/protein expression level of the Evi1 pathway signature in each training subject exceeds the sample expression value, wherein the sample expression value achieves a maximum measure of difference between the two or more groups;
repeating steps (ii)-(iii) by varying the sample copy number in a range of copy numbers identified in the training subjects and obtaining a copy number distribution curve for the genes; and
(iv) selecting a copy number threshold value as the copy number associated with the largest maximum measure of difference between the two or more groups of a cohort and selecting expression threshold values as the expression values determined for the groups obtained with the copy number threshold value.

56. The method according to claims 54, wherein the measure of difference between the two or more groups comprises a measure of difference between survival curves of the two or more groups.

57. The method according to claim 56, for determining survival prognosis of the subject, wherein the survival time of the training subject is based on the last follow-up time for the training subject and the method further comprises the steps of:
(i) parametrization of a dependence between a patient cohort fraction and the copy number of EVI1 in the set of training subjects;
(ii) parametrization of a dependence between the patient cohort fraction and the survival time in the set of training subjects; and
(iii) using the copy number of the subject to determine the patient cohort fraction from the dependence of (i) and using the patient cohort fraction of the subject to determine an estimated survival time of the subject from dependence (ii).

58. The method according to claim 46, comprising:
(i) for each of a plurality of Evi1 pathway signature genes, obtaining an indication score of survival prognosis or treatment effectiveness; and
(ii) determining a consensus score from the indication scores of step (i), using an independent voting method,
and wherein the expression and/or copy number threshold values are either of the individual cohort threshold values given in Table 12 and/or Table 13.

59. The method according to claim 46, wherein the M expression signature threshold values and/or M copy number signature threshold values, are consensus threshold values derived from each of N training groups of EOC patient tumor samples; the M consensus threshold values classifying the samples of each training group into two or more survival risk sub-groups according to the methods for determining survival prognosis mentioned or treatment effectiveness, the consensus threshold values being generated by:
i) generating, for each of the N training groups, a set of M threshold values for a set of M Evi1 pathway signature genes, the M threshold values dividing the samples of each training group into two or more survival risk subgroups; the N*M evaluated threshold values representing M consensus thresholds defined in an N-dimensional space by the following approximation procedure;
ii) generating a best-fit approximating function of the M threshold values in the N dimentional space;
iii) generating M evaluated threshold values derived by orthogonal projection of the M threshold values in the N-dimentional space onto the best-fit approximating function; the yielded M points on the approximating function represent the consensus threshold value.

60. The method according to claim 59, for determining survival prognosis of the subject, wherein the method further comprises the steps of:
(i) measuring the M expression signature values or M copy number signature values in the subject sample;
(ii) determining the coefficient (one for all the measured signature values) that scales the measurement in the subject sample with the measurements of the same M signatures in each of the N training groups, yielding N scaling coefficients and N*M scaled signature values;
(iii) for each of the M scaled signature values, each represented as a point in the N-dimentional space of signature measurements, determining the orthogonal projection of the signature value onto the best-fit approximating function, yielding M subject points on the best-fit approximating function;
(iv) for each of the M subject points on the best-fit approximating function, determining the difference along the approximating function from the given point to the consensus threshold value of the same signature, yielding the M-dimensional prognostic score of the subject;
(v) based on a given voting rule, determine the classification of the subject into of the given survival prognosis or treatment effectiveness groups.

61. The method according to claim 60, wherein the patients are stratified into classes by
a) theft diagnostic features; or
b) their treatment outcomes.

62. The method according to claim 46, wherein the threshold values are copy number threshold values and the threshold values are each value and/or the consensus threshold values in Table 12 and/or Table 13.

63. A kit for determining determining survival prognosis of a subject with EOC, the kit comprising at least one probe capable of specifically hybridising with an Evi1 pathway signature gene, said gene having within the gene start vicinity at least one Evi1 binding motif with a nucleic add sequence selected from GAGACAG (SEQ. ID NO: 1) and TAATCCCAGC (SEQ ID NO: 2) and/or a MECOM locus expression product in a sample of the subject, and optionally instructions for carrying out a method
i) obtaining information in relation to a medical condition of a subject, the method comprising:
determining in a sample of the subject an expression level of at least one Evi1 pathway signature gene or protein, said at least one gene having within the gene start vicinity at least one Evi1 binding motif with a nucleic add sequence selected from GAGACAG (SEQ ID NO: 1) and TAATCCCAGC (SEQ ID NO: 2);
wherein the expression level of the, or each, gene/protein, when compared to a respective expression threshold value, is indicative of said information, and wherein the information is selected from the group consisting of:
whether the subject has epithelial EOC and/or a predisposition to epithelial EOC survival prognosis of the subject when the subject has EOC; and
effectiveness of treatment of EOC in the subject; or
ii) determining effectiveness of treatment of EOC in a subject, the method comprising:
determining in a sample of the subject the expression level of at least one Evi1 pathway signature gene or protein, said at least one gene having having within the gene start vicinity at least one Evi1 binding motif with a nucleic add sequence selected from GAGACAG (SEQ ID NO: 1) and TAATCCCAGC (SEQ ID NO: 2);
wherein the expression level of the, or each, gene/protein, when compared to a respective expression threshold value, is indicative of the effectiveness of the treatment; or
iii) determining survival prognosis of a subject with EOC, the method comprising:
determining in a sample of the subject a (such as DNA) copy number of at least one Evi1 pathway signature gene locus, said at least one gene locus having within the gene start vicinity at least one Evi1 binding motif with a nucleic add sequence selected from GAGACAG (SEQ ID NO: 1) and TAATCCCAGC (SEQ ID NO: 2);
wherein the (DNA) copy number of the, or each, locus, when compared to a respective copy number threshold value, is indicative of survival prognosis of the subject with EOC; or
iv) determining effectiveness of treatment of EOC in a subject, the method comprising:
determining in a sample of the subject the (such as DNA) copy number of at least one Evi1 pathway signature gene locus, said at least one gene locus having within the gene start vicinity at least one Evi1 binding motif with a nucleic acid sequence selected from GAGACAG (SEQ ID NO: 1) and TAATCCCAGC (SEQ ID NO: 2);
wherein the (DNA) copy number of the, or each, locus, when compared to a respective copy number threshold value, is indicative of the effectiveness of the treatment.

64. The kit according to claim 63, comprising at least one probe that can identify copy number of at least one MECOM locus gene in the sample.

65. The kit according to claim 64, wherein the copy number is determined using at least one method selected from the group consisting of: quantitative PCR assay, in situ hybridization, Southern blotting, multiplex ligation-dependent probe amplification (MLPA) and Quantitative Multiplex PCR of Short Fluorescent Fragments (QMPSF).

66. The kit according to claim 65, wherein the at least one probe comprises;
a) at least one aptamer that binds to at least one MECOM locus-encoded proteins and/or one or more proteins corresponding to Evi1 pathway signature genes; or
b) at least one antibody that binds to at least one MECOM locus-encoded proteins and/or one or more proteins corresponding to Evi1 pathway signature genes.

67. An assay system comprising;
a) a measurement device for measuring gene/protein levels;
b) a data transformation device that acquires marker expression level data and performs data transformation to calculate whether or not the level determined is increased, decreased or equal to a threshold or reference value for the marker in question from the sample;
c) an output interface device such as a user interface output device to output data to a user, for use in a method for:
i) obtaining information in relation to a medical condition of a subject, the method comprising:
determining in a sample of the subject an expression level of at least one Evi1 pathway signature gene or protein, said at least one gene having within the gene start vicinity at least one Evi1 binding motif with a nucleic acid sequence selected from GAGACAG (SEQ ID NO: 1) and TAATCCCAGC (SEQ ID NO: 2);
wherein the expression level of the, or each, gene/protein, when compared to a respective expression threshold value, is indicative of said information, and wherein the information is selected from the group consisting of:
whether the subject has epithelial EOC and/or a predisposition to epithelial EOC;
survival prognosis of the subject when the subject has EOC; and
effectiveness of treatment of EOC in the subject; or
ii) determining effectiveness of treatment of EOC in a subject, the method comprising:
determining in a sample of the subject the expression level of at least one Evi1 pathway signature gene or protein, said at least one gene having having within the gene start vicinity at least one Evi1 binding motif with a nucleic acid sequence selected from GAGACAG (SEQ ID NO: 1) and TAATCCCAGC (SEQ ID NO: 2);
wherein the expression level of the, or each, gene/protein, when compared to a respective expression threshold value, is indicative of the effectiveness of the treatment; or
iii) determining survival prognosis of a subject with EOC, the method comprising:
determining in a sample of the subject a (such as DNA) copy number of at least one Evi1 pathway signature gene locus, said at least one gene locus having within the gene start vicinity at least one Evi1 binding motif with a nucleic acid sequence selected from GAGACAG (SEQ ID NO: 1) and TAATCCCAGC (SEQ ID NO: 2);
wherein the (DNA) copy number of the, or each, locus, when compared to a respective copy number threshold value, is indicative of survival prognosis of the subject with EOC; or
iv) determining effectiveness of treatment of EOC in a subject, the method comprising:
determining in a sample of the subject the (such as DNA) copy number of at least one Evi1 pathway signature gene locus, said at least one gene locus having within the gene start vicinity at least one Evi1 binding motif with a nucleic add sequence selected from GAGACAG SEQ ID NO: 1) and TAATCCCAGC (SEQ ID NO: 2);
wherein the (DNA) copy number of the, or each, locus, when compared to a respective copy number threshold value, is indicative of the effectiveness of the treatment.

68. The assay system according to claim 67, further including a database of threshold or reference values, wherein the device identifies a good, medium or poor prognosis upon analysis of the collective expression of the markers, wherein the device provides treatment information in the database for the good, medium or poor prognosis and outputs the treatment information to the user interface output device, wherein the user interface output device provides an output to the user, comprising notification such that the subject's gene expression is increased or decreased in relation to the threshold/reference value, that this relates to a good, medium or poor prognosis and if they should administer a suitable therapy, such as radiotherapy, chemotherapy, anti-angiogenic compounds or surgery.

69. A method of treating EOC, the method comprising administering one or more binding agents which are capable of specifically binding to a nucleic acid sequence comprising the sequence GAGACAG (SEQ ID NO: 1) or TAATCCAGC (SEQ ID NO: 2) and disrupting the binding of Evi1 to said sequence(s).

70. One or more binding agents which are capable of specifically binding to a nucleic add sequence comprising the sequence GAGACAG (SEQ ID NO: 1) or TAATCCCAGC (SEQ ID NO: 2) and disrupting the binding of Evi1 to said sequence(s) for use in a method of treating ECSC.

71. A method of using of one or more binding agents which are capable of specifically binding to a nucleic add sequence comprising the sequence GAGACAG (SEQ ID NO: 1) or TAATCCCAGC (SEQ ID NO: 2) and disrupting the binding of Evi1 to said sequence(s) for the manufacture of a medicament for treating EOC.

72. The method of claim 24, wherein the binding agent is an aptamer, antibody or antibody binding fragment which is capable of specifically binding to a nucleic acid sequence comprising the sequence GAGACAG (SEQ ID NO: 1) or TAATCCCAGC (SEQ ID NO: 2) and disrupting the binding of Evi1 to said sequence(s).

73. A pharmaceutical formulation comprising an aptamer, antibody or antibody fragment together with a pharmaceutically acceptable excipient, wherein the aptamer, antibody or antibody fragment is capable of specifically binding to a nucleic acid sequence comprising the sequence GAGACAG (SEQ ID NO: 1) or TAATCCCAGC (SEQ ID NO: 2) and disrupting the binding of Evi1 to said sequence(s).
